SEQUENCE LISTING (3 sequences)

SEQ ID NO: 1 - DNA, 18531 bp, synthetic construct
  source 1..18531: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcctgggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaaaaacttttagtgaactaatcctaaagttatcattttaatcttggaggaataaatttaaaccctaatctaattggtttatatgtgtattaactaaattacgagatattagtttttgacactttttttctcgttgagttacagagatgtaactctgtaactcctgatgagtccgtgaggacgaaacgagaaaaaaagtgtcaaaagtcgaccgtagcataaccccttggggcctctaaacgggtcttgaggggttttttgctgaaaggaggaactatataagctttgcagtagcataaccccttggggcctctaaacgggtcttgaggggttttttgctgaaaggaggaactatataacgcgtctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgacgtcatgaccggtagattcgaaattgaaaccattattcctataaaaataggcgcatcacgaggcccctttcgtcttgtaatacgactcactataggg

SEQ ID NO: 2 - DNA, 18531 bp, synthetic construct
  source 1..18531: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcctgggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgttgagttacagagatgtaactctgtaactcctgatgagtccgtgaggacgaaacgagaaaaaaagtgtcaaaagtcgaccgtagcataaccccttggggcctctaaacgggtcttgaggggttttttgctgaaaggaggaactatataagctttgcagtagcataaccccttggggcctctaaacgggtcttgaggggttttttgctgaaaggaggaactatataacgcgtctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgacgtcatgaccggtagattcgaaattgaaaccattattcctataaaaataggcgcatcacgaggcccctttcgtcttgtaatacgactcactataggg

SEQ ID NO: 3 - protein, 2164 aa, synthetic construct
MDPIINGNSANVYLTDSYLKGVISFSECNALGSYIFNGPYLKNDYTNLISRQNPLIEHMNLKKLNITQSLISKYHKGEIKLEEPTYFQSLLMTYKSMTSSEQIATTNLLKKIIRRAIEISDVKVYAILNKLGLKEKDKIKSNNGQDEDNSVITTIIKDDILSAVKDNQSHLKADKNHSTKQKDTIKTTLLKKLMCSMQHPPSWLIHWFNLYTKLNNILTQYRSNEVKNHGFTLIDNQTLSGFQFILNQYGCIVYHKELKRITVTTYNQFLTWKDISLSRLNVCLITWISNCLNTLNKSLGLRCGFNNVILTQLFLYGDCILKLFHNEGFYIIKEVEGFIMSLILNITEEDQFRKRFYNSMLNNITDAANKAQKNLLSRVCHTLLDKTVSDNIINGRWIILLSKFLKLIKLAGDNNLNNLSELYFLFRIFGHPMVDERQAMDAVKINCNETKFYLLSSLSMLRGAFIYRIIKGFVNNYNRWPTLRNAIVLPLRWLTYYKLNTYPSLLELTERDLIVLSGLRFYREFRLPKKVDLEMIINDKAISPPKNLIWTSFPRNYMPSHIQNYIEHEKLKFSESDKSRRVLEYYLRDNKFNECDLYNCVVNQSYLNNPNHVVSLTGKERELSVGRMFAMQPGMFRQVQILAEKMIAENILQFFPESLTRYGDLELQKILELKAGISNKSNRYNDNYNNYISKCSIITDLSKFNQAFRYETSCICSDVLDELHGVQSLFSWLHLTIPHVTIICTYRHAPPYIGDHIVDLNNVDEQSGLYRYHMGGIEGWCQKLWTIEAISLLDLISLKGKFSITALINGDNQSIDISKPIRLMEGQTHAQADYLLALNSLKLLYKEYAGIGHKLKGTETYISRDMQFMSKTIQHNGVYYPASIKKVLRVGPWINTILDDFKVSLESIGSLTQELEYRGESLLCSLIFRNVWLYNQIALQLKNHALCNNKLYLDILKVLKHLKTFFNLDNIDTALTLYMNLPMLFGGGDPNLLYRSFYRRTPDFLTEAIVHSVFILSYYTNHDLKDKLQDLSDDRLNKFLTCIITFDKNPNAEFVTLMRDPQALGSERQAKITSEINRLAVTEVLSTAPNKIFSKSAQHYTTTEIDLNDIMQNIEPTYPHGLRVVYESLPFYKAEKIVNLISGTKSITNILEKTSAIDLTDIDRATEMMRKNITLLIRILPLDCNRDKREILSMENLSITELSKYVRERSWSLSNIVGVTSPSIMYTMDIKYTTSTISSGIIIEKYNVNSLTRGERGPTKPWVGSSTQEKKTMPVYNRQVLTKKQRDQIDLLAKLDWVYASIDNKDEFMEELLGTLGLTYEKAKKLFPQYLSVNYLHRLTVSSRPCEFPASIPAYRTTNYHFDTSPINRILTEKYGDEDIDIVFQNCISFGLSLMSVVEQFTNVCPNRIILIPKLNEIHLMKPPIFTGDVDIHKLKQVIQKQHMFLPDKISLTQYVELFLSNKTLKSGSHVNSNLILAHKISDYFHNTYILSTNLAGHWILIIQLMKDSKGIFEKDWGEGYITDHMFINLKVFFNAYKTYLLCFHKGYGKAKLECDMNTSDLLCVLELIDSSYWKSMSKVFLEQKVIKYILSQDASLHRVKGCHSFKLWFLKRLNVAEFTVCPWVVNIDYHPTHMKAILTYIDLVRMGLINIDRIHIKNKHKFNDEFYTSNLFYINYNFSDNTHLLTKHIRIANSELENNYNKLYHPTPETLENILANPIKSNDKKTLNDYCIGKNVDSIMLPLLSNKKLIKSSAMIRTNYSKQDLYNLFPMVVIDRIIDHSGNTAKSNQLYTTTSHQISLVHNSTSLYCMLPWHHINRFNFVFSSTGCKISIEYILKDLKIKDPNCIAFIGEGAGNLLLRTVVELHPDIRYIYRSLKDCNDHSLPIEFLRLYNGHINIDYGENLTIPATDATNNIHWSYLHIKFAEPISLFVCDAELSVTVNWSKIIIEWSKHVRKCKYCSSVNKCMLIVKYHAQDDIDFKLDNITILKTYVCLGSKLKGSEVYLVLTIGPANIFPVFNVVQNAKLILSRTKNFIMPKKADKESIDANIKSLIPFLCYPITKKGINTALSKLKSVVSGDILSYSIAGRNEVFSNKLINHKHMNILKWFNHVLNFRSTELNYNHLYMVESTYPYLSELLNSLTTNELKKLIKITGSLLYNFHNE

(19) United States
(12) Patent Application Publication
(10) Pub. No.: US 2024/0398932 A1
(43) Pub. Date: Dec. 5, 2024

(54) RESPIRATORY SYNCYTIAL VIRUS VACCINE AND METHODS OF USE

(71) Applicant: SANOFI PASTEUR INC., Swiftwater, PA (US)

(72) Inventors: Sebastien Carayol, Grézieu la Varenne (FR); William Scott Gallichan, Burlington (CA); Michael Hrynyk, Bolton (CA); Olubukola Temitope Idoko, Francheville (FR); Qinglian Li, Aurora (CA); Nausheen Rahman, Toronto (CA); Sanie Samuel Sogoyan Sesay, Albigny sur Saone (FR)

(21) Appl. No.: 18/660,581

(22) Filed: May 10, 2024

Related U.S. Application Data
(60) Provisional application No. 63/501,497, filed on May 11, 2023.
(60) Provisional application No. 63/627,541, filed on Jan. 31, 2024.

(51) Int. Cl. A61K 39/155; A61K 9/00; A61K 39/00; A61P 31/14; C12N 7/00

(52) U.S. Cl. CPC A61K 39/155; A61K 9/0043; A61P 31/14; C12N 7/00; A61K 2039/5254; A61K 2039/543; A61K 2039/545; A61K 2039/55; C12N 2760/18521; C12N 2760/18522; C12N 2760/18534

(57) ABSTRACT

Disclosed herein are respiratory syncytial virus vaccines and methods of immunization to deliver respiratory syncytial virus vaccines in subjects.

### CROSS-REFERENCE TO RELATED APPLICATIONS

This application claims the benefit of priority to U.S. Provisional Application No. 63/501,497, filed May 11, 2023, and U.S. Provisional Application No. 63/627,541, filed Jan. 31, 2024, the contents of each of which are incorporated by reference herein in their entirety.

### CRADA STATEMENT

This invention was created in the performance of a Cooperative Research and Development Agreement with the National Institutes of Health, an Agency of the Department of Health and Human Services. The Government of the United States has certain rights in this invention.

### TECHNICAL FIELD

The subject matter disclosed herein relates to respiratory syncytial virus vaccines and methods of immunization using respiratory syncytial virus vaccines in pediatric subjects.

### SEQUENCE LISTING

The present application contains a Sequence Listing which has been submitted electronically in XML format and is hereby incorporated by reference in its entirety. Said XML copy, created on May 8, 2024, is named 01121-0051-00US-ST26.xml and is 42,873 bytes in size.

### BACKGROUND

RSV is the most important cause of severe acute lower respiratory illness (LRI) in infants and children and the most common cause of severe pneumonia requiring hospital admission in children worldwide. According to global estimates, RSV caused approximately 33 million cases of LRI and approximately 118,000 deaths in children <5 years of age in 2015. Greater than 80% of all RSV-associated LRIs (RSV-LRIs) and more than 50% of the RSV-associated deaths in low- and middle-income countries were estimated to occur in infants ≥6 months old.

Live-attenuated intranasal RSV vaccines are an attractive option for pediatric immunization because they mimic mild natural infections and induce durable cellular, humoral, local, and systemic immunity. Several trials of live-attenuated RSV vaccine candidates have indicated that these vaccines do not cause the vaccine-associated enhanced RSV disease observed in children who received formalin-inactivated RSV.

Progress has been made in the understanding of the RSV gene function and the use of reverse genetic systems to engineer rationally designed attenuated RSV strains, including strains attenuated through deletion of the NS2 gene, like the RSV ΔNS2/Δ1313/I1314L vaccine candidates provided herein. RSV NS2 is a virally encoded type I and III interferon antagonist that interferes with interferon induction and signaling. In chimpanzee, intranasal and intratracheal inoculation with a recombinant strain of wild-type RSV-A2 with the NS2 gene deleted resulted in reduced replication in the upper and lower respiratory tract compared to wild-type (wt)-RSV, and significant resistance to consequent challenge with wild-type RSV. Deletion of the NS2 gene leads to increased interferon response to RSV infection which has been demonstrated for bovine RSV with NS1 or NS2 deletion in calves. NS2 also functions as a pathogenicity factor, promoting epithelial cell shedding in vitro and in the hamster model, and its deletion may potentially reduce small airways obstruction, improving the safety profile of such vaccine candidates. Deletion of the NS2 gene may be beneficial for vaccine safety, and the additional deletion of codon 1313 in the polymerase (L) gene, which also confers mild temperature sensitivity (shutoff temperature of 38° C.-39° C. [100.4° F.-102.2° F.]) for added safety. Substitution of leucine (L) for isoleucine (I) at codon 1314 further stabilizes the deletion of codon 1313 genetically and phenotypically.

### SUMMARY

Provided herein, inter alia, are the following embodiments:

Disclosed herein are methods of immunizing a pediatric subject against a respiratory syncytial virus (RSV) infection, the methods comprising administering a dose of an atomized RSV vaccine to the pediatric subject, the RSV vaccine comprising an effective amount of a live-attenuated RSV ΔNS2/Δ1313/I1314L.

Disclosed herein are methods of immunizing a pediatric subject against a respiratory syncytial virus (RSV) infection, the methods comprising administering a dose of an RSV vaccine using an intranasal atomization delivery device to the pediatric subject, the RSV vaccine comprising an effective amount of a live-attenuated RSV ΔNS2/Δ1313/I1314L.

Disclosed herein are methods of preventing or reducing the likelihood of an RSV infection or preventing or reducing at least one symptom of an RSV infection in a pediatric subject, the methods comprising administering a dose of an RSV vaccine to the pediatric subject, the RSV vaccine comprising an effective amount of a live-attenuated RSV ΔNS2/Δ1313/I1314L.

Disclosed herein are methods of immunizing a pediatric subject against a respiratory syncytial virus (RSV) infection, the methods comprising administering a dose of an atomized RSV vaccine to the pediatric subject, the RSV vaccine comprising an effective amount of a live-attenuated RSV ΔNS2/Δ1313/I1314L, wherein the effective amount of the RSV comprises about 5 to about 9 log₁₀ plaque forming units (PFU) per dose, optionally wherein the dose is about 5.6 log₁₀ PFU per dose.

Disclosed herein are methods of immunizing a pediatric subject against a respiratory syncytial virus (RSV) infection, the methods comprising administering a dose of an atomized RSV vaccine to the pediatric subject, the RSV vaccine comprising an effective amount of a live-attenuated RSV ΔNS2/Δ1313/I1314L, wherein the effective amount of the RSV comprises about 5.4 log₁₀ PFU per dose, about 5.6 log₁₀ PFU per dose, about 6.2 log₁₀ PFU per dose, about 6.4 log₁₀ PFU per dose, or about 7.0 log₁₀ PFU per dose.

Disclosed herein are methods of immunizing a pediatric subject against a respiratory syncytial virus (RSV) infection, the methods comprising administering a dose of an RSV vaccine to the pediatric subject, the RSV vaccine comprising an effective amount of a live-attenuated RSV ΔNS2/Δ1313/I1314L, wherein the effective amount of the RSV comprises about 5 to about 9 log₁₀ plaque forming units (PFU) per dose, optionally wherein the dose is about 5.6 log₁₀ PFU per dose.

Disclosed here in are methods of immunizing a pediatric subject against a respiratory syncytial virus (RSV) infection, the methods comprising administering a dose of an RSV vaccine to the pediatric subject, the RSV vaccine comprising an effective amount of a live-attenuated RSV ΔNS2/Δ1313/I1314L, wherein the effective amount of the RSV comprises about 5.4 log₁₀ PFU per dose, about 5.6 log₁₀ plaque forming unites (PFU) per dose, about 6.2 log₁₀ PFU per dose, about 6.4 log₁₀ PFU per dose, or about 7.0 log₁₀ PFU per dose.

In some embodiments, the effective amount of the RSV comprises about 5 log₁₀ PFU to about 9 log₁₀ PFU per dose, optionally about 5.4 log₁₀ PFU per dose, about 5.6 log₁₀ PFU per dose, about 6.2 log₁₀ PFU per dose, about 6.4 log₁₀ PFU per dose, or about 7.0 log₁₀ PFU per dose. In some embodiments, the RSV vaccine is delivered intranasally with about ½ of the dose delivered to each nostril of the pediatric subject. In some embodiments, the dose of the RSV vaccine is delivered in about 0.2 mL, and wherein about 0.1 mL is delivered to each nostril of the pediatric subject. In some embodiments, the RSV vaccine is delivered to each nostril of the pediatric subject sequentially or simultaneously.

In some embodiments, the methods comprise delivering a second dose of the RSV vaccine. In some embodiments, the second dose comprises about 5.4 log₁₀ PFU. In some embodiments, the second dose comprises about 5.6 log₁₀ PFU. In some embodiments, the second dose comprises about 6.2 log₁₀ PFU. In some embodiments, the second dose comprises about 6.4 log₁₀ PFU. In some embodiments, the second dose comprises about 7.0 log₁₀ PFU. In some embodiments, the second dose comprises about 5 log₁₀ PFU to about 9 log₁₀ PFU. In some embodiments, the second dose is administered at about 40-50, 45-55, 55-60, 52-60, or 60-65 days after the initial dose. In some embodiments, the second dose is administered at least 56 days after the initial dose. In some embodiments, the second dose is delivered intranasally with about ½ of the dose delivered to each nostril of the pediatric subject. In some embodiments, the second dose of the RSV vaccine is delivered in about 0.2 mL, and wherein about 0.1 mL is delivered to each nostril of the pediatric subject. In some embodiments, the RSV vaccine is delivered to each nostril of the pediatric subject sequentially or simultaneously.

In some embodiments, the pediatric subject is about 6 months to about 22 months of age. In some embodiments, the pediatric subject is about 6 months to about 18 months of age. In some embodiments, the pediatric subject is at least 6 months of age. In some embodiments, the pediatric subject was born at term. In some embodiments, the pediatric subject was born preterm.

In some embodiments, the dose of the RSV vaccine is administered to the pediatric subject using an intranasal atomization delivery device. In some embodiments, the intranasal atomization delivery device comprises a spray nozzle to atomize the RSV vaccine for administration to the pediatric subject. In some embodiments, the intranasal atomization delivery device comprises a barrel, a plunger, and a dose divider. In some embodiments, the method comprises advancing the plunger a first distance within the barrel to deliver about ½ of the dose of the RSV vaccine to a first nostril of the pediatric subject. In some embodiments, the method comprises removing the dose divider from the plunger. In some embodiments, the method comprises advancing the plunger a second distance within the barrel to deliver about ½ of the dose to a second nostril of the pediatric subject.

In some embodiments, the intranasal atomization delivery device delivers an average droplet size Dᵥ₅₀ of about 10-120 μm. In some embodiments, an average droplet size Dᵥ₅₀ delivered to each nostril of the pediatric subject is about 10-120 μm, about 30-120 μm, about 50-110 μm, about 70-110 μm, or about 80-110 μm. In some embodiments, the intranasal atomization delivery device delivers an average droplet size Dᵥ₅₀ of at least 30 μm, at least 50 μm, at least 70 μm, at least 80 μm, at least 110 μm, or at least 120 μm. In some embodiments, an average shot weight delivered to each nostril of the pediatric subject is about 30 mg to about 200 mg, about 50 mg to about 175 mg, about 70 mg to about 160 mg, about 80 mg to about 150 mg, 95 mg to about 135 mg, about 100 mg to about 130 mg, about 100 mg to about 130 mg, or between about 105 mg to about 130. In some embodiments, an average shot volume delivered to each nostril of the pediatric subject is about 85 μL to about 120 μL, about 90 μL to about 115 μL, or about 95 μL to about 115 μL.

In some embodiments, a codon in the live-attenuated RSV that encodes a serine at position 1313 of the L protein is deleted resulting in the deletion of the amino acid in the L protein (Δ1313). In some embodiments, an amino acid residue substitution of leucine for isoleucine at position 1314 in the live-attenuated RSV results in a genetically stabilizing mutation in the L gene (I1314L). In some embodiments, the live-attenuated RSV comprises a large polymerase protein (L), a phosphoprotein (P), a nucleocapsid protein (N), an M2-1 protein nonstructural protein 1 (NS1), a glycoprotein (G), a fusion protein (F), a matrix protein (M), an M2-2 protein, and a small hydrophobic protein (SH); and a genome or antigenome comprising a deletion of the codon that encodes the serine at position 1313, or a corresponding position, of the L protein; a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG; a deletion of the NS2 gene; and a nucleotide modification at position 14456 with reference to SEQ ID NO: 1 that represents a change from a thymine (T) to an adenine (A).

In some embodiments, the at least one symptom is selected from development or onset of an upper respiratory tract RSV infection, development or onset of a lower respiratory tract RSV infection, development or onset of otitis media, progression of an upper respiratory tract RSV infection to a lower respiratory tract RSV infection, or progression to otitis media. In some embodiments, the at least one symptom is selected from asthma, wheezing, or a combination thereof.

Use of an intranasal atomization delivery device for administering a dose of an RSV vaccine to a pediatric subject, the RSV vaccine comprising an effective amount of RSV ΔNS2/Δ1313/I1314L is disclosed herein.

Use of an RSV vaccine for the manufacture of a medicament for preventing or reducing the likelihood of infection by RSV virus in a pediatric subject, the RSV vaccine comprising an effective amount of RSV ΔNS2/Δ1313/I1314L is disclosed herein.

In some embodiments, the effective amount of the RSV comprises about 5 log₁₀ PFU to about 9 log₁₀ PFU per dose. In some embodiments, the effective amount of the RSV comprises about 5.4 log₁₀ PFU, about 5.6 log₁₀ PFU, about 6.2 log₁₀ PFU, about 6.4 log₁₀ PFU, or about 7.0 log₁₀ PFU. In some embodiments, the RSV vaccine is delivered intranasally with about ½ of the dose delivered to each nostril of the pediatric subject. In some embodiments, the dose of the RSV vaccine is delivered in about 0.2 mL, and wherein about 0.1 mL is delivered to each nostril of the pediatric subject. In some embodiments, the RSV vaccine is delivered to each nostril of the pediatric subject sequentially or simultaneously.

In some embodiments, the use comprises delivering a second dose of the RSV vaccine. In some embodiments, the second dose comprises about 5.4 log₁₀ PFU. In some embodiments, the second dose comprises about 5.6 log₁₀ PFU. In some embodiments, the second dose comprises about 6.2 log₁₀ PFU. In some embodiments, the second dose comprises about 6.4 log₁₀ PFU. In some embodiments, the second dose comprises about 7.0 log₁₀ PFU. In some embodiments, the second dose comprises about 5 log₁₀ PFU to about 9 log₁₀ PFU. In some embodiments, the second dose is administered about 40-50, 45-55, 55-60, 52-60, or 60-65 days after the initial dose. In some embodiments, the second dose is administered at least 56 days after the initial dose. In some embodiments, the second dose is delivered intranasally with about ½ of the dose delivered to each nostril of the pediatric subject. In some embodiments, the second dose of the RSV vaccine is delivered in about 0.2 mL, and wherein about 0.1 mL is delivered to each nostril of the pediatric subject. In some embodiments, the RSV vaccine is delivered to each nostril of the pediatric subject sequentially or simultaneously.

In some embodiments, the pediatric subject is about 6 months to about 22 months of age. In some embodiments, the pediatric subject is about 6 months to about 18 months of age. In some embodiments, the pediatric subject is at least 6 months of age. In some embodiments, the pediatric subject was born at term. In some embodiments, the pediatric subject was born preterm.

In some embodiments, the dose of the RSV vaccine is administered to the pediatric subject using an intranasal atomization delivery device. In some embodiments, the intranasal atomization delivery device comprises a spray nozzle to atomize the RSV vaccine for administration to the pediatric subject. In some embodiments, the intranasal atomization delivery device comprises a barrel, a plunger, and a dose divider. In some embodiments, the method comprises advancing the plunger a first distance within the barrel to deliver about ½ of the dose of the RSV vaccine to a first nostril of the pediatric subject. In some embodiments, the use comprises removing the dose divider from the plunger. In some embodiments, the use comprises advancing the plunger a second distance within the barrel to deliver about ½ of the dose to a second nostril of the pediatric subject.

In some embodiments, the intranasal atomization delivery device delivers an average droplet size Dᵥ₅₀ of about 10-120 μm. In some embodiments, an average droplet size Dᵥ₅₀ delivered to each nostril of the pediatric subject is about 10-120 μm, about 30-120 μm, about 50-110 μm, about 70-110 μm, or about 80-110 μm. In some embodiments, the intranasal atomization delivery device delivers an average droplet size Dᵥ₅₀ of at least 30 μm, at least 50 μm, at least 70 μm, at least 80 μm, at least 110 μm, or at least 120 μm. In some embodiments, an average shot weight delivered to each nostril of the pediatric subject is about 30 mg to about 200 mg, about 50 mg to about 175 mg, about 70 mg to about 160 mg, about 80 mg to about 150 mg, 95 mg to about 135 mg, about 100 mg to about 130 mg, about 100 mg to about 130 mg, or between about 105 mg to about 130. In some embodiments, an average shot volume delivered to each nostril of the pediatric subject is about 85 μL to about 120 μL, about 90 μL to about 115 μL, or about 95 μL to about 115 μL.

In some embodiments, a codon in the live-attenuated RSV that encodes a serine at position 1313 of the L protein is deleted resulting in the deletion of the amino acid in the L protein (Δ1313). In some embodiments, an amino acid residue substitution of leucine for isoleucine at position 1314 in the live-attenuated RSV results in a genetically stabilizing mutation in the L gene (I1314L). In some embodiments, the live-attenuated RSV comprises a large polymerase protein (L), a phosphoprotein (P), a nucleocapsid protein (N), an M2-1 protein nonstructural protein 1 (NS1), a glycoprotein (G), a fusion protein (F), a matrix protein (M), an M2-2 protein, and a small hydrophobic protein (SH); and a genome or antigenome comprising a deletion of the codon that encodes the serine at position 1313, or a corresponding position, of the L protein; a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG; a deletion of the NS2 gene; and a nucleotide modification at position 14456 with reference to SEQ ID NO: 1 that represents a change from a thymine (T) to an adenine (A).

Disclosed herein are kits comprising a dose of an RSV vaccine comprising an effective amount of a live-attenuated RSV ΔNS2/Δ1313/I1314L and an intranasal atomization delivery device for administering the RSV vaccine to a pediatric subject.

In some embodiments, the effective amount of the RSV comprises about 5 log₁₀ PFU to about 9 log₁₀ PFU per dose. In some embodiments, the effective amount of the RSV comprises about 5.4 log₁₀ PFU and/or about 5.6 log₁₀ PFU and/or about 6.2 log₁₀ PFU and/or about 6.4 log₁₀ PFU and/or about 7.0 log₁₀ PFU. In some embodiments, the kit further comprises a second dose of the RSV vaccine. In some embodiments, the second dose comprises an effective amount of the RSV comprising about 5 log₁₀ PFU to about 9 log₁₀ PFU per second dose. In some embodiments, the second dose comprises an effective amount of the RSV comprising about 5.4 log₁₀ PFU and/or about 5.6 log₁₀ PFU and/or about 6.2 log₁₀ PFU and/or about 6.4 log₁₀ PFU and/or about 7.0 log₁₀ PFU per second dose. In some embodiments, the first dose and/or the second dose comprises a volume of about 0.2 mL.

In some embodiments, the intranasal atomization delivery device comprises a spray nozzle to atomize the RSV vaccine. In some embodiments, the intranasal atomization delivery device comprises a barrel, a plunger, and a dose divider. In some embodiments, the intranasal atomization delivery device delivers an average droplet size Dᵥ₅₀ of about 10-120 μm. In some embodiments, the intranasal atomization delivery device delivers an average droplet size Dᵥ₅₀ of at least 30 μm, at least 50 μm, at least 70 μm, at least 80 μm, at least 110 μm, or at least 120 μm. In some embodiments, the intranasal atomization delivery device delivers an average shot weight for a ½ dose of about 30 mg to about 200 mg, about 50 mg to about 175 mg, about 70 mg to about 160 mg, about 80 mg to about 150 mg, 95 mg to about 135 mg, about 100 mg to about 130 mg, about 100 mg to about 130 mg, or between about 105 mg to about 130. In some embodiments, the intranasal atomization delivery device delivers an average shot volume for a ½ dose of about 85 μL to about 120 μL, about 90 μL to about 115 μL, or about 95 μL to about 115 μL.

Disclosed herein is a recombinant infectious respiratory syncytial virus comprising a large polymerase protein (L), a phosphoprotein (P), a nucleocapsid protein (N), an M2-1 protein nonstructural protein 1 (NS1), a glycoprotein (G), a fusion protein (F), a matrix protein (M), an M2-2 protein, and a small hydrophobic protein (SH); and a genome or antigenome comprising a deletion of the codon that encodes the serine at position 1313, or a corresponding position, of the L protein; a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG; a deletion of the NS2 gene; and a nucleotide modification at position 14456 with reference to SEQ ID NO: 1 that represents a change from a thymine (T) to an adenine (A).

### BRIEF DESCRIPTION OF THE DRAWINGS

FIG. 1 shows a Phase I/II study protocol in accordance with an embodiment of the disclosure.

FIG. 2 shows an intranasal atomization delivery device in accordance with an embodiment of the disclosure.

FIG. 3 shows a dose divider in accordance with an embodiment of the disclosure.

FIG. 4 shows an intranasal atomization delivery device with a dose divider in accordance with an embodiment of the disclosure.

FIG. 5 illustrates the genome structure of RSV ΔNS2/Δ1313/I1314L.

### DETAILED DESCRIPTION

Described herein are methods of immunizing a subject against a respiratory syncytial virus (RSV) infection. The methods may include using an intranasal atomization delivery device to administer an RSV vaccine to the subject.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

### I. Definitions

Unless stated otherwise, the following terms and phrases as used herein are intended to have the following meanings:

As used herein, a “live-attenuated virus” is a virus that is viable and demonstrates reduced, weakened, or no clinical signs of disease when administered to a subject. A “live-attenuated vaccine” comprises a live-attenuated virus.

A “vector” or “vector-based” virus or vaccine, as used herein, comprises a virus that is modified to express one or more heterologous antigens. In some instances, a “vector” or “vector-based” vaccine is also a live-attenuated vaccine where the vaccine demonstrates reduced, weakened, or no clinical signs of disease when administered to a subject.

“Prevention,” as used herein, refers to prophylaxis, avoidance of disease manifestation, a delay of onset, and/or reduction in frequency and/or severity of one or more symptoms of a particular disease, disorder, or condition (e.g., infection with RSV). In some embodiments, prevention is assessed on a population basis such that an agent is considered to “prevent” a particular disease, disorder, or condition if a statistically significant decrease in the development, frequency, and/or intensity of one or more symptoms of the disease, disorder, or condition is observed in a population susceptible to the disease, disorder, or condition.

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

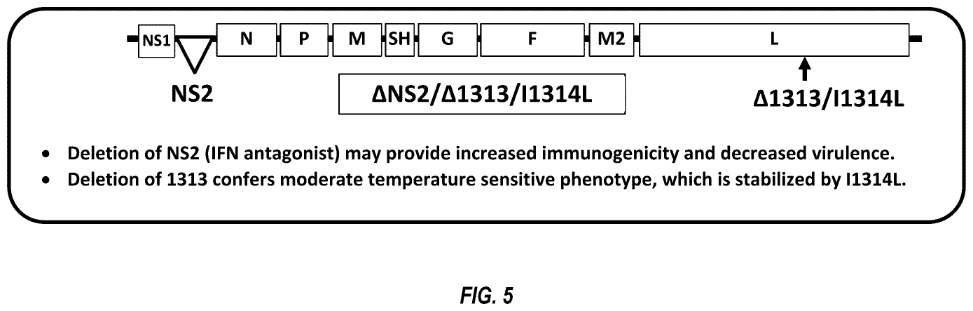

As used herein, “RSV ΔNS2/Δ1313/I1314L” refers to an RSV ΔNS2/Δ1313/I1314L (NIH) or an RSV ΔNS2/Δ1313/I1314L (Sanofi). Each of the ΔNS2/Δ1313/I1314L (NIH) and the RSV ΔNS2/Δ1313/I1314L (Sanofi) comprise a live-attenuated RSV with (i) a 523 nucleotide (nt) deletion of the NS2 gene (ΔNS2), (ii) an amino acid deletion in the L protein, and (iii) a genetically stabilizing mutation in the L gene (see FIG. 5). The live-attenuated RSV of the RSV ΔNS2/Δ1313/I1314L (Sanofi) also includes a nucleotide modification at position 14456 with reference to SEQ ID NO: 1 that represents a change from a thymine (T) to an adenine (A) in a non-coding region.

As used herein, “RSV ΔNS2/Δ1313/I1314L vaccine” refers to an “RSV ΔNS2/Δ1313/I1314L (NIH) vaccine” or an “RSV ΔNS2/Δ1313/I1314L (Sanofi) vaccine.” An RSV ΔNS2/Δ1313/I1314L (NIH) vaccine comprises an effective amount of RSV ΔNS2/Δ1313/I1314L (NIH). An RSV ΔNS2/Δ1313/I1314L (Sanofi) vaccine comprises an effective amount of RSV ΔNS2/Δ1313/I1314L (Sanofi).

“Immune response,” as used herein, refers to a response of a cell of the immune system, such as a B cell, T cell, dendritic cell, macrophage, or polymorphonucleocyte to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses include, for example, measuring proliferation and/or activity of lymphocytes (such as B or T cells), secretion of cytokines or chemokines, inflammation, antibody production, and the like. An “antibody response” is an immune response in which antibodies are produced.

“Immunizing” and the like, as used herein, refer to the act of eliciting an immune response in a subject or protecting a subject against infection.

“Adjuvant,” as used herein, refers to a substance or vehicle that non-specifically enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-like receptor (TLR) agonists and costimulatory molecules. Exemplary biological adjuvants include, but are not limited to, IL-2, RANTES, GM-CSF, TNF-α, IFN-γ, G-CSF, LFA-3, CD72, B7-1, B7-2, OX-40L, 4-1BBL, or combinations thereof.

As used herein, “liquid” is given its traditional meaning and “frozen liquid” is a solid state liquid distinguished from liquid state.

“Formulation” refers to a composition containing an active pharmaceutical or biological ingredient, along with one or more additional components. The term “formulation” is used interchangeably with the terms “pharmaceutical composition,” “vaccine composition,” and “vaccine formulation” herein. Additional components that may be included as appropriate include pharmaceutically acceptable excipients, additives, diluents, buffers, sugars, amino acids (such as glycine, glutamine, asparagine, arginine, or lysine), chelating agents, surfactants, polyols, bulking agents, stabilizers, lyoprotectants, solubilizers, emulsifiers, salts, adjuvants, tonicity enhancing agents (such as alkali metal halides, for example, sodium or potassium chloride, mannitol, or sorbitol), delivery vehicles, and anti-microbial preservatives.

As used herein, “treat,” “treating,” and “treatment” refer to any administration or application of a therapeutic for disease or disorder in a subject, and includes inhibiting the disease, arresting its development, relieving one or more symptoms of the disease, curing the disease, or preventing reoccurrence of one or more symptoms of the disease. In some instances, treatment includes reduction or amelioration of the progression, severity, and/or duration of an upper and/or lower respiratory tract RSV infection, otitis media, or a symptom or respiratory condition related thereto (such as asthma, wheezing, or a combination thereof).

As used herein, a “therapeutically effective amount” or “effective amount” is the amount of a composition, or an active component thereof, sufficient to provide a beneficial effect or to otherwise reduce a detrimental nonbeneficial event to the individual to whom the composition is administered. By “therapeutically effective dose” or “effective dose,” as used herein, is meant a dose that produces one or more desired or desirable (e.g., beneficial) effects for which it is administered, such administration occurring one or more times over a given period of time. The exact dose will depend on the purpose of the treatment and will be ascertainable using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or +0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. All ranges set forth herein are intended to be inclusive of the lower and upper limit of the range.

The term “pediatric subject” is a subject aged 21 or younger at the time of immunization. Pediatric subpopulations are further characterized as: (i) neonates—from birth through the first 28 days of life; (ii) infants and toddlers—from 29 days to less than 2 years; (iii) children-2 years to less than 12 years; and (iv) adolescents-aged 12 years through 21 years. In some aspects, the pediatric subject may be 21, 20, 19, 18, 17, 16, 15, 14, 13, 12, 11, 10, 9, 8, 7, 6, 5, 4, 3, 2, or 1 year(s) of age or less. In some aspects, the pediatric subject may be at least 6 months of age. In some aspects, the pediatric subject may be 6 months to 18 years of age. In some aspects, the pediatric subject may be 6 months to 10 years of age. In some aspects, the pediatric subject may be 4 months to 4 years of age. In some aspects, the pediatric subject may be 6 to 18 months of age. In some aspects, the pediatric subject may be 6 to 22 months of age. In some aspects, the pediatric subject may be from 6 months to less than 24 months of age. Additional age ranges may also be included for pediatric subjects. In some aspects, the pediatric subject was born at term as defined herein as ≥37 weeks of gestation. In some aspects, the pediatric subject was born preterm as defined herein as 28 through 36 weeks of gestation.

Reference will now be made in detail to certain embodiments, examples of which are illustrated in the accompanying drawings. While the embodiments are described in conjunction with the illustrated embodiments, it will be understood that they are not intended to limit the disclosure to those embodiments. On the contrary, the disclosure is intended to cover all alternatives, modifications, and equivalents, which may be included within the disclosure as defined by the appended claims and included embodiments.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Exemplary Methods and Uses

In certain embodiments, provided are methods of immunizing subjects against a respiratory syncytial virus (RSV) infection, the methods comprising administering an atomized dose of an RSV vaccine. In some embodiments, provided are methods of immunizing subjects against an RSV infection, the method comprising administering a dose of an RSV vaccine using an intranasal atomization delivery device.

In some embodiments, the methods of immunizing subjects against RSV infection comprise administering an atomized dose of an RSV vaccine that comprises a live attenuated RSV. In some embodiments, the methods of immunizing subjects against RSV infection comprise administering a dose of an RSV vaccine that comprises a live attenuated RSV using an intranasal atomization delivery device. In some embodiments, provided are methods of immunizing a subject against an RSV infection, the method comprising administering an atomized dose of an RSV vaccine, optionally using an intranasal atomization delivery device, the RSV vaccine comprising an effective amount of a live-attenuated RSV with (i) a 523 nucleotide (nt) deletion of the NS2 gene (ΔNS2), (ii) an amino acid deletion in the L protein, and (iii) a genetically stabilizing mutation in the L gene (RSV ΔNS2/Δ1313/I1314L (NIH)) or RSV that further comprises a nucleotide modification at position 14456 with reference to SEQ ID NO: 1, which is in a non-coding region that represents a change from a thymine (T) to an adenine (A) (RSV ΔNS2/Δ1313/I1314L (Sanofi)). In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (I1314L).

In some embodiments, provided are methods of immunizing pediatric subjects against an RSV infection, the method comprising administering an atomized dose of an RSV vaccine. In some embodiments, provided are methods of immunizing pediatric subjects against an RSV infection, the method comprising administering a dose of an RSV vaccine using an intranasal atomization delivery device. In certain embodiments, the pediatric subject may be at least 6 months of age. In some embodiments, the pediatric subject may be 6 months to 18 months of age. In some embodiments, the pediatric subject may be 6 months to 22 months of age. In various embodiments, the pediatric subject may be from 6 months to less than 24 months of age. In some embodiments, the pediatric subject was born at term. In some embodiments, the pediatric subject was born preterm.

In some embodiments, provided are methods of preventing or reducing the likelihood of an RSV infection or preventing or reducing at least one symptom of an RSV infection in a subject, the method comprising administering an atomized dose of an RSV vaccine. In some embodiments, provided are methods of preventing or reducing the likelihood of an RSV infection or preventing or reducing at least one symptom of an RSV infection in a subject, the method comprising administering a dose of an RSV vaccine using an intranasal atomization delivery device.

In some embodiments, the methods of preventing or reducing the likelihood of an RSV infection or preventing or reducing at least one symptom of an RSV infection in a subject comprise administering an atomized dose of an RSV vaccine that comprises a live attenuated RSV. In some embodiments, the methods of preventing or reducing the likelihood of an RSV infection or preventing or reducing at least one symptom of an RSV infection in a subject comprise administering a dose of an RSV vaccine that comprises a live attenuated RSV using an intranasal atomization delivery device. In some embodiments, provided are methods of preventing or reducing the likelihood of an RSV infection or preventing or reducing at least one symptom of an RSV infection in a subject, the method comprising administering an atomized dose of an RSV vaccine, optionally using an intranasal atomization delivery device, the RSV vaccine comprising an effective amount of RSV ΔNS2/Δ1313/I1314L. In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (I1314L).

In some embodiments, provided are methods of preventing or reducing the likelihood of an RSV infection or preventing or reducing at least one symptom of an RSV infection in a pediatric subject, the method comprising administering an atomized dose of an RSV vaccine. In some embodiments, provided are methods of preventing or reducing the likelihood of an RSV infection or preventing or reducing at least one symptom of an RSV infection in a pediatric subject, the method comprising administering a dose of an RSV vaccine using an intranasal atomization delivery device. In certain embodiments, the pediatric subject may be at least 6 months of age. In some embodiments, the pediatric subject may be 6 months to 18 months of age. In some embodiments, the pediatric subject may be 6 months to 22 months of age. In some embodiments, the pediatric subject may be from 6 months to less than 24 months of age. In some embodiments, the pediatric subject was born at term. In some embodiments, the pediatric subject was born preterm.

In some embodiments, the pediatric subject is 6 months to 21 years of age. In some embodiments, the pediatric subject is 21 years of age or less. In some embodiments, the pediatric subject is 10 to 21 years of age. In some embodiments, the pediatric subject is 5 to 10 years of age. In some embodiments, the pediatric subject is 12 months to 5 years of age. In some embodiments, the pediatric subject may be at least 6 months of age. In some embodiments, the pediatric subject is 6 months to 36 months of age. In certain embodiments, the pediatric subject is from 6 months to less than 24 months of age. In some embodiments, the pediatric subject is 6 months to 18 months of age. In some embodiments, the pediatric subject is 6 months to 22 months of age. In some embodiments, the pediatric subject is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months of age. In some embodiments, the pediatric subject is 1, 2, 3, 4, 5, 6, 7, 8, 9, 10, 11, 12, 13, 14, 15, 16, 17, 18, 19, 20, or 21 years of age. In some embodiments, the pediatric subject was born at term. In some embodiments, the pediatric subject was born preterm. In some embodiments, the pediatric subject previously had an RSV infection. In some embodiments, the pediatric subject did not have an RSV infection.

In some embodiments, used with any of the methods described herein, an effective amount of the RSV comprises about 5.4 log₁₀ plaque forming units (PFU) per dose. In some embodiments, used with any of the methods described herein, an effective amount of the RSV comprises about 5.6 log₁₀ plaque forming units (PFU) per dose. In some embodiments, used with any of the methods described herein, an effective amount of the RSV comprises about 6.2 log₁₀ plaque forming units (PFU) per dose. In some embodiments, used with any of the methods described herein, an effective amount of the RSV comprises about 6.4 log₁₀ plaque forming units (PFU) per dose. In some embodiments, used with any of the methods described herein, an effective amount of the RSV comprises about 7.0 log₁₀ plaque forming units (PFU) per dose. In certain embodiments, used with any of the methods described herein, an effective amount of the RSV comprises about 5.4 log₁₀ PFU to 7.0 log₁₀ PFU. In certain embodiments, used with any of the methods described herein, an effective amount of the RSV comprises about 5.6 log₁₀ PFU to 6.2 log₁₀ PFU per dose. In certain embodiments, used with any of the methods described herein, an effective amount of the RSV comprises about 5.6 log₁₀ PFU to 6.4 log₁₀ PFU per dose. In certain embodiments, used with any of the methods described herein, an effective amount of the RSV comprises about 5 log₁₀ PFU, 5.1 log₁₀ PFU, 5.2 log₁₀ PFU, 5.3 log₁₀ PFU, 5.4 log₁₀ PFU, 5.5 log₁₀ PFU, 5.6 log₁₀ PFU, 5.7 log₁₀ PFU, 5.8 log₁₀ PFU, 5.9 log₁₀ PFU, 6 log₁₀ PFU, 6.1 log₁₀ PFU, 6.2 log₁₀ PFU, 6.3 log₁₀ PFU, 6.4 log₁₀ PFU, 6.5 log₁₀ PFU, 6.6 log₁₀ PFU, 6.7 log₁₀ PFU, 6.8 log₁₀ PFU, 6.9 log₁₀ PFU, 7 log₁₀ PFU, 7.1 log₁₀ PFU, 7.2 log₁₀ PFU, 7.3 log₁₀ PFU, 7.4 log₁₀ PFU, 7.5 log₁₀ PFU, 7.6 log₁₀ PFU, 7.7 log₁₀ PFU, 7.8 log₁₀ PFU, 7.9 log₁₀ PFU, 8 log₁₀ PFU, 8.1 log₁₀ PFU, 8.2 log₁₀ PFU, 8.3 log₁₀ PFU, 8.4 log₁₀ PFU, 8.5 log₁₀ PFU, 8.6 log₁₀ PFU, 8.7 log₁₀ PFU, 8.8 log₁₀ PFU, 8.9 log₁₀ PFU, and/or 9 log₁₀ PFU per dose. In certain embodiments, used with any of the methods described herein, an effective amount of the RSV comprises about 5 log₁₀ PFU to 9 log₁₀ PFU per dose. In some embodiments, used with any of the methods described herein, an effective amount of the RSV comprises about 5 log₁₀, 6 log₁₀, 7 log₁₀, 8 log₁₀ or 9 log₁₀ PFU per dose. In some embodiments, used with any of the methods described herein, an effective amount of the RSV comprises about 5 log₁₀ to about 9 log₁₀ PFU per dose.

In some embodiments, the RSV vaccine is delivered intranasally in an atomized dose. In some embodiments, the RSV vaccine is delivered intranasally using the intranasal atomization delivery device. In some embodiments, about ½ dose is delivered to each nostril. In some embodiments, the RSV vaccine is delivered intranasally so that the whole dose is delivered to one nostril. In some embodiments, the RSV vaccine is delivered intranasally with ½ dose delivered to one nostril and the other ½ dose is delivered to the same nostril, sequentially or at the same time. In some embodiments, the RSV vaccine is delivered intranasally delivering a dose in unequal amounts to one or both nostrils. In some embodiments, the RSV vaccine is delivered intranasally with ¾ dose delivered to one nostril with ¼ dose delivered to the other nostril or the same nostril. In some embodiments, the RSV vaccine is delivered intranasally with 1/10, 1/9, ⅛, 1/7, ⅙, ⅕, ¼, ⅓, ½, or the whole dose delivered to one nostril and the other 9/10, 8/9, ⅞, ⅚, ⅘, ¾, ⅔, or ½ of the dose is delivered to the other nostril or the same nostril.

In some embodiments, the RSV vaccine is delivered intranasally in a liquid formulation. In some embodiments, the RSV vaccine dose is delivered intranasally in a volume of about 0.2 mL. In some embodiments, the 0.2 mL dose is delivered intranasally wherein about 0.1 mL is delivered to each nostril. In some embodiments, the RSV vaccine dose is delivered intranasally using the intranasal atomization delivery device in a volume of about 0.1 mL, 0.2 mL, 0.3 mL, 0.4 mL, 0.5 mL, 0.6 mL, 0.7 mL, 0.8 mL, 0.9 mL, or 1.0 mL. As described above, the dose may be divided evenly between two nostrils, divided substantially evenly between two nostrils, divided unevenly between two nostrils, or delivered all to one nostril in one or more deliveries. In some embodiments used with any of the methods described herein, the RSV vaccine is delivered intranasally using an intranasal atomization delivery device. In some embodiments, the intranasal atomization delivery device may include the RSV vaccine in a prefilled barrel.

In some embodiments, provided are methods of immunizing subjects against a respiratory syncytial virus (RSV) infection, the methods comprising administering a first dose of an RSV vaccine and administering a second dose of the RSV vaccine. In some embodiments, the methods of immunizing subjects against RSV infection may comprise administering one or more doses using an intranasal atomization delivery device.

In some embodiments, methods of immunizing subjects against RSV infection are provided that comprise administering a first dose of an RSV vaccine that comprises a live attenuated RSV, optionally using an intranasal atomization delivery device and administering a second dose of the RSV vaccine, optionally using an intranasal atomization delivery device.

In some embodiments, provided are methods of preventing or reducing the likelihood of an RSV infection or preventing or reducing at least one symptom of an RSV infection in a subject, the method comprising administering a first dose of an RSV vaccine and administering a second dose of the RSV vaccine. In some embodiments, provided are methods of preventing or reducing the likelihood of an RSV infection or preventing or reducing at least one symptom of an RSV infection in a subject, wherein one or more doses of an RSV vaccine are administered using an intranasal atomization delivery device.

In some embodiments, the methods of preventing or reducing the likelihood of an RSV infection or preventing or reducing at least one symptom of an RSV infection in a subject comprise administering a dose of an RSV vaccine that comprises a live attenuated RSV, optionally using an intranasal atomization delivery device and administering a second dose of the RSV vaccine, optionally using an intranasal atomization delivery device.

In some embodiments, used with any of the methods described herein, an effective amount of the RSV in a second dose comprises about 5.4 log₁₀ PFU per dose. In some embodiments, used with any of the methods described herein, an effective amount of the RSV in a second dose comprises about 5.6 log₁₀ PFU per dose. In some embodiments, used with any of the methods described herein, an effective amount of the RSV in a second dose comprises about 6.2 log₁₀ PFU per dose. In some embodiments, used with any of the methods described herein, an effective amount of the RSV in a second dose comprises about 6.4 log₁₀ PFU per dose. In some embodiments, used with any of the methods described herein, an effective amount of the RSV in a second dose comprises about 7.0 log₁₀ PFU per dose. In certain embodiments, used with any of the methods described herein, an effective amount of the RSV comprises about 5.4 log₁₀ PFU to 7.0 log₁₀ PFU. In certain embodiments, used with any of the methods described herein, an effective amount of the RSV in a second dose comprises about 5.4 log₁₀ PFU to 7.0 log₁₀ PFU. In certain embodiments, used with any of the methods described herein, an effective amount of the RSV in a second dose comprises about 5.6 log₁₀ PFU to 6.2 log₁₀ PFU per dose. In some embodiments, used with any of the methods described herein, an effective amount of the RSV in a second dose comprises about 5.4 log₁₀ PFU per dose. In some embodiments, used with any of the methods described herein, an effective amount of the RSV in a second dose comprises about 5.6 log₁₀ PFU per dose. In some embodiments, used with any of the methods described herein, an effective amount of the RSV in a second dose comprises about 6.2 log₁₀ PFU per dose. In some embodiments, used with any of the methods described herein, an effective amount of the RSV in a second dose comprises about 6.4 log₁₀ PFU per dose. In some embodiments, used with any of the methods described herein, an effective amount of the RSV in a second dose comprises about 7.0 log₁₀ PFU per dose. In certain embodiments, used with any of the methods described herein, an effective amount of the RSV in a second dose comprises about 5.2 log₁₀ PFU to 7.0 log₁₀ PFU per dose. In certain embodiments, used with any of the methods described herein, an effective amount of the RSV in a second dose comprises about 5.6 log₁₀ PFU to 6.4 log₁₀ PFU per dose. In certain embodiments, used with any of the methods described herein, an effective amount of the RSV in a second dose comprises about 5 log₁₀ PFU, 5.1 log₁₀ PFU, 5.2 log₁₀ PFU, 5.3 log₁₀ PFU, 5.4 log₁₀ PFU, 5.5 log₁₀ PFU, 5.6 log₁₀ PFU, 5.7 log₁₀ PFU, 5.8 log₁₀ PFU, 5.9 log₁₀ PFU, 6 log₁₀ PFU, 6.1 log₁₀ PFU, 6.2 log₁₀ PFU, 6.3 log₁₀ PFU, 6.4 log₁₀ PFU, 6.5 log₁₀ PFU, 6.6 log₁₀ PFU, 6.7 log₁₀ PFU, 6.8 log₁₀ PFU, 6.9 log₁₀ PFU, 7 log₁₀ PFU, 7.1 log₁₀ PFU, 7.2 log₁₀ PFU, 7.3 log₁₀ PFU, 7.4 log₁₀ PFU, 7.5 log₁₀ PFU, 7.6 log₁₀ PFU, 7.7 log₁₀ PFU, 7.8 log₁₀ PFU, 7.9 log₁₀ PFU, 8 log₁₀ PFU, 8.1 log₁₀ PFU, 8.2 log₁₀ PFU, 8.3 log₁₀ PFU, 8.4 log₁₀ PFU, 8.5 log₁₀ PFU, 8.6 log₁₀ PFU, 8.7 log₁₀ PFU, 8.8 log₁₀ PFU, 8.9 log₁₀ PFU, and/or 9 log₁₀ PFU per dose. In some embodiments, used with any of the methods described herein, an effective amount of the RSV in a second dose comprises about 5 log₁₀ to about 9 log₁₀ PFU per dose. In some embodiments, an effective amount of the RSV in a second dose comprises about 5 log₁₀, about 5.4 log₁₀, about 5.6 log₁₀, about 6 log₁₀, about 6.2 log₁₀, about 6.4 log₁₀, about 7 log₁₀, about 8 log₁₀, or about 9 log₁₀ PFU per dose. In some embodiments, the first dose and the second dose of the RSV vaccine are the same. In some embodiments, the first dose and the second dose of the RSV vaccine administered are different. In some embodiments, an effective amount of the RSV in a first dose and a second dose comprises about 5.4 log₁₀ PFU per dose. In some embodiments, an effective amount of the RSV in a first dose and a second dose comprises about 5.6 log₁₀ PFU per dose. In some embodiments, an effective amount of the RSV in a first dose and a second dose comprises about 6.2 log₁₀ PFU per dose. In some embodiments, an effective amount of the RSV in a first dose and a second dose comprises about 6.4 log₁₀ PFU per dose. In some embodiments, an effective amount of the RSV in a first dose and a second dose comprises about 7.0 log₁₀ PFU per dose. In some embodiments, an effective amount of the RSV in a first dose and a second dose comprises about 5 log₁₀ to about 9 log₁₀ PFU per dose. In some embodiments, an effective amount of the RSV in a first dose comprises about 5.4 log₁₀ PFU per dose and a second dose comprises about 5.6 log₁₀ PFU per dose. In some embodiments, an effective amount of the RSV in a first dose comprises about 5.4 log₁₀ PFU per dose and a second dose comprises about 6.2 log₁₀ PFU per dose. In some embodiments, an effective amount of the RSV in a first dose comprises about 5.4 log₁₀ PFU per dose and a second dose comprises about 6.4 log₁₀ PFU per dose. In some embodiments, an effective amount of the RSV in a first dose comprises about 5.4 log₁₀ PFU per dose and a second dose comprises about 6.4 log₁₀ PFU per dose. In some embodiments, an effective amount of the RSV in a first dose comprises about 5.4 log₁₀ PFU per dose and a second dose comprises about 7.0 log₁₀ PFU per dose. In some embodiments, an effective amount of the RSV in a first dose comprises about 5.6 log₁₀ PFU per dose and a second dose comprises about 5.2 log₁₀ PFU per dose. In some embodiments, an effective amount of the RSV in a first dose comprises about 5.6 log₁₀ PFU per dose and a second dose comprises about 6.2 log₁₀ PFU per dose. In some embodiments, an effective amount of the RSV in a first dose comprises about 5.6 log₁₀ PFU per dose and a second dose comprises about 6.4 log₁₀ PFU per dose. In some embodiments, an effective amount of the RSV in a first dose comprises about 5.6 log₁₀ PFU per dose and a second dose comprises about 7.0 log₁₀ PFU per dose. In some embodiments, an effective amount of the RSV in a first dose comprises about 6.2 log₁₀ PFU per dose and a second dose comprises about 5.4 log₁₀ PFU per dose. In some embodiments, an effective amount of the RSV in a first dose comprises about 6.2 log₁₀ PFU per dose and a second dose comprises about 5.6 log₁₀ PFU per dose. In some embodiments, an effective amount of the RSV in a first dose comprises about 6.2 log₁₀ PFU per dose and a second dose comprises about 6.4 log₁₀ PFU per dose. In some embodiments, an effective amount of the RSV in a first dose comprises about 6.2 log₁₀ PFU per dose and a second dose comprises about 7.0 log₁₀ PFU per dose. In some embodiments, an effective amount of the RSV in a first dose comprises about 6.4 log₁₀ PFU per dose and a second dose comprises about 5.4 log₁₀ PFU per dose. In some embodiments, an effective amount of the RSV in a first dose comprises about 6.4 log₁₀ PFU per dose and a second dose comprises about 5.6 log₁₀ PFU per dose. In some embodiments, an effective amount of the RSV in a first dose comprises about 6.4 log₁₀ PFU per dose and a second dose comprises about 6.2 log₁₀ PFU per dose. In some embodiments, an effective amount of the RSV in a first dose comprises about 6.4 log₁₀ PFU per dose and a second dose comprises about 7.0 log₁₀ PFU per dose. In some embodiments, an effective amount of the RSV in a first dose comprises about 7.0 log₁₀ PFU per dose and a second dose comprises about 5.4 log₁₀ PFU per dose. In some embodiments, an effective amount of the RSV in a first dose comprises about 7.0 log₁₀ PFU per dose and a second dose comprises about 5.6 log₁₀ PFU per dose. In some embodiments, an effective amount of the RSV in a first dose comprises about 7.0 log₁₀ PFU per dose and a second dose comprises about 6.2 log₁₀ PFU per dose. In some embodiments, an effective amount of the RSV in a first dose comprises about 7.0 log₁₀ PFU per dose and a second dose comprises about 6.4 log₁₀ PFU per dose. In some embodiments, an effective amount of the RSV in a first dose comprises about 5 log₁₀, about 5.4 log₁₀, about 5.6 log₁₀, about 6 log₁₀, about 6.2 log₁₀, about 6.4 log₁₀, about 7 log₁₀, about 8 log₁₀ or about 9 log₁₀ PFU per dose and a second dose comprises about 5 log₁₀, about 5.2 log₁₀, about 5.6 log₁₀, about 6 log₁₀, about 6.2 log₁₀, about 6.4 log₁₀, about 7 log₁₀, about 8 log₁₀, or about 9 log₁₀ PFU per dose.

In some embodiments, single or multiple doses of a live attenuated RSV vaccine may be administered to the subjects described herein. Multiple doses (e.g., 2, 3, 4 or more doses) may be used in a primary immunization schedule and/or in a booster immunization schedule. Multiple doses will typically be administered at least 1 week apart (e.g., about 2 weeks, about 3 weeks, about 4 weeks, about 6 weeks, about 8 weeks, about 12 weeks, about 16 weeks, etc.). In some embodiments, the RSV vaccine is administered in a single dose. In some embodiments, a second RSV vaccine is administered one or more times, optionally about 2 weeks, about 3 weeks, about 4 weeks, about 6 weeks, about 8 weeks, about 12 weeks, or about 16 weeks apart, or other appropriate interval. In some embodiments, a second RSV vaccine is administered about 7 weeks to about 9 weeks after the first dose. In some embodiments, a second dose is administered about 40-50, 45-55, 55-60, 52-60, or 60-65 days after the first dose. In some embodiments, two doses are administered about 56 days (8 weeks) apart. In some embodiments, two doses are administered at least 56 days apart. In some embodiments the single or multiple doses of the live attenuated RSV vaccine may be administered using an intranasal atomization delivery device.

In some embodiments, administration of the RSV vaccine reduces the incidence of laboratory confirmed RSV. In some embodiments, administration of the RSV vaccine reduces at least one symptom of RSV infection in a subject where the symptoms are selected from development or onset of an upper and/or lower respiratory tract RSV infection, otitis media or a respiratory condition related thereto, or the progression of an upper respiratory tract RSV infection to a lower respiratory tract RSV infection, or the progression to otitis media or a respiratory condition related thereto. In some embodiments, administration of the RSV vaccine reduces at least one symptom of RSV infection where the symptoms are selected from asthma, wheezing, or a combination thereof.

In some embodiments, provided are methods of immunizing subjects against a respiratory syncytial virus (RSV) infection, the methods comprising administering a first dose of an RSV vaccine using an intranasal atomization delivery device, wherein the intranasal atomization delivery device comprises a spray nozzle to atomize the RSV vaccine. In some embodiments, a spray plume from the spray nozzle may be directed toward a top of a nasal passageway into a nasal cavity. In some embodiments, provided are methods of preventing or reducing the likelihood of an RSV infection or preventing or reducing at least one symptom of an RSV infection in a pediatric subject, the methods comprising administering a dose of an RSV vaccine using an intranasal atomization delivery device, wherein the intranasal atomization delivery device comprises a spray nozzle to atomize the RSV vaccine. In some embodiments, the intranasal atomization delivery device comprises a barrel operably connected to the spray nozzle and a plunger movable within the barrel to advance the RSV vaccine through the spray nozzle. In some embodiments, the intranasal atomization delivery device further includes a dose divider for splitting the dose of the RSV vaccine into two or more deliveries. In some embodiments, the dose divider may divide the dose of the RSV vaccine in about half of a volume to be delivered to a subject. In some embodiments the dose divider is used to deliver ½ dose to each nostril of the subject.

In some embodiments, the intranasal atomization delivery device delivers an average droplet size Dᵥ₅₀ of 10-120 μm. In some embodiments, the intranasal atomization delivery device delivers an average droplet size Dᵥ₅₀ of about 10-120 μm, about 20-120 μm, about 30-120 μm, about 40-120 μm, about 50-110 μm, about 60-110 μm, about 70-110 μm, or about 80-110 μm. In some embodiments, the intranasal atomization delivery device delivers an average droplet size Dᵥ₅₀ of at least 100 μm, at least 110 μm, or at least 120 μm. In some embodiments, the intranasal atomization delivery device delivers an average droplet size Dᵥ₅₀ of at least 30 μm, at least 50 μm, at least 70 μm, at least 80 μm, or at least 110 μm. In some embodiments, the intranasal atomization delivery device delivers an average shot weight of about 30 mg to about 200 mg, about 50 mg to about 175 mg, about 70 mg to about 160 mg, about 80 mg to about 150 mg, 95 mg to about 135 mg, about 100 mg to about 130 mg, about 100 mg to about 130 mg, or between about 105 mg to about 130. In some embodiments, the intranasal atomization delivery device delivers an average shot volume between about 25 μL to about 200 μL, about 50 μL to about 175 μL, about 60 μL to about 150 μL, about 75 μL to about 130 μL, about 85 μL to about 120 μL, about 90 μL to about 115 μL, or about 95 μL to about 115 μL.

In some embodiments, use of an intranasal atomization delivery device described herein for administering a dose of an RSV vaccine to a subject is provided, wherein the RSV vaccine comprises an effective amount of a live-attenuated RSV. In some embodiments, the use includes a live-attenuated RSV ΔNS2/Δ1313/I1314L.

In some embodiments, use of an RSV vaccine for the manufacture of a medicament for preventing or reducing the likelihood of infection by RSV virus in a subject is provided, the RSV vaccine comprising an effective amount of a live-attenuated RSV. In some embodiments, the use includes RSV ΔNS2/Δ1313/I1314L.

### III. RSV Vaccine

In some embodiments, the RSV vaccine may comprise a live-attenuated vaccine. In one aspect, the RSV vaccine described as the “RSV ΔNS2/Δ1313/I1314L vaccine” is a live-attenuated vaccine based on deletion of the gene encoding the RSV interferon/apoptosis antagonist NS2 protein (ΔNS2). Deletion of NS2 gene attenuates the virus and may enhance immunogenicity. The RSV ΔNS2/Δ1313/I1314L vaccine additionally includes a genetically stabilized attenuating and temperature sensitivity mutation in the L protein (codon deletion Δ1313, as well as a missense mutation I1314L that prevents a de-attenuating mutation that otherwise can occur at position 1314), and as a result RSV ΔNS2/Δ1313/I1314L is temperature sensitive, with a shut-off temperature for virus replication of 38° C.-39° C. (100.4° F.-102.2° F.).

The RSV ΔNS2/Δ1313/I1314L vaccine is a recombinant infectious respiratory syncytial virus including a large polymerase protein (L), phosphoprotein (P), nucleocapsid protein (N), a M2-1 protein nonstructural protein 1 (NS1), a glycoprotein (G), a fusion protein (F), a matrix protein (M), an M2-2 protein, and a small hydrophobic protein (SH), and a genome or antigenome having: a deletion of the codon that encodes the serine at position 1313, or a corresponding position, of the L protein, a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG and a deletion of the NS2 gene. The RSV ΔNS2/Δ1313/I1314L vaccine and methods of making the vaccine, are described in WO 2013/154728 A1, which is incorporated by reference in its entirety for any purpose.

### IV. Intranasal Atomization Delivery Device

### A. Device

In some embodiments, an intranasal atomization delivery device may be used to deliver a vaccine described herein. The intranasal atomization delivery device may be any suitable device for atomizing a live-attenuated RSV vaccine. In some embodiments, the delivery device may provide an average droplet size Dᵥ₅₀ of about 10 μm to 120 μm. In some embodiments, the delivery device is suitable for delivery to a pediatric subject. In certain embodiments, the delivery device is suitable for delivery to a pediatric subject 6 months to 18 months of age. In various embodiments, the delivery device is suitable for delivery to a pediatric subject from 6 months to less than 24 months of age. In some embodiments, the delivery device is suitable for delivery to a pediatric subject who is at least 6 months of age. In various embodiments, the delivery device may be configured to deliver two approximately equal doses, one to each nostril. In some embodiments, both doses are delivered sequentially or simultaneously.

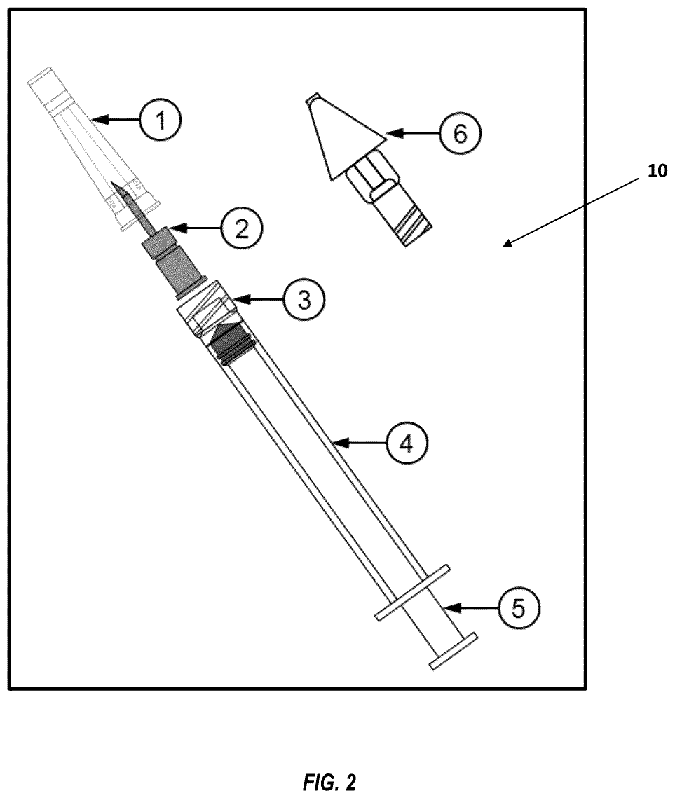

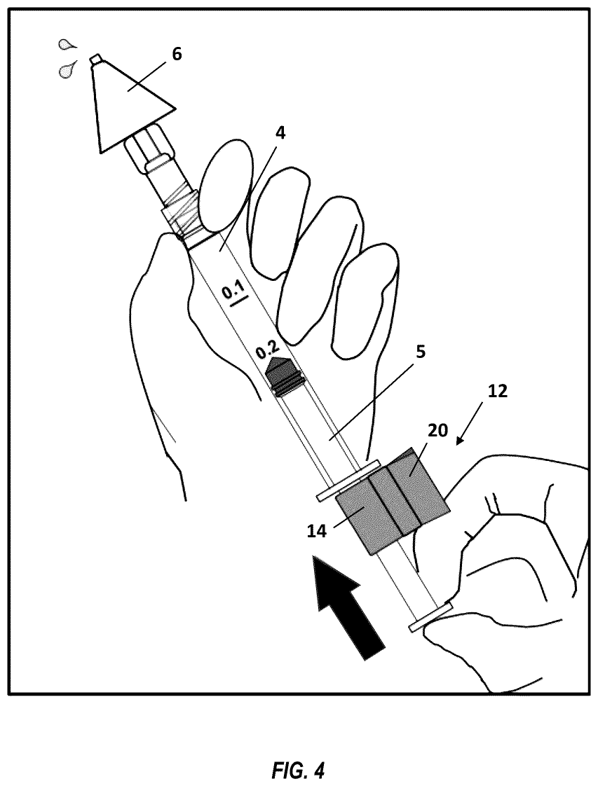

By way of non-limiting example, a suitable intranasal atomization delivery device 10 is described. As shown in FIG. 2, the intranasal atomization delivery device 10 may include a barrel 4 for receiving the vaccine for delivery to the subject. The delivery device 10 may also include a luer lock 3 adapted to receive a vial access cannula 2 and/or an atomizer 6. The delivery device may include a cap 1 to cover the vial access cannula 2. The delivery device 10 may also include a plunger 5 sized and shaped to fit within the barrel 4. The barrel 4 may also include markings to identify a volume within the barrel 4 for delivering a fluid volume of the vaccine as shown in FIG. 4. By way of non-limiting example, the markings may be 0.05 mL markings, 0.1 mL markings, 0.2 mL markings, or other markings depending on the dose to be delivered and whether administration is to one nostril or both nostrils. In some embodiments, the barrel 4 may be a 1 mL syringe. By way of non-limiting example, the syringe may be a 1 mL Luer Lock disposable plastic syringe, such as, B. Braun Omnifix®-F. In some embodiments, the barrel 4 may be a smaller or larger volume syringe. The vial access cannula 2 may be used to withdraw the vaccine from a vial at the medical center for delivery to the subject. In some embodiments, the vial access cannula 2 may be a semi-blunt fill needle or a pointed fill needle. By way of non-limiting example, the semi-blunt fill needle may be an 18 G×40 mm (B. Braun Sterican® MIX). Once the barrel 4 is filled with the appropriate vaccine dose, the vial access cannula 2 may be removed and the atomizer 6 may be connected to the barrel 4 via the luer lock 3. The atomizer 6 may be primed before the vaccine dose is administered intranasally. The atomizer 6 may be generally conically shaped with a wider base narrowing to an end for insertion into a nostril so that a spray plume is emitted from the atomizer 6 as described below. In some embodiments, the barrel 4 may be pre-filled with the vaccine dose to be delivered.

In some embodiments, the intranasal delivery device 10 may have a typical droplet size of about 10 μm to 120 μm and a system dead space of about 0.1 mL to 0.2 mL. In some embodiments, the system dead space may be about 0.15 mL. The atomizer 6 may have a tip diameter of about 3.5 mm to 5 mm to facilitate delivery into the nasal cavity. In some embodiments, the tip diameter may be about 4 mm to 4.5 mm, and may be about 4.3 mm. By way of non-limiting example, a Teleflex MAD130 Nasal™ Device system or a Teleflex Vaxinator™ (VAX300) device may be used to deliver a vaccine intranasally to a subject. In some embodiments, the atomizer 6 may be an Aptar, LuerVax® device.

### B. Dose Divider

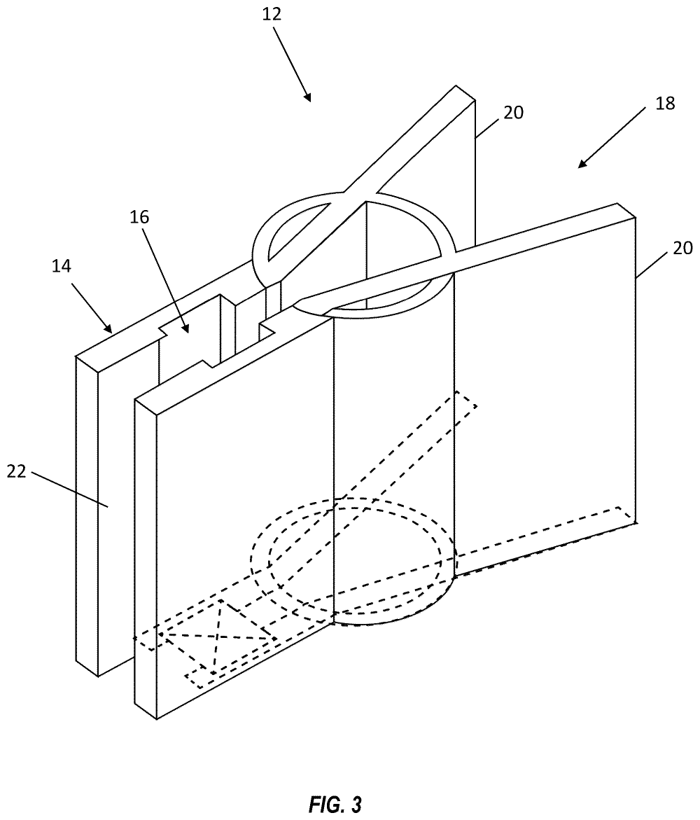

In some embodiments, a dose divider 12 (see FIGS. 3 and 4) may be provided with the intranasal atomization delivery device 10 of FIG. 2. The dose divider 12 may be used to divide the vaccine dose provided in the intranasal atomization delivery device 10. In some embodiments, the dose divider 12 may be used to divide the vaccine dose into two approximately equal half volumes of the vaccine dose (e.g., a first half dose and a second half dose), one half volume for each nostril or two half volumes to the same nostril, delivered separately. Other dose divisions are also possible and depend on the height of the dose divider. As shown in FIGS. 3 and 4, the dose divider 12 may include a first portion 14 having a height that corresponds to the partial dose to be delivered to the first nostril. The first portion 14 may also include a notch 16 that is sized and shaped to correspond to the size and shape of the plunger 5. The dose divider 12 can also include a second portion 18 that includes two protrusions 20 that may be used to expand an opening 22 in the first portion 14 so the notch 16 may be positioned over the plunger 5 of the delivery device 10. The two protrusions 20 may be released to close the first portion 14 over the plunger 5 to control the volume of the first partial dose delivered to the first nostril. The dose divider 12 may be removed from the plunger 5 for delivery of the second partial dose to the second nostril of the subject. By way of non-limiting example, a dose divider (such as dose divider 12) may be used to deliver about a 0.01 mL, 0.02 mL, 0.05 mL, 0.075 mL, 0.1 mL, 0.2 mL, 0.3 mL, 0.4 mL, 0.5 mL, 0.6 mL, 0.7 mL, 0.8 mL, 0.9 mL, or 1.0 mL volume per nostril. Other volumes may also be used. In some embodiments, the dose divider 12 may divide the dose into two about equal half doses.

### C. Dose Delivery

The vaccine may be delivered to the subject intranasally using the delivery device 10. In some embodiments, the dose may be delivered approximately in half to each nostril, a larger amount to the first nostril and a smaller amount to the second nostril, or two administrations of the same volume or different volumes to the same nostril. Dose delivery is described herein for the delivery of approximately a half dose to each nostril but is not limited thereto. The full dose to be delivered to the subject can be loaded into the delivery device 10 using the vial access cannula 2 to withdraw the vaccine from a container (e.g., a vial; not shown) containing one or more doses of the vaccine. Once the full dose is loaded into the barrel 4 of the delivery device 10, the vial access cannula 2 can be removed from the luer lock 3 and the atomizer 6 can be coupled or connected to the luer lock 3. Furthermore, the atomizer 6 can be primed and the dose divider 12 can be connected to the plunger 5 as described above. With the atomizer 6 inserted into the first nostril, the plunger 5 can be advanced a first distance within the barrel 4 to force the first half dose through the atomizer 6, atomizing the vaccine to form a spray plume. The spray plume can include a fine mist of droplets ranging in size from about 10 μm to about 120 μm. A distal region of the atomizer 6 may be generally conically shaped. The spray plume may be directed generally towards the top of the nasal passage through the nasal valve into the nasal cavity, including nasal turbinate areas (including inferior, middle and superior nasal turbinate areas). The plunger 5 can be advanced the first distance within the barrel 4 until an end of the plunger 5 contacts the dose divider 12 and stops advancing to deliver the first half dose to the first nostril.

Following completion of delivery of the first half dose to the first nostril, the dose divider 12 can be removed from the plunger 5. Then, the atomizer 6 can be inserted into the second nostril and the plunger 5 advanced a second distance within the barrel 4 to deliver the second half dose to the second nostril. The second half dose advanced the second distance may be completed when the plunger 5 contacts an end of the barrel 4.

### EXAMPLES

The following examples are provided to illustrate certain disclosed embodiments and are not to be construed as limiting the scope of this disclosure in any way.

### Example 1. Safety, Immunogenicity, Infectivity, and Dose-Finding Study of an Investigational Live-Attenuated RSV Vaccine in Infants and Toddlers

### Executive Summary

### Investigational Product

Recombinant Live-Attenuated Respiratory Syncytial Virus (RSV) RSV ΔNS2/Δ1313/I1314L (Sanofi) vaccine.

### Active Substance

Live-attenuated RSV with (i) a 523 nucleotide (nt) deletion of the NS2 gene (ΔNS2), (ii) an amino acid deletion in the L protein (Δ1313; deletion of S1313), and (iii) a genetically stabilizing mutation in the L gene (I1314L) (RSV ΔNS2/Δ1313/I1314L (Sanofi)).

### Study Sites

A multi-center, multinational study including approximately 30 sites in the United States (US), approximately 2 sites in Canada, approximately 6 sites in Latin America (Argentina, Chile, and Honduras), and approximately 2 sites in South Africa.

### Phase I/II Study

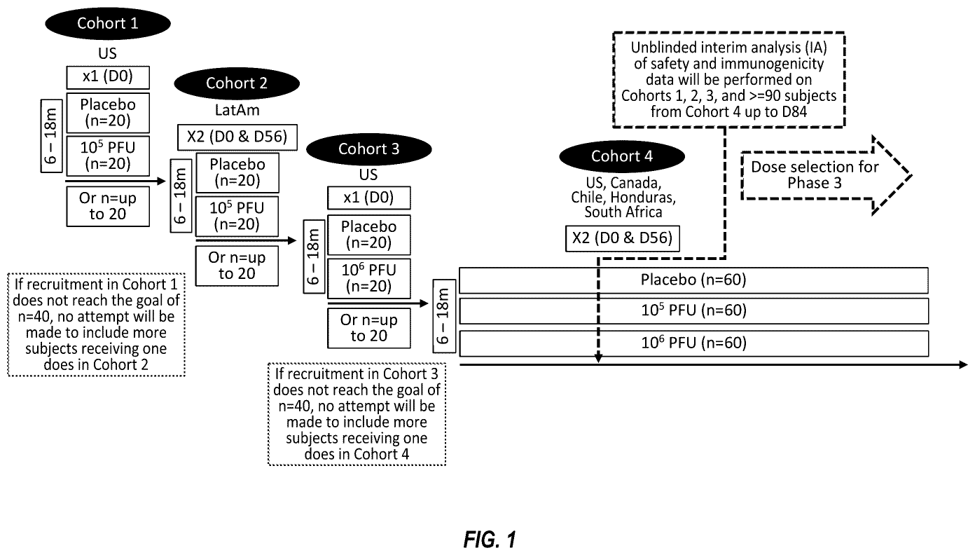

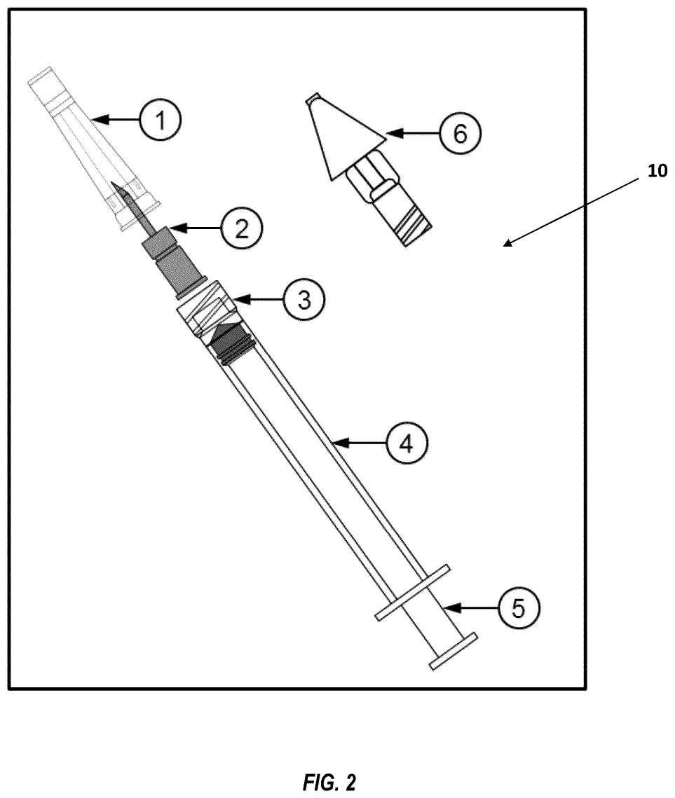

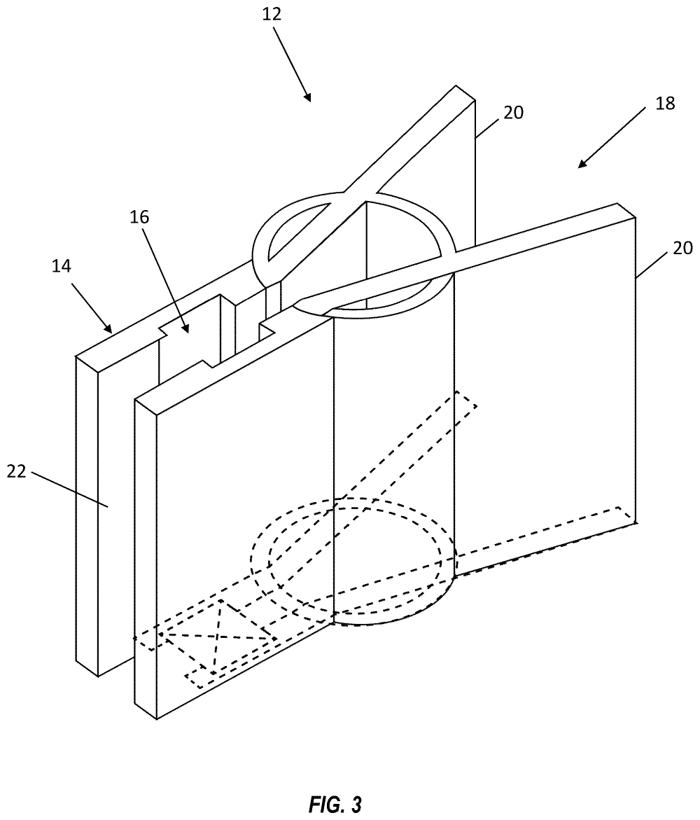

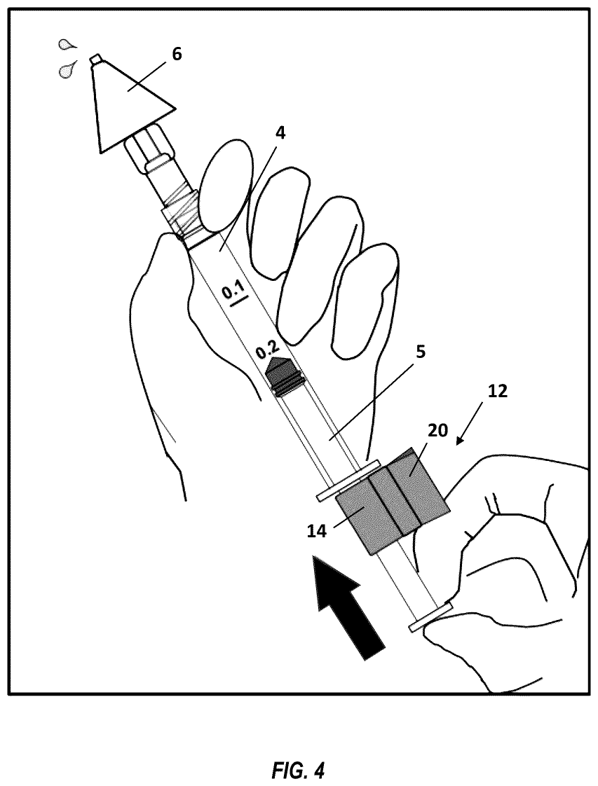

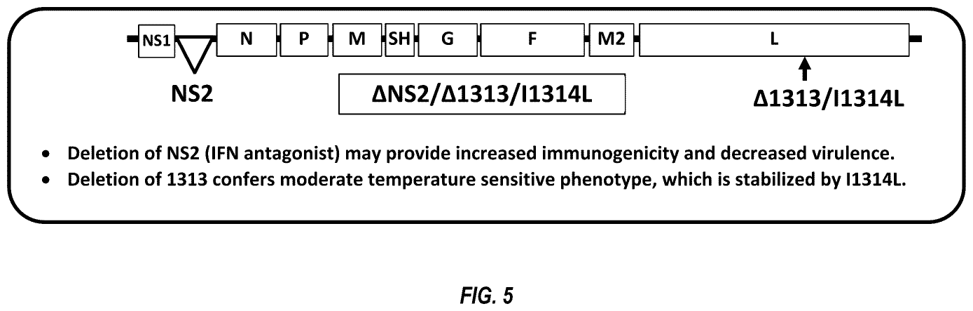

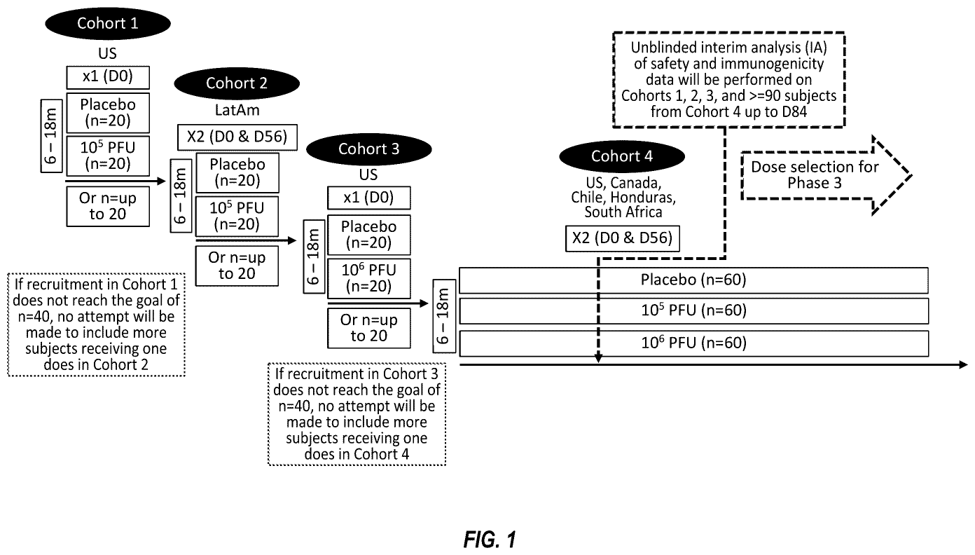

A Phase I/II, randomized, observer-blind, placebo-controlled, multi-center, dose-finding study to evaluate the safety, immunogenicity, infectivity, and vaccine virus shedding after 1 or 2 administrations of a live-attenuated RSV ΔNS2/Δ1313/I1314L (Sanofi) vaccine using an intranasal atomization delivery device in infants and toddlers 6 months to 18 months (Cohorts 1 to 4) of age in the United States, Canada, Latin America (Argentina, Chile, and Honduras), and South Africa was conducted. An example study protocol is shown in FIG. 1 where the doses may be any of the doses described in Example 1.

### Vaccination:

A total of 300 infants and toddlers 6 to 18 months of age were enrolled into 1 of 4 cohorts, sequentially, and within each cohort were randomized to receive intranasal administration of their assigned study product using an intranasal atomization delivery device as follows:
Cohort 1-40 infants and toddlers received 1 administration, 1:1 ratio of RSV ΔNS2/Δ1313/I1314L (Sanofi) 5.6 log₁₀ PFU (low-dose) or placebo (same formulation buffer as the RSV ΔNS2/Δ1313/I1314L (Sanofi) vaccine).
Cohort 2-40 infants and toddlers received 2 administrations, 1:1 ratio of RSV ΔNS2/Δ1313/I1314L (Sanofi) 5.6 log₁₀ PFU (low-dose) or placebo.
Cohort 3-40 infants and toddlers received 1 administration, 1:1 ratio of RSV ΔNS2/Δ1313/I1314L (Sanofi) 6.2 log₁₀ PFU (high-dose) or placebo.
Cohort 4-180 infants and toddlers received 2 administrations, 1:1:1 of RSV ΔNS2/Δ1313/I1314L (Sanofi) 5.6 log₁₀ PFU (low-dose), RSV ΔNS2/Δ1313/I1314L (Sanofi) 6.2 log₁₀ PFU (high-dose), or placebo.

TABLE 1
Vaccination Cohorts

| | | Cohort | | | |
|---|---|---|---|---|---|
| | Total | 1 | 2 | 3 | 4 |
| Vaccine Groups | (n) | (n) | (n) | (n) | (n) |
| Administration(s) | | x1 | x2 | x1 | x2 |
| RSV ΔNS2/Δ1313/I1314L (Sanofi) 5.6 | 100 | 20 | 20 | | 60 |
| log₁₀ PFU per administration (low-dose) | | | | | |
| RSV ΔNS2/Δ1313/I1314L (Sanofi) 6.2 | 80 | | | 20 | 60 |
| log₁₀ PFU per administration (high- | | | | | |
| dose) | | | | | |
| Placebo | 120 | 20 | 20 | 20 | 60 |
| TOTAL | 300 | 40 | 40 | 40 | 180 |

| Duration of | The duration of active participation of each |
|---|---|
| Participation | subject in each cohort is up to a maximum of |
| in the Study: | 12 months. |
| Investigational | Respiratory Syncytial Virus (RSV) RSV ΔNS2/ |
| Product: | Δ1313/I1314L (Sanofi) Vaccine 5.6 log₁₀ |
| | PFU/0.2 mL |
| Form: | Suspension of virus |
| Composition: | Each 0.2 mL dose of RSV ΔNS2/Δ1313/I1314L |
| | (Sanofi) contains the live-attenuated RSV |
| | with (i) a 523-nucleotide deletion of the |
| | NS2 gene, (ii) an amino acid deletion in |
| | the L protein (Δ1313; deletion of S1313), |
| | and (iii) genetically stabilizing mutation |
| | in the L gene (I1314L), 5.6 log₁₀ PFU, |
| | approximately 0.1 mL to be delivered as a |
| | fine mist of droplets (range 10-120 μm in |
| | size) per nostril, using an intranasal |
| | atomizer device. |
| Route: | Intranasal |
| Investigational | Respiratory Syncytial Virus (RSV) RSV |
| Product: | ΔNS2/Δ1313/I1314L (Sanofi) Vaccine 6.2 |
| | log₁₀ PFU/0.2 mL |
| Form: | Suspension of virus |
| Composition: | Each 0.2 mL dose of RSV ΔNS2/Δ1313/I1314L |
| | (Sanofi) contains the live-attenuated RSV |
| | with (i) a 523-nucleotide deletion of the |
| | NS2 gene, (ii) an amino acid deletion in |
| | the L protein (Δ1313; deletion of S1313), |
| | and (iii) genetically stabilizing mutation |
| | in the L gene (I1314L), 6.2 log₁₀ PFU, |
| | approximately 0.1 mL to be delivered as a |
| | fine mist of droplets (range 10-120 μm in |
| | size) per nostril, using an intranasal |
| | atomizer device. |
| Route: | Intranasal |
| Non-Investigational | Placebo |
| Product: | |
| Form: | Solution |
| Composition: | Same formulation buffer as the RSV |
| | ΔNS2/Δ1313/I1314L (Sanofi) vaccine, to be |
| | delivered as approximately 0.1 mL per nostril |
| Route: | Intranasal |
| Device: | A US licensed, commercially available |
| | Atomization Device is used for |
| | administration of the experimental RSV |
| | ΔNS2/Δ1313/I1314L (Sanofi) vaccine in |
| | the Phase I/II clinical trial. |

Table of Study Procedures for Cohorts 1 and 3
Phase I/II Study, 4 Planned Visits, 30 (32) non-visit telephone contacts, 1 Vaccination, 3 Planned Blood Samples,
1 Planned Nasal Swab, up to a Maximum of 12 Months of Participation per Subject

| | Visit (V)/Contact | | | | | | | | | |
|---|---|---|---|---|---|---|---|---|---|---|
| | | | | | | | | Phone | | Illness |
| | | Phone | | Phone | Phone | Phone | | contacts | | Visit(s) |
| | Visit | contacts | Visit | contacts | Contact | contact | Visit | 19-30 | Visit | Visit |
| | 01 | 1-6 | 02 | 7-16 | 17 | 18 | 03 | (32) ^{‡‡} | 04^{§§} | 99*** |
| | Study timelines - Days(D) | | | | | | | | | |
| | | | | Three | | | | | | |
| | | | | times | | | | | 1-30 | |
| | | | | weekly | | | | Every 2 | April | |
| | D0 | Daily (×6) | D7 | (×10) | D29 (+1) | D42 (+1) | D56 (+7) | weeks | (NH) | |
| In-person visit | X | | X | | | | X | | X | X |
| Non-visit contact* | | X | | X | X | X | | X | | |
| Informed consent | X | | | | | | | | | |
| Demography data | X | | | | | | | | | |
| Inclusion/Exclusion | X | | | | | | | | | |
| criteria | | | | | | | | | | |
| Medical history | X | | | | | | | | | |
| and physical | | | | | | | | | | |
| examination | | | | | | | | | | |
| Vital signs^{†} | X | | X | | | | X | | | X |
| COVID-19 test | X | | | | | | | | | X |
| Interim history | | | X | | | | X | | | X |
| Focused clinical | | | X | | | | X | | | X |
| examination | | | | | | | | | | |
| Collection of | | | | | | | X | | | X |
| reportable concomitant | | | | | | | | | | |
| medications/vaccinations | | | | | | | | | | |
| Allocation | X | | | | | | | | | |
| subject number | | | | | | | | | | |
| Randomization/Dose | X | | | | | | | | | |
| number | | | | | | | | | | |
| Blood samples | BL0001 | | | | | | BL0002 | | BL00P2 | |
| Nasal swab‡ | | | NS0001 | | | | | | | NS0001 or |
| | | | | | | | | | | UN0001 |
| | | | | | | | | | | to |
| | | | | | | | | | | UN000X |
| Vaccination | X | | | | | | | | | |
| Immediate surveillance | X | | | | | | | | | |
| (30 min) ^{§} | | | | | | | | | | |
| Diary Card (DC) | DC1/eDC | | DC2 | | | | | | | |
| electronic DC (eDC) | | | | | | | | | | |
| provided | | | | | | | | | | |
| DC/eDC reviewed | | DC1/eDC ** | DC1/eDC | DC1/eDC ** | DC2/eDC ** | DC2/eDC ** | DC2 | | X | X |
| DC collected | | | | | | | DC1/DC2 | | | |
| Memory aid (MA) | | | | | | | X | | | |
| provided | | | | | | | | | | |
| MA reviewed | | | | | | | | | X | X |
| Collection of | X | X | X | X | X | | | | | |
| solicited local | | | | | | | | | | |
| respiratory and | | | | | | | | | | |
| systemic reactions^{††} | | | | | | | | | | |
| Collection of | X | X | X | X | X | | | | | |
| unsolicited adverse | | | | | | | | | | |
| events^{††} | | | | | | | | | | |
| Collection of | X | X | X | X | X | | | | | |
| adverse events of | | | | | | | | | | |
| special interest^{††} | | | | | | | | | | |
| Collection of | X | X | X | X | X | | | | | |
| medical attended | | | | | | | | | | |
| adverse events^{††} | | | | | | | | | | |

| | |
|---|---|
| Collection of serious | X |
| adverse events | |

*Non-site visit contacts are to be made by phone at scheduled timepoints in the study.
^{†}Vital signs were collected in the eCRF.
‡All subjects provided a nasal swab sample for quantification of vaccine virus shedding at D7, i.e., 7 days after vaccination. The same nasal swab specimens may also be tested for respiratory pathogens (including COVID-19), if the subject is ill at the time of D7 visit.
^{§} Any unsolicited systemic adverse events occurring within the 30 minutes from vaccine administration were recorded as immediate unsolicited systemic adverse events in the case report book.
** Diary Card (DC)/Electronic Diary Card (eDC) were reviewed by telephone contact.
^{††}The subject's parent/guardian/legally authorized representative recorded information in a DC/eDC about solicited reactions, unsolicited AEs, AESIs and MAAEs from DO to D28 after vaccine administration.
^{‡‡} Phone contact during the RSV season every 2 weeks.
^{§§}Visit 04: Post-RSV season visit in the month after the cessation of the RSV season.
***Illness Visits: Additional nasal swab specimens for the detection of RSV and respiratory pathogens (including COVID-19) were also collected from subjects during illness visits and 48 hours later at any other protocol specified time point in the study including medically attended visits during RSV season. If a subject visited any other non-study doctor/hospital for a serious adverse event (SAE) at any time in the study, a nasal swab sample was obtained at the study site once the subject is discharged, if deemed appropriate by the study investigator. All the nasal swab specimens were collected in the recommended viral transport media tube and were be stored between −60° C. and −80° C. until ready to ship. The requirement for an onsite or at home illness visit was evaluated first by video call to enable remote evaluation of severity and remote management of mild (Grade 1) illness, as deemed appropriate by the study investigator.

Table of Study Procedures for Cohorts 2 and 4 - Vaccination 1 (D0 to D55)
Phase I/II Study, 6 Planned Visits, 40 (42) Non-visit Telephone Contacts, 2 Vaccinations, 4 Planned Blood Samples,
2 Planned Nasal Swabs, up to a Maximum of 12 Months of Participation per Subject

| | Visit (V)/Contact | | | | | | |
|---|---|---|---|---|---|---|---|
| | Visit | Phone contacts | | Phone contacts | Phone contact | Phone contact | Illness Visit(s) ‡ |
| | 01 | 1-6 | Visit 02 | 7-16 | 17 | 18 | Visit 99 |
| | Study timelines - Days (D) | | | | | | |
| | | | | Three times | | | |
| | | | | weekly (×10) | | | Any time between |
| | | | | between D8 and | | | Visit 01 and |
| | D0 | Daily (×6) | D7 | D28 | D29 (+1) | D42 (+1) | Visit 06 |
| In-person visit | X | | X | | | | X |
| Non-visit contact* | | X | | X | X | X | |
| Informed consent | X | | | | | | |
| Demography data | X | | | | | | |
| Inclusion/Exclusion criteria | X | | | | | | |
| Medical history and | X | | | | | | |
| physical examination | | | | | | | |
| Vital signs^{†} | X | | X | | | | X |
| COVID-19 test | X | | | | | | X |
| Interim history | | | X | | | | X |
| Focused clinical examination | | | X | | | | X |
| Collection of | | | | | | | X |
| reportable concomitant | | | | | | | |
| medications/vaccinations | | | | | | | |
| Allocation of subject number | X | | | | | | |
| Randomization/Dose number | X | | | | | | |
| Blood samples | BL0001 | | | | | | |
| Nasal swabs^{‡} | | | NS0001 | | | | NS0001 or UN0001 to |
| | | | | | | | UN0000X |
| Vaccination (1st administration) | X | | | | | | |
| Immediate surveillance | X | | | | | | |
| (30 min) ^{§} | | | | | | | |
| Diary Card (DC)/electronic | DC1/eDC | | DC2 | | | | |
| DC (eDC) provided | | | | | | | |
| DC/eDC reviewed | | DC1/eDC ** | DC1/eDC | DC1/eDC ** | DC2/eDC ** | DC2/eDC ** | X |
| Collection of solicited | X | X | X | X | X | | |
| local respiratory and | | | | | | | |
| systemic reactions** | | | | | | | |
| Collection of | X | X | X | X | X | | |
| unsolicited adverse events** | | | | | | | |
| Collection of adverse | X | X | X | X | X | | |
| events of special interest** | | | | | | | |
| Collection of medical | X | X | X | X | X | | |
| attended adverse events** | | | | | | | |
| Collection of | | | | X | | | |
| serious adverse events | | | | | | | |

*Non-site visit contacts are to be made by phone at scheduled timepoints in the study.
^{†}Vital signs were collected in the eCRF.
‡ All subjects provided a nasal swab sample for quantification of vaccine virus shedding at D7, i.e., 7 days after vaccination 1. The same nasal swab specimen may also be tested for respiratory pathogens (including COVID-19), if the subject is ill at the time of D7 visit.
^{§} Any unsolicited systemic adverse events occurring within the 30 minutes from vaccine administration were recorded as immediate unsolicited systemic adverse events in the case report book.
**Diary Card/Electronic Diary Card (DC/eDC) was reviewed by telephone contact.
††The subject's parent/guardian/legally authorized representative recorded information in a DC/eDC about solicited reactions, unsolicited AEs, AESIs and MAAEs from D0 to D28 after vaccine administration 1 (Acute phase 1).
‡‡Illness Visits (between V01 and V06): Additional nasal swab specimens for the detection of RSV and respiratory pathogens (including COVID-19) were also collected from subjects during illness visits and 48 hours later at any other protocol specified time point in the study including medically attended visits during RSV season. If a subject visited any other non-study doctor/hospital for a serious adverse event (SAE) at any time in the study, a nasal swab sample was obtained at the study site once the subject is discharged, if deemed appropriate by the study investigator. All the nasal swab specimens were collected in the recommended viral transport media tube and were stored between −60° C. and −80° C. until ready to ship. The requirement for an at home or onsite illness visit was evaluated first by video call to enable remote evaluation of severity and remote management of mild (Grade 1) illness, as deemed appropriate by the study investigator.

TABLE of Study Procedures for Cohorts 2 and 4 - Vaccination 2 (D56 to End of Study) Phase I/II Study, 6 Planned Visits, 40 (42) Non-visit Telephone
Contacts, 2 Vaccinations, 4 Planned Blood Samples, 2 Planned Nasal Swabs, up to a Maximum of 12 Months of Participation per Subject

| | Visit (V)/Contact | | | | | | | |
|---|---|---|---|---|---|---|---|---|
| | | | | | | | | Illness |
| | | Phone contacts | | Phone contacts | | Phone contact | | Visit(s) *** |
| | Visit 03 | 19-24 | Visit 04 | 25-30 | Visit 05^{†††} | 31-40 (42)^{‡‡} | Visit 06^{‡‡} | Visit 99 |
| | Study timelines - Days (D) | | | | | | | |
| | | | | | | | 1-31 | |
| | | | | | | | October (SH) | |
| | | | | Twice weekly | | | 1-30 April | |
| | | | | (×6) | | | (NH) | Any time |
| | | | | between | | Every 2 weeks | or >5 months | between |
| | | | | D64 and | | between Visit | after last | Visit 01 and |
| | D56 + 7 | Daily (×6) | D63 (+7) | D83 | D84 (+8) | 05 and Visit 06 | vaccine admin. | Visit 06 |
| In-person visit | X | | X | | X | | X | X |
| Non-visit contact* | | X | | X | | X | | |
| Vital signs^{†} | X | | X | | X | | | X |
| Interim history | X | | X | | X | | | X |
| Focused clinical examination | X | | X | | X | | | X |
| COVID-19 test | X | | | | | | | X |
| Collection of | X | | | | | | | X |
| reportable concomitant | | | | | | | | |
| medications/vaccinations | | | | | | | | |
| Dose Number | X | | | | | | | |
| Blood samples | BL0002 | | | | BL0003 | | BL00P2 | |
| Nasal swabs ^{‡} | | | NS0002 | | | | | NS0002 or |
| | | | | | | | | UN0001 to |
| | | | | | | | | UN000X |
| Vaccination | X | | | | | | | |
| (2nd administration) | | | | | | | | |
| Immediate surveillance | X | | | | | | | |
| (30 min) ^{§} | | | | | | | | |
| Diary Card (DC1) | DC3 | | | | | | | |
| DC/eDC reviewed** | DC2/eDC | DC3/eDC ** | DC3/eDC | DC3/eDC ** | DC3/eDC | | X | X |
| DC collected | DC1/DC2 | | | | DC3 | | | |
| Memory aid (MA) provided | | | | | X | | | |
| MA reviewed | | | | | | | X | X |
| Collection of | X | X | X | X | X | | | |
| solicited local | | | | | | | | |
| respiratory | | | | | | | | |
| and systemic reactions^{††} | | | | | | | | |
| Collection of | X | X | X | X | X | | | |
| unsolicited adverse | | | | | | | | |
| events^{††} | | | | | | | | |
| Collection of adverse | X | X | X | X | X | | | |
| events of special | | | | | | | | |
| interest^{††} | | | | | | | | |
| Collection of medical | X | X | X | X | X | | | |
| attended adverse | | | | | | | | |
| events^{††} | | | | | | | | |

| | |
|---|---|
| Collection of serious | X |
| adverse events | |

*Non-site visit contacts are to be made by phone at scheduled timepoints in the study.
^{†}Vital signs were collected in the eCRF.
^{‡} All subjects provided a nasal swab sample for quantification of vaccine virus shedding at D63, i.e., 7 days after vaccination 2. The same nasal swab specimen was also tested for respiratory pathogens (including COVID-19), if the subject was ill at the time of D63 visit.
^{§} Any unsolicited systemic adverse events occurring within the 30 minutes from vaccine administration were recorded as immediate unsolicited systemic adverse events in the case report book.
**Diary Card (DC)/Electronic Diary Card (eDC) was reviewed by telephone contact.
^{††}The subject's parent/guardian/legally authorized representative recorded information in a DC/eDC about solicited reactions, unsolicited AEs, AESIs and MAAEs from D56 to D84 (Acute phase 2).
^{‡‡}Phone contact during the RSV season, every 2 weeks.
§§Visit 06: Post-RSV season visit - In the month after the cessation of the RSV season in sites with differing seasons or at least 5 months after last vaccine administration. Study subjects enrolled during the routine RSV season (i.e., November to March for NH and May to September for SH) were followed up for at least 5 months after the last vaccine administration.
*** Illness Visits (between V01 and V06): Additional nasal swab specimen for the detection of RSV and respiratory pathogens (including COVID-19) were also collected from subjects during illness visits and 48 hours later at any other protocol specified time point in the study including medically attended visits during the RSV season. If a subject visited any other non-study doctor/hospital for a serious adverse event (SAE) at any time in the study, a nasal swab sample was obtained at the study site once the subject is discharged, if deemed appropriate by the study investigator. All the nasal swab specimens were collected in the recommended viral transport media tube and were stored between −60° C. and −80° C. until ready to ship. The requirement for an at home or onsite illness visit was evaluated first by video call to enable remote evaluation of severity and remote management of mild (Grade 1) illness, as deemed appropriate by the study investigator.
^{†††}Visit 05 is to occur 84 days after the first vaccination at Visit 01 and 28 days after the second vaccination visit at Visit 03.

List of Abbreviations

| | |
|---|---|
| AE | adverse event |
| AESI | adverse event of special interest |
| AR | adverse reaction |
| CDC | Centers for Disease Control |
| CDM | Clinical Data Management |
| CI | confidence interval |
| COVID-19 | Coronavirus Disease 2019 |
| CQA | Clinical Quality Assessment |
| CRA | Clinical Research Associate |
| CRB | (electronic) case report book [all the case |
| | report forms for a subject] |
| CRF | (electronic) case report form |
| D | Day |
| DAIDS | Division of AIDS |
| DC | diary card |
| eDC | Electronic Diary Card |
| EDC | electronic data capture |
| EENT | eye, ear, nose, throat |
| ELISA | enzyme linked immunosorbent assay |
| ESDR | early safety data review |
| FAS | full analysis set |
| FDA | Food and Drug Administration |
| FVFS | first visit, first subject |
| FVLS | first visit, last subject |
| Gcc | G (glycoprotein) central conserved region |
| GCI | Global Clinical Immunology |
| GCP | Good Clinical Practice |
| GMTs | geometric mean titers |
| GMTRs | geometric mean titers ratios |
| GPV | Global Pharmacovigilance |
| HIV | human immunodeficiency virus |
| IATA | International Air Transport Association |
| ICF | informed consent form |
| ICH | International Council for Harmonisation of |
| | Technical Requirements for Pharmaceuticals |
| | for Human Use |
| IDMC | Independent Data Monitoring Committee |
| IEC | Independent Ethics Committee |
| Ig | immunoglobulin |
| IME | important medical event |
| IND | investigational new drug (application) |
| IRB | Institutional Review Board |
| IRT | interactive response technology |
| LCLS | last contact, last subject |
| LID | Laboratory of Infectious Diseases |
| LLOQ | lower limit of quantification |
| LLT | lowest level term |
| LRI | lower respiratory illness |
| MA | memory aid |
| MAAE | medically attended adverse event |
| MAARI | medically attended acute respiratory illness |
| MAD | Mucosal Atomization Device |
| MAALRI | medically attended acute lower respiratory |
| | illness |
| MedDRA | Medical Dictionary for Regulatory Activities |
| mL | milliliter |
| MN | microneutralization |
| nAb | neutralizing antibody |
| NC | Negative Control |
| NIAID | National Institute of Allergy and Infectious |
| | Diseases |
| NIH | National Institutes of Health |
| NS | Nasal Swab |
| NSAID | non-steroidal anti-inflammatory drug |
| PFU | plaque forming units |
| PC | Positive Control |
| PCR | polymerase chain reaction |
| PSB | Product Safety Board |
| POC | Point of care |
| PPAS | per-protocol analysis set |
| PT | preferred term |
| RCDCs | Reverse cumulative distribution curves |
| RSV | Respiratory Syncytial Virus |
| RSV | Recombinant Live-Attenuated Respiratory |
| | Syncytial Virus (RSV) |
| ΔNS2/Δ1313/I1314L | ΔNS2/Δ1313/I1314L (NIH) or ΔNS2/Δ1313/ |
| | I1314L (Sanofi) Vaccine |
| RSV-LRI | RSV-associated lower respiratory illness |
| RSV MAARI | RSV-associated, medically attended acute |
| | respiratory illness |
| RSV MAALRI | RSV-associated, medically attended acute |
| | lower respiratory illness |
| RMO | Responsible Medical Officer |
| RT-PCR | reverse transcriptase-polymerase chain reaction |
| QRT-PCR | quantitative real-time reverse transcriptase- |
| | polymerase chain reaction |
| rRT-PCR | real-time reverse transcriptase-polymerase |
| | chain reaction |
| SAE | serious adverse event |
| SafAS | safety analysis set |
| SMT | Safety Management Team |
| SOC | system organ class |
| TBD | to be determined |
| TMF | trial master file |
| ULOQ | upper limit of quantification |
| URI | upper respiratory tract illness |
| US | United States |
| VAC | Vaccine Adjudication Committee |
| VTM | viral transport medium |
| WHO | World Health Organization |
| wt | wild type |

### Details:

### Rationale for the Study

Prior to RSV ΔNS2/Δ1313/I1314L vaccines as provided herein, 3 RSV vaccine viruses with the NS2 gene deletion (rA2cpΔNS2, rA2cp248/404ΔNS2, and rA2cp530/1009ΔNS2) had been evaluated in clinical studies (Wright et al., 2006, J. Infect Dis., 193 (4): 573-81). The rA2cpΔNS2 candidate was over-attenuated for adults but under-attenuated for use in young children, whereas both rA2cp248/404ΔNS2 and rA2cp530/1009ΔNS2 were over-attenuated and insufficiently immunogenic in seronegative children. Based on these results RSV ΔNS2/Δ1313/I1314L (NIH) was developed. In a recent Phase Ia study sponsored by the National Institutes of Health (NIH) in RSV seronegative children aged 6 to 24 months, (n=20) (Karron et al., 2020, J Infect Dis.; 222 (1): 82-91), at the 10⁶ PFU dose, RSV ΔNS2/Δ1313/I1314L (NIH) was safe, had good infectivity (100% of vaccinated subjects infected) and immunogenicity (80% of subjects with a >4-fold rise in neutralizing antibody titers) and primed for a strong anamnestic response to wild type (wt) RSV infection in the post-RSV season surveillance.

### Study Objectives

### Primary Objectives

To assess the safety profile of each dose of RSV ΔNS2/Δ1313/I1314L (Sanofi) after each and any administration using an intranasal atomization delivery device in all infants and toddlers regardless of baseline serostatus.

To characterize the RSV A serum neutralizing antibody responses to the study product in each vaccine group after vaccination 1 (D56) for Cohorts 1, 2, 3, and 4, and after vaccination 2 (D84) for Cohorts 2 and 4 in RSV naïve subjects.

### Secondary Objectives

To quantify the amount of vaccine virus shed by each participant on D7 for Cohorts 1, 2, 3, and 4, and D63 for Cohorts 2 and 4, measured by quantitative reverse transcriptase polymerase chain reaction (RT-PCR) by baseline serostatus.

To determine the proportion of vaccinated infants and toddlers in each vaccine group infected with the vaccine virus after vaccination 1 (D56) for Cohorts 1, 2, 3, and 4, and after vaccination 2 (D84) for Cohorts 2 and 4 by baseline serostatus. (Infection defined as detection of vaccine in nasal swab sample by polymerase chain reaction (PCR) and/or a ≥4-fold rise in RSV A serum neutralizing antibody titers, or RSV serum anti-F IgG antibody titers.)

To characterize the RSV A serum neutralizing antibody responses to the study product in each vaccine group after vaccination 1 (D56) for Cohorts 1, 2, 3, and 4, and after vaccination 2 (D84) in Cohorts 2 and 4 in RSV experienced subjects.

To characterize RSV serum anti-F IgG antibody responses to the study product in each vaccine group after vaccination 1 (D56) for Cohorts 1, 2, 3, and 4, and after vaccination 2 (D84) for Cohorts 2 and 4 by baseline serostatus.

To characterize RSV serum antibody responses (RSV A-neutralizing and anti-RSV F IgG) to the study product in each vaccine group after the RSV surveillance season or at least 5 months after the last vaccine administration by baseline serostatus.

### Exploratory Objectives

### Exploratory Safety Objective

To assess the safety profile of each dose of RSV after each and any administration by baseline serostatus.

### Exploratory Immunogenicity Objectives:

To characterize the RSV A serum neutralizing antibody responses to the study product converted to IU/mL values in each vaccine group after vaccination 1 (D56) for Cohorts 1, 2, 3, and 4, and after vaccination 2 (D84) for Cohorts 2 and 4 by baseline serostatus.
To characterize the RSV A serum neutralizing antibody responses to the study product converted to IU/mL values after the RSV season or at least 5 months after the last vaccine administration by baseline serostatus.
To characterize the RSV.B serum neutralizing antibody responses to the study product in each vaccine group after vaccination 1 (D56) for Cohorts 1, 2, 3, and 4, and after vaccination 2 (D84) for Cohorts 2 and 4 by antibody serostatus.
To characterize the RSV B serum neutralizing antibody responses to the study product converted to IU/mL values in each vaccine group after vaccination 1 (D56) for Cohorts 1, 2, 3, and 4, and after vaccination 2 (D84) for Cohorts 2 and 4 by antibody serostatus.
To characterize the RSV.B serum neutralizing antibody responses to the study product in each vaccine group after the RSV season or at least 5 months after the last vaccine administration by antibody serostatus.
To characterize the RSV B serum neutralizing antibody responses to the study product converted to IU/mL values after the RSV season or at least 5 months after the last vaccine administration by antibody serostatus.
To characterize RSV A and RSV B serum anti-protein G central conserved region (Gcc) IgG antibody responses to the study product in each vaccine group after vaccination 1 (D56) for Cohorts 1, 2, 3, and 4, and after vaccination 2 (D84) for Cohorts 2 and 4 by baseline serostatus.
To characterize RSV A and RSV B serum anti-Gcc IgG antibody responses to the study product in each vaccine group after the RSV season or at least 5 months after the last vaccine administration by antibody serostatus.
To characterize the RSV serum anti-F IgA antibody responses after vaccination 1 (D56) for Cohorts 1, 2, 3, and 4, and after vaccination 2 (D84) for Cohorts 2 and 4 by baseline serostatus.
To characterize the RSV serum anti-F IgA antibody responses after the RSV season or at least 5 months after the last vaccine administration by baseline serostatus.

### Exploratory Efficacy Objective

To describe the frequency and severity of RSV-associated, medically attended, acute respiratory illness (RSV MAARI) and RSV-associated, medically attended, acute lower respiratory illness (RSV MAALRI) in all infants and toddlers in each vaccine group during the RSV season or at least 5 months after last vaccine administration.

### Primary Safety Endpoints:

In all infants and toddlers regardless of baseline serostatus:
Occurrence of any unsolicited systemic AEs reported in the 30 minutes after each and any vaccination.
Occurrence of solicited (i.e., pre-listed in the subject's DC/eDC and in the CRB) administration site and systemic reactions within 28 days after each and any vaccination (i.e., Acute Phase).
Occurrence of any unsolicited (spontaneously reported) AEs within 28 days after each and any vaccination.
Occurrence of any AESIs within 28 days after each and any vaccination.
Occurrence of any MAAEs within 28 days after each and any vaccination.
Occurrence of any SAEs throughout the study.
Other safety endpoints will be recorded or derived as described in the statistical analysis plan. Depending on the item, these could include: nature (Medical Dictionary for Regulatory Activity [MedDRA] preferred term), time of onset, duration, number of days of occurrence, intensity, relationship to vaccine, action taken, whether the AE led to early termination from the study, seriousness, or outcome.

### Primary Immunogenicity Endpoint:

RSV A serum neutralizing antibody titers by D56 for Cohorts 1, 2, 3, and 4 and by D84 for Cohorts 2 and 4 in RSV naïve subjects.

For Sponsor's Phase I/II study, data will be analyzed according to baseline serostatus. Baseline serostatus will be retrospectively determined from serum samples collected at baseline (V01). Participants will be categorized into RSV-experienced or RSV-naïve based on the presence or absence of detectable RSV serum anti-F IgA antibodies. This biomarker has been chosen since it is produced only in response to RSV infection and not transferred trans placentally from mother to child.

### Investigational Plan

### Description of the Overall Study Design and Plan

### Study Design

This is a Phase I/II, randomized, observer-blind, placebo-controlled, multi-center, dose-finding study to evaluate the safety, immunogenicity, infectivity, and vaccine virus shedding after 1 or 2 administrations of a live-attenuated RSV ΔNS2/Δ1313/I1314L (Sanofi) vaccine using an intranasal atomization delivery device in infants and toddlers 6 months to 18 months (Cohorts 1 to 4) of age in the United States, Canada, Latin America (Argentina, Chile, and Honduras), and South Africa.

### Vaccination:

A total of 300 infants and toddlers 6 to 18 months of age were or will be enrolled into 1 of 4 cohorts, sequentially, and within each cohort were or will be randomized to receive intranasal administration of their assigned study product using an intranasal atomization delivery device as follows:
Cohort 1—40 infants and toddlers received or will receive 1 administration, 1:1 ratio of RSV ΔNS2/Δ1313/I1314L (Sanofi) 5.6 log₁₀ PFU (low-dose) or placebo (same formulation buffer as the RSV ΔNS2/Δ1313/I1314L (Sanofi) vaccine)
Cohort 2—40 infants and toddlers received or will receive 2 administrations, 1:1 ratio of RSV ΔNS2/Δ1313/I1314L (Sanofi) 5.6 log₁₀ PFU (low-dose) or placebo
Cohort 3—40 infants and toddlers received or will receive 1 administration, 1:1 ratio of RSV ΔNS2/Δ1313/I1314L (Sanofi) 6.2 log₁₀ PFU (high-dose) or placebo
Cohort 4—180 infants and toddlers received 2 administrations, 1:1:1 of RSV ΔNS2/Δ1313/I1314L (Sanofi) 5.6 log₁₀ PFU (low-dose), RSV ΔNS2/Δ1313/I1314L (Sanofi) 6.2 log₁₀ PFU (high-dose) or placebo.

### Cohort 1 (Northern Hemisphere):

1:1 randomized, placebo-controlled, 1 dose of RSV ΔNS2/Δ1313/I1314L (Sanofi) (5.6 log₁₀ PFU), 1 administration (0.2 mL in total per administration) at Day (D) 0, n=20 per vaccine group. Subject enrollment was initiated at approximately 15 sites in the US. Vaccine administration for enrolled subjects was completed at least 5 days before the beginning of RSV season (the average 5-month RSV season in the northern hemisphere is 1 November to 31 March). Since recruitment in Cohort 1 did not reach the goal of n=40, no attempt was made to include more subjects in subsequent cohorts.

### Cohort 2 (Southern Hemisphere):

1:1 randomized, placebo-controlled, 1 dose of RSV ΔNS2/Δ1313/I1314L (Sanofi) (5.6 log₁₀ PFU), 2 administrations (0.2 mL in total per administration) at D0 and D56, n=20 per vaccine group. Subject enrollment was initiated at approximately 4 sites in Latin America (Argentina and Chile) at the earliest in November/December 2020. The first and the second vaccine administrations (DO and D56, respectively) for enrolled subjects were completed at least 5 days before the beginning of the RSV season (the average 5-month RSV season in the southern hemisphere is 1 May to 30 September). If recruitment in Cohort 1 did not reach the goal of n=40, no attempt was made to include more participants in subsequent cohorts.

### Cohort 3 (Northern Hemisphere):

1:1 randomized, placebo-controlled, 1 dose of RSV ΔNS2/Δ1313/I1314L (Sanofi) (6.2 log₁₀ PFU), 1 administration (0.2 mL per administration in total) at D0, n=20 per vaccine group. Subject enrollment was initiated at approximately 30 sites in the US in April 2021. Vaccine administrations for enrolled subjects were completed before 31 May 2021. Since recruitment in Cohort 3 did not reach the goal of n=40, no attempt was made to include more subjects receiving 1 administration in Cohort 4.

### Cohort 4 (Northern and Southern Hemisphere):

1:1:1 randomized, placebo-controlled, 2 doses of RSV ΔNS2/Δ1313/I1314L (Sanofi) (5.6 log₁₀ PFU or 6.2 log₁₀ PFU), 2 administrations (0.2 mL per administration in total) at DO and D56, n=60 per vaccine group. Subject enrollment was initiated at approximately 30 sites in the US at the earliest in June 2021, approximately 2 sites in Canada at the earliest in May 2022, approximately 2 sites in Chile at the earliest in November 2021, approximately 2 sites in Honduras at the earliest in April 2022, and approximately 2 sites in South Africa at the earliest in June 2022.

The first and the second vaccine administrations for enrolled subjects was completed at any time of the year, including the winter RSV season, regardless of whether normal RSV seasonality is restored after the disruption due to COVID-19 non-pharmaceutical interventions.

### Blood Sampling:

All subjects screened for inclusion in Cohorts 1, 2, 3, and 4 provided a blood sample at enrollment (Visit 01) for baseline RSV serum antibody testing.

All subjects in each vaccine group in Cohorts 1, 2, 3, and 4 provided a blood sample at the D56 Visit (before vaccination 2 for Cohorts 2 and 4) for the measurement of post-vaccination serum antibody titers to RSV. All subjects in each vaccine group in Cohorts 2 and 4 provided a blood sample at the D84 visit (28 days post-vaccination 2) for the measurement of post-vaccination 2 serum antibodies to RSV. All subjects in Cohorts 1, 2, 3, and 4 provided a blood sample during the month following the RSV season or at least 5 months after the last vaccine administration for the measurement of post-season RSV antibody titers to determine if a 4-fold or greater rise in RSV antibody titers has occurred during the RSV season, indicating infection with wild-type RSV that was not detected by surveillance.

### Nasal Swab Samples:

Nasal swab samples were collected in all enrolled subjects at D7 for Cohorts 1, 2, 3, and 4; and on D63 for Cohorts 2 and 4 for the following:
Quantification of vaccine virus shedding on D7 for Cohorts 1, 2, 3, and 4, and D63 for Cohorts 2 and 4.
The same nasal swab specimens were tested for respiratory pathogens if the subject was ill at the same time points (D7 for Cohorts 1, 2, 3, and 4, and D63 for Cohorts 2 and 4).
A nasal swab specimen for the detection of RSV and respiratory pathogens were collected from subjects during illness visits and 48 hours later at any other protocol-specified time point in the study including the medically attended surveillance during the RSV season.

### Collection of Safety Data:

The Acute Phase for the first vaccine administration begins on DO post-vaccination and ends at midnight on D28. The Acute Phase for the second vaccine administration begins on D56 post-vaccination and ends at midnight on D84. Any adverse reactions, AEs, AESIs, MAAEs, or SAEs that begin within the Acute Phase (i.e., within 28 days after a vaccination) but are diagnosed by a medical professional after 28 days were still considered as occurring within the Acute Phase.

The Post-Acute Phase for subjects receiving 1 administration begins at 12:01 am on D29 and ends at midnight on D56. The first Post-Acute Phase for subjects receiving 2 administrations begins at 12:01 am on D29 and ends at midnight on D56, except if second administration is exactly on D56 when it ends immediately before receipt of the second administration. The second Post-Acute Phase for subjects receiving 2 administrations begins at 12:01 am on D85 and ends at midnight on D112.

All subjects were observed for 30 minutes after each vaccine administration and any unsolicited systemic adverse events (AEs) occurring at that time were recorded as immediate unsolicited systemic AEs in the case report book (CRB).

All subjects were followed for solicited administration site reactions and systemic reactions, unsolicited adverse events, adverse events of special interest (AESIs) and medically attended adverse events (MAAEs) within 28 days after each and any vaccination, and from vaccination to the end of study participation for serious adverse events (SAEs).

The subject's parent/guardian/legally authorized representative recorded information in a Diary Card (DC)/Electronic Diary Card (eDC) to capture solicited reactions, unsolicited AEs, AESIs and MAAEs from DO to D28 for Cohorts 1, 2, 3, and 4 and D56 to D84 for Cohorts 2 and 4. Parents/guardians/legally authorized representatives alerted the study site regarding reactions, AEs and any symptoms suggestive of respiratory tract illness. The study sites followed up via phone calls. The eDC allowed daily safety monitoring of the study participant also. In certain cases, when an eDC cannot be used, a paper diary card was used for daily safety monitoring. All solicited reactions were graded by the Sponsor Intensity scale, except for runny nose/rhinorrhea and stuffy or blocked nose/nasal congestion which were graded by the Division of AIDS (DAIDS) scale. Adverse events of special interest were graded by the DAIDS scale, except for wheeze, which was graded according to Brighton Collaboration specifications.

Based on previous National Institutes of Health (NIH) clinical experience with live-attenuated RSV vaccine candidates and Sponsor Safety Guideline Standard Practice for the Collection, Analysis, and Reporting of Safety Data, the following predefined solicited reactions were assessed during the Acute Phase for each vaccine administration: administration site reactions including runny nose and stuffy or blocked nose/nasal congestion and systemic reactions including fever, vomiting, crying abnormally, drowsiness, appetite loss, and irritability.

The following AESIs were assessed during the Acute Phases of the study: Acute otitis media, upper respiratory tract illness (URI) including pharyngitis and cough without lower respiratory tract illness (LRI), lower respiratory tract illness (LRI) including stridor, rales tachypnea, acute wheeze, pneumonia, and laryngotracheobronchitis. Immediate hypersensitivity reactions including urticaria, anaphylaxis, or other immunoglobulin (Ig) E-mediated responses are possible as with any vaccine. MAAEs were collected during the Acute Phase for either vaccination using the same process as other AEs. SAEs were recorded throughout the subject's participation in the study. The subject's parent/guardian/legally authorized representative was asked to notify the site immediately about any potential SAE at any time during the study. In addition, the subject's parent/guardian/legally authorized representative recorded information in a DC/eDC about SAEs from the DO to D56 visits (Cohorts 1 and 3), and from DO to D84 visits (Cohorts 2 and 4). Subject's parent/guardian/legally authorized representative received a memory aid (MA) to record the SAEs from the D56 visit until the end of the study (Cohorts 1 and 3) and from the D84 visit until the end of the study (Cohorts 2 and 4). The completed DC/eDC or MA were reviewed with the subject's parent/guardian/legally authorized representative at each visit.

### Justification of the Study Design

For this Phase I/II trial, data will be analyzed according to serostatus. Baseline serostatus will be retrospectively determined from serum samples collected at baseline. Participants will be categorized into RSV-experienced or RSV-naïve based on the presence or absence of detectable serum RSV anti-F IgA antibodies. This biomarker has been chosen since it is produced only in response to RSV infection and not transferred trans-placentally from mother to child.

### Evaluation of Post-Vaccination Immunogenicity on Day 56 for Cohorts 1, 2, 3, and 4 and Day 84 for Cohorts 2 and 4:

To date, all studies with live attenuated RSV candidate vaccines have used Day 56 as the time point to assess serum antibody response post-vaccination with a single dose administration. This time point is used for two reasons: 1) since this is a primary response (rather than an anamnestic [memory] response), serum antibody response may not have reached maximal levels by Day 28 and 2) maximal replication of these highly attenuated vaccine viruses does not typically occur until Day 7, which may also delay induction of the immune response. In subjects receiving 2 administrations of vaccine, this is no longer likely to be a primary response and rather an anamnestic (memory) response and serum antibody response is likely to be maximal by Day 28 after the second vaccination.

### RSV Season or Post-Vaccination RSV Surveillance for Cohort 4:

Based on previous data regarding the seasonality of RSV in the US, Canada, Chile, Honduras, and South Africa, surveillance for RSV-associated disease was largely conducted during the NH (1st November to 31st March) and SH (1st May to 30 September) RSV seasons respectively, adjusted for local site RSV seasonality. Due to the disruption of the seasonal pattern of RSV globally, it is unclear when the normal seasonal transmission will resume though there is some suggestion of a rebound in the winter of 2021-2022 in the NH. Because of the unpredictability of the RSV season due to the impact of COVID-19 nonpharmaceutical interventions, study subject enrollment and vaccination continued regardless of RSV activity. Study subjects enrolled before the routine winter RSV season for that locality were followed up until the consequent month after the end of the routine season (i.e., April for NH and October for SH). Study subjects enrolled during the routine RSV season (i.e., November to March for NH and May to September for SH) were followed up for at least 5 months after the last vaccine administration. During the RSV season or post-vaccination RSV surveillance, study subjects were monitored for symptomatic medically attended respiratory illness. Note, this surveillance may overlap with the Acute and Post-Acute phases. In this case, evaluations required for each of the relevant phases of the study were conducted.

Evaluation of Two Vaccine Doses 5.6 Log₁₀ and 6.2 Log₁₀ PFU:

Previous Phase Ia studies done by the NIH have demonstrated that the 10⁶ PFU dose of RSV ΔNS2/Δ1313/I1314L (NIH) vaccine resulted in better infectivity and neutralizing antibody seroresponse than the 10⁵ PFU dose, with no evidence of an excess of respiratory tract illness associated with receipt of the higher dose (10⁶ PFU). However, in these studies, the vaccine was administered intranasally using a sterile, needle-less 1 mL oral syringe, at a volume of 0.5 mL as nasal drops (approximately 0.25 mL per nostril). In this study, the vaccine is administered at a total volume of 0.2 mL as a fine nasal mist (approximately 0.1 mL per nostril) by an intranasal atomizer, the MAD Nasal 130 device, requiring a re-evaluation of the safety of both the 10⁵ PFU and 10⁶ PFU doses when administered by the intranasal atomizer. Use of an intranasal atomizer may improve the overall delivery of the vaccine and may increase replication in both doses. Use of the atomizer may also improve the infectivity of the low dose (10⁵ PFU). The 10⁵ PFU and 10⁶ PFU doses were the minimal doses explored using the MAD Nasal 130 device. The target was at least as high 5.6 log₁₀ PFU and 6.2 log₁₀ PFU, and therefore the doses were most likely higher due to manufacturing requirements/variability.

### Evaluation of Two Administrations of Vaccine Doses:

There are several published studies evaluating 2 or 3 administrations of attenuated RSV or parainfluenza virus type 3 (PIV3) candidate vaccines. Generally, there was a high level of restriction of the second administration, and limited serum immune responses. The main effect was vaccine infectivity in the individuals who did not respond to the first administration. Two administrations of RSV ΔNS2/Δ1313/I1314L (Sanofi) may improve the infectivity of either dose.

### Selection of Vaccine Dose Using Safety, Infectivity, Viral Shedding and Immunogenicity in Infants and Toddlers Regardless of Baseline RSV Serostatus:

An unblinded interim analysis for dose selection of RSV ΔNS2/Δ1313/I1314L (Sanofi) vaccine for subjects from Cohorts 1, 2, and 3 and at least 90 subjects enrolled in Cohort 4 is included. The interim analysis took place when subjects provided safety data up to the D84 timepoint and D84 immunogenicity results were available. While RSV seronegative infants and toddlers are most likely to be RSV naïve and exhibit sufficient vaccine virus infectivity, shedding and immunogenicity (from serum neutralizing antibody titers); a vaccine dose was selected based on a benefit: risk assessment based on data on our target population for this vaccine (i.e., all infants and toddlers regardless of serostatus). The selected dose will be used for future studies.

### Placebo Group:

Placebo recipients were included in each cohort to establish the background rates of respiratory and febrile illnesses that occur in infants and toddlers.

### Clinical Hypothesis:

Both doses of the live attenuated RSV ΔNS2/Δ1313/I1314L (Sanofi) vaccine are anticipated to be safe and immunogenic in infants and toddlers. However, administration of either dose may be associated with adverse events that become apparent only when analyzed in a larger number of subjects than the numbers enrolled in Laboratory of Infectious Disease (LID)/National Institute of Allergy and Infectious Diseases (NIAID)/NIH Phase Ia studies, where each study assessed 10 to 40 vaccinees. Additionally, the rate, magnitude, and durability of antibody responses may differ according to dose, as well as the magnitude of the memory (anamnestic) seroresponse observed following naturally occurring RSV infection. The intranasal atomizer device used in this study may also impact the safety and immunogenicity of the vaccine. Future dose selection will be based on a descriptive comparison of the safety, infectivity, and immunogenicity of 1 or 2 administrations of either dose category.

### Safety Plan:

TABLE 2
Schedule of blood sample collection
(volume in mL) - Cohorts 1 and 3

| | Visit Number | | | |
|---|---|---|---|---|
| | Visit 01 | Visit 02 | Visit 03 | Visit 04 |
| | Study Timelines | | | |
| | | | | Post-RSV |
| | D 0 | D 7 | D 56 + 7 | season* |
| | Time Windows (Days) | | | |
| Vaccination | X | | | |
| Blood Samples (mL) | 5† | — | 5 | 5* |
| Total volume of | 5 | — | 5 | 5 |
| blood collected (mL) | | | | |

†All subjects screened provided a blood sample up to 5 mL at enrollment (Visit 01) prior to receipt of the first administration of assigned study product, for baseline RSV serum antibody testing.
*All subjects provided a blood sample up to 5 mL during the month following the RSV season or at least five months after the last vaccine administration for the measurement of post-season RSV antibody titers, respectively, to determine if a 4-fold or greater rise in RSV antibody titers has occurred during the RSV season.

TABLE 3
Schedule of blood sample collection (volume in mL) - Cohorts 2 and 4

| | Visit Number | | | | | |
|---|---|---|---|---|---|---|
| | Visit 01 | Visit 02 | Visit 03 | Visit 04 | Visit 05 | Visit 06 |
| | Study Timelines | | | | | |
| | | | | | | Post-RSV |
| | D0 | D7 | D56 + 7 | D63 + 7 | D84 + 8 | season^{‡} |
| | Time Windows (Days) | | | | | |
| Vaccination | X | | X | | | |
| Blood samples (mL) | 5† | — | 5* | — | 5* | 5^{‡} |
| Total volume of blood | 5 | — | 5 | — | 5 | 5 |
| collected (mL) | | | | | | |

†All subjects screened provided a blood sample up to 5 mL at enrollment (Visit 01) prior to receipt of the first administration of assigned study product, for baseline RSV serum antibody testing.
*All the subjects provided a blood sample up to 5 mL at the D56 Visit (before vaccination 2), and then at D84 Visit for the measurement of post-vaccination serum antibodies to RSV.
^{‡}All subjects provided a blood sample up to 5 mL during the month following the RSV season or at least five months after the last vaccine administration for the measurement of post-season RSV antibody titers, to determine if a 4-fold or greater rise in RSV antibody titers has occurred during the RSV season.

### Visit Procedures

### In-Person Visits

### Visit 01 (Day 0) for Cohorts 1, 2, 3, and 4

Timelines for the end of enrollment (Visit 01), vaccination, and RSV surveillance per hemisphere are shown in Table 4.

TABLE 4
Timelines for end of enrollment and vaccination per hemisphere

| | Infants and Toddlers 6 to 18 Months of Age | | | |
|---|---|---|---|---|
| | | Southern Hemisphere | | Northern and Southern |
| | Northern Hemisphere | (Latin America) | Northern Hemisphere | Hemispheres (USA and |
| | (USA) Cohort 1 | Cohort 2 | (USA) Cohort 3 | Latin America) Cohort 4 |
| | (1 administration) | (2 administrations) | (1 administration) | (2 administrations) |
| Earliest day for enrollment | 1 Aug. 2020 | 1 Nov. 2020 | 1 Apr. 2021 | USA - 1 Jun. 2021 |
| (Visit 01) | | | | Canada - May 2022 |
| | | | | Chile - November 2021 |
| | | | | Honduras - April 2022 |
| | | | | South Africa - June 2022 |
| Last day for enrollment | 5 days before the start of | 61 days before the start of | 31 May 2021 | Not applicable |
| (Visit 01) | the RSV season | the RSV season | | |
| Last day for Visit 03 | — | 5 days before the start of | — | Not applicable |
| (D56) Vaccination 2 | | the RSV season | | |

### Visit 01 (Day 0): Enrollment, Randomization, and Vaccination Procedures for Cohorts 1, 2, 3, and 4 (all Subjects)

1) Give the subject's parent/guardian/legally authorized representative information about the study, obtain written informed consent, and give him/her a signed copy.
2) Collect demographic data (including date of birth, sex, race, and ethnicity).
3) Check inclusion and exclusion criteria for eligibility.
4) Obtain verbal medical history about the subject, including past and ongoing medication, any signs and symptoms, and developmental delays.
5) Review any past medical records including the immunization record to ensure subject will not meet exclusion criteria 2, 3, and 21.
6) Prior to randomization, conduct a full physical examination including body temperature, heart rate, respiratory rate, weight, length and assessment of HEENT [head, ears, eyes, nose, and throat], lungs, heart, and abdomen, musculoskeletal, age-appropriate neurological, and skin exam.
7) Perform Coronavirus Disease 2019 (COVID-19) point of care diagnostic test (POC).
8) Contact the IRT for subject number, randomization, and dose number based on age sub-group.
9) Obtain the first blood sample to test the baseline RSV antibody serostatus, i.e., serum RSV-nAb titer
10) Administer the appropriate study product, follow the instructions provided for the intranasal administration of study vaccine using the MAD Nasal 130 device.
11) Keep the subject under observation for 30 minutes and record any adverse reaction in the source document.
12) Give or download the parent/guardian/legally authorized representative a diary card (DC1)/eDC and a thermometer and go over the instructions for their use, and remind to complete the Day 0-7 pages of DC1/eDC. The parent/guardian/legally acceptable representative will record the child's temperature and signs of illness (solicited reactions and unsolicited AEs, including AESIs, MAAEs, and SAEs) on the DC1/eDC.
13) Remind the parent/guardian/legally authorized representative to expect a telephone call from the study site nurse daily after this immunization Visit 01 to review what they have entered daily in the DC1/eDC; and to complete the remaining entries of DC1/eDC and bring to Visit 02 at the specified date.
14) Remind the parent/guardian/legally authorized representative to notify the site in case of an SAE or any illness. The site staff, upon evaluation, will schedule an Illness Visit within the stipulated time frame of site notification, if required.
15) Complete the relevant case report forms (CRFs) for this visit.

### Visit 02: Day 07 Post-Vaccination 1 Visit Procedures for Cohorts 1, 2, 3, and 4 (all Subjects)

1) Review medical history, including past and ongoing medication since the last visit.
2) Perform a focused clinical examination including temperature, pulse, and respirations, ears, eyes, nose, and throat (EENT), respiratory, cardiac, and lymphatic system.
3) Obtain a nasal swab sample for viral quantification of vaccine virus shedding. If the subject meets criteria for an Illness Visit at Day 7, the same nasal swab specimens may also be tested for respiratory pathogens.
4) Review the solicited reactions and unsolicited AEs, AESIs, MAAEs, and SAEs in the completed DC1/eDC with the subject's parent/guardian/legally authorized representative. Provide instruction to complete DC2.
5) If an SAE occurred, follow the instructions for reporting. Remind to notify the site in case of an SAE.
6) Complete the relevant CRFs for this visit.
7) Remind the parent/guardian/legally acceptable representative of the following visit.

### Visit 03: Day 56+7 Post-Vaccination 1 Visit Procedures for Cohort 1 and 3

1) Review medical history, including past and ongoing medication since the last visit. Check for contraindications.
2) Perform a focused clinical examination including temperature, pulse, and respirations, EENT, respiratory, cardiac, and lymphatic system.
3) Obtain a blood sample for serum antibodies to RSV.
4) Collect and review SAEs in the completed DC1 and DC2/eDC with the subject's parent/guardian/legally authorized representative. Provide MA.
5) If an SAE had occurred, follow the instructions herein for reporting it. Remind to notify the site in case of an SAE.
6) Complete the relevant CRFs for this visit.
7) Remind the parent/guardian/legally authorized representative of the following visit.

### Visit 03: Day 56+7 Post-Vaccination 1 Visit Procedures for Cohorts 2 and 4

1) Obtain verbal medical history about the subject, including past and ongoing medication and immunizations.
2) Check for contraindications for receipt of second vaccination.
3) Prior to vaccination, conduct a focused clinical examination including temperature, pulse, and respirations, EENT, respiratory, cardiac, and lymphatic system.
4) Perform COVID-19 POC diagnostic test.
5) Collect reportable concomitant medications/vaccinations.
6) Contact the IRT for dose number.
7) Obtain a blood sample for serum antibodies to RSV.
8) Administer the appropriate study product, follow the instructions provided for the intranasal administration of study vaccine using the MAD Nasal 130 device.
9) Keep the subject under observation for 30 minutes and record any adverse reaction in the source document.
10) Collect and review solicited reactions and unsolicited AEs, including AESIs, MAAEs, and SAEs in the completed DC1 and DC2/eDC with the subject's parent/guardian/legally authorized representative.
11) Give or download the parent/guardian/legally authorized representative a diary card (DC3)/eDC and a thermometer and go over the instructions for their use, and remind to complete the Day 0-7 pages of DC3/eDC. The parent/guardian/legally authorized representative will record the child's temperature and signs of illness (solicited reactions and unsolicited AEs, including AESIs, MAAEs, and SAEs) on the DC3/eDC.
12) Remind the parent/guardian/legally authorized representative to expect a telephone call from the study site nurse daily after this Visit 04 to review what they have entered daily in the DC3/eDC, and to complete the entries of DC3 and bring to Visit 05 at the specified date and time.
13) Remind the parent/guardian/legally authorized representative to notify the site in case of an SAE or any illness. The site staff, upon evaluation, will schedule an Illness Visit within the stipulated time frame of site notification, if required.
14) Complete the relevant CRFs for this visit.
   Post-RSV Season or ≥5 Months after Last Vaccine Administration Visit for all Cohorts (Visit 04 for Cohorts 1 and 3 or Visit 06 for Cohorts 2 and 4)
1) Obtain a blood sample for serum antibodies to RSV.
2) Review the MA for SAEs with the subject's parent/guardian/legally authorized representative.
3) If an SAE had occurred, follow the instructions herein for reporting it. Remind to notify the site in case of an SAE.
4) Complete the relevant CRFs for this visit and the End of Study CRF.

### Visit 04: D63+7 Post-Vaccination 1 (Day 07 Post-Vaccination 2) Visit Procedures for Cohorts 2 and 4

1) Review medical history, including past and ongoing medication since the last visit.
2) Perform a focused clinical examination including temperature, pulse, and respirations, EENT, respiratory, cardiac, and lymphatic system.
3) Obtain a nasal swab sample for quantification of vaccine virus shedding. If the subject meets criteria for an Illness Visit, the same nasal swab specimens may also be tested for respiratory pathogens.
4) Review the solicited reactions and unsolicited AEs, AESIs, MAAEs, and SAEs in the completed DC3/eDC with the subject's parent/guardian/legally authorized representative.
5) If an SAE occurred, follow the instructions herein for reporting it. Remind to notify the site in case of an SAE.
6) Complete the relevant CRFs for this visit.

### Visit 05: D84+8 Post-Vaccination 1 (Day 28+8 Post-Vaccination 2) Visit Procedures for Cohorts 2 and 4

1) Review medical history, including past and ongoing medication and immunizations since the last visit.
2) Perform a focused clinical examination including temperature, pulse, and respirations, EENT, respiratory, cardiac, and lymphatic system.
3) Obtain a blood sample for serum antibodies to RSV.
4) Collect and review the solicited reactions and unsolicited AEs, AESIs, MAAEs, and SAEs in the completed DC3/eDC with the subject's parent/guardian/legally authorized representative. Provide MA.
5) If an SAE had occurred, follow the instructions herein for reporting it. Remind to notify the site in case of an SAE.
6) Complete the relevant CRFs for this visit.

### Visit 99: Illness Visits

Illness visits can be conducted remotely, as a home visit or an onsite visit depending on the judgment of the investigator. The requirement for an illness visit and which type of illness visit were evaluated first by video call. The video call was used to initially and remotely evaluate the study subject's clinical status and whether the illness can be managed remotely especially for mild (Grade 1) illness. This staged management of illness visits is required during the current COVID-19 pandemic situation to limit the number of contacts between the study subject and study site staff to only those that are absolutely required.

The timeframe after site notification in which the Illness Visit must occur, if deemed necessary by the Investigator, depends on grading of the fever and respiratory symptoms and the phase of the study. If the Illness Visit occurs on a day concurrent with a routine Study Visit when a nasal swab specimen is to be collected, the same nasal swab specimen would be used to test for respiratory pathogens. A second nasal swab should be collected 48 hours later. If the Illness Visits occurs on a day concurrent with a routine Study Visit when a nasal swab specimen is not collected, a nasal swab specimen is required to test for respiratory pathogens. A second nasal swab should be collected 48 hours later.

If illnesses/safety events occur, the study subject should be managed according to the site/local standard of care, including the conduct of lab investigations required for diagnosis and/or management of the study subject. Following an Illness Visit, medical personnel should continue to follow subjects until resolution.

Illness visits may occur at any time during the study. The Illness visit timeframes are summarized in Table 5.

TABLE 5
Illness visit timeframe

| | | | Visit |
|---|---|---|---|
| Phase | Symptom | Grade | timeframe |
| Acute (post-vaccination | Fever, acute otitis | 1 | Within 3 days |
| to midnight 28 days | media, or URI | >1 | Within 2 days |
| after each vaccination)* | LRI | Any | Within 2 days |
| | SAEs | Any | Within 2 days |
| Post-acute (12:01 am | SAEs | Any | Within 2 days |
| on 29^{th} day after post- | | | |
| vaccination to midnight | | | |
| 56 days after each | | | |
| vaccination)* | | | |
| RSV surveillance | Medically attended | >2 | Within 3 days |
| season (on average, | fever, acute otitis | | |
| 1 November to 31 | media, URI, or LRI | | |
| March for NH and 1 | SAEs | Any | Within 2 days |
| May to 30 September | | | |
| for SH)* | | | |
| Post-RSV season (on | SAEs | Any | Within 2 days |
| average, 1 to 31 | | | |
| April for NH and 1 | | | |
| to 31 October for SH) | | | |
| or at least 5 months | | | |
| after last vaccine | | | |
| administration | | | |

LRI, lower respiratory tract illness;
NH, Northern hemisphere;
RSV, Respiratory Syncytial Virus;
SAE, serious adverse event;
SH, Southern hemisphere;
URI, upper respiratory tract illness
*In some circumstances, acute phase or post-acute phase may overlap with the RSV surveillance season, in which case the more conservative visit timeframe should be considered.

### Illness Visit Procedures are as Follows:

1) Obtain verbal medical history about the subject, including past and ongoing medication and immunizations.
2) Determine whether the subject can be managed remotely or if an at home visit or an onsite illness visit is required.
3) If an at home or onsite visit, perform a focused clinical examination including temperature, pulse, and respirations, EENT, respiratory, cardiac, and lymphatic system.
4) Collect and review the solicited and unsolicited AEs, AESIs, MAAEs, and SAEs in the completed DC/eDC or memory aid with the subject's parent/guardian/legally authorized representative.
5) If an SAE had occurred, follow the instructions herein for reporting it. Remember to notify the site in case of an SAE, if illness starts within the 28 days of vaccination.
6) Obtain a nasal swab sample for RSV viral detection and quantification and real-time reverse transcriptase-polymerase chain reaction (rRT-PCR) for respiratory pathogens.
7) COVID-19 testing if required according to the opinion of the investigator and CDC COVID testing algorithm (for CDC COVID testing algorithm, see cdc.gov/coronavirus/2019-ncov/lab/resources/Antigen_Testing_Algorithm_2020-12-14_v03_NO_DRAFT_SPW_508.pdf).
8) Complete the relevant CRFs for this visit.
9) Schedule follow-up as appropriate, including a visit for the collection of a confirmation nasal swab 48 hour later.
10) If illnesses/safety events occur, the subject should be managed according to the site/local standard of care, including the conduct of lab investigations required for diagnosis and/or management of the study subject.

Follow-up of subjects with solicited reactions or with AEs that led to study/vaccination discontinuation:

Unless a subject or subject's parent/guardian/legally authorized representative refuses further contact, each subject who experiences an AE (whether serious or non-serious) during the study must be followed until the condition resolves, becomes stable, or becomes chronic (even after the end of the subject's participation in the study) if either of the following is true:
1. The AE is considered by the Investigator to be related to the product administered.
2. The AE caused the discontinuation of the subject from the study or from vaccination.

### Non-Visit Telephone Contacts

### Acute Phase Telephone Contacts to Review the DCs Up to D29+1 (Cohorts 1, 2, 3, and 4) and D84+8 (Cohorts 2 and 4)

Note: If any of the Acute Phase non-visit contacts fall on a weekend or a holiday, the telephone call may be made on the following business day. All telephone contacts with the subject's parent/guardian/legally authorized representative must be made by a qualified person, such as a physician or qualified study nurse.
1) Review the information entered in the DC for solicited reactions and unsolicited AEs, AESIs, MAAEs, and SAEs. The site staff upon evaluation, if required, will schedule an Illness Visit within the appropriate time window of site notification.
2) Record relevant information concerning the subject's health status including any changes in medications and immunizations on the telephone contact form, and in the CRF, if required. The site staff, upon evaluation, will schedule an Illness Visit within the stipulated time frame of site notification, if required.
3) If an SAE had occurred, follow the instructions herein for reporting it. Also, remind to notify the site in case of an SAE.
4) Remind the parent/guardian/legally authorized representative to do the following:
   Complete the remaining pages of the diary card/electronic diary card and bring them to next visit.
   Notify the site in case of an SAE.

### Day 42+1 Telephone Contact (Cohorts 1, 2, 3, and 4)

1) Record relevant information concerning the subject's health status including any changes in medications and immunizations on the telephone contact form, and in the CRF, if required. The site staff, upon evaluation, will schedule an Illness Visit within the stipulated time frame of site notification, if required.
2) If an SAE had occurred, follow the instructions herein for reporting it. Also, remind to notify the site in case of an SAE.
3) Remind the parent/guardian/legally authorized representative to do the following:
   Complete the remaining pages of the diary card/electronic diary card and bring them to next visit.
   Notify the site in case of an SAE.

### Telephone Contacts During the RSV Season or Post-Vaccination RSV Surveillance

During the RSV season (1 November to 31 March in the northern hemisphere and 1 May to 30 September in the southern hemisphere) or post-vaccination RSV surveillance (at least 5 months after last vaccine administration), make phone contacts every 2 weeks with the parent/guardian/legally authorized representative.
1) Record relevant information concerning the subject's health status including any changes in medications and immunizations on the telephone contact form.
2) Schedule an Illness Visit within the guidelines and stipulated time frame from the phone contact for a medically attended illness of the following types: fever, URI, LRI, or otitis media. If this illness overlaps with the Acute or Post-Acute Phases, the time frames specified for the relevant Acute or Post-Acute phase should be used.
3) If a subject had visited any other non-study doctor/hospital for a serious adverse event (SAE) (at any time in the study), schedule a site visit once the subject is discharged to obtain a nasal swab sample for RSV viral detection and quantification and rRT-PCR for respiratory pathogens.
4) If an SAE had occurred, follow the instructions herein for reporting it. Remind to notify the site in case of an SAE.

### Early Safety Data Review

The safety of the investigational product was continuously monitored by the Sponsor. To allow for a cautious, stepwise approach to vaccine administration, early safety data review (ESDR) was performed during scheduled SMT meetings:
Cohort 1
   D7 post-vaccination ESDR/SMT
   D28 post-vaccination ESDR/SMT
   D56 post-vaccination ESDR/SMT
Cohort 2
   D7 post-vaccination ESDR/SMT
   D28 post-vaccination ESDR/SMT
   D84 post-vaccination ESDR/SMT
Cohort 3
   D7 post-vaccination ESDR/SMT
   D28 post-vaccination ESDR/SMT
   D56 post-vaccination ESDR/SMT
Cohort 4
   D7 post-vaccination ESDR/SMT
   D28 post-vaccination ESDR/SMT
   D84 post-vaccination ESDR/SMT

The safety data collected was entered into the CRBs and summarized by the Sponsor in a blinded manner for each ESDR. The ESDR was performed by the Sponsor during the SMT meetings. Enrollment was not paused during SMT reviews.

It is understood that all reviews are based on preliminary data that have not been subjected to validation and database lock. The usual and ongoing process of monitoring safety signals outside of those specified in the protocol-defined early interim safety analysis will continue unchanged.

If at any other time point in the study the predetermined alert threshold for a safety event is reached, regardless of the study time point, the trial would have been paused for enrollment in order for an ad-hoc SMT to be convened.

During the safety evaluation, a Bayesian approach based on Posterior Distribution may be used to assess the difference between each RSV formulation and placebo on the following safety endpoints:
Any Grade 3 lower respiratory tract illness, e.g., wheezing, pneumonia, sustained tachypnea, laryngotracheobronchitis
Grade 3 fever

The probability that the difference of percentages of events between one RSV formulation and Placebo is greater than a pre-specified margin (δ) can be calculated based on posterior distribution, using non-informative prior Beta (1,1) and observed binomial data:

Proba (% RSV - % Placebo > δ)

If this probability is high (e.g., >=80%) then it may be recommended to drop the formulation. Thus, it can help decision making for safety evaluation by SMT and/or IDMC (if applicable) and can also be applied at the end of the study.

In cases where the review of data by the SMT does not result in the identification of a potential safety signal, SMT oversight proceeds as assigned in the SMT charter. When a safety signal is identified, the SMT investigates the event(s), completes the Safety Analysis and/or Evaluation Report and decides about the safety signal and further actions and/or recommendations including:
Extending or pausing the trial and escalate to the Vaccine Adjudication Committee (VAC)
Investigate and develops a report
Recommends continuing the trial

Within the investigation, the team may consult with non-clinical safety experts and research team members on whether there is a possible underlying mechanism for the event and review of toxicology data or other internal experts depending on the need.

In case of disagreement in the team, the core team seeks respective functional management advice from the VAC shortly after the SMT meeting. The Vaccine Pharmacovigilance Global Business Unit (PV GBU) Head or Vaccines GBU Clinical & Sciences Head may be also consulted, especially in instance where escalation of the signal for PSB review is in question.

Once the SMT core team agrees that there is a safety signal that can potentially impact the subject's safety, study conduct (pause, extended pause, or termination) or design (modifications needed), the SMT PM informs the Vaccine PV GBU Head who validates the signal and urgently contacts the Vaccines GBU Clinical & Sciences Head and the Chair of the PSB. The decision is made on whether to convene a PSB or not.

Based on the outcomes and recommendations from the PSB, the SMT may carry out any of the following operational actions related to the conduct and oversight of the study including:
Continue the trial
Extend the pause to conduct a further evaluation, e.g., an ad-hoc IDMC
Modify the trial design, or
Stop the trial.

An ad-hoc IDMC, which ideally will include at least 2 pediatric respiratory virus vaccine experts, will review upon request of the SMT unblinded study data. The ad-hoc IDMC will upon request review the relevant safety information and available data, including magnitude of vaccine virus shedding. If an ad-hoc IDMC meeting is required, the ad-hoc IDMC will review the data on safety available at that time point to determine if attributable to an etiology, a cause, or a diagnosis unrelated to the study vaccine, and if the adverse event(s) is associated with shedding of vaccine virus at the time of the event (even if another is identified). If the ad-hoc IDMC decides there is no proven causal relationship between the adverse events and study vaccine, the study can continue unchanged. Based on evaluation of the IDMC, the Sponsor SMT will decide if there is a causal relationship between the adverse event and the study vaccine, then SMT will decide whether to:
Conduct internal signal detection processes (SOP #RDWIN-000390)
Escalate validated signals to PSB as needed
Continue the trial
Modify the trial design (with PSB and ad-hoc IDMC input) or,
Stop the trial (with PSB and ad-hoc IDMC input).

The following safety parameters are assessed as part of the early safety review:
Immediate reactions
Solicited respiratory and systemic reactions
Unsolicited reactions, i.e., AEs reported as related by the Investigator
SAEs, MAAEs, and AESIs
   Enrollment was not paused during the review. The data was examined for the following:
1) Any deaths, regardless of causality.
2) Any vaccine related SAEs.
3) Grade 3 fever reported in >2 subjects.
4) Any Grade 3 or above solicited adverse events other than fever.
5) >1 subject experiencing lower respiratory tract illness of >Grade 2 DAIDS scale (except for wheeze by Brighton Collaboration Severity Scale) during the acute phase (i.e., within 28 days post-vaccination).
6) Any subject experiencing an AESI of Grade 3 or above by DAIDS scale (except for wheeze by Brighton Collaboration Severity Scale), including lower respiratory tract illness during the acute phase (i.e., within 28 days post-vaccination).
7) >1 subject with a Grade 3 unsolicited AE during the acute phase (i.e., within 28 days post-vaccination).
8) Any other pattern of research laboratory values or clinical symptoms other than fever that is considered a significant safety issue by the Investigator.

If any of the above criteria are met, a decision will be made as to whether enrollment in the study will be allowed to resume.

Case unblinding may be performed if necessary, as determined by the SMT.

### Enrollment and Retention of Study Population

### Recruitment Procedures

Before the start of the study, the Investigator and/or study staff determined the recruitment strategy to be used at their site for this study (e.g., advertising, database, direct mailing, or word of mouth referrals). Using the relevant methods, they contacted an appropriate pool of potential parents/guardians/legally authorized representatives and invited them to participate in the study. The sites ensured that any materials used to recruit subjects (e.g., information brochures, letters, pamphlets, posters, other advertisements, etc.) were submitted to the Sponsor prior to submission to the IRB for approval.

In addition, a parent/guardian/legally authorized representative who brings a child to the study site for routine visit was invited to enroll the subject in the study, if eligible. Subjects were also recruited from the general population.

### Informed Consent Procedures

Informed consent is the process by which a subject's guardian or appropriate legally acceptable representative voluntarily confirms his or her willingness to participate in a particular study. Informed consent must be obtained before any study procedures are performed. The process is documented by means of a written, signed, and dated ICF.

In accordance with GCP, prior to signing and dating the consent form, the subject's guardian/representative must be informed by appropriate study personnel about all aspects of the study that are relevant to making the decision to participate and must have sufficient time and opportunity to ask any questions.

If the subject's guardian/legally authorized representative is not able to read and sign the ICF, then it must be signed and dated by an impartial witness who is independent of the Investigator. A witness who signs and dates the consent form is certifying that the information in this form and any other written information had been accurately explained to and understood by the subject or his/her guardian/representative.

The actual ICF used at each center may differ, depending on local regulations and IEC/IRB requirements. However, all versions must contain the standard information found in the sample ICF provided by the Sponsor. Any change to the content of the ICF must be approved by the Sponsor and the IEC/IRB prior to the form being used.

If new information becomes available that may be relevant to the subject's guardian/legally authorized representative willingness to continue participation in the study, this was communicated to him/her in a timely manner. Such information was provided via a revised ICF or an addendum to the original ICF.

Informed consent forms were provided in duplicate, or a photocopy of the signed consent was made. The original was kept by the Investigator, and the copy was kept by the subject's guardian/legally acceptable representative.

Documentation of the consent process was recorded in the source documents.

### Rationale for Including Subjects Unable to Give Consent:

The rationale for conducting this study in pediatric population is included above.

### Screening Criteria

There are no screening criteria other than the inclusion and exclusion criteria. The Investigator must review the inclusion and exclusion criteria at enrollment (Visit 01).

### Inclusion Criteria:

An individual must fulfill all of the following criteria to be eligible for study enrollment at Visit 01:
1. Aged 6 through 18 months at DO. (6 through 18 months means from 6th month after birth to the day before the 19th month after birth.)
2. Informed consent form has been signed and dated by the parent(s)/guardian/or other legally authorized representative (and by independent witness if required by local regulations).
3. Subject and parent/guardian/legally authorized representative are able to attend all scheduled visits and to comply with all trial procedures.

### Exclusion Criteria:

An individual fulfilling any of the following criteria is to be excluded from trial enrollment:
1. Participation at the time of study enrollment (or in the 6 weeks preceding the first trial vaccination) or planned participation during the present trial period in another clinical trial investigating a vaccine, drug, medical device, or medical procedure.
2. Receipt of any of the following vaccines prior to enrollment:
   any influenza vaccine within 7 days prior, or
   any inactivated vaccine within the 14 days prior, or
   live-attenuated rotavirus vaccine within the 14 days prior, or
   any live vaccine, other than rotavirus vaccine, within the 28 days prior, or
   another investigational vaccine or investigational drug within 28 days prior.
3. Previous receipt of a licensed or investigational RSV vaccine or previous receipt or planned administration of any anti-RSV product (such as ribavirin or RSV immune globulin [IG] or RSV monoclonal antibody).
4. Receipt of immune globulins, blood, or blood-derived products in the past 6 months prior to enrollment.
5. Known or suspected congenital or acquired immunodeficiency; or receipt of immunosuppressive therapy, such as anti-cancer chemotherapy or radiation therapy, within the preceding 6 months; or long-term systemic corticosteroid therapy (prednisone or equivalent for more than 2 consecutive weeks within the past 3 months).
6. Probable or confirmed case of Coronavirus Disease 2019 (COVID-19).
7. Known systemic hypersensitivity to any of the vaccine components, or history of a life-threatening reaction to the vaccine used in the trial or to a vaccine containing any of the same substances.
8. Any chronic illness.
   Chronic illness may include, but is not limited to, cardiac disorders, lung disease (including any history of reactive airway disease, receipt of bronchodilator therapy, or medically diagnosed wheezing), renal disorders, auto-immune disorders, diabetes, psychomotor diseases, and known congenital or genetic diseases
9. Any history of medically diagnosed wheezing.
10. Any acute febrile, respiratory, or gastrointestinal illness in the past 24 hours that according to investigator judgment is significant enough to interfere with successful inoculation on the day of vaccination. A prospective subject should not be included in the study until the condition has resolved or the febrile event has subsided.
11. Receipt of any of the following medications within 3 days prior to study enrollment:
   systemic antibacterial, antiviral, antifungal, anti-parasitic, or antituberculous agents, whether for treatment or prophylaxis, or
   intranasal medications, or
   other prescription medication except as permitted concomitant medications (prescription or non-prescription) including nutritional supplements, medications for gastroesophageal reflux, eye drops, and topical medications, including (but not limited to) cutaneous (topical) steroids, topical antibiotics, and topical antifungal agents.
12. Receipt of salicylate (aspirin) or salicylate-containing products within the 28 days prior to enrollment.
13. Deprived of freedom in an emergency setting or hospitalized involuntarily.
14. Identified as a natural or adopted child of the Investigator or employee with direct involvement in the proposed study.
15. Any previous anaphylactic reaction.
16. Any previous vaccine-associated adverse reaction that was Grade 3 or above. Note: if grading is not possible, determine if the reaction was considered severe or life-threatening; if so, it is exclusionary.
17. Member of a household that contains, or will contain, an infant who is less than 6 months of age at the enrollment date (or in the 6 weeks preceding the first trial vaccination) through Day 28.
18. Member of a household that contains another child/other children who is/are, or is/are scheduled to be, enrolled in this study in the same year AND the date of enrollment will not be concurrent with the other participant(s) living in the household (i.e., all eligible children from the same household must be enrolled on the same date).
19. Member of a household that contains an immunocompromised individual, including, but not limited to:
   a person who is HIV infected
   a person who has received chemotherapy within the 12 months prior to enrollment
   a person receiving immunosuppressant agents
   a person living with a solid organ or bone marrow transplant.
20. Attends a daycare facility and shares a daycare room with infants less than 6 months of age, and parent/guardian/legally authorized representative is unable or unwilling to suspend daycare for 28 days following inoculation.
21. Scheduled administration of the following after planned inoculation:
   any influenza vaccine within 7 days after, or
   inactivated vaccine or live-attenuated rotavirus vaccine within the 14 days after, or
   any live vaccine other than rotavirus in the 28 days after, or
   another investigational vaccine or investigational drug in the 56 days after.
22. Born at less than 34 weeks gestation.
23. Born at less than 37 weeks gestation and less than 1 year of age at the time of enrollment.
24. Current suspected or documented developmental disorder, delay, or other developmental problem.
25. Any previous receipt of supplemental oxygen therapy in a home or hospital setting, except the temporary receipt of supplemental oxygen for transient tachypnea in newborn.

### Medical History

Prior to enrollment, subjects were assessed for pre-existing conditions and illnesses, both past and ongoing. Any such conditions were documented in the source document. Significant (clinically relevant) medical history (reported as diagnosis) including conditions/illnesses for which the subject is or has been followed by a physician or conditions/illnesses that could resume during the course of the study or lead to an SAE or to a repetitive outpatient care were collected in the CRB. The significant medical history section of the CRB contains a core list of body systems and disorders that could be used to prompt comprehensive reporting, as well as space for the reporting of specific conditions and illnesses.

For each condition, the data collected was limited to:
Diagnosis (this is preferable to reporting signs and symptoms)
Presence or absence of the condition at enrollment
The reporting of signs and symptoms in lieu of a diagnosis is strongly discouraged.

Dates, medications, and body systems are not to be recorded, and the information collected was not coded. The purpose of limited data is to assist in the later interpretation of safety data collected during the study.

### Contraindications for Subsequent Vaccinations

### Temporary Contraindications

Should a subject experience one of the conditions listed below at Day 0 (Cohorts 1, 2, 3, and 4) or Day 56 (Cohorts 2 and 4), the Investigator postponed further vaccination until the condition is resolved. Postponement must still be within the timeframe for vaccination, i.e., within 5 days of randomization for Day 0 (Cohorts 1, 2, 3, and 4) and within 7 days of Day 56 (Cohorts 2 and 4).

Any acute febrile illness (rectal temperature ≥38.0° C. [≥100.4° F.]), acute otitis media, upper and lower respiratory signs or symptoms (including, but not limited to, rhinorrhea, cough, and pharyngitis), or nasal congestion in the past 24 hours that according to investigator's judgment is significant enough to interfere with successful absorption of the study product.

Receipt of any of the following before any study vaccination or scheduled administration after any study vaccination:
any influenza vaccine within 7 days prior, or
any inactivated vaccine or live-attenuated rotavirus vaccine within the 14 days prior, or
any live vaccine, other than rotavirus vaccine, within the 28 days prior, or
another investigational vaccine or investigational drug within 28 days prior.

All eligible subjects from the same household who are enrolled on the same date must receive the study product on the same date, and therefore if one child experiences illness as above, product administration should be deferred for both children.

### Definitive Contraindications

If a subject experienced one of the conditions listed below, the Investigator discontinued vaccination:
An anaphylactic or any other significant allergic reaction to the previous dose of vaccine.
Any AE greater than Grade 2, LRI of any grade or SAE assessed by the investigator as related to the previous dose of vaccine.
Diagnosis of COVID-19

Subjects with a definitive contraindication were continued to be followed up for the study-defined safety and immunogenicity assessments, as applicable.

In the event of a local or national immunization program with a pandemic vaccine, e.g., influenza, subjects who received pandemic vaccine at any time during the study were to be withdrawn from the study.

### Conditions for Withdrawal

Parents/guardians/legally authorized representatives were informed that they have the right to withdraw their child from the study at any time. Any participant who has received study product was encouraged to remain in study follow-up for the duration of the study even if sample collection is refused.

A subject may be withdrawn from the study:
At the request of the parent/guardian/legally authorized representative either verbally or in writing (i.e., withdrawal of consent/dropout).
At the discretion of the Investigator or Sponsor due to safety concern of significant non-compliance with the protocol (based on the Investigator's judgment), without the parent's/guardian's/legally authorized representative's permission (i.e., non-compliant with the protocol/withdrawal).

The reason for a withdrawal or dropout should be clearly documented in the source documents and in the CRB.

The Investigator must determine whether voluntary withdrawal is due to safety concerns (in which case, the reason for discontinuation was noted as “Adverse Event”) or for another reason.

Withdrawn subjects were to be replaced.

For any participant who withdraws or is terminated from the study prior to scheduled completion of follow-up, study staff documented the reason for the withdrawal or termination in detail and made every effort to complete the following final evaluations:
1) Obtain verbal medical history about the subject, including past and ongoing medication and immunizations.
2) Obtain a blood sample for serum antibodies to RSV if possible.
3) Obtain a nasal swab sample for quantification of vaccine virus shedding (if in the Acute Phase), other respiratory pathogens including a COVID-19 POC test on the discretion of the investigator.
4) Complete the relevant section of the CRF.

### Lost to Follow-Up Procedures

In the case of subjects who failed to return for a follow-up examination, documented reasonable effort (i.e., documented telephone calls and certified mail) was undertaken to locate or recall them, or at least to determine their health status while fully respecting their rights. These efforts were documented in the source documents.

### Classification of Subjects Who Discontinued the Study

For any subject who discontinued the study prior to completion, the most significant reason for early termination was checked in the CRB. Reasons are listed below from the most significant to the least significant:

| | |
|---|---|
| Adverse Event | To be used when the subject is permanently |
| | terminated from the study because of an AE |
| | (including an SAE), as defined herein. |
| | This category also applies if the subject |
| | experiences a definitive contraindication |
| | that is an SAE or AE. |
| | IMPORTANT NOTE: in case subjects are |
| | discontinued from the study due to COVID-19 |
| | infection related event, “Adverse Event” should |
| | be selected and an Adverse Event Form completed |
| | if within the protocol defined period for safety |
| | collection. |
| Lost to Follow-up | To be used when the subject cannot be found or |
| | contacted in spite of efforts to locate him/her |
| | before the date of his/her planned last visit, |
| | as outlined herein. The certified letter was |
| | sent by the investigator and returned unsigned, |
| | and the subject or parent/guardian/legally |
| | authorized representative did not give any other |
| | news and did not come to any following visit. |
| Protocol | To be used: |
| Deviation | In case of significant non-compliance with the |
| | protocol (e.g., deviation of the Inclusion/ |
| | Exclusion criteria, non-compliance with time |
| | windows, blood sampling or vaccination refusal, |
| | missed injection/treatment, or error in the |
| | vaccine/treatment administration or any issue |
| | leading to definitely discontinue the subject |
| | from the study due to COVID-19 pandemic, e.g., |
| | quarantine, transportation restriction). |
| | If the subject experiences a definitive |
| | contraindication that is not an SAE or AE. |
| | The subject or the parent/guardian/legally |
| | authorized representative signed the certified |
| | letter sent by the investigator but did not |
| | give any other news and did not come to any |
| | following visit. |
| Withdrawal by | To be used: |
| Subject or | When the subject or parent/guardian/legally |
| Parent/Guardian/ | authorized representative indicated |
| Legally authorized | unwillingness to continue in the study |
| Representative | When the subject or parent/guardian/legally |
| | authorized representative made the decision |
| | to discontinue participation in the study |
| | for any personal reason other than an SAE/AE |
| | (e.g., subject is relocating, inform consent |
| | withdrawal, including fear of exposure to |
| | COVID-19 during pandemic period, etc.) |

### Follow-Up of Discontinuations

The site completed all scheduled safety follow-ups and contacted any subject who prematurely terminated the study because of an AE or a protocol deviation.

For subjects where the reason for early termination was lost to follow-up or if the subject withdrew informed consent and specified that they do not want to be contacted again and it is documented in the source document, the site did not attempt to obtain further safety information.

If the subject's status at the end of the study is “Withdrawal by Subject or Parent/Guardian/Legally Authorized Representative”, the site did attempt to contact them for the post-RSV season visit except if they specified that they do not want to be contacted again and it is documented in the source document.

Follow-Up of Subjects with COVID-19 in the Study

If a subject develops COVID-19 during the study, the subject was followed up according to national/regional/local health authority guidelines for as long as possible. All attempts were made to monitor the safety of the study subject and collect key samples whilst complying with national/regional/local health authority guidelines.

### Safety Emergency Call

If, as per the Investigator's judgment, a subject experiences a medical emergency, the Investigator may contact the Sponsor's RMO for advice on how to address any study-related medical question or problem. If the RMO is not available, then the Investigator may contact the Call Center—available 24 hours a day, 7 days a week—that forwarded all safety emergency calls to the appropriate primary or back-up Sponsor contact, as needed.

This process does not replace the need to report an SAE. The Investigator is still required to follow the protocol-defined process for reporting SAEs to the Global Pharmacovigilance (GPV) Department.

In case of emergency code-breaking, the Investigator is required to follow the code-breaking procedures.

### Modification of the Study and Protocol

Any amendments to this study plan and protocol must be discussed with and approved by the Sponsor. If agreement is reached concerning the need for an amendment, it was produced in writing by the Sponsor, the amended version of the protocol will replace the earlier version. All substantial amendments (e.g., those that affect the conduct of the study or the safety of subjects) require IEC/IRB approval and must also be forwarded to regulatory authorities.

An administrative amendment to a protocol is one that modifies some administrative, logistical, or other aspect of the study but does not affect its scientific quality or have an impact on the subjects' safety. The IECs/IRBs should only be notified, no formal approval is required.

The Investigator is responsible for ensuring that changes to an approved study, during the period for which IEC/IRB approval has already been given, are not initiated without IEC/IRB review and approval, except to eliminate apparent immediate hazards to subjects.

### Interruption of the Study

The study may be discontinued if new data about the investigational product resulting from this or any other studies become available; or for administrative reasons; or on advice of the Sponsor, the Investigators, the IECs/IRBs, or the governing regulatory authorities in the countries where the study is taking place.

If the study is prematurely terminated or suspended, the Sponsor shall promptly inform the Investigators, the IECs/IRBs, the regulatory authorities, and any contract research organization(s) used in the study of the reason for termination or suspension, as specified by applicable regulatory requirements. The Investigator shall promptly inform the subjects' parents/guardians/legally authorized representatives and should assure appropriate subject therapy and/or follow-up.

### Products Administered

### Identity of the Investigational Product(s)

The identity of the investigational products is described in the sections below for Cohorts 1 through 4.

### Identity of Study Product 1

Respiratory Syncytial Virus (RSV) RSV ΔNS2/Δ1313/I1314L (Sanofi) Vaccine 5.6 log₁₀ PFU/0.2 mL, suspension of virus.

### Composition

Each 0.2 mL dose of RSV ΔNS2/Δ1313/I1314L (Sanofi) vaccine contains the following components:

Live-attenuated RSV with (i) a 523-nucleotide deletion of the NS2 gene, (ii) an amino acid deletion in the L protein (Δ1313; deletion of S1313), and (iii) a genetically stabilizing mutation in the L gene (I1314L), 5.6 log₁₀ PFU, approximately 0.1 mL delivered as a fine mist of droplets (range 10-120 μm in size) per nostril, using an intranasal atomizer device.

### Preparation and Administration

A commercially available intranasal mucosal atomization device (MAD) was used for administration of the experimental RSV ΔNS2/Δ1313/I1314L (Sanofi) vaccine in the Phase I/II clinical trial. The selected device, MAD130 system, comes with a spray nozzle (MAD 300 atomizer), a 1 ml plastic syringe and a plastic vial access cannula. For the Phase I/II clinical study, the experimental RSV ΔNS2/Δ1313/I1314L (Sanofi) vaccine produced by the Sponsor was filled and stored in vials. At the clinical trial site, the experimental RSV ΔNS2/Δ1313/I1314L (Sanofi) vaccine was withdrawn from the vial at a specified volume into the syringe, the device/nozzle was fitted to the syringe head and primed, and the dose was administered intranasally with approximately half given in each nostril using the MAD130 Device.

Prior to administration, all study products must be inspected visually for cracks, broken seals, correct label content, and extraneous particulate matter and/or discoloration, whenever solution and container permit. If any of these conditions exists, the vaccine was not e administered. A replacement dose was used, and the event reported to the Sponsor.

Subjects must be kept under observation for 30 minutes after each vaccination to ensure their safety, and any reactions during this period were documented in the CRB. Appropriate medical equipment and emergency medications, including epinephrine (1:1000), was available on site in the event of an anaphylactic, vasovagal, or other immediate allergic reaction.

### Dose Selection and Timing

Subjects in Cohort 1 received 1 administration of RSV ΔNS2/Δ1313/I1314L (Sanofi) 5.6 log₁₀ PFU vaccine on Day 0.

Subjects in Cohorts 2 and 4 received 2 administrations: 1 administration of RSV ΔNS2/Δ1313/I1314L (Sanofi) 5.6 log₁₀ PFU vaccine on Day 0 and a subsequent administration on Day 56.

### Vaccination Device

A US licensed, commercially available intranasal mucosal atomization device (MAD), manufactured by Teleflex, was the vaccine device. The selected device, MAD130 system, comes with a spray nozzle (MAD 300 atomizer), a 1 ml plastic syringe and a plastic vial access cannula. For the Phase I/II clinical study, the experimental RSV ΔNS2/Δ1313/I1314L (Sanofi) vaccine or control product produced by the Sponsor was filled and stored in vials. At the clinical trial site, the experimental RSV ΔNS2/Δ1313/I1314L (Sanofi) vaccine or control product was withdrawn from the vial at a specified volume into the syringe, the device/nozzle was fitted to the syringe head and primed, and the dose was administered intranasally with approximately half given in each nostril using the MAD130 Device.

The mechanism of action is as follows: when manual pressure is applied to the syringe, the plunger pushes the liquid product through the spray nozzle and atomizes the drug product into a spray form. The conical shaped plug is engineered to direct the spray plume more consistently towards the top of the nasal passage through the nasal valve into the nasal cavity.

### Teleflex MAD Nasal™ Intranasal Mucosal Atomization Device Manufacturing Information:

Made from radiation-stable medical-grade polycarbonate material, compliant with United States Pharmacopeia Class VI and ISO 10993 requirements.

Manufactured in an ISO Class 7 cleanroom environment.

Manufacturing process is compliant with FDA 21 CFR part 820, ISO 13485, and EU MDD.

Non-pyrogenic and does not contain latex.

The Teleflex MAD Nasal™ Intranasal Mucosal Atomization Device is ISO-594 compliant and can be used with any ISO-594 compliant luer lock syringe.

### Manufacturer's Specifications:

The MAD130 Intranasal Mucosal Atomization Device has the following manufacturer's specifications as documented in the product insert:
Typical Droplet Size: 30-100 μm
System Dead Space: 0.15 mL
Tip Diameter: 4.3 mm

### Identity of Study Product 2

Respiratory Syncytial Virus (RSV) RSV ΔNS2/Δ1313/I1314L (Sanofi) Vaccine 6.2 log₁₀ PFU/0.2 mL, suspension of virus.

### Composition

Each 0.2 mL dose of RSV ΔNS2/Δ1313/I1314L (Sanofi) vaccine contains the following components:

Live-attenuated RSV with (i) a 523-nucleotide deletion of the NS2 gene, (ii) an amino acid deletion in the L protein (Δ1313; deletion of S1313), and (iii) genetically stabilizing mutation in the L gene (I1314L), 6.2 log₁₀ PFU, approximately 0.1 mL to be delivered as a fine mist of droplets (range 10-120 μm in size) per nostril, using an intranasal atomizer device.

### Preparation and Administration

The procedures for preparing and administering the control product are the same as those described for the study product.

### Dose Selection and Timing

Subjects in Cohort 3 received 1 administration of RSV ΔNS2/Δ1313/I1314L (Sanofi) 6.2 log₁₀ PFU vaccine on Day 0.

Subjects in Cohort 4 received 2 administrations: 1 administration of RSV ΔNS2/Δ1313/I1314L (Sanofi) 6.2 log₁₀ PFU vaccine on Day 0 and a subsequent administration on Day 56.

### Vaccination Device

The vaccination device is described herein.

### Identity of Control Product(s)

### Placebo

### Composition

Same formulation buffer as the RSV ΔNS2/Δ1313/I1314L (Sanofi) vaccine, to be delivered as approximately 0.1 mL per nostril.

### Preparation and Administration

The procedures for preparing and administering the control product are the same as those described for the study product herein.

### Dose Selection and Timing

Subjects in Cohort 1 and 3 received 1 administration of placebo on Day 0.

Subjects in Cohorts 2 and 4 received 2 administrations: 1 administration of placebo on Day 0 and a subsequent administration on Day 56.

### Vaccination Device

The vaccination device is described herein.

### Product Logistics

### Labeling and Packaging

The investigational and placebo products in single-dose vials were supplied with investigational labeling and packaging according to national regulations. Each single dose of investigational or placebo product was identified by a unique number on the primary label and on the outer carton label. The carton label also had a detachable label for the sites to attach to the source documents.

The investigational and placebo products are blinded at the level of the carton.

### Product Shipment, Storage, and Accountability

### Product Shipment

The Clinical Study Manager or designee contacted the Investigator or a designee to determine the dates and times of delivery of products.

Each vaccine shipment included a temperature-monitoring device to verify maintenance of the cold chain during transit. On delivery of the product to the site, the person in charge of product receipt followed the instructions, including checking that the cold chain was maintained during shipment (i.e., verification of the temperature recorders). If there is an indication that the cold chain was broken, this person should immediately quarantine the product, alert the Sponsor representative, and request authorization from the Sponsor to use the product.

### Product Storage

The Investigator was personally responsible for product management or designated a staff member to assume this responsibility.

At the site, products must be kept in a secure place with restricted access. Vaccines were stored in a freezer at <−60° C. (<−76° F.) and protected from light. The temperature must be monitored and documented for the entire time that the vaccine is at the study site. In case of accidental disruption of the cold chain, vaccines must not be administered and must be quarantined, and the Investigator or authorized designee should contact the Sponsor representative for further instructions.

### Product Accountability

The person in charge of product management at the site maintained records of product delivery to the study site, product inventory at the site, the dose(s) given to each subject, and the disposal of or return to the Sponsor of unused doses. Because the vaccine and placebo differ in appearance, an unblinded coordinator(s) verified the product accountability and also dispensed the vaccine.

The necessary information on the product labels was entered into the source document and the CRB. If applicable, information was also entered into the subject's vaccination card.

The Sponsor's monitoring staff verified the study site's product accountability records against the record of administered doses in the CRBs and the communication from the IRT (if applicable).

In case of any expected or potential shortage of product during the study, the Investigator or an authorized designee alerted the Sponsor representative as soon as possible, and a shipment of extra doses was arranged.

### Replacement Doses

If a replacement dose was required (e.g., because the syringe broke or particulate matter was observed in the syringe), the site personnel must have contacted the IRT to receive the new dose allocation.

### Disposal of Unused Products

Unused or wasted products were returned to the Sponsor. Product accountability was verified throughout the study period.

### Recall of Products

If the Sponsor made a decision to launch a retrieval procedure, the Investigator(s) was/were informed of what needed to be done.

### Blinding and Code-Breaking Procedures

The study was performed in an observer-blind fashion:
Investigators and study staff who conducted the safety assessment and the subject did not know which vaccine was administered.
Only the study staff who prepared and administered the vaccine and were not involved with the safety evaluation knew which vaccine was administered.

The parent/guardian/legally authorized representative, the Investigator and study staff members who collected safety data, and laboratory personnel who analyzed the blood samples, did not know which product was administered. The vaccinator was in charge of preparing and administering the products and was not authorized to collect any safety data. In addition, the vaccinator or authorized designee ensured that the documents on randomization were stored in a secure place where only she/he had access.

The code may be broken in the event of an AE only when the identification of the vaccine received could influence the treatment of the subject. Code breaking should be limited to the subject(s) experiencing the AE.

The blind can be broken by the Investigator or a delegate through the IRT system. Once the emergency has been addressed by the site, the Investigator or a delegate must notify the Sponsor RMO if a subject's code was broken. All contact attempts with the Sponsor prior to unblinding are to be documented in the source documents, and the code breaking CRF is to be completed.

A request for the code to be broken may also be made:
by the GPV Department through an internal system for reporting to Health authorities in the case of an SAE as described in International Council for Harmonisation of Technical Requirements for Pharmaceuticals for Human Use (ICH) E2A. In this case, the code will be broken only for the subject(s) in question. The information resulting from code-breaking (i.e., the subject's vaccine or group assignment) will not be communicated to either the Investigator or the immediate team working on the study, except for the GPV representative.
by the ad-hoc IDMC if needed to facilitate their assessment of safety.

The IEC/IRB must be notified of the code breaking. All documentation pertaining to the event must be retained in the site's study records and, in the Sponsor files. Any intentional or unintentional code breaking must be reported, documented, and explained, and the name of the person who requested it must be provided to the Sponsor.

An unblinded interim analysis for dose selection of RSV ΔNS2/Δ1313/I1314L (Sanofi) vaccine was planned on subjects from Cohorts 1, 2, and 3, and at least 90 subjects enrolled in Cohort 4. The interim analysis took place when subjects provided safety data up to the D84 timepoint and have D84 immunogenicity results available. This unblinded interim analysis required the unblinding of data; a specific process was implemented to maintain the blind at the subject and Investigator levels.

Testing performed within the Sponsor's laboratory and the Sponsor's outsourced laboratories were blinded with respect to study treatment group assignment. The code(s) linking information on sample vials to study treatment group assignment was retained by the Clinical Department and could not be accessed by the Sponsor or contract laboratory testing personnel.

### Randomization and Allocation Procedures

An IRT was implemented to assign the subject numbers and allocate vaccine groups with dose numbers to the subjects at Visit 01.

At Visit 01 visit, subjects who met the inclusion/exclusion criteria and whose parent/guardian/legally authorized representative signed the ICF were randomly assigned to one of the vaccine groups, according to the cohort:
Cohorts 1 and 2: RSV ΔNS2/Δ1313/I1314L (Sanofi) 5.6 log₁₀ PFU (RSV low-dose) or Placebo in a 1:1 ratio
Cohort 3: RSV ΔNS2/Δ1313/I1314L (Sanofi) 6.2 log₁₀ PFU (RSV high-dose) or Placebo in a 1:1 ratio
Cohort 4: RSV ΔNS2/Δ1313/I1314L (Sanofi) 6.2 log₁₀ PFU (RSV high-dose) or RSV ΔNS2/Δ1313/I1314L (Sanofi) 6.2 log₁₀ PFU (RSV low-dose) or Placebo in a 1:1:1 ratio

At Visit 01, randomization was stratified by cohort and by age sub-group (<12 months/≥12 months).

Site staff connected to the IRT, entered the identification and security information, and confirmed a minimal amount of data in response to IRT prompts. The IRT then provided the dose number assignment and had the site staff confirm it. If the subject is not eligible to participate in the study, then the information was only recorded on the subject recruitment log.

Subject numbers assigned by the IRT consisted of a 12-digit string (a 3-digit country identifier, a 4-digit study center identifier, and a 5-digit subject identifier). For example, Subject 840000100005 is the fifth subject enrolled in Center Number 1 in the US (840 being the US country code).

### Treatment Compliance

The following measures ensured that the vaccine doses administered complied with those planned, and that any non-compliance was documented so that it can be accounted for in the data analyses:
All vaccinations were administered by qualified study personnel.
The person in charge of product management at the site maintained accountability records of product delivery to the study site, product inventory at the site, dose(s) given to each subject, and the disposal of unused or wasted doses.

### Concomitant Medications and Other Therapies

At the time of enrollment, ongoing medications and other therapies (e.g., blood products) should be recorded in the source document as well as new medications prescribed for new medical conditions/AEs during study participation.

Documentation in the CRB of ongoing concomitant medication(s) was limited to specific categories of medication(s) of interest beginning on the day of first vaccination. This may include medications of interest that were started prior to the day of vaccination.

Reportable medications were collected in the CRB from the day of each vaccination to the end of the solicited and unsolicited follow-up period.

Reportable medications include medications that impact or may impact the consistency of the safety information collected after any vaccination and/or the immune response to vaccination. Three standard categories of reportable medications are defined:
Medications impacting or that may have an impact on the evaluation of the safety (e.g., antipyretics, analgesics, and non-steroidal anti-inflammatory drugs [NSAIDs], steroids/corticosteroids).
Medications impacting or that may have an impact on the immune response (e.g., other vaccines, blood products, antibiotic classes that may interfere with bioassays used by the GCI department, steroids/corticosteroids, immune-suppressors, immune-modulators with immunosuppressive properties, anti-proliferative drugs such as DNA synthesis inhibitors).
Medications impacting or that may have an impact on both the safety and the immune response (e.g., steroids/corticosteroids)

The information reported in the CRB for each reported medication was limited to:
Trade name
Origin of prescription: prophylaxis Yes/No. Medication(s) prescribed for AE prophylaxis was recorded in the Action Taken of the AE collection tables.
Start and stop dates
Reason for treatment

Dosage and administration route, homeopathic medication, topical and inhaled steroids, as well as topical, ophthalmic, and ear treatments were not recorded. Topical analgesics were not applied at the site of vaccination; however, if they were applied inadvertently to the vaccination site, they were recorded as a Category 1 medication in this specific instance.

Medications given in response to an AE was captured in the “Action Taken” section of the AE CRF only. No details were recorded in the concomitant medication CRF unless the medication(s) received belongs to one of the pre-listed categories.

Medications were coded.

### Prohibited Concomitant Medications

The use of the following is prohibited:
Prophylactic antipyretics, decongestants, or antihistamines during the Acute Phase (28 days following study product administration)—note that use of these medications for treatment of symptoms is allowed.
An investigational drug or investigational vaccine other than the study product within 56 days after receiving study product.

### Precautionary Concomitant Medications

Due to their potentially confounding effect on immunogenicity results, the following treatments should be avoided after study product administration unless clinically indicated:
Systemic corticosteroids for more than 14 days at a dosage equivalent to prednisone at >2 mg/kg or 20 mg daily or other immune-modifying drugs.
Immunoglobulins and/or any blood products.

The following should be avoided after study product administration unless indicated in an outbreak setting:
Licensed inactivated vaccine or live-attenuated rotavirus vaccine within 14 days after receiving study product.
Licensed live virus vaccine, other than rotavirus vaccine, within 28 days after receiving study product

### Management of Samples

Blood samples for the assessment of antibody responses were collected at Visits 01, 03, and 04 for Cohorts 1 and 3 subjects who received 1 administration and Visits 01, 03, 05, and 06 for Cohorts 2 and 4 subjects who received 2 administrations. See the Table of Study Procedures herein for details of the sampling schedule.

Nasal swab samples for confirmation of RSV were collected at Visit 02 (Day 7) for Cohorts 1 and 3 subjects who received 1 administration and at Visit 02 (Day 7) and Visit 04 (Day 63) for Cohorts 2 and 4 subjects who received 2 administrations. A nasal swab specimen for the detection of RSV and respiratory pathogens was collected from subjects during illness visits.

All collection of samples in the US adhered to the Centers for Disease Control (CDC) recommended Interim Infection Prevention and Control Recommendations for Patients with Suspected or Confirmed Coronavirus Disease 2019 (COVID-19) in Healthcare Settings. Collection of samples in the sites outside the US complied with local regulatory guidelines for COVID-19.

See the Table of Study Procedures herein for details of the sampling schedule.

### Sample Collection

At the above-mentioned visits, up to 5 mL of blood was collected in tubes provided by or recommended by the Sponsor.

At the above-mentioned visits, nasal swab samples were collected and transferred into tubes provided by or recommended by the Sponsor. Staff collecting the samples used adequate infection prevention and control measures.

Immediately prior to the blood draw or nasal swab, the staff member performing the procedure verified the subject's identity as well as the assigned subject's number and sampling stage on the pre-printed label and attached the label to the tube.

### Sample Preparation

### Serum Samples

An overview of the procedures is provided here.

Following the blood draw, the tubes are left undisturbed, positioned vertically and not shaken, for a minimum of 1 hour and a maximum of 24 hours to allow the blood to clot. Samples can be stored at room temperature for up to 2 hours; beyond 2 hours, they must be refrigerated at a temperature of +2° C. to +8° C. (+35.6° F. to +46.4° F.) after the period of clotting at room temperature and must be centrifuged within a maximum of 24 hours.

The samples are then centrifuged, and the serum is transferred to the appropriate number of aliquoting tubes. These tubes are pre-labeled with adhesive labels that identify the study code, the subject's number and the sampling stage or visit number.

The subject's number and the date of sampling, the number of aliquots obtained, the date and time of preparation, and the subject's consent for future use of his/her samples are to be specified on a sample identification list and recorded in the source document. Space is provided on this list for comments on the quality of samples.

### Nasal Swab Samples

An overview of the procedures is provided herein.

The subject's identification number and any other required information, the date of sampling, and the date and time of preparation was clearly documented.

### Sample Storage and Shipment

### Serum Samples

During storage, serum tubes are to be kept in a freezer whose temperature is set and maintained at −20° C. (−4° F.) or below. The temperature was monitored and documented on the appropriate form during the entire study. If it rose above −10° C. (14° F.) for any period of time, the Clinical Logistics Coordinator was notified.

Shipments to the laboratories were made only after appropriate monitoring and following notification of the Clinical Logistics Coordinator. Sera was shipped frozen, using dry ice to maintain them in a frozen state, in the packaging container provided by the carrier. Again, temperatures were monitored. Shipments must be compliant with the United Nations (UN) Class 6.2 specifications and the International Air Transport Association (IATA) 602 packaging instructions.

Samples were shipped to Sample, Reagent, & Animal Services within R&D Global Operations at the Sponsor.

### Nasal Swab Samples

Nasal swab samples were shipped frozen, using dry ice to maintain them in a frozen state, in the packaging container provided by the carrier.

### Future Use of Stored Biological Samples for Research

Any unused part of the serum samples and nasal swab samples were securely stored at the Sponsor for at least 25 years after the end of the study. These samples are being retained in long-term storage to support answers to regulatory questions related to the product's licensure and the potential revalidation of the study results. In addition, these samples will also be used for assay development activities related to RSV or other respiratory pathogens.

The other biological samples collected to qualify the subjects for inclusion in the study or to monitor their health are dedicated for immediate use. In case they were not completely used up, they were destroyed at the latest at the end of the study or after the time requested by local law.

In addition, parents/guardians/legally authorized representatives were asked to indicate in the ICF whether they permit the future use of any unused stored serum samples for other tests. If they refused permission, the samples were not used for any testing other than that directly related to this study. If they agreed to this use, they were not paid for giving permission. Anonymity of samples was ensured. The aim of any possible future research is unknown today and may not be related to this particular study. It may be to improve the knowledge of vaccines or infectious diseases, or to improve existing tests or develop new tests to assess vaccines. Human genetic tests will never be performed on these samples without specific individual informed consent.

### Clinical Supplies

The Sponsor supplied the study sites with protocols, ICFs, CRBs, SAE reporting forms, diary cards, memory aids and other study documents, as well as with the following study materials: all study vaccines including the device for vaccine administration, blood collection tubes, cryotubes, cryotube storage boxes, cryotube labels, temperature recorders, shipping containers, and digital thermometers.

The means for performing Electronic Data Capture (EDC) were defined by the Sponsor. If a computer was provided by the Sponsor, it was retrieved at the end of the study.

The Investigator supplied all vaccination supplies, phlebotomy, and centrifugation equipment, including biohazard and/or safety supplies. The biohazard and safety supplies include needles and syringes, examination gloves, laboratory coats, sharps disposal containers, and absorbent countertop paper. The site ensured that all biohazard wastes were autoclaved and disposed of in accordance with local practices. The Investigator also supplied appropriate space in a temperature-monitored refrigerator for the storage of the products and for the blood samples, and appropriate space in a temperature-monitored freezer for serum aliquots.

In the event that additional supplies were required, study staff contacted the Sponsor, indicating the quantity required.

### Endpoints and Assessment Methods

### Primary Endpoints and Assessment Methods

### Safety

### Safety Definitions

The following definitions are taken from the ICH E2A Guideline for Clinical

### Safety Data Management: Definitions and Standards for Expedited Reporting.

### Adverse Event (AE):

An AE is any untoward medical occurrence in a patient or in a clinical investigation subject administered a medicinal product and which does not necessarily have a causal relationship with this treatment. An AE can therefore be any unfavorable and unintended sign (including an abnormal laboratory finding, for example), symptom or disease temporally associated with the use of a medicinal product, whether or not considered related to the medicinal product.

Therefore, an AE may be:
A new illness
The worsening of a pre-existing condition
An effect of the vaccination, including the comparator
A combination of the above

All AEs include serious and non-serious AEs.

Surgical procedures are not AEs; they are the actions taken to treat a medical condition. It is the condition leading to the action taken that is the AE (if it occurs during the study period).

Pre-existing medical conditions are not to be reported as AEs. However, if a pre-existing medical condition worsens following study interventions in frequency or intensity, or if according to the Investigator there is a change in its clinical significance, this change should be reported as an AE (exacerbation). This applies equally to recurring episodes of pre-existing conditions (e.g., asthma) if the frequency or intensity increases post-vaccination.

### Serious Adverse Event (SAE):

Serious and severe are not synonymous. The term severe is often used to describe the intensity of a specific event as corresponding to Grade 3. This is not the same as serious, which is based on subject/event outcome or action criteria usually associated with events that pose a threat to a subject's life or functioning. Seriousness, not severity, serves as a guide for defining regulatory reporting obligations.

An SAE is any untoward medical occurrence that at any dose
Results in death
Is life-threatening
Requires inpatient hospitalization or prolongation of existing hospitalization
Results in persistent or significant disability/incapacity
Is a congenital anomaly/birth defect
Is an important medical event (IME)

The term “life-threatening” refers to an event in which the subject was at risk of death at the time of the event; it does not refer to an event which hypothetically might have caused death if it were more severe.

All medical events leading to hospitalizations were recorded and reported as SAEs, with the exception of: hospitalization planned before inclusion into the study or outpatient treatment with no hospitalization.

“Persistent or significant disability or incapacity” means that there is a substantial disruption of a person's ability to carry out normal life functions.

Medical and scientific judgment should be exercised in deciding whether expedited reporting is appropriate in other situations, such as IMEs that may not be immediately life-threatening or result in death or hospitalization but may jeopardize the health of the subject or may require intervention to prevent one of the other outcomes listed in the definition above. These IMEs should also usually be considered serious. Examples of such events include allergic bronchospasm requiring intensive treatment in an emergency room or at home, blood dyscrasias or convulsions that do not result in inpatient hospitalization, or the development of drug dependency or drug abuse, new-onset diabetes, or auto-immune disease.

### Adverse Reaction:

All noxious and unintended responses to a medicinal product related to any dose should be considered adverse reactions (AR).

(The phrase “responses to a medicinal product” means that a causal relationship between a medicinal product and an AE is at least a reasonable possibility)

The following additional definitions are used by the Sponsor:

### Immediate Event/Reaction:

Immediate events are recorded to capture medically relevant unsolicited systemic AEs (including those related to the product administered) that occur within the first 30 minutes after vaccination.

### Solicited Reaction:

A solicited reaction is an “expected” adverse reaction (sign or symptom) observed and reported under the conditions (nature and onset) pre-listed in the protocol and CRB (e.g., fever and runny nose occurring between DO and D28 post-vaccination).

By definition, solicited reactions are to be considered as being related to the product administered.

For vaccines administered nasally, solicited reactions can either be solicited administration site reactions or solicited systemic reactions.

### Unsolicited AE/AR:

An unsolicited AE is an observed AE that does not fulfill the conditions pre-listed in the CRB in terms of diagnosis and/or onset window post-vaccination. For example, if fever between DO and D28 is a solicited reaction (i.e., pre-listed in the protocol and CRB), then a fever starting on D28 is a solicited reaction, whereas fever starting on D29 post-vaccination is an unsolicited AE. Unsolicited AEs includes both serious (SAEs) and non-serious unsolicited AEs.

### Medically Attended AE (MAAE):

An MAAE is a new onset or a worsening of a condition that prompts the subject or subject's parent/guardian/legally authorized representative to seek unplanned medical advice at a physician's office or Emergency Department. A physician contact made over the phone or by e-mail was considered a physician office visit for the purpose of MAAE collection. This definition excludes pre-planned medical office visits for routine medical care, as well as pediatric check-ups or follow-up visits of chronic conditions with an onset prior to entry in the study. An AE discovered during a planned routine visit (e.g., upper respiratory tract infection, otitis) was collected as an MAAE.

### Administration Site Reaction:

An administration site reaction is an AR at and around the administration site, i.e., the intranasal mucosa. They are considered related to the product administered.

### Systemic AE:

Systemic AEs are all AEs that are not injection or administration site reactions. They therefore include systemic manifestations such as headache, fever, as well as localized or topical manifestations that are not associated with the vaccination or administration site (e.g., conjunctivitis that is localized but that is not occurring at the administration site).

### Adverse Event of Special Interest (AESI):

An adverse event of special interest is one of scientific and medical concern specific to the Sponsor's product or program, for which ongoing monitoring and rapid communication by the investigator to the sponsor can be appropriate. Such an event might warrant further investigation in order to characterize and understand it. Depending on the nature of the event, rapid communication by the study Sponsor to other parties (e.g., regulators) might also be warranted. AESIs include both serious (SAEs) and non-serious unsolicited AEs.

The following definitions are for the different phases of safety follow post-vaccine administration:

### Acute Phase:

The Acute Phase begins with vaccine administration on DO and ends at midnight on the 28^{th} day after vaccine administration (D28). The Acute Phase for the second vaccine administration begins on D56 post-vaccination and ends at midnight on D84

During the Acute Phase of the study, a study healthcare professional was available by telephone 24 hours a day for consultation with parents/guardians/legally authorized representatives regarding any illnesses that may occur during this period. Study personnel had daily contact with the parents/guardians/legally authorized representatives for the first 7 days after each vaccine administration and then 3 times weekly after Day 07 up to 28 days after each vaccine administration. This 28-day Acute Phase of safety follow-up is consistent with the duration of shedding of live attenuated RSV viruses in RSV seronegative infants and toddlers. If the parent/guardian/legally authorized representative reports an SAE, a safety event that meets the study pause or stop criteria described herein, or symptoms suggestive of a respiratory tract illness, then an illness visit should be scheduled as described herein. During the Acute Phase, the eDC allows daily safety monitoring of the study participant and were programmed to send an alert to the parent/guardian/legally authorized representative and study site when there are symptoms suggestive of respiratory tract illness.

### Post-Acute Phase:

The Post-Acute Phase for subjects receiving 1 administration begins at 12:01 am on D29 and ends at midnight on D56. The first Post-Acute Phase for subjects receiving 2 administrations begins at 12:01 am on D29 and ends at midnight on D56, except if second administration is exactly on D56 when it ends immediately before receipt of the second administration. The second Post-Acute Phase for subjects receiving 2 administrations begins at 12:01 am on D85 and ends at midnight on D112.

During the Post-Acute Phase of the study, parents/guardians/legally authorized representatives were instructed to monitor for and contact study staff if their child has had symptoms that are suggestive of a serious adverse event. If the parent/guardian/legally authorized representative reports an SAE or safety event that meets the study pause or stop criteria described herein, then an illness visit should be scheduled.

### Safety Endpoints

The primary endpoints for the evaluation of safety are as follows (in all infants and toddlers regardless of baseline serostatus):
Occurrence of any unsolicited systemic AEs reported in the 30 minutes after each and any vaccination.
Occurrence of solicited (i.e., pre-listed in the subject's DC/eDC and in the CRB) administration site and systemic reactions within 28 days after each and any vaccination (i.e., Acute Phase).
Occurrence of any unsolicited (spontaneously reported) AEs within 28 days after each and any vaccination (i.e., Acute Phase)
Occurrence of any AESIs within 28 days after each and any vaccination.
Occurrence of any MAAEs within 28 days after each and any vaccination.
Occurrence of any SAEs throughout the study period.
Other safety endpoints were recorded or derived as described in the statistical analysis plan. Depending on the item, these could include nature (Medical Dictionary for Regulatory Activity [MedDRA] preferred term), time of onset, duration, number of days of occurrence, intensity, relationship to vaccine, action taken, whether the AE led to early termination from the study, seriousness, or outcome.

### Safety Assessment Methods

At each vaccination in-person or non-visit contact, the Investigator or a delegate either performed a focused medical examination (in-person visit) or asked the parent/guardian/legally authorized representative about any solicited reactions and unsolicited AEs recorded in the diary card/electronic diary card, as well as about any other AEs that may have occurred since the previous visit. All relevant data was transcribed into the CRB according to the instructions provided by the Sponsor.

This study monitors safety, infectivity, replication, and immunogenicity of both doses of RSV ΔNS2/Δ1313/I1314L (Sanofi), with attention to vaccine virus infectivity and replication (i.e., percentage of participants shedding virus, vaccine virus titer in nasal swab) 7 days post-vaccination 1 and 2, which is the most quantifiable metric for the level of attenuation of the vaccine virus.

### Immediate Post-Vaccination Observation Period

Subjects were kept under observation for 30 minutes after each vaccination to ensure their safety. The post-vaccination observation should be documented in the source document. Any AE that occurs during this period was noted on the source document and recorded in the CRB, as follows:
Unsolicited systemic AEs were recorded as immediate AEs in the CRB (presence marked as “yes” and details collected).
Solicited and unsolicited administration site reactions and solicited systemic reactions were recorded in the CRB in the same way as any reactions starting on the day of vaccination.
SAEs were recorded in the CRB and reported to the Sponsor in the same way as any other SAEs.
   Reactogenicity (Solicited Reactions from Day 0 to Day 28 after Each Vaccination)

After each vaccination, the subject's parents/guardians/legally authorized representatives were provided with a DC/eDC, and a digital thermometer, and were instructed how to use them. The following items were recorded by the subjects in the diary card/electronic diary card on the day of vaccination and for the next 28 days (i.e., Day 0 through Day 28) until resolution:
Daily temperature, with the route by which it was taken
Daily measurement and intensity grade of any solicited administration site reactions (e.g., runny nose) and systemic reactions (e.g., fever)
Action taken for each event (e.g., medication)

The action(s) taken by the parent/guardian/legally authorized representative to treat and/or manage any solicited reactions were classified in the CRB using the following list (all applicable items should be checked):
None
Medication
Health care provider contact
Hospitalized
Discontinuation of study vaccination

Parents/guardians/legally authorized representatives of study subjects were contacted by telephone each day for the first 7 days after each vaccination and then 3 times weekly after the D07 visit to remind them to record all safety information in the diary card/electronic diary card.

If the timing of the telephone call should fall on a weekend or a holiday, the call should be made on the next business day. If contact is not made on the designated day, study staff continued calling until contact is made. Every telephone attempt and its outcome was documented in the source document.

Table 6 and Table 7 present, respectively, the administration site reaction that is pre-listed in the diary cards and CRB, together with the intensity scales.

TABLE 6
Solicited administration site reactions: terminology, definitions, and intensity scales

| CRB term | | |
|---|---|---|
| (MedDRA lowest | | |
| level term | | |
| [LLT]) | Rhinorrhea | Nasal congestion |
| Diary card term | Runny nose | Stuffy or blocked nose |
| Definition | Two or more consecutive days of clear, | Two or more consecutive days of |
| | mucous or purulent discharge from the | stuffy or blocked nose. |
| | nose. | Note: Not associated with crying, |
| | Note: Not associated with crying, | change of room temperature, or |
| | change of room temperature, or eating | eating or drinking. |
| | and drinking. | |
| Intensity scale* | Grade 1 (mild): No medical | Grade 1 (mild): No medical |
| | intervention required; may include use | intervention required; may include |
| | of over-the-counter medications | use of over-the-counter |
| | managed by the caregiver for treatment | medications managed by the |
| | of symptoms | caregiver for treatment of symptoms |
| | Grade 2 (moderate): Outpatient | Grade 2 (moderate): Outpatient |
| | medical intervention by a health care | medical intervention by a health care |
| | provider required; may include use of | provider required; may include use of |
| | over-the-counter and/or prescription | over-the-counter and/or prescription |
| | medications | medications |
| | Grade 3 (severe): Prolonged medical | Grade 3 (severe): Prolonged medical |
| | intervention and/or hospitalization | intervention and/or hospitalization |
| | required | required |
| | Grade 4 (life-threatening): Illness | Grade 4 (life-threatening): Illness |
| | requiring hospitalization with intensive | requiring hospitalization with |
| | care | intensive care |
| | Grade 5 (death): Event resulting in | Grade 5 (death): Event resulting in |
| | fatal outcome to the participant | fatal outcome to the participant |

*For the measurable reactions of rhinorrhea and nasal congestion, the parent/guardian/legally authorized representative decided whether or not medical intervention was required and if so the type of intervention, and the classification as Grade 1-5 was assigned at the time of review of the diary card.

TABLE 7
Solicited systemic reactions: terminology, definitions, and intensity scales

| CRB term | | | | | | |
|---|---|---|---|---|---|---|
| (MedDRA | | | | | | |
| lowest level | | | | | | |
| term | | | Crying | | Appetite | |
| [LLT]) | Fever | Vomiting | abnormal | Drowsiness | lost | Irritability |
| Diary card | Temperature | Vomiting | Abnormal | Drowsiness | Loss of | Irritability |
| term | | | crying | | appetite | |
| Definition | Elevation of | Vomiting | Inconsolable | Reduced | See intensity | An |
| | temperature | does not | crying | interest in | scale | excessive |
| | to ≥38.0° C. | include | without a | surroundings, | | response to |
| | (≥100.4° F.) | spitting up | determined | or increased | | stimuli: |
| | | | reason | sleeping | | increased |
| | | | | | | fussiness, |
| | | | | | | whining, |
| | | | | | | and |
| | | | | | | fretfulness |
| | | | | | | despite |
| | | | | | | attempts to |
| | | | | | | comfort the |
| | | | | | | infant and |
| | | | | | | despite |
| | | | | | | caregiver |
| | | | | | | responses |
| | | | | | | that would |
| | | | | | | normally be |
| | | | | | | soothing |
| Intensity | Grade 1: ≥38.0° C. | Grade 1: 1 | Grade 1: <1 | Grade 1: | Grade 1: | Grade 1: |
| scale* | to ≤38.5° C. | episode per | hour | Sleepier than | Eating less | Easily |
| | or ≥100.4° F. | 24 hours | Grade 2: 1-3 | usual or less | than normal | consolable |
| | to ≤101.3° F. | Grade 2: 2-5 | hours | interested in | Grade 2: | Grade 2: |
| | Grade 2: >38.5° C. | episodes per | Grade 3: >3 | surroundings | Missed 1 or 2 | Requiring |
| | to ≤39.5° C. | 24 hours | hours | Grade 2: Not | feeds/meals | increased |
| | or >101.3° F. | Grade 3: ≥6 | | interested in | completely | attention |
| | to ≤103.1° F. | episodes per | | surroundings | Grade 3: | Grade 3: |
| | Grade 3: >39.5° C. | 24 hours or | | or did not | Refuses ≥3 | Inconsolable |
| | or >103.1° F. | requiring | | wake up for a | feeds/meals | |
| | | parental | | feed/meal | or refuses | |
| | | hydration | | Grade 3: | most | |
| | | | | Sleeping | feeds/meals | |
| | | | | most of the | | |
| | | | | time or | | |
| | | | | difficult to | | |
| | | | | wake up | | |

For all reactions but fever, the parent/guardian/legally authorized representative recorded the intensity level (Grade 1, 2, or 3) in the diary card. For fever, they recorded the body temperature, and the classification as Grade 1, 2, or 3 was assigned based on the information in the diary card.

### Important Notes for the Accurate Assessment of Temperature:

Parents/guardians/legally authorized representatives measured body temperature once per day, preferably always at the same time. The optimal time for measurement is the evening, when body temperature is the highest. Temperature was also measured at the time of any apparent fever. The observed daily temperature and the route of measurement were recorded in the diary card, and the highest temperature was recorded by the site in the CRB. The preferred route for this study is rectal. Pre-vaccination temperature is also systematically collected by the investigator on the source document. Tympanic thermometers must not be used.

### Unsolicited Adverse Events

In addition to recording solicited reactions, parents/guardians/legally authorized representatives were instructed to record any other medical events that may occur during the 28-day period after each vaccination. Space was provided in the diary card for this purpose.

Information on SAEs was collected and assessed throughout the study, from the time of enrollment until up the last visit. Any SAE occurring at any time during the study was reported by the Investigator in the CRB according to the completion instructions provided by the Sponsor; this includes checking the “Serious” box on the AE CRF and completing the appropriate Safety Complementary Information CRFs. All information concerning the SAE was reported either as part of the initial reporting or during follow-up reporting if relevant information became available later (e.g., outcome, medical history, results of investigations, copy of hospitalization reports and verbal autopsy questionnaire if used). In case a subject experiences febrile convulsion (neurological event associating fever and seizure), the assessment was performed according to the “Guideline for definition and collection of cases of febrile convulsion”, and this event was considered an SAE.

For each unsolicited AE (whether serious or non-serious), the following information was recorded:
Start and stop dates (The stop date of all related AEs were actively solicited. For other events, the investigator provided the stop date when it becomes available. AEs for which no stop date was obtained during the course of the study was considered as ongoing at the end of the study.)
Intensity of the event:
   a. The temperature for fever was collected and analyzed based on the corresponding scale used for solicited reactions.
   b. All other unsolicited AEs were classified according to the following intensity scale:
      i. Grade 1: A type of adverse event that is usually transient and may require only minimal treatment or therapeutic intervention. The event does not generally interfere with usual activities of daily living.
      ii. Grade 2: A type of adverse event that is usually alleviated with additional therapeutic intervention. The event interferes with usual activities of daily living, causing discomfort but poses no significant or permanent risk of harm to the research participant.
      iii. Grade 3: A type of adverse event that interrupts usual activities of daily living, or significantly affects clinical status, or may require intensive therapeutic intervention.
Whether the AE was related to the investigational product (for unsolicited systemic AEs)

The Investigator assessed the causal relationship between the AE and the investigational product as either “Not related” or “Related”, as described in herein.
Action taken for each AE (e.g., medication)

The action(s) taken by the parents/guardians/legally authorized representatives to treat and/or manage any unsolicited AEs was classified in the CRB using the following list (all applicable items should be checked):
None
Medication
Health care provider contact
Hospitalized
Discontinuation of study vaccination.
Whether the AE was an AESI, MAAE, and/or SAE.

For each SAE, the investigator completed all seriousness criteria that apply (outcome, elapsed time, and relationship to study procedures)
Whether the AE caused study discontinuation

### Medically Attended Adverse Events

Any MAAEs occurring within the first 28 days post-vaccination (DO to D28 for all subjects in Cohorts 1, 2, 3, and 4 and D56 to D84 for subjects in Cohorts 2 and 4) was collected using the same process as other AEs. Medically attended acute respiratory illness (MAARI) and medically attended acute lower respiratory illness (MAALRI) events occurring within this time frame and outside the RSV surveillance season were classified as MAAEs.

### Adverse Events of Special Interest (AESI)

Any AESIs occurring within the first 28 days post-vaccination (DO to D28 for all subjects in Cohorts 1, 2, 3, and 4 and D56 to D84 for subjects in Cohorts 2 and 4) was collected using the same process as other AEs.

All adverse events of special interest in the study would be graded by the Division of AIDS (DAIDS) Table for Grading the Severity of Adult and Pediatric Adverse Events except for acute wheeze, which would be graded by the Brighton Collaboration Wheeze severity grading system. The following adverse events of special interest are derived from previous NIH research experience with similar candidates and were assessed in this study:
Acute otitis media
URI
   Pharyngitis
   Cough without LRI
LRI
   Stridor
   Rales
   Tachypnea
   Acute wheeze
   Pneumonia
   Laryngotracheobronchitis

### Acute Otitis Media:

Loss of tympanic membrane landmarks accompanied by erythema and loss of mobility. May or may not be associated with fever or other respiratory symptoms. Confirmed with tympanometry if possible. This diagnosis must be made by a medical professional.

### Pharyngitis:

Pharyngeal erythema accompanied by exudate or pharyngeal erythema with enlarged tender lymph nodes. Note: May be associated with sore throat, or painful or difficult swallowing. This diagnosis must be made by a medical professional.

Cough without LRI:

Two or more consecutive days of 3 or more episodes of cough during a 15-minute timed observation period, or cough awakens child from sleep. There should be no associated lower respiratory tract illness. This diagnosis must be made by a medical professional. Note: Not associated with eating, drinking, or choking.

### Stridor:

Harsh medium-pitched inspiratory sound associated with obstruction of the laryngeal area or the extra-thoracic trachea, often accompanied by croupy cough and hoarse voice. This diagnosis must be made by a medical professional.

### Rales:

Abnormal lung sound heard through a stethoscope. Rales may be sibilant (whistling), dry (crackling), or wet (more sloshy) depending on the amount and density of fluid refluxing back and forth in the air passages. Must be sustained over 20 minutes and assessed and diagnosed by a medical professional with confirmation by a second medical professional, if possible.

### Tachypnea:

Increased respiratory rate >40 breaths per minute in infants aged 6-12 months and >30 breaths per minute in toddlers aged 1-3 years. This diagnosis must be made by a medical professional.

### Acute Wheeze:

According to the Brighton Collaboration case definition, for all levels of diagnostic certainty, acute wheeze is a clinical sign defined by:
Sudden onset, occurred unexpectedly and without warning, leading to a marked change in a subject's previously stable condition, AND
Breath sound audible on auscultation, AND
Audible during expiration, the sound is primarily expiratory though an inspiratory component is possible in severe forms different from stridor, AND

Characterized by the following criteria defining the level of diagnostic certainty:

### Level 1

Classified as wheeze based on digital stethoscope recording as compared with reference audio file, OR
Classified as wheeze by 2 health care providers with specific training, e.g., pulmonologist or formal auscultation training based on standard wheeze training tool
   Level 2a (One Health Care Provider with Specific Training)
Classified as wheeze by 1 health care provider with specific training, e.g., pulmonologist or formal auscultation training based on standard wheeze training tool, AND
Immediate response to bronchodilator treatment, i.e., absence of wheeze following treatment or improvement of wheeze severity documented by a healthcare provider

### Level 2b (2 Health Care Providers)

Classified as wheeze by 2 health care providers without specific training, i.e., neither a pulmonologist nor someone with formal auscultation training based on standard wheeze training tool, AND
Immediate response to bronchodilator treatment, i.e., absence of wheeze following treatment or improvement of wheeze severity documented by a healthcare provider, OR
Infant diagnosed with acute bronchiolitis

### Level 3 (Pre-Existing Diagnosis)

Classified as wheeze by 1 health care provider OR caretaker (e.g., parent/guardian/legally authorized representative) without specific training, AND
Pre-existing physician diagnoses of a respiratory disease of which wheeze is a key symptom

The full Brighton Collaboration case definition is listed here but Level 3 of diagnostic certainty will not be required in this study and children with a pre-existing diagnosis of wheeze would not be enrolled in this study.

### Pneumonia:

Rales and crackles sustained over 20 minutes, originating in the lower respiratory tract, usually accompanied by tachypnea, which do not clear with cough. This may be confirmed by x-ray showing areas of consolidation. Clinical assessment and diagnosis must be made by a medical professional with confirmation by a second medical professional, if possible.

### Laryngotracheobronchitis (Croup):

Barking cough, hoarseness, and inspiratory stridor and sustained over 20 minutes assessed and diagnosed by a medical professional with confirmation by a second medical professional, if possible.

Table 8 presents the DAIDS severity scale for AESIs, except wheeze, Grade 1 through 4.

TABLE 8
DAIDS severity scale for AESIs, except wheeze

| | | | Grade 4 Potentially |
|---|---|---|---|
| Grade 1 Mild | Grade 2 Moderate | Grade 3 Severe | Life-Threatening |
| Mild symptoms | Moderate symptoms | Severe symptoms | Potentially life-threatening |
| causing no or minimal | causing greater than | causing inability to | symptoms causing inability to |
| interference with usual | minimal interference | perform usual social & | perform basic self-care |
| social & functional | with usual social & | functional activities | functions with intervention |
| activities with | functional activities | with intervention or | indicated to prevent permanent |
| intervention not | with intervention | hospitalization | impairment, persistent |
| indicated | indicated | indicated | disability, or death |

All deaths related to an AESI are to be classified as Grade 5.

Table 9 presents the Brighton Collaboration severity grading system for wheeze.

TABLE 9
Brighton Collaboration severity grading system for wheeze

| Grade 1 (mild) | Grade 2 (moderate) | Grade 3 (severe) |
|---|---|---|
| Wheeze only | Wheeze with respiratory | Wheeze with danger |
| | distress | signs |
| Wheeze*† | Wheeze | Wheeze‡ |
| | AND | AND |
| | Lower chest wall indrawing | Oxygen saturation <92% |
| | OR | OR |
| | prolonged expirium (i.e., | Central cyanosis |
| | I/E ratio >1:3) | OR |
| | | Confusion or drowsiness |
| | | OR |
| | | Inability to speak or drink |

I/E, Inspiratory/Inspiratory
*There may even be tachypnea without other signs of respiratory distress
†There may be tachycardia without other signs of respiratory distress
‡or silent chest on auscultation in a patient with history of wheeze

### Assessment of Causality

The Investigator assessed the causal relationship between each unsolicited systemic AE and the product administered as either not related or related, based on the following definitions:
Not related—The AE is clearly/most probably caused by other etiologies such as an underlying condition, therapeutic intervention, or concomitant therapy; or the delay between vaccination and the onset of the AE is incompatible with a causal relationship; or the AE started before the first vaccination
Related—There is a “reasonable possibility” that the AE was caused by the product administered, meaning that there is evidence or arguments to suggest a causal relationship

Note: By convention, all AEs reported at the administration site (whether solicited or unsolicited) and all solicited systemic AEs are considered to be related to the administered product and therefore are referred to as reactions and do not require the Investigator's opinion on relatedness.

Adverse events likely to be related to the product, whether serious or not, that persist at the end of the study were followed up by the Investigator until their complete disappearance or the stabilization of the subject's condition. The Investigator informed the Sponsor of the date of final disappearance of the event or the date of “chronicity” establishment.

### Infectivity

There are no primary objectives for infectivity.

### Immunogenicity

### Immunogenicity Definition

### RSV Serostatus

For this Phase I/II study, serum IgA detection was chosen as a biomarker for RSV encounter in infants and toddlers. IgA serostatus, i.e., RSV-naïve and RSV-experienced, is defined as undetectable or detectable serum anti-RSV A IgA antibodies, respectively.

### RSV IgA Serostatus Assessment

The RSV IgA ELISA for serostatus determination will be performed at the Sponsor or at a qualified contract laboratory for the Sponsor.

The IgA serostatus method to be used is summarized below.

### RSV IgA Method Description

IgA antibodies to RSV F antigen are measured using the anti RSV F IgA ELISA. RSV F protein antigen is coated on the surface of microtiter plates. The plates are blocked, then unadsorbed coating antigen is washed from the wells and serially diluted human serum samples (test samples, reference, and quality controls) are incubated in the wells. Anti-RSV F protein specific antibodies in the serum samples bind to the immobilized RSV F protein antigen. Unbound antibodies are washed from the wells and horseradish peroxidase (HRP)-conjugated goat anti-human IgA enzyme conjugate is added. The conjugate binds to the antigen-antibody complexes. Excess conjugate is washed away, and a colorimetric substrate is added. Bound enzyme catalyzes a hydrolytic reaction which causes color development. The intensity of the color generated is proportional to the amount of antigen specific IgA antibody bound to the wells. The results are read on a spectrophotometer.

The concentration of IgA antibodies to RSV F antigen is calculated over 6 serial two-fold dilutions with an assigned value in ELISA Units (EU)/mL.

### Immunogenicity Endpoints

The primary endpoint for the evaluation of immunogenicity is:
RSV A serum neutralizing antibody titers by D56 for Cohorts 1, 2, 3, and 4, and by D84 for Cohorts 2 and 4 in RSV-naïve subjects.

RSV-experienced and RSV-naïve subjects are defined herein.

### Immunogenicity Assessment Methods

Immunogenicity of the vaccine candidate was evaluated to measure RSV A serum neutralizing antibody titers (by Microneutralization assay).

### RSV Neutralizing Antibody Assessment

The RSV Microneutralization (MN) assay was performed at the Sponsor or at a qualified contract laboratory for the Sponsor.

The MN method used is summarized below. Development and qualification of this method is completed; this method will be validated prior to Phase III clinical testing.

### RSV MN Method Description

RSV neutralizing antibodies were measured using a MN assay. Serial, two-fold dilutions of serum tested (previously heat-inactivated) are mixed with a constant concentration of the RSV-A2 strain (ATCC VR-1540). The mixtures are inoculated into wells of a 96-well microplate with permissive HEp-2 cells (ATCC CCL-23) and incubated for 2 days. A reduction in virus infectivity (viral antigen production) due to neutralization by antibody present in serum samples is detected by enzyme linked immunosorbent assay (ELISA). After washing and fixation, RSV antigen production in cells is detected by successive incubations with an RSV-specific mAb, horse radish peroxidase anti-mouse IgG conjugate, and a chromogenic substrate. The resulting Optical Density is measured using a microplate reader. The reduction in RSV infectivity as compared to that in the virus control wells constitutes a positive neutralization reaction indicating the presence of neutralizing antibodies in the serum sample.

### Secondary Endpoints and Assessment Methods

### Safety

### Safety Definitions

The safety definitions are presented above.

### Safety Endpoints

The secondary endpoints for the evaluation of safety are:
Titer of vaccine virus shedding on D7 for Cohorts 1, 2, 3, and 4, and D63 for Cohorts 2 and 4 measured by RT-PCR.

### Safety Assessment Methods

Shedding of the attenuated RSV vaccine strain in nasal swab samples was evaluated by RSV quantitative Reverse Transcriptase Polymerase Chain Reaction (qRT-PCR) assay, which can specifically detect and quantify the RSV ΔNS2/Δ1313/I1314L (Sanofi) vaccine strain in human nasal swab samples. Based on the evaluation of precision (intra-assay and intermediate precision), dilutional accuracy, linearity, and specificity, RSV ΔNS2/Δ1313/I1314L (Sanofi) qRT-PCR assay is suitable for clinical testing of human nasal swab samples in support of the development of RSV Vaccine candidates.

### RSV ΔNS2/Δ1313/I1314L (Sanofi) RT-qPCR Method

The RSV ΔNS2/Δ1313/I1314L (Sanofi) RT-qPCR method was performed at the Sponsor or at a qualified contract laboratory for the Sponsor.

The RSV ΔNS2/Δ1313/I1314L (Sanofi) RT-qPCR assay method was used to measure virus shedding from nasal swab samples from study subjects. Development and qualification of this method is completed; this method will be validated prior to Phase III clinical testing.

### RSV ΔNS2/Δ1313/I1314L (Sanofi) RT-qPCR Method Description

To quantify viral shedding in infants vaccinated with the RSV ΔNS2/Δ1313/I1314L (Sanofi) vaccine, a qRT-PCR assay was developed that would specifically detect and quantify RSV ΔNS2/Δ1313/I1314L (Sanofi) in nasal swab samples.

RSV ΔNS2/Δ1313/I1314L (Sanofi) qRT-PCR assay was designed using the Light Cycler Probe system from Sigma. This system includes two hybridization probes that are designed so that they bind the target 1-5 nucleotides apart. Probe 1 (donor) is labeled 3′ end with a donor reporter. Probe 2 (acceptor) is labeled at the 5′ end with an acceptor reporter. During the annealing step, the PCR primers and the LightCycler Probes hybridize to their specific target regions, bringing the probes in proximity. When this happens, the donor dye is excited by the LightCycler, and energy is transferred from the donor to the acceptor dye. The acceptor reporter's emission is detected by the Light Cycler at 640 nm.

If the probes bind, but are not in proximity, no signal is produced. The Light Cycler probes for the RSV ΔNS2/Δ1313/I1314L (Sanofi) qRT-PCR assay target the deletion site of the NS2 gene in RSV ΔNS2/Δ1313/I1314L (Sanofi). Probe 1 binds before the deletion and Probe 2 binds across the deletion site. While the primers and probes may bind wild-type RSV A due to high sequence similarity, the two probes would not bind in close enough proximity to create signal as the NS2 gene is over 500 nucleotides long, making this method highly specific.

Collection of nasal swab samples from infants can be difficult and sample quality cannot be visually verified. Therefore, an RNase P assay, based on one developed by the CDC will be used in conjunction with the RSV ΔNS2/Δ1313/I1314L (Sanofi) qRT-PCR assay to evaluate sample quality.

RNase P is a human housekeeping gene. Use of the RNase P assay will limit the impact of false negatives because of poor sample collection or handling. Amplification in the RNase P assay indicates the presence of human cells in a sample. An RNase P Cp of ≤37 indicates that the sample was appropriately collected, and that sample integrity was maintained. RNase P testing will be performed only when qRT-PCR (to detect and quantify the RSV ΔNS2/Δ1313/I1314L (Sanofi) vaccine strain) yields negative results.

### Infectivity

### Infectivity Endpoint

The secondary endpoint for the evaluation of infectivity is:
Vaccinee infection with the vaccine virus. Infection is defined as detection of vaccine virus in a nasal swab by RT-PCR and/or a ≥4-fold rise in RSV A serum neutralizing antibody titers, or in RSV serum anti-F IgG antibody titers.

### Infectivity Assessment Methods

The infectivity assessment methods are presented above.

### Immunogenicity

### Immunogenicity Endpoints

The secondary endpoints for the evaluation of immunogenicity are:
RSV serum neutralizing antibody titers by D56 for Cohorts 1, 2, 3, and 4, and by D84 for Cohorts 2 and 4 in RSV experienced subjects.
RSV serum anti-F IgG antibody titers by D56 for Cohorts 1, 2, 3, and 4, and by D84 for Cohorts 2 and 4.
RSV A serum neutralizing and serum anti-RSV F IgG antibody titers after the RSV season or at least five months after last vaccine administration.

### Immunogenicity Assessment Methods

RSV-experienced and RSV-naïve subjects are defined above.

Immunogenicity of the vaccine candidate were evaluated to measure RSV F protein binding antibody levels (by ELISA).

### RSV Anti-F IgG ELISA

The anti-RSV F IgG ELISA measurements were performed at the Sponsor or at a qualified contract laboratory for the Sponsor.

The ELISA method used is summarized below.

### RSV Anti-F IgG ELISA Method Description

Antibodies to RSV F antigen were measured using the anti RSV F IgG ELISA. Briefly, RSV F antigen (from strain RSV A2) is coated onto a microtiter plate and serial 2-fold dilutions of human serum samples are added and incubated to allow binding to the RSV F antigen. Then an HRP-conjugated anti-human IgG detection antibody is added followed by colorimetric substrate. The concentration of IgG antibodies to RSV F antigen is calculated over 6-serial dilutions relative to qualified internal reference calibrated against WHO International standard (1st International Standard for antiserum for Respiratory Syncytial Virus) with an assigned value (International Units/mL).

### Data Management

### Management of SAE

During the study, SAE data (reported on the AE, Death, and Safety Complementary Information CRFs) was integrated into the Sponsor's centralized GPV database upon receipt of these forms and after a duplicate check. Each case was assigned a case identification number. Each case was assessed by the case management platform or its delegate before being reported to the relevant authorities as necessary. The assessment of related cases was done in collaboration with the Global Safety Officer and the RMO. Follow-up information concerning a completed case was entered into the GPV database, and a new record of the case was created.

The information from the GPV database cases was reconciled with that in the clinical database.

### Management of Clinical and Laboratory Data

Clinical data, defined as all data reported in the CRB, and laboratory data was handled by the Sponsor's Clinical Data Management (CDM) platform or authorized representative.

During the study, clinical data reported in the CRBs was integrated into the clinical database under the responsibility of the Sponsor CDM platform. Data monitoring at the sites and quality control in the form of computerized logic and/or consistency checks was systematically applied to detect errors or omissions. In addition, data reviews were performed several times by the Sponsor's staff in the course of the study. Any questions pertaining to the reported clinical data was submitted to the investigator for resolution using the EDC system. Each step of this process was monitored through the implementation of individual passwords to maintain appropriate database access and to ensure database integrity.

The validation of the immunogenicity data was performed at the laboratory level following the laboratory's procedures. Information from the laboratory was checked for consistency before integration into the clinical Datawarehouse.

After integration of all corrections in the complete set of data, and after the SAE information available from CDM and the GPV Department has been reconciled, the database was released for statistical analysis.

### Data Review

A blind review of the data was conducted through the data review process led by Data Management before database lock.

### Statistical Methods and Determination of Sample Size

### Statistical Methods for Primary Objectives

The statistical methodology was based on the use of two-sided 95% confidence intervals (CI).

The 95% CIs of point estimates were calculated using the exact binomial distribution (Clopper-Pearson method) for proportions.

For immunogenicity data, assuming that log₁₀ transformation of the titers/titer ratio follows a normal distribution, first, the mean and 95% CIs were calculated on log₁₀ (titers/titers ratio) using the usual calculation for normal distribution. Then antilog transformations were applied to the results of calculations, to compute geometric mean titers (GMTs) and geometric mean titers ratios (GMTRs) and their 95% CIs.

### Safety

Solicited adverse reactions (ARs), unsolicited AEs (including SAEs), MAAEs, and AESIs were summarized. The main parameters were described with 95% CI. At least the following parameters were presented by vaccine group after each and any vaccination, in all subjects regardless of baseline serostatus:
Unsolicited systemic AEs occurring within 30 minutes of administration (immediate unsolicited AEs)
Solicited administration site reactions and solicited systemic reactions within 28 days after each and any administration according to occurrence, time to onset, intensity, number of days of occurrence, action taken, and whether the reaction led to early termination from the study. When more than 1 intensity level is reported within a time period, the highest intensity was used.
Unsolicited AEs occurring within 28 days after each and any administration by system organ class (SOC) and preferred term (PT), relationship, intensity, time to onset, duration and whether the AE led to early termination from the study.
All SAEs that occur throughout the study by SOC and PT, seriousness criteria, time to onset, outcome, relationship, and whether the SAE led to early termination throughout the study
All MAAEs and AESIs reported within 28 days after each and any vaccination by SOC and PT and relationship

A Bayesian approach based on Posterior Distribution may be used to assess the difference between each RSV formulation and placebo on the following safety endpoints:
Any Grade 3 lower respiratory tract illness e.g., wheezing, pneumonia, sustained tachypnea, laryngotracheobronchitis
Grade 3 fever

### Immunogenicity

The point estimates and their 95% CI of the following parameters were presented for RSV A neutralizing antibody titers by D56 for Cohorts 1, 2, 3, and 4, and by D84 for Cohorts 2 and 4, for each vaccine group in RSV naïve subjects:
GMT
GMTR based on the baseline antibody titer
Seroresponse rates defined as percentage of subjects with a ≥4-fold rise in RSV A serum neutralizing antibody titers based on the baseline value.
Reverse cumulative distribution curves (RCDCs) were presented
Pairwise comparisons between vaccine groups may be conducted as exploratory analyses to compare seroresponse rates or GMTs for descriptive purpose.

The 95% CIs of the difference of proportions between 2 groups were computed using the Wilson Score method without continuity correction. CIs of ratio of GMTs between 2 groups were computed from the difference in means of log₁₀ transformed titers between 2 groups with normal approximation.

### Statistical Methods for Secondary Objectives

### Statistical Methods Safety

The point estimates and their 95% CI of the following parameter were presented 7 days after each vaccination (D7 for Cohorts 1, 2, 3, and 4, and D63 for Cohorts 2 and 4) for each vaccine group by baseline serostatus:
GMT of vaccine virus shedding measured by RT-PCR

### Infectivity

The point estimate and its 95% CI of the following parameters were presented after vaccination 1 (D56) for Cohorts 1, 2, 3, and 4, and after vaccination 2 (D84) for Cohorts 2 and 4 for each vaccine group by baseline serostatus:
Proportion of vaccinees infected with the vaccine virus. Infection defined as detection of vaccine in nasal swab by polymerase chain reaction (PCR) and/or a ≥4-fold rise in RSV A serum neutralizing antibody titers or RSV serum anti-F IgG antibody titers.

### Immunogenicity

The point estimates of GMT and GMTR and their 95% CI were presented by vaccine group for the following endpoints:
RSV A serum neutralizing antibody titers by D56 for Cohorts 1, 2, 3, and 4, and by D84 for Cohorts 2 and 4, in RSV-experienced subjects
RSV serum anti-F IgG antibody titers by D56 for Cohorts 1, 2, 3, and 4, and by D84 for Cohorts 2 and 4 by baseline serostatus.
RSV A serum neutralizing and anti-RSV F IgG antibody titers after RSV season or at least 5 months after last vaccine administration by baseline serostatus.

Seroresponse was also presented, as applicable.

### Analysis Sets

### Full Analysis Set

The full analysis set (FAS) is defined as the subset of randomized subjects who received at least 1 administration of the study vaccine. The data for any subject infected by laboratory confirmed wt RSV will be excluded from immunogenicity and viral shedding analyses from the date of wt RSV infection.

### Safety Analysis Set

The safety analysis set (SafAS) is defined as those subjects who have received at least 1 administration of the study vaccine. All subjects had their safety analyzed after each administration according to the vaccine they actually received and after any vaccination according to the vaccine received at the first administration.

Safety data recorded for a vaccine received out of the protocol design was excluded from the analysis (and listed separately).

### Per-Protocol Analysis Set

The per-protocol analysis set (PPAS) is a subset of the FAS. Two specific PPAS were defined: PPAS1 after 1 administration (for subjects in Cohorts 1, 2, 3, and 4) and PPAS2 after 2 administrations (for subjects in Cohorts 2 and 4).

The subjects presenting with at least one of the following relevant protocol deviations were excluded from the PPAS:
Baseline serology blood sample was not collected at visit 01 (DO).
Subject with temporary contraindication did not receive vaccination 1 in the proper time window from randomization.
Subject did not meet all protocol-specified inclusion criteria or met at least one of the protocol-specified exclusion criteria
Subject did not receive vaccine/did not complete the vaccination schedule
Subject received a vaccine other than the one that he/she was randomized
Preparation and/or administration of vaccine not done as per-protocol
Subject received a protocol-prohibited therapy before the post-vaccination serology blood sample Subject with confirmed diagnosis of wild-type (wt) RSV before the post-vaccination serology blood sample
Subject with an emergency unblinding performed by the Investigator

The subjects presenting with at least one of the following relevant protocol deviations were excluded from the PPAS1 only:
Post-vaccination 1 serology blood sample not collected at visit 03
Post-vaccination 1 serology blood sample not collected at visit 03 in the proper time window

The subjects presenting with at least one of the following relevant protocol deviations were excluded from the PPAS2 only (for subjects in Cohorts 2 and 4):
Subject did not receive vaccination 2 at visit 03 in the proper time window
Post-vaccination 2 serology blood sample not collected at visit 05
Post-vaccination 2 serology blood sample not collected at visit 05 in the proper time window

In addition to the reasons listed above, subjects were also excluded from the PPAS if their baseline serology sample or their post-vaccination serology sample did not produce a valid test result (i.e., result for RSV A serum neutralizing antibody titers is missing).

Note: For PPAS1 (after 1 administration), timepoints considered are: DO (visit 01) for vaccination and D56 (visit 03) for the post-vaccination serology sample. For PPAS2 (after 2 administrations), timepoints considered are: both DO (visit 01) and D56 (visit 03) for vaccinations and D84 (visit 05) for the post-vaccination serology sample.

### Other Analysis Set(s)

### Randomized Subjects

A randomized subject is a subject for whom a vaccine group has been allocated.

### Populations Used in Analyses

The safety analysis was performed on the SafAS. Subjects were analyzed after each vaccination according to the vaccine they actually received, and after any vaccination according to the vaccine received at the first administration.

Immunogenicity analyses were performed on the Full Analysis Set, and on the Per-Protocol Analysis Set for main immunogenicity parameters. In the FAS, subjects were analyzed by the vaccine group to which they were randomized. In the PPAS, subjects were analyzed according to the vaccine they actually received.

### Handling of Missing Data and Outliers

### Safety

No replacement was done. Nevertheless, missing relationship was considered as related at the time of the statistical analysis. No search for outliers was performed. In all subject listings, partial and missing data was clearly indicated as missing.

### Immunogenicity

Missing data was not imputed. No test or search for outliers was performed.

For the calculation of GMT and proportion of subjects with nAb titers above thresholds, any pre-vaccination or post-vaccination value reported as <lower limit of quantification (LLOQ) was converted to a value of ½ LLOQ.

For the calculation of GMTR, any pre-vaccination value reported as <LLOQ were converted to LLOQ, and any post-vaccination value reported as <LLOQ was converted to a value of ½ LLOQ when only either the numerator or the denominator is <LLOQ. If both numerator and denominator are <LLOQ, then both were converted in the same way so that individual titer ratio=1.

Any value reported as >upper limit of quantification (ULOQ) was converted to ULOQ.

### Efficacy

Missing data was not imputed. No test or search for outliers was performed.

### Interim/Preliminary Analysis

The analysis was performed with a stepwise approach.

Several blinded early safety data reviews were performed on safety data collected in subjects from each cohort at specific timepoints.

An unblinded interim analysis is described below on subjects from Cohorts 1, 2, and 3, and at least 90 subjects enrolled in Cohort 4. The interim analysis involves subjects that have provided safety data up to the D84 timepoint and have D84 immunogenicity results available. A specific process was implemented to maintain the blind at the subject and Investigator levels.

An unblinded early analysis is planned on participants from all Cohorts (Cohorts 1, 2, 3, and 4). The early analysis will take place when all participants have provided safety data up to the D84 timepoint and have D84 immunogenicity results available. This early analysis requires unblinding of data; a specific process will be implemented to maintain the blind at the participants and Investigator levels. Based on the results of this analysis, a dose will be confirmed for future studies.

The final unblinded statistical analysis will address the objectives on all subjects (Cohorts 1, 2, 3, and 4) including post season data.

No statistical adjustment is necessary because no hypotheses will be tested.

### Determination of Sample Size and Power Calculation

No sample size calculation was done as there are no statistical hypotheses in this study.

A total of 300 subjects were planned to be enrolled into 1 of the 4 cohorts sequentially:
Cohort 1 (1 administration): 40 subjects, i.e., 20 per vaccine group (RSV low-dose or placebo)
Cohort 2 (2 administrations): 40 subjects, i.e., 20 per vaccine group (RSV low-dose or placebo)
Cohort 3 (1 administration): 40 subjects, i.e., 20 per vaccine group (RSV high-dose or placebo)
Cohort 4 (2 administrations): 180 subjects, i.e., 60 per vaccine group (RSV high-dose or RSV low-dose or placebo).
The actual number of subjects enrolled in each cohort is discussed below in the interim results.

Although there are no statistically powered hypotheses and no sample size computation in this study, the sample size of 100 subjects in the RSV low-dose group (20 from Cohort 1, 20 from Cohort 2, and 60 from Cohort 4) provides a probability of 95% to observe an event that has a true incidence of 3%. The sample size of 80 subjects in the RSV high-dose group (20 from Cohort 3 and 60 from Cohort 4) provides a probability of 95% to observe an event that has a true incidence of 3.75%.

A total of 300 subjects were planned to be enrolled in the study. This corresponds to 120 subjects in Placebo group, 100 subjects in RSV low-dose group and 80 subjects in RSV high-dose group. Table 10 below presents the number of subjects by serostatus, in total and by RSV group, according to varying possible RSV-experienced rates ranging from 5% to 35% (derived from LID/NIH screening data) and provides a global overview of the proportion of RSV-naïve/RSV-experienced subjects that enrolled in the study.

TABLE 10
Number of Subjects by Serostatus, in Total and by RSV Group, According
to Varying Possible RSV-experienced Rates Ranging from 5% to 35%

| | Total (N = 300) | | RSV low-dose group (N = 100) | | RSV high-dose group (N = 80) | |
|---|---|---|---|---|---|---|
| Seropositivity | # RSV- | | # RSV- | | # RSV- | |
| rate | experienced | # RSV-naïve | experienced | # RSV-naïve | experienced | # RSV-naïve |
| 5% | 15 | 285 | 5 | 95 | 4 | 76 |
| 10% | 30 | 270 | 10 | 90 | 8 | 72 |
| 15% | 45 | 255 | 15 | 85 | 12 | 68 |
| 20% | 60 | 240 | 20 | 80 | 16 | 64 |
| 25% | 75 | 225 | 25 | 75 | 20 | 60 |
| 30% | 90 | 210 | 30 | 70 | 24 | 56 |
| 35% | 105 | 195 | 35 | 65 | 28 | 52 |

### Example 2: Interim Results of VAD00001 Study

In the VAD00001 study, approximately 155 study participants (infants and toddlers) have received the investigational product, at any dose or dosing regimen, as full unblinding has not taken place, the final number of investigational product recipients is unknown. The vaccine candidate was found to be well-tolerated, showed high infectivity, genetic stability, and a robust immune response in the VAD00001 interim results.

The ongoing Phase I/II trial (VAD00001) is being conducted in the US and South America (Chile and Honduras). In this trial, the safety, immunogenicity and infectivity of two dose levels (5.6 log₁₀ PFU and 6.2 log₁₀ PFU) of RSV ΔNS2/Δ1313/I1314L (Sanofi) vaccine was and will continue to be assessed in sequential cohorts.

The study was conducted to ensure a safe stepwise approach to reaching the doses in cohort 4. Cohort 4 was designed with both 5.6 and 6.2 log₁₀ PFU per dose to 1) confirm, as suggested by data from NIH trials, that the 6.2 log₁₀ PFU per dose could be utilized for Phase III (with the fall back of 5.6 log₁₀ PFU per dose) and 2) explore acceptability of safety, infectivity, and immunogenicity results of the two doses tested to justify clinically the future dose range boundaries of the non-frozen liquid formulation for final commercialization.

Study participants, 6-18 months of age, were and will continue to be allocated to the following interventions:
Cohort 1 (planned 40, enrolled 36): 1 administration of 5.6 log₁₀ PFU RSV ΔNS2/Δ1313/I1314L (Sanofi) or placebo
Cohort 2 (planned 40, enrolled 21): 2 administrations of 5.6 log₁₀ PFU RSV ΔNS2/Δ1313/I1314L (Sanofi) or placebo, 2 months apart
Cohort 3 (planned 40, enrolled 22): 1 administration of 6.2 log₁₀ PFU RSV ΔNS2/Δ1313/I1314L (Sanofi) or placebo
Cohort 4 (planned 180, enrolled 180): 2 administrations of 5.6 log₁₀ PFU RSV ΔNS2/Δ1313/I1314L (Sanofi), 6.2 log₁₀ PFU RSV ΔNS2/Δ1313/I1314L (Sanofi) or placebo, 2 months apart
Baseline RSV serostatus: R+/R− (see below for the definitions used in the study).

Blood samples in Cohorts 1 and 3 were collected before the study vaccine administration and 56 days later. Blood samples for Cohorts 2 and 4 were collected before each administration and 28 days after the second dose. All participants in Cohorts 1, 2, 3, and 4 are to provide a blood sample during the month following the end of RSV season or at least 5 months after the last vaccine administration for the measurement of post-season RSV antibody titers, to determine if a 4-fold or greater rise in RSV antibody titers has occurred during the RSV season, indicating infection with wild-type RSV that was not detected by surveillance and to explore residual antibody titers after 1 RSV season.

Nasal swab samples were and will continue to be collected in all participants 7 days after the first vaccine administration for Cohorts 1, 2, 3, and 4; and 7 days after the second vaccine administration for Cohorts 2 and 4. The samples were and will continue to be tested for vaccine shedding, and in case of illness for respiratory pathogens. All study participants were and will continue to provide nasal swabs when an illness visit was reported and 48 hours later.

Safety follow-up included immediate surveillance for 30 minutes post-vaccination, collection of solicited adverse reactions, unsolicited AEs, MAAEs, and AESIs for 28 days after each vaccination, and SAEs throughout the trial (up to 12 months).

An unblinded interim analysis for dose selection of the RSV ΔNS2/Δ1313/I1314L (Sanofi) vaccine was conducted on participants from Cohorts 1, 2, and 3 and the first 91 participants enrolled in Cohort 4, as planned in the protocol. The interim analysis occurred and will continue to occur after participants have provided safety data up to 28 days post the second vaccine administration timepoint and have immunogenicity results available at this timepoint. The dose selection to proceed for future development (i.e., 6.2 log₁₀ PFU; targeted midpoint of the future operative range) has been based on a descriptive comparison of the safety, infectivity, and immunogenicity of either dose in all participants up to 28 days post second vaccine administration. The two doses in the trial were assessed for inclusion in the future operative range. The outcome of the interim analysis is summarized below.

Infants with detectable serum IgA to RSV F at baseline were considered RSV experienced while infants without detectable serum IgA at to RSV F baseline were considered RSV naïve. This approach also permits the separation of RSV experienced participants from RSV naïve participants that have placentally transferred serum antibodies as a more discriminatory assay. The rationale for clearly defining naïve vs RSV experienced participants in the Phase I/II study is to thoroughly assess the safety of the live attenuated vaccine (LAV) in a naïve population, since prior RSV infection may impact the infectivity of subsequent RSV infections, including LAV. Considering IgA testing a more discriminant assay, results are given based on the assessment of serostatus at baseline of participants with this serum IgA assay to RSV F as follows:
R−; RSV naïve: participants with titers <limit of detection [LOD]
R+; RSV experienced: participants with titers ≥LOD
Undetermined otherwise

The interim results presented below include 167 participants (50.3% female, mean age 11.0±3.86 months and 5.4% Black/African American, 38.3% Hispanic/Latino) assessed for safety and 139 participants, for whom IgA serostatus data was available, assessed for infectivity and immunogenicity. Three participants of the 170 recruited at the point of this interim analysis did not receive the study intervention and were therefore not included. Participants with documented RSV respiratory disease prior to an evaluation time point were excluded from the analysis for immunogenicity at the corresponding time point.

### Safety, Immunogenicity, and Infectivity Conclusions

### Safety

The safety profile of each dose of RSV ΔNS2/Δ1313/I1314L (Sanofi) vaccine after each and any administration in all infants and toddlers regardless of baseline serostatus was assessed. Symptoms reported post vaccination were comparable between the groups and between cohorts. In this modest-sized group of participants at the point of interim analysis, for solicited administration site reactions, there was a trend to observe these in a higher proportion of participants in the high dose group post vaccination 1 while for AESIs following any vaccination showed a trend to be more common in the low dose group.

Two non-related SAEs were reported during the study, outside of the acute phase: one grade 3 in the low dose group (left hand cellulite) and one grade 2 in the placebo group (RSV-MAARI). Two AESIs in each of the low dose (nasopharyngitis), high dose (croup and stridor in the same participant) and placebo (nasopharyngitis and cough) groups following any vaccination were classed as related by the investigator. All VAD00001 study participants were and will continue to be followed for one RSV season before start of the Phase III program. Data from swabs taken during illness visits is still being expected and will be presented with subsequent analysis. Given the effects of the Covid 19 pandemic on RSV circulation during Cohort 1, 2, and 3 study participation, it is likely that laboratory-confirmed disease in these cohorts will be lower than in Cohort 4. While we do not expect enhanced disease with this candidate due to the nature of the attenuation and available data to date, we will comment on available data on RSV illness following vaccination when this becomes available.

The overall safety profile was comparable between RSV naïve and experienced participants.

No deaths have been reported in the study as of 30 Nov. 2022.

Overall, the safety data indicate an acceptable safety profile for the candidate at both dose levels (see Table 11 and Table 12: Safety overview after vaccination 2-Safety Analysis Set).

### Immunogenicity

Approximately seventy percent (70%) of vaccine recipients attained a 4-fold response in neutralizing antibody titer following the second vaccine dose in both low and high dose groups, compared to 61 and 47% following one vaccine dose in low and high dose RSV naïve participants. This increase in the percentage of participants attaining a 4-fold response following a second administration supports the use of a second vaccine administration in this population. The 70% attaining this fold rise post second vaccine administration aligns with the expected clinical efficacy goal of 70% for the candidate. After one or two vaccinations, 75% attained a 4-fold rise in serum neutralizing antibody titers. The percentage of RSV experienced participants (36% and 22% in the low and high dose groups respectively) also attaining a 4-fold response in neutralizing antibody titers post vaccination 1 suggests potential benefit for this sub-group as well. It must be noted that this comes from a modest sample of participants (n=20 of 97 vaccine recipients) at this point of interim analysis.

Taken together, these data strongly support the use of RSV ΔNS2/Δ1313/I1314L (Sanofi) vaccine at an operative range study for the candidate including doses at 5.6 and 6.2 log PFU/dose. (See Tables 13 and 14)

### Vaccine Virus Shedding and Infectivity

Following each vaccine administration, the shedding of vaccine virus was considered in addition to the fold rise in neutralizing antibody titers or serum IgG as vaccine infectivity. The high level of vaccine infectivity (over 80% and 70% in RSV naïve following the first and second vaccine administration respectively) is supportive of a promising vaccine candidate associated with good vaccine infectivity. When infectivity was considered after either vaccine administration, over 90% of participants had evidence of infection. In the small cohort of RSV experienced participants, relatively high infectivity (80% and 33.3% in low and high dose recipients following one vaccine administration and 60 and 50% following a second vaccine administration) was found, implicating promise for this group. In addition, the marked drop in the percentage of vaccine virus shedders in RSV naïve participants after the second vaccine administration (roughly 20%) compared to the first vaccine administration (over 70%) is as previously documented with other efficacious live attenuated mucosal viral vaccines where subsequent ‘challenge’ in the form of a second vaccine dose is characterized by a marked reduction in vaccine virus shedding. Of note, the shedding data available for this cohort was from data at a single timepoint following each vaccination (seven days post vaccination). While this coincides with the point of peak viral shedding documented in other RSV live attenuated vaccine (LAV) trials, it is likely that some shedders may have been missed. This limitation in the available shedding data makes the results obtained particularly encouraging. (See Tables 15 and 16)

### Overall Conclusions

Interim analysis results showed a promising safety, immunogenicity, and infectivity profile of the RSV ΔNS2/Δ1313/I1314L (Sanofi) candidate.

No safety concerns were identified after 1- and 2-dose administrations of either dose level of the investigational RSV ΔNS2/Δ1313/I1314L (Sanofi) vaccine or by baseline serostatus.

The vaccine virus shedding, and immunogenicity conclusions based on the IgA serostatus at baseline show marked vaccine take demonstrated at both dose levels, and 70% of vaccine RSV-naïve recipients attained a 4-fold response in serum neutralizing antibody responses post the second vaccine administration for both dose levels in RSV-naïve participants.

The results support an operative range for the candidate that includes doses at 5.6 log₁₀ PFU/dose and 6.2 log₁₀ PFU/dose and support the future development of the candidate. An early analysis after all participants have completed the day 28 timepoint post vaccination 2 will be conducted when all the results are unblinded.

TABLE 11
Safety overview after vaccination 1 - Safety Analysis Set

| | Cohort 1 | | | | | | Cohort 2 | | |
|---|---|---|---|---|---|---|---|---|---|
| | RSV low dose | | | Placebo | | | RSV low dose | | |
| Period/ | (N = 17) | | | (N = 18) | | | (N = 10) | | |
| Participants experiencing | | | (95% | | | (95% | | | (95% |
| at least one: | n/M | % | CI) | n/M | % | CI) | n/M | % | CI) |

Baseline IgA serostatus: RSV-naïve

| | | | | | | | | | |
|---|---|---|---|---|---|---|---|---|---|
| Within 30 minutes after | | | | | | | | | |
| vaccination 1 | | | | | | | | | |
| Immediate unsolicited | 0/9 | 0 | (0; | 0/7 | 0 | (0; | 0/8 | 0 | (0; |
| systemic AE | | | 33.6) | | | 41.0) | | | 36.9) |
| Immediate unsolicited | 0/9 | 0 | (0; | 0/7 | 0 | (0; | 0/8 | 0 | (0; |
| systemic AR | | | 33.6) | | | 41.0) | | | 36.9) |
| Within 28 days after | | | | | | | | | |
| vaccination 1 | | | | | | | | | |
| Solicited | 9/9 | 100 | (66.4; | 4/7 | 57.1 | (18.4; | 7/8 | 87.5 | (47.3; |
| administration site | | | 100) | | | 90.1) | | | 99.7) |
| reaction | | | | | | | | | |
| Solicited systemic | 9/9 | 100 | (66.4; | 6/7 | 85.7 | (42.1; | 7/8 | 87.5 | (47.3; |
| reaction | | | 100) | | | 99.6) | | | 99.7) |
| Unsolicited AE | 2/9 | 22.2 | (2.8; | 1/7 | 14.3 | (0.4; | 1/8 | 12.5 | (0.3; |
| | | | 60.0) | | | 57.9) | | | 52.7) |
| Unsolicited AR | 0/9 | 0 | (0; | 0/7 | 0 | (0; | 1/8 | 12.5 | (0.3; |
| | | | 33.6) | | | 41.0) | | | 52.7) |
| AESI | 1/9 | 11.1 | (0.3; | 0/7 | 0 | (0; | 0/8 | 0 | (0; |
| | | | 48.2) | | | 41.0) | | | 36.9) |
| MAAE | 1/9 | 11.1 | (0.3; | 1/7 | 14.3 | (0.4; | 0/8 | 0 | (0; |
| | | | 48.2) | | | 57.9) | | | 36.9) |
| AE leading to study | 0/9 | 0 | (0; | 0/7 | 0 | (0; | 0/8 | 0 | (0; |
| discontinuation | | | 33.6) | | | 41.0) | | | 36.9) |
| SAE | 0/9 | 0 | (0; | 0/7 | 0 | (0; | 0/8 | 0 | (0; |
| | | | 33.6) | | | 41.0) | | | 36.9) |
| Death | 0/9 | 0 | (0; | 0/7 | 0 | (0; | 0/8 | 0 | (0; |
| | | | 33.6) | | | 41.0) | | | 36.9) |

Baseline IgA serostatus: RSV-experienced

| | | | | | | | | | |
|---|---|---|---|---|---|---|---|---|---|
| Within 30 minutes after | | | | | | | | | |
| vaccination 1 | | | | | | | | | |
| Immediate unsolicited | 0/5 | 0 | (0; | 0/5 | 0 | (0; | 0/2 | 0 | (0; |
| systemic AE | | | 52.2) | | | 52.2) | | | 84.2) |
| Immediate unsolicited | 0/5 | 0 | (0; | 0/5 | 0 | (0; | 0/2 | 0 | (0; |
| systemic AR | | | 52.2) | | | 52.2) | | | 84.2) |
| Within 28 days after | | | | | | | | | |
| vaccination 1 | | | | | | | | | |
| Solicited | 4/5 | 80.0 | (28.4; | 5/5 | 100 | (47.8; | 1/2 | 50.0 | (1.3; |
| administration site | | | 99.5) | | | 100) | | | 98.7) |
| reaction | | | | | | | | | |
| Solicited systemic | 3/5 | 60.0 | (14.7; | 4/5 | 80.0 | (28.4; | 2/2 | 100 | (15.8; |
| reaction | | | 94.7) | | | 99.5) | | | 100) |
| Unsolicited AE | 3/5 | 60.0 | (14.7; | 1/5 | 20.0 | (0.5; | 1/2 | 50.0 | (1.3; |
| | | | 94.7) | | | 71.6) | | | 98.7) |
| Unsolicited AR | 0/5 | 0 | (0; | 0/5 | 0 | (0; | 0/2 | 0 | (0; |
| | | | 52.2) | | | 52.2) | | | 84.2) |
| AESI | 2/5 | 40.0 | (5.3; | 0/5 | 0 | (0; | 0/2 | 0 | (0; |
| | | | 85.3) | | | 52.2) | | | 84.2) |
| MAAE | 1/5 | 20.0 | (0.5; | 0/5 | 0 | (0; | 0/2 | 0 | (0; |
| | | | 71.6) | | | 52.2) | | | 84.2) |
| AE leading to study | 0/5 | 0 | (0; | 0/5 | 0 | (0; | 0/2 | 0 | (0; |
| discontinuation | | | 52.2) | | | 52.2) | | | 84.2) |
| SAE | 0/5 | 0 | (0; | 0/5 | 0 | (0; | 0/2 | 0 | (0; |
| | | | 52.2) | | | 52.2) | | | 84.2) |
| Death | 0/5 | 0 | (0; | 0/5 | 0 | (0; | 0/2 | 0 | (0; |
| | | | 52.2) | | | 52.2) | | | 84.2) |

Baseline IgA serostatus: Undetermined

| Within 30 minutes after | | | | | | | | | |
|---|---|---|---|---|---|---|---|---|---|
| vaccination 1 | | | | | | | | | |
| Immediate unsolicited | 0/3 | 0 | (0; | 0/6 | 0 | (0; | 0/0 | NC | (NC; |
| systemic AE | | | 70.8) | | | 45.9) | | | NC) |
| Immediate unsolicited | 0/3 | 0 | (0; | 0/6 | 0 | (0; | 0/0 | NC | (NC; |
| systemic AR | | | 70.8) | | | 45.9) | | | NC) |
| Within 28 days after | | | | | | | | | |
| vaccination 1 | | | | | | | | | |
| Solicited | 2/3 | 66.7 | (9.4; | 3/6 | 50.0 | (11.8; | 0/0 | NC | (NC; |
| administration site | | | 99.2) | | | 88.2) | | | NC) |
| reaction | | | | | | | | | |
| Solicited systemic | 2/3 | 66.7 | (9.4; | 6/6 | 100 | (54.1; | 0/0 | NC | (NC; |
| reaction | | | 99.2) | | | 100) | | | NC) |
| Unsolicited AE | 0/3 | 0 | (0; | 0/6 | 0 | (0; | 0/0 | NC | (NC; |
| | | | 70.8) | | | 45.9) | | | NC) |
| Unsolicited AR | 0/3 | 0 | (0; | 0/6 | 0 | (0; | 0/0 | NC | (NC; |
| | | | 70.8) | | | 45.9) | | | NC) |
| AESI | 0/3 | 0 | (0; | 0/6 | 0 | (0; | 0/0 | NC | (NC; |
| | | | 70.8) | | | 45.9) | | | NC) |
| MAAE | 0/3 | 0 | (0; | 0/6 | 0 | (0; | 0/0 | NC | (NC; |
| | | | 70.8) | | | 45.9) | | | NC) |
| AE leading to study | 0/3 | 0 | (0; | 0/6 | 0 | (0; | 0/0 | NC | (NC; |
| discontinuation | | | 70.8) | | | 45.9) | | | NC) |
| SAE | 0/3 | 0 | (0; | 0/6 | 0 | (0; | 0/0 | NC | (NC; |
| | | | 70.8) | | | 45.9) | | | NC) |
| Death | 0/3 | 0 | (0; | 0/6 | 0 | (0; | 0/0 | NC | (NC; |
| | | | 70.8) | | | 45.9) | | | NC) |
| | Cohort 2 | | | Cohort 3 | | | | | |
| | Placebo | | | RSV high dose | | | Placebo | | |
| Period/ | (N = 10) | | | (N = 10) | | | (N = 12) | | |
| Participants experiencing | | | (95% | | | (95% | | | (95% |
| at least one: | n/M | % | CI) | n/M | % | CI) | n/M | % | CI) |

Baseline IgA serostatus: RSV-naïve

| | | | | | | | | | |
|---|---|---|---|---|---|---|---|---|---|
| Within 30 minutes after | | | | | | | | | |
| vaccination 1 | | | | | | | | | |
| Immediate unsolicited | 0/10 | 0 | (0; | 0/8 | 0 | (0; | 0/9 | 0 | (0; |
| systemic AE | | | 30.8) | | | 36.9) | | | 33.6) |
| Immediate unsolicited | 0/10 | 0 | (0; | 0/8 | 0 | (0; | 0/9 | 0 | (0; |
| systemic AR | | | 30.8) | | | 36.9) | | | 33.6) |
| Within 28 days after | | | | | | | | | |
| vaccination 1 | | | | | | | | | |
| Solicited | 6/10 | 60.0 | (26.2; | 7/8 | 87.5 | (47.3; | 5/9 | 55.6 | (21.2; |
| administration site | | | 87.8) | | | 99.7) | | | 86.3) |
| reaction | | | | | | | | | |
| Solicited systemic | 8/10 | 80.0 | (44.4; | 7/8 | 87.5 | (47.3; | 8/9 | 88.9 | (51.8; |
| reaction | | | 97.5) | | | 99.7) | | | 99.7) |
| Unsolicited AE | 5/10 | 50.0 | (18.7; | 0/8 | 0 | (0; | 2/9 | 22.2 | (2.8; |
| | | | 81.3) | | | 36.9) | | | 60.0) |
| Unsolicited AR | 3/10 | 30.0 | (6.7; | 0/8 | 0 | (0; | 1/9 | 11.1 | (0.3; |
| | | | 65.2) | | | 36.9) | | | 48.2) |
| AESI | 0/10 | 0 | (0; | 0/8 | 0 | (0; | 1/9 | 11.1 | (0.3; |
| | | | 30.8) | | | 36.9) | | | 48.2) |
| MAAE | 3/10 | 30.0 | (6.7; | 0/8 | 0 | (0; | 1/9 | 11.1 | (0.3; |
| | | | 65.2) | | | 36.9) | | | 48.2) |
| AE leading to study | 0/10 | 0 | (0; | 0/8 | 0 | (0; | 0/9 | 0 | (0; |
| discontinuation | | | 30.8) | | | 36.9) | | | 33.6) |
| SAE | 0/10 | 0 | (0; | 0/8 | 0 | (0; | 0/9 | 0 | (0; |
| | | | 30.8) | | | 36.9) | | | 33.6) |
| Death | 0/10 | 0 | (0; | 0/8 | 0 | (0; | 0/9 | 0 | (0; |
| | | | 30.8) | | | 36.9) | | | 33.6) |

Baseline IgA serostatus: RSV-experienced

| | | | | | | | | | |
|---|---|---|---|---|---|---|---|---|---|
| Within 30 minutes after | | | | | | | | | |
| vaccination 1 | | | | | | | | | |
| Immediate unsolicited | 0/0 | NC | (NC; | 0/1 | 0 | (0; | 0/1 | 0 | (0; |
| systemic AE | | | NC) | | | 97.5) | | | 97.5) |
| Immediate unsolicited | 0/0 | NC | (NC; | 0/1 | 0 | (0; | 0/1 | 0 | (0; |
| systemic AR | | | NC) | | | 97.5) | | | 97.5) |
| Within 28 days after | | | | | | | | | |
| vaccination 1 | | | | | | | | | |
| Solicited | 0/0 | NC | (NC; | 1/1 | 100 | (2.5; | 0/1 | 0 | (0; |
| administration site | | | NC) | | | 100) | | | 97.5) |
| reaction | | | | | | | | | |
| Solicited systemic | 0/0 | NC | (NC; | 1/1 | 100 | (2.5; | 0/1 | 0 | (0; |
| reaction | | | NC | | | 100) | | | 97.5) |
| Unsolicited AE | 0/0 | NC | (NC; | 0/1 | 0 | (0; | 0/1 | 0 | (0; |
| | | | NC) | | | 97.5) | | | 97.5) |
| Unsolicited AR | 0/0 | NC | (NC; | 0/1 | 0 | (0; | 0/1 | 0 | (0; |
| | | | NC) | | | 97.5) | | | 97.5) |
| AESI | 0/0 | NC | (NC; | 0/1 | 0 | (0; | 0/1 | 0 | (0; |
| | | | NC) | | | 97.5) | | | 97.5) |
| MAAE | 0/0 | NC | (NC; | 0/1 | 0 | (0; | 0/1 | 0 | (0; |
| | | | NC) | | | 97.5) | | | 97.5) |
| AE leading to study | 0/0 | NC | (NC; | 0/1 | 0 | (0; | 0/1 | 0 | (0; |
| discontinuation | | | NC) | | | 97.5) | | | 97.5) |
| SAE | 0/0 | NC | (NC; | 0/1 | 0 | (0; | 0/1 | 0 | (0; |
| | | | NC) | | | 97.5) | | | 97.5) |
| Death | 0/0 | NC | (NC; | 0/1 | 0 | (0; | 0/1 | 0 | (0; |
| | | | NC) | | | 97.5) | | | 97.5) |

Baseline IgA serostatus: Undetermined

| | | | | | | | | | |
|---|---|---|---|---|---|---|---|---|---|
| Within 30 minutes after | | | | | | | | | |
| vaccination 1 | | | | | | | | | |
| Immediate unsolicited | 0/0 | NC | (NC; | 0/1 | 0 | (0; | 0/2 | 0 | (0; |
| systemic AE | | | NC) | | | 97.5) | | | 84.2) |
| Immediate unsolicited | 0/0 | NC | (NC; | 0/1 | 0 | (0; | 0/2 | 0 | (0; |
| systemic AR | | | NC) | | | 97.5) | | | 84.2) |
| Within 28 days after | | | | | | | | | |
| vaccination 1 | | | | | | | | | |
| Solicited | 0/0 | NC | (NC; | 1/1 | 100 | (2.5; | 1/1 | 100 | (2.5; |
| administration site | | | NC) | | | 100) | | | 100) |
| reaction | | | | | | | | | |
| Solicited systemic | 0/0 | NC | (NC; | 1/1 | 100 | (2.5; | 1/1 | 100 | (2.5; |
| reaction | | | NC) | | | 100) | | | 100) |
| Unsolicited AE | 0/0 | NC | (NC; | 0/1 | 0 | (0; | 0/2 | 0 | (0; |
| | | | NC) | | | 97.5) | | | 84.2) |
| Unsolicited AR | 0/0 | NC | (NC; | 0/1 | 0 | (0; | 0/2 | 0 | (0; |
| | | | NC) | | | 97.5) | | | 84.2) |
| AESI | 0/0 | NC | (NC; | 0/1 | 0 | (0; | 0/2 | 0 | (0; |
| | | | NC) | | | 97.5) | | | 84.2 |
| MAAE | 0/0 | NC | (NC; | 0/1 | 0 | (0; | 0/2 | 0 | (0; |
| | | | NC) | | | 97.5) | | | 84.2) |
| AE leading to study | 0/0 | NC | (NC; | 0/1 | 0 | (0; | 0/2 | 0 | (0; |
| discontinuation | | | NC) | | | 97.5) | | | 84.2) |
| SAE | 0/0 | NC | (NC; | 0/1 | 0 | (0; | 0/2 | 0 | (0; |
| | | | NC) | | | 97.5) | | | 84.2) |
| Death | 0/0 | NC | (NC; | 0/1 | 0 | (0; | 0/2 | 0 | (0; |
| | | | NC) | | | 97.5) | | | 84.2) |

| | Cohort 4 | | | | | | | | | |
|---|---|---|---|---|---|---|---|---|---|---|
| | RSV low dose | | | RSV high dose | | | Placebo | | | Cohorts 1 to 4 (pooled) |
| Period/ | (N = 31) | | | (N = 29) | | | (N = 30) | | | RSV low dose |
| Participants experiencing | | | (95% | | | (95% | | | (95% | (N = 58) |
| at least one: | n/M | % | CI) | n/M | % | CI) | n/M | % | CI) | n/M |

Baseline IgA serostatus: RSV-naïve

| | | | | | | | | | | |
|---|---|---|---|---|---|---|---|---|---|---|
| Within 30 minutes after | | | | | | | | | | |
| vaccination 1 | | | | | | | | | | |
| Immediate unsolicited | 0/21 | 0 | (0; | 0/15 | 0 | (0; | 0/19 | 0 | (0; | 0/38 |
| systemic AE | | | 16.1) | | | 21.8) | | | 17.6) | |
| Immediate unsolicited | 0/21 | 0 | (0; | 0/15 | 0 | (0; | 0/19 | 0 | (0; | 0/38 |
| systemic AR | | | 16.1) | | | 21.8) | | | 17.6) | |
| Within 28 days after | | | | | | | | | | |
| vaccination 1 | | | | | | | | | | |
| Solicited | 17/20 | 85.0 | (62.1; | 15/15 | 100 | (78.2; | 11/19 | 57.9 | (33.5; | 33/37 |
| administration site | | | 96.8) | | | 100) | | | 79.7) | |
| reaction | | | | | | | | | | |
| Solicited systemic | 18/20 | 90.0 | (68.3; | 15/15 | 100 | (78.2; | 16/19 | 84.2 | (60.4; | 34/37 |
| reaction | | | 98.8) | | | 100) | | | 96.6) | |
| Unsolicited AE | 13/21 | 61.9 | (38.4; | 10/15 | 66.7 | (38.4; | 8/19 | 42.1 | (20.3; | 16/38 |
| | | | 81.9) | | | 88.2) | | | 66.5) | |
| Unsolicited AR | 1/21 | 4.8 | (0.1; | 1/15 | 6.7 | (0.2; | 1/19 | 5.3 | (0.1; | 2/38 |
| | | | 23.8) | | | 31.9) | | | 26.0) | |
| AESI | 5/21 | 23.8 | 8.2; | 2/15 | 13.3 | (1.7; | 3/19 | 15.8 | (3.4; | 6/38 |
| | | | 47.2) | | | 40.5) | | | 39.6) | |
| MAAE | 8/21 | 38.1 | (18.1; | 5/15 | 33.3 | (11.8; | 3/19 | 15.8 | (3.4; | 9/38 |
| | | | 61.6) | | | 61.6) | | | 39.6) | |
| AE leading to study | 0/21 | 0 | (0; | 0/15 | 0 | (0; | 0/19 | 0 | (0; | 0/38 |
| discontinuation | | | 16.1) | | | 21.8) | | | 17.6) | |
| SAE | 0/21 | 0 | (0; | 0/15 | 0 | (0; | 0/19 | 0 | (0; | 0/38 |
| | | | 16.1) | | | 21.8) | | | 17.6) | |
| Death | 0/21 | 0 | (0; | 0/15 | 0 | (0; | 0/19 | 0 | (0; | 0/38 |
| | | | 16.1) | | | 21.8) | | | 17.6) | |

Baseline IgA serostatus: RSV-experienced

| | | | | | | | | | | |
|---|---|---|---|---|---|---|---|---|---|---|
| Within 30 minutes after | | | | | | | | | | |
| vaccination 1 | | | | | | | | | | |
| Immediate unsolicited | 0/4 | 0 | (0; | 0/8 | 0 | (0; | 0/7 | 0 | (0; | 0/11 |
| systemic AE | | | 60.2) | | | 36.9) | | | 41.0) | |
| Immediate unsolicited | 0/4 | 0 | (0; | 0/8 | 0 | (0; | 0/7 | 0 | (0; | 0/11 |
| systemic AR | | | 60.2) | | | 36.9) | | | 41.0) | |
| Within 28 days after | | | | | | | | | | |
| vaccination 1 | | | | | | | | | | |
| Solicited | 4/4 | 100 | (39.8; | 8/8 | 100 | (63.1; | 7/7 | 100 | (59.0; | 9/11 |
| administration site | | | 100) | | | 100) | | | 100) | |
| reaction | | | | | | | | | | |
| Solicited systemic | 3/4 | 75.0 | (19.4; | 7/8 | 87.5 | (47.3; | 6/7 | 85.7 | (42.1; | 8/11 |
| reaction | | | 99.4) | | | 99.7) | | | 99.6) | |
| Unsolicited AE | 2/4 | 50.0 | (6.8; | 4/8 | 50.0 | (15.7; | 7/7 | 100 | (59.0; | 6/11 |
| | | | 93.2) | | | 84.3) | | | 100) | |
| Unsolicited AR | 1/4 | 25.0 | (0.6; | 0/8 | 0 | (0; | 0/7 | 0 | (0; | 1/11 |
| | | | 80.6) | | | 36.9) | | | 41.0) | |
| AESI | 1/4 | 25.0 | (0.6; | 0/8 | 0 | (0; | 2/7 | 28.6 | (3.7; | 3/11 |
| | | | 80.6) | | | 36.9) | | | 71.0) | |
| MAAE | 1/4 | 25.0 | (0.6; | 2/8 | 25.0 | (3.2; | 2/7 | 28.6 | (3.7; | 2/11 |
| | | | 80.6) | | | 65.1) | | | 71.0) | |
| AE leading to study | 0/4 | 0 | (0; | 0/8 | 0 | (0; | 0/7 | 0 | (0; | 0/11 |
| discontinuation | | | 60.2) | | | 36.9) | | | 41.0) | |
| SAE | 0/4 | 0 | (0; | 0/8 | 0 | (0; | 0/7 | 0 | (0; | 0/11 |
| | | | 60.2) | | | 36.9) | | | 41.0) | |
| Death | 0/4 | 0 | (0; | 0/8 | 0 | (0; | 0/7 | 0 | (0; | 0/11 |
| | | | 60.2) | | | 36.9) | | | 41.0) | |

Baseline IgA serostatus: Undetermined

| | | | | | | | | | | |
|---|---|---|---|---|---|---|---|---|---|---|
| Within 30 minutes after | | | | | | | | | | |
| vaccination 1 | | | | | | | | | | |
| Immediate unsolicited | 0/6 | 0 | (0; | 0/6 | 0 | (0; | 1/4 | 25.0 | (0.6; | 0/9 |
| systemic AE | | | 45.9) | | | 45.9) | | | 80.6) | |
| Immediate unsolicited | 0/6 | 0 | (0; | 0/6 | 0 | (0; | 1/4 | 25.0 | (0.6; | 0/9 |
| systemic AR | | | 45.9) | | | 45.9) | | | 80.6) | |
| Within 28 days after | | | | | | | | | | |
| vaccination 1 | | | | | | | | | | |
| Solicited | 5/6 | 83.3 | (35.9; | 3/4 | 75.0 | (19.4; | 1/4 | 25.0 | (0.6; | 7/9 |
| administration site | | | 99.6) | | | 99.4) | | | 80.6) | |
| reaction | | | | | | | | | | |
| Solicited systemic | 5/6 | 83.3 | (35.9; | 1/5 | 20.0 | (0.5; | 2/4 | 50.0 | (6.8; | 7/9 |
| reaction | | | 99.6) | | | 71.6) | | | 93.2) | |
| Unsolicited AE | 4/6 | 66.7 | (22.3; | 1/6 | 16.7 | (0.4; | 2/4 | 50.0 | (6.8; | 4/9 |
| | | | 95.7) | | | 64.1) | | | 93.2) | |
| Unsolicited AR | 1/6 | 16.7 | (0.4; | 1/6 | 16.7 | (0.4; | 1/4 | 25.0 | (0.6; | 1/9 |
| | | | 64.1) | | | 64.1) | | | 80.6) | |
| AESI | 2/6 | 33.3 | (4.3; | 1/6 | 16.7 | (0.4; | 0/4 | 0 | (0; | 2/9 |
| | | | 77.7) | | | 64.1) | | | 60.2) | |
| MAAE | 3/6 | 50.0 | (11.8; | 1/6 | 16.7 | (0.4; | 2/4 | 50.0 | (6.8; | 3/9 |
| | | | 88.2) | | | 64.1) | | | 93.2) | |
| AE leading to study | 0/6 | 0 | (0; | 0/6 | 0 | (0; | 0/4 | 0 | (0; | 0/9 |
| discontinuation | | | 45.9) | | | 45.9) | | | 60.2) | |
| SAE | 0/6 | 0 | (0; | 0/6 | 0 | (0; | 0/4 | 0 | (0; | 0/9 |
| | | | 45.9) | | | 45.9) | | | 60.2) | |
| Death | 0/6 | 0 | (0; | 0/6 | 0 | (0; | 0/4 | 0 | (0; | 0/9 |
| | | | 45.9) | | | 45.9) | | | 60.2) | |

| | Cohorts 1 to 4 (pooled) | | | | | | | |
|---|---|---|---|---|---|---|---|---|
| | RSV low dose | | RSV high dose | | | Placebo | | |
| Period/ | (N = 58) | | (N = 39) | | | (N = 70) | | |
| Participants experiencing | | (95% | | | (95% | | | (95% |
| at least one: | % | CI) | n/M | % | CI) | n/M | % | CI) |
| Baseline IgA serostatus: RSV-naïve | | | | | | | | |
| Within 30 minutes after | | | | | | | | |
| vaccination 1 | | | | | | | | |
| Immediate unsolicited | 0 | (0; | 0/23 | 0 | (0; | 0/45 | 0 | (0; |
| systemic AE | | 9.3) | | | 14.8) | | | 7.9) |
| Immediate unsolicited | 0 | (0; | 0/23 | 0 | (0; | 0/45 | 0 | (0; |
| systemic AR | | 9.3) | | | 14.8) | | | 7.9) |
| Within 28 days after | | | | | | | | |
| vaccination 1 | | | | | | | | |
| Solicited | 89.2 | (74.6; | 22/23 | 95.7 | (78.1; | 26/45 | 57.8 | (42.2; |
| administration site | | 97.0) | | | 99.9) | | | 72.3) |
| reaction | | | | | | | | |
| Solicited systemic | 91.9 | (78.1; | 22/23 | 95.7 | (78.1; | 38/45 | 84.4 | (70.5; |
| reaction | | 98.3) | | | 99.9) | | | 93.5) |
| Unsolicited AE | 42.1 | (26.3; | 10/23 | 43.5 | (23.2; | 16/45 | 35.6 | (21.9; |
| | | 59.2) | | | 65.5) | | | 51.2) |
| Unsolicited AR | 5.3 | (0.6; | 1/23 | 4.3 | (0.1; | 5/45 | 11.1 | (3.7; |
| | | 17.7) | | | 21.9) | | | 24.1) |
| AESI | 15.8 | (6.0; | 2/23 | 8.7 | (1.1; | 4/45 | 8.9 | (2.5 |
| | | 31.3) | | | 28.0) | | | 21.2) |
| MAAE | 23.7 | (11.4; | 5/23 | 21.7 | (7.5; | 8/45 | 17.8 | (8.0; |
| | | 40.2) | | | 43.7) | | | 32.1) |
| AE leading to study | 0 | (0; | 0/23 | 0 | (0; | 0/45 | 0 | (0; |
| discontinuation | | 9.3) | | | 14.8) | | | 7.9) |
| SAE | 0 | (0; | 0/23 | 0 | (0; | 0/45 | 0 | (0; |
| | | 9.3) | | | 14.8) | | | 7.9) |
| Death | 0 | (0; | 0/23 | 0 | (0; | 0/45 | 0 | (0; |
| | | 9.3) | | | 14.8) | | | 7.9) |
| Baseline IgA serostatus: RSV-experienced | | | | | | | | |
| Within 30 minutes after | | | | | | | | |
| vaccination 1 | | | | | | | | |
| Immediate unsolicited | 0 | (0; | 0/9 | 0 | (0; | 0/13 | 0 | (0; |
| systemic AE | | 28.5) | | | 33.6) | | | 24.7) |
| Immediate unsolicited | 0 | (0; | 0/9 | 0 | (0; | 0/13 | 0 | (0; |
| systemic AR | | 28.5) | | | 33.6) | | | 24.7) |
| Within 28 days after | | | | | | | | |
| vaccination 1 | | | | | | | | |
| Solicited | 81.8 | (48.2; | 9/9 | 100 | (66.4; | 12/13 | 92.3 | (64.0; |
| administration site | | 97.7) | | | 100) | | | 99.8) |
| reaction | | | | | | | | |
| Solicited systemic | 72.7 | (39.0; | 8/9 | 88.9 | (51.8; | 10/13 | 76.9 | (46.2; |
| reaction | | 94.0) | | | 99.7) | | | 95.0) |
| Unsolicited AE | 54.5 | (23.4; | 4/9 | 44.4 | (13.7; | 8/13 | 61.5 | (31.6; |
| | | 83.3) | | | 78.8) | | | 86.1) |
| Unsolicited AR | 9.1 | (0.2; | 0/9 | 0 | (0; | 0/13 | 0 | (0; |
| | | 41.3) | | | 33.6) | | | 24.7) |
| AESI | 27.3 | (6.0; | 0/9 | 0 | (0; | 2/13 | 15.4 | (1.9; |
| | | 61.0) | | | 33.6) | | | 45.4) |
| MAAE | 18.2 | (2.3; | 2/9 | 22.2 | (2.8; | 2/13 | 15.4 | (1.9; |
| | | 51.8) | | | 60.0) | | | 45.4) |
| AE leading to study | 0 | (0; | 0/9 | 0 | (0; | 0/13 | 0 | (0; |
| discontinuation | | 28.5) | | | 33.6) | | | 24.7) |
| SAE | 0 | (0; | 0/9 | 0 | (0; | 0/13 | 0 | (0; |
| | | 28.5) | | | 33.6) | | | 24.7) |
| Death | 0 | (0; | 0/9 | 0 | (0; | 0/13 | 0 | (0; |
| | | 28.5) | | | 33.6) | | | 24.7) |
| Baseline IgA serostatus: Undetermined | | | | | | | | |
| Within 30 minutes after | | | | | | | | |
| vaccination 1 | | | | | | | | |
| Immediate unsolicited | 0 | (0; | 0/7 | 0 | (0; | 1/12 | 8.3 | (0.2; |
| systemic AE | | 33.6) | | | 41.0) | | | 38.5) |
| Immediate unsolicited | 0 | (0; | 0/7 | 0 | (0; | 1/12 | 8.3 | (0.2; |
| systemic AR | | 33.6) | | | 41.0) | | | 38.5) |
| Within 28 days after | | | | | | | | |
| vaccination 1 | | | | | | | | |
| Solicited | 77.8 | (40.0; | 4/5 | 80.0 | (28.4; | 5/11 | 45.5 | (16.7; |
| administration site | | 97.2) | | | 99.5) | | | 76.6) |
| reaction | | | | | | | | |
| Solicited systemic | 77.8 | (40.0; | 2/6 | 33.3 | (4.3; | 9/11 | 81.8 | (48.2; |
| reaction | | 97.2) | | | 77.7) | | | 97.7) |
| Unsolicited AE | 44.4 | (13.7; | 1/7 | 14.3 | (0.4; | 2/12 | 16.7 | (2.1; |
| | | 78.8) | | | 57.9) | | | 48.4) |
| Unsolicited AR | 11.1 | (0.3; | 1/7 | 14.3 | (0.4; | 1/12 | 8.3 | (0.2; |
| | | 48.2) | | | 57.9) | | | 38.5) |
| AESI | 22.2 | (2.8; | 1/7 | 14.3 | (0.4; | 0/12 | 0 | (0; |
| | | 60.0) | | | 57.9) | | | 26.5) |
| MAAE | 33.3 | (7.5; | 1/7 | 14.3 | (0.4; | 2/12 | 16.7 | (2.1; |
| | | 70.1) | | | 57.9) | | | 48.4) |
| AE leading to study | 0 | (0; | 0/7 | 0 | (0; | 0/12 | 0 | (0; |
| discontinuation | | 33.6) | | | 41.0) | | | 26.5) |
| SAE | 0 | (0; | 0/7 | 0 | (0; | 0/12 | 0 | (0; |
| | | 33.6) | | | 41.0) | | | 26.5) |
| Death | 0 | (0; | 0/7 | 0 | (0; | 0/12 | 0 | (0; |
| | | 33.6) | | | 41.0) | | | 26.5) |

n: number of participants experiencing the endpoint listed in the first two columns
M: number of participants with available data for the relevant endpoint
AR: Reactions related to study vaccine. Missing relationship (for the definition of AR) will be considered at the time of analysis as related to the vaccine.
“AE leading to study discontinuation” row includes those participants who have either on the termination form, the reason for early termination “Adverse Event” checked or lists an AE (solicited, unsolicited, or SAE) that has “Caused Study Discontinuation” checked that is at least Grade 1 and is within the time period indicated.
Baseline IgA serostatus is determined using serum anti-F IgA Ab titers measured by ELISA at D 0, as follows: “RSV-naïve” for participants with titers <4.8 EU/mL (<LOD), “RSV-experienced” for participants with titers >=4.8 EU/mL (>=LOD), “Undetermined” otherwise.
NC: Not Computed (if M <= 5 or STD = 0)
Safety analysis set: All participants receiving at least one administration of investigational product

TABLE 12
Safety overview after vaccination 2 - Safety Analysis Set

| | Cohort 2 | | | | | | Cohort 4 | | | | | | | | |
|---|---|---|---|---|---|---|---|---|---|---|---|---|---|---|---|
| Period/ | RSV low dose | | | Placebo | | | RSV low dose | | | RSV high dose | | | Placebo | | |
| Participants | (N = 8) | | | (N = 8) | | | (N = 27) | | | (N = 28) | | | (N = 27) | | |
| experiencing | | | (95% | | | (95% | | | (95% | | | (95% | | | (95% |
| at least one: | n/M | % | CI) | n/M | % | CI) | n/M | % | CI) | n/M | % | CI) | n/M | % | CI) |

Baseline IgA serostatus: RSV-naïve

| | | | | | | | | | | | | | | | |
|---|---|---|---|---|---|---|---|---|---|---|---|---|---|---|---|
| Within 30 minutes after | | | | | | | | | | | | | | | |
| vaccination 2 | | | | | | | | | | | | | | | |
| Immediate unsolicited | 0/6 | 0 | (0; | 0/8 | 0 | (0; | 0/18 | 0 | (0; | 0/14 | 0 | (0; | 0/19 | 0 | (0; |
| systemic AE | | | 45.9) | | | 36.9) | | | 18.5) | | | 23.2) | | | 17.6) |
| Immediate unsolicited | 0/6 | 0 | (0; | 0/8 | 0 | (0; | 0/18 | 0 | (0; | 0/14 | 0 | (0; | 0/19 | 0 | (0; |
| systemic AR | | | 45.9) | | | 36.9) | | | 18.5) | | | 23.2) | | | 17.6) |
| Within 28 days after | | | | | | | | | | | | | | | |
| vaccination 2 | | | | | | | | | | | | | | | |
| Solicited administration | 2/6 | 33.3 | (4.3; | 4/8 | 50.0 | (15.7; | 15/17 | 88.2 | (63.6; | 14/14 | 100 | (76.8; | 14/19 | 73.7 | (48.8; |
| site reaction | | | 77.7) | | | 84.3) | | | 98.5) | | | 100) | | | 90.9) |
| Solicited systemic | 3/6 | 50.0 | (11.8; | 4/8 | 50.0 | (15.7; | 13/17 | 76.5 | (50.1; | 11/14 | 78.6 | (49.2; | 12/19 | 63.2 | (38.4; |
| reaction | | | 88.2) | | | 84.3) | | | 93.2) | | | 95.3) | | | 83.7) |
| Unsolicited AE | 0/6 | 0 | (0; | 2/8 | 25.0 | (3.2; | 6/18 | 33.3 | (13.3; | 5/14 | 35.7 | (12.8; | 8/19 | 42.1 | (20.3; |
| | | | 45.9) | | | 65.1) | | | 59.0) | | | 64.9) | | | 66.5) |
| Unsolicited AR | 0/6 | 0 | (0; | 2/8 | 25.0 | (3.2; | 2/18 | 11.1 | (1.4; | 1/14 | 7.1 | (0.2; | 2/19 | 10.5 | (1.3; |
| | | | 45.9) | | | 65.1) | | | 34.7) | | | 33.9) | | | 33.1) |
| AESI | 0/6 | 0 | (0; | 0/8 | 0 | (0; | 4/18 | 22.2 | (6.4; | 1/14 | 7.1 | (0.2; | 3/19 | 15.8 | (3.4; |
| | | | 45.9) | | | 36.9) | | | 47.6) | | | 33.9) | | | 39.6) |
| MAAE | 0/6 | 0 | (0; | 2/8 | 25.0 | (3.2; | 4/18 | 22.2 | (6.4; | 3/14 | 21.4 | (4.7; | 5/19 | 26.3 | (9.1; |
| | | | 45.9) | | | 65.1) | | | 47.6) | | | 50.8) | | | 51.2) |
| AE leading to study | 0/6 | 0 | (0; | 0/8 | 0 | (0; | 0/18 | 0 | (0; | 0/14 | 0 | (0; | 0/19 | 0 | (0; |
| discontinuation | | | 45.9) | | | 36.9) | | | 18.5) | | | 23.2) | | | 17.6) |
| SAE | 0/6 | 0 | (0; | 0/8 | 0 | (0; | 0/18 | 0 | (0; | 0/14 | 0 | (0; | 0/19 | 0 | (0; |
| | | | 45.9) | | | 36.9) | | | 18.5) | | | 23.2) | | | 17.6) |
| Death | 0/6 | 0 | (0; | 0/8 | 0 | (0; | 0/18 | 0 | (0; | 0/14 | 0 | (0; | 0/19 | 0 | (0; |
| | | | 45.9) | | | 36.9) | | | 18.5) | | | 23.2) | | | 17.6) |

Baseline IgA serostatus: RSV-experienced

| | | | | | | | | | | | | | | | |
|---|---|---|---|---|---|---|---|---|---|---|---|---|---|---|---|
| Within 30 minutes after | | | | | | | | | | | | | | | |
| vaccination 2 | | | | | | | | | | | | | | | |
| Immediate unsolicited | 0/2 | 0 | (0; | 0/0 | NC | (NC; | 0/4 | 0 | (0; | 0/9 | 0 | (0; | 0/6 | 0 | (0; |
| systemic AE | | | 84.2) | | | NC) | | | 60.2) | | | 33.6) | | | 45.9) |
| Immediate unsolicited | 0/2 | 0 | (0; | 0/0 | NC | (NC; | 0/4 | 0 | (0; | 0/9 | 0 | (0; | 0/6 | 0 | (0; |
| systemic AR | | | 84.2) | | | NC) | | | 60.2) | | | 33.6) | | | 45.9) |
| Within 28 days after | | | | | | | | | | | | | | | |
| vaccination 2 | | | | | | | | | | | | | | | |
| Solicited administration | 0/2 | 0 | (0; | 0/0 | NC | (NC; | 2/3 | 66.7 | (9.4; | 6/8 | 75.0 | (34.9; | 5/6 | 83.3 | (35.9; |
| site reaction | | | 84.2) | | | NC) | | | 99.2) | | | 96.8) | | | 99.6) |
| Solicited systemic | 1/2 | 50.0 | (1.3; | 0/0 | NC | (NC | 2/3 | 66.7 | (9.4; | 6/8 | 75.0 | (34.9; | 2/6 | 33.3 | (4.3; |
| reaction | | | 98.7) | | | NC) | | | 99.2) | | | 96.8) | | | 77.7) |
| Unsolicited AE | 0/2 | 0 | (0; | 0/0 | NC | (NC; | 1/4 | 25.0 | (0.6; | 3/9 | 33.3 | (7.5; | 3/6 | 50.0 | (11.8; |
| | | | 84.2) | | | NC) | | | 80.6) | | | 70.1) | | | 88.2) |
| Unsolicited AR | 0/2 | 0 | (0; | 0/0 | NC | (NC; | 0/4 | 0 | (0; | 0/9 | 0 | (0; | 0/6 | 0 | (0; |
| | | | 84.2) | | | NC) | | | 60.2) | | | 33.6) | | | 45.9) |
| AESI | 0/2 | 0 | (0; | 0/0 | NC | (NC; | 0/4 | 0 | (0; | 1/9 | 11.1 | (0.3; | 0/6 | 0 | (0; |
| | | | 84.2) | | | NC) | | | 60.2) | | | 48.2) | | | 45.9) |
| MAAE | 0/2 | 0 | (0; | 0/0 | NC | (NC; | 1/4 | 25.0 | (0.6; | 1/9 | 11.1 | (0.3; | 1/6 | 16.7 | (0.4; |
| | | | 84.2) | | | NC) | | | 80.6) | | | 48.2) | | | 64.1) |
| AE leading to study | 0/2 | 0 | (0; | 0/0 | NC | (NC; | 0/4 | 0 | (0; | 0/9 | 0 | (0; | 0/6 | 0 | (0; |
| discontinuation | | | 84.2) | | | NC) | | | 60.2) | | | 33.6) | | | 45.9) |
| SAE | 0/2 | 0 | (0; | 0/0 | NC | (NC; | 0/4 | 0 | (0; | 0/9 | 0 | (0; | 0/6 | 0 | (0; |
| | | | 84.2) | | | NC) | | | 60.2) | | | 33.6) | | | 45.9) |
| Death | 0/2 | 0 | (0; | 0/0 | NC | (NC; | 0/4 | 0 | (0; | 0/9 | 0 | (0; | 0/6 | 0 | (0; |
| | | | 84.2) | | | NC) | | | 60.2) | | | 33.6) | | | 45.9) |

Baseline IgA serostatus: Undetermined

| | | | | | | | | | | | | | | | |
|---|---|---|---|---|---|---|---|---|---|---|---|---|---|---|---|
| Within 30 minutes after | | | | | | | | | | | | | | | |
| vaccination 2 | | | | | | | | | | | | | | | |
| Immediate unsolicited | 0/0 | NC | (NC; | 0/0 | NC | (NC; | 0/5 | 0 | (0; | 0/5 | 0 | (0; | 0/2 | 0 | (0; |
| systemic AE | | | NC) | | | NC) | | | 52.2) | | | 52.2) | | | 84.2) |
| Immediate unsolicited | 0/0 | NC | (NC; | 0/0 | NC | (NC; | 0/5 | 0 | (0; | 0/5 | 0 | (0; | 0/2 | 0 | (0; |
| systemic AR | | | NC) | | | NC) | | | 52.2) | | | 52.2) | | | 84.2) |
| Within 28 days after | | | | | | | | | | | | | | | |
| vaccination 2 | | | | | | | | | | | | | | | |
| Solicited administration | 0/0 | NC | (NC; | 0/0 | NC | (NC; | 3/4 | 75.0 | (19.4; | 2/3 | 66.7 | (9.4; | 1/2 | 50.0 | (1.3; |
| site reaction | | | NC) | | | NC) | | | 99.4) | | | 99.2) | | | 98.7) |
| Solicited systemic | 0/0 | NC | (NC; | 0/0 | NC | (NC; | 3/4 | 75.0 | (19.4; | 2/3 | 66.7 | (9.4; | 1/2 | 50.0 | (1.3; |
| reaction | | | NC) | | | NC) | | | 99.4) | | | 99.2) | | | 98.7) |
| Unsolicited AE | 0/0 | NC | (NC; | 0/0 | NC | (NC; | 3/5 | 60.0 | (14.7; | 1/5 | 20.0 | (0.5; | 0/2 | 0 | (0; |
| | | | NC) | | | NC) | | | 94.7) | | | 71.6) | | | 84.2) |
| Unsolicited AR | 0/0 | NC | (NC; | 0/0 | NC | (NC; | 2/5 | 40.0 | (5.3; | 1/5 | 20.0 | (0.5; | 0/2 | 0 | (0; |
| | | | NC) | | | NC) | | | 85.3) | | | 71.6) | | | 84.2) |
| AESI | 0/0 | NC | (NC; | 0/0 | NC | (NC; | 1/5 | 20.0 | (0.5; | 0/5 | 0 | (0; | 0/2 | 0 | (0; |
| | | | NC) | | | NC) | | | 71.6) | | | 52.2) | | | 84.2) |
| MAAE | 0/0 | NC | (NC; | 0/0 | NC | (NC; | 2/5 | 40.0 | (5.3; | 1/5 | 20.0 | (0.5; | 0/2 | 0 | (0; |
| | | | NC) | | | NC) | | | 85.3) | | | 71.6) | | | 84.2) |
| AE leading to study | 0/0 | NC | (NC; | 0/0 | NC | (NC; | 0/5 | 0 | (0; | 0/5 | 0 | (0; | 0/2 | 0 | (0; |
| discontinuation | | | NC) | | | NC) | | | 52.2) | | | 52.2) | | | 84.2) |
| SAE | 0/0 | NC | (NC; | 0/0 | NC | (NC; | 0/5 | 0 | (0; | 0/5 | 0 | (0; | 0/2 | 0 | (0; |
| | | | NC) | | | NC) | | | 52.2) | | | 52.2) | | | 84.2) |
| Death | 0/0 | NC | (NC; | 0/0 | NC | (NC; | 0/5 | 0 | (0; | 0/5 | 0 | (0; | 0/2 | 0 | (0; |
| | | | NC) | | | NC) | | | 52.2) | | | 52.2) | | | 84.2) |

| | Cohorts 2 and 4 (pooled) | | | | | | | | |
|---|---|---|---|---|---|---|---|---|---|
| Period/ | RSV low dose | | | RSV high dose | | | Placebo | | |
| Participants | (N = 35) | | | (N = 28) | | | (N = 35) | | |
| experiencing | | | (95% | | | (95% | | | (95% |
| at least one: | n/M | % | CI) | n/M | % | CI) | n/M | % | CI) |

Baseline IgA serostatus: RSV-naïve

| | | | | | | | | | |
|---|---|---|---|---|---|---|---|---|---|
| Within 30 minutes after | | | | | | | | | |
| vaccination 2 | | | | | | | | | |
| Immediate unsolicited | 0/24 | 0 | (0; | 0/14 | 0 | (0; | 0/27 | 0 | (0; |
| systemic AE | | | 14.2) | | | 23.2) | | | 12.8) |
| Immediate unsolicited | 0/24 | 0 | (0; | 0/14 | 0 | (0; | 0/27 | 0 | (0; |
| systemic AR | | | 14.2) | | | 23.2) | | | 12.8) |
| Within 28 days after | | | | | | | | | |
| vaccination 2 | | | | | | | | | |
| Solicited administration | 17/23 | 73.9 | (51.6; | 14/14 | 100 | (76.8; | 18/27 | 66.7 | (46.0; |
| site reaction | | | 89.8) | | | 100) | | | 83.5) |
| Solicited systemic | 16/23 | 69.6 | (47.1; | 11/14 | 78.6 | (49.2; | 16/27 | 59.3 | (38.8; |
| reaction | | | 86.8) | | | 95.3) | | | 77.6) |
| Unsolicited AE | 6/24 | 25.0 | (9.8; | 5/14 | 35.7 | (12.8; | 10/27 | 37.0 | (19.4; |
| | | | 46.7) | | | 64.9) | | | 57.6) |
| Unsolicited AR | 2/24 | 8.3 | (1.0; | 1/14 | 7.1 | (0.2; | 4/27 | 14.8 | (4.2; |
| | | | 27.0) | | | 33.9) | | | 33.7) |
| AESI | 4/24 | 16.7 | (4.7; | 1/14 | 7.1 | (0.2; | 3/27 | 11.1 | (2.4; |
| | | | 37.4) | | | 33.9) | | | 29.2) |
| MAAE | 4/24 | 16.7 | (4.7; | 3/14 | 21.4 | (4.7; | 7/27 | 25.9 | (11.1; |
| | | | 37.4) | | | 50.8) | | | 46.3) |
| AE leading to study | 0/24 | 0 | (0; | 0/14 | 0 | (0; | 0/27 | 0 | (0; |
| discontinuation | | | 14.2) | | | 23.2) | | | 12.8) |
| SAE | 0/24 | 0 | (0; | 0/14 | 0 | (0; | 0/27 | 0 | (0; |
| | | | 14.2) | | | 23.2) | | | 12.8) |
| Death | 0/24 | 0 | (0; | 0/14 | 0 | (0; | 0/27 | 0 | (0; |
| | | | 14.2) | | | 23.2) | | | 12.8) |

Baseline IgA serostatus: RSV-experienced

| | | | | | | | | | |
|---|---|---|---|---|---|---|---|---|---|
| Within 30 minutes after | | | | | | | | | |
| vaccination 2 | | | | | | | | | |
| Immediate unsolicited | 0/6 | 0 | (0; | 0/9 | 0 | (0; | 0/6 | 0 | (0; |
| systemic AE | | | 45.9) | | | 33.6) | | | 45.9) |
| Immediate unsolicited | 0/6 | 0 | (0; | 0/9 | 0 | (0; | 0/6 | 0 | (0; |
| systemic AR | | | 45.9) | | | 33.6) | | | 45.9) |
| Within 28 days after | | | | | | | | | |
| vaccination 2 | | | | | | | | | |
| Solicited administration | 2/5 | 40.0 | (5.3; | 6/8 | 75.0 | (34.9; | 5/6 | 83.3 | (35.9; |
| site reaction | | | 85.3) | | | 96.8) | | | 99.6) |
| Solicited systemic | 3/5 | 60.0 | (14.7; | 6/8 | 75.0 | (34.9; | 2/6 | 33.3 | (4.3; |
| reaction | | | 94.7) | | | 96.8) | | | 77.7) |
| Unsolicited AE | 1/6 | 16.7 | (0.4; | 3/9 | 33.3 | (7.5; | 3/6 | 50.0 | (11.8; |
| | | | 64.1) | | | 70.1) | | | 88.2) |
| Unsolicited AR | 0/6 | 0 | (0; | 6/0 | 0 | (0; | 0/6 | 0 | (0; |
| | | | 45.9) | | | 33.6) | | | 45.9) |
| AESI | 0/6 | 0 | (0; | 1/9 | 11.1 | (0.3; | 0/6 | 0 | (0; |
| | | | 45.9) | | | 48.2) | | | 45.9) |
| MAAE | 1/6 | 16.7 | (0.4; | 1/9 | 11.1 | (0.3; | 1/6 | 16.7 | (0.4; |
| | | | 64.1) | | | 48.2) | | | 64.1) |
| AE leading to study | 0/6 | 0 | (0; | 0/9 | 0 | (0; | 0/6 | 0 | (0; |
| discontinuation | | | 45.9) | | | 33.6) | | | 45.9) |
| SAE | 0/6 | 0 | (0; | 0/9 | 0 | (0; | 0/6 | 0 | (0; |
| | | | 45.9) | | | 33.6) | | | 45.9) |
| Death | 0/6 | 0 | (0; | 0/9 | 0 | (0; | 0/6 | 0 | (0; |
| | | | 45.9) | | | 33.6) | | | 45.9) |

Baseline IgA serostatus: Undetermined

| | | | | | | | | | |
|---|---|---|---|---|---|---|---|---|---|
| Within 30 minutes after | | | | | | | | | |
| vaccination 2 | | | | | | | | | |
| Immediate unsolicited | 0/5 | 0 | (0; | 0/5 | 0 | (0; | 0/2 | 0 | (0; |
| systemic AE | | | 52.2) | | | 52.2) | | | 84.2) |
| Immediate unsolicited | 0/5 | 0 | (0; | 0/5 | 0 | (0; | 0/2 | 0 | (0; |
| systemic AR | | | 52.2) | | | 52.2) | | | 84.2) |
| Within 28 days after | | | | | | | | | |
| vaccination 2 | | | | | | | | | |
| Solicited administration | 3/4 | 75.0 | (19.4; | 2/3 | 66.7 | (9.4; | 1/2 | 50.0 | (1.3; |
| site reaction | | | 99.4) | | | 99.2) | | | 98.7) |
| Solicited systemic | 3/4 | 75.0 | (19.4; | 2/3 | 66.7 | (9.4; | 1/2 | 50.0 | (1.3; |
| reaction | | | 99.4) | | | 99.2) | | | 98.7) |
| Unsolicited AE | 3/5 | 60.0 | (14.7; | 1/5 | 20.0 | (0.5; | 0/2 | 0 | (0; |
| | | | 94.7) | | | 71.6) | | | 84.2) |
| Unsolicited AR | 2/5 | 40.0 | (5.3; | 1/5 | 20.0 | (0.5; | 0/2 | 0 | (0; |
| | | | 85.3) | | | 71.6) | | | 84.2) |
| AESI | 1/5 | 20.0 | (0.5; | 0/5 | 0 | (0; | 0/2 | 0 | (0; |
| | | | 71.6) | | | 52.2) | | | 84.2) |
| MAAE | 2/5 | 40.0 | (5.3; | 1/5 | 20.0 | (0.5; | 0/2 | 0 | (0; |
| | | | 85.3) | | | 71.6) | | | 84.2) |
| AE leading to study | 0/5 | 0 | (0; | 0/5 | 0 | (0; | 0/2 | 0 | (0; |
| discontinuation | | | 52.2) | | | 52.2) | | | 84.2) |
| SAE | 0/5 | 0 | (0; | 0/5 | 0 | (0; | 0/2 | 0 | (0; |
| | | | 52.2) | | | 52.2) | | | 84.2) |
| Death | 0/5 | 0 | (0; | 0/5 | 0 | (0; | 0/2 | 0 | (0; |
| | | | 52.2) | | | 52.2) | | | 84.2) |

n: number of participants experiencing the endpoint listed in the first two columns
M: number of participants with available data for the relevant endpoint
AR: Reactions related to study vaccine. Missing relationship (for the definition of AR) will be considered at the time of analysis as related to the vaccine.
“AE leading to study discontinuation” row includes those participants who have either on the termination form, the reason for early termination “Adverse Event” checked or lists an AE (solicited, unsolicited, or SAE) that has “Caused Study Discontinuation” checked that is at least Grade 1 and is within the time period indicated.
Baseline IgA serostatus is determined using serum anti-F IgA Ab titers measured by ELISA at D 0, as follows: “RSV-naïve” for participants with titers <4.8 EU/mL (<LOD), “RSV-experienced” for participants with titers >=4.8 EU/mL (>=LOD), “Undetermined” otherwise.
NC: Not Computed (if M <= 5 or STD = 0)

TABLE 13
Summary of 4-fold rise seroresponse of RSV A neutralizing antibody titers, in
RSV naïve and experienced participants - MN assay - 1/dil - FAS*

| Cohort 1 | | Cohort 2 | |
|---|---|---|---|
| RSV low dose | Placebo | RSV low dose | Placebo |
| (N = 17) | (N = 18) | (N = 10) | (N = 10) |

| | | | | | | | | | | | | |
|---|---|---|---|---|---|---|---|---|---|---|---|---|
| | | | (95% | | | (95% | | | (95% | | | (95% |
| Time Point | n/M | % | CI) | n/M | % | CI) | n/M | % | CI) | n/M | % | CI) |

RSV naïve

| | | | | | | | | | | | | | |
|---|---|---|---|---|---|---|---|---|---|---|---|---|---|
| D 0 (pre- | <LLOQ | 0/9 | 0 | (0; | 3/7 | 42.9 | (9.9; | 1/8 | 12.5 | (0.3; | 1/10 | 10.0 | (0.3; |
| vaccination | | | | 33.6) | | | 81.6) | | | 52.7) | | | 44.5) |
| 1) | >=LLOQ | 9/9 | 100 | (66.4; | 4/7 | 57.1 | (18.4; | 7/8 | 87.5 | (47.3; | 9/10 | 90.0 | (55.5; |
| | | | | 100) | | | 90.1) | | | 99.7) | | | 99.7) |
| D 56/D 0 | 4-fold rise | 6/9 | 66.7 | (29.9; | 1/5 | 20.0 | (0.5; | 6/7 | 85.7 | (42.1; | 0/8 | 0 | (0; |
| | seroresponse | | | 92.5) | | | 71.6) | | | 99.6) | | | 36.9) |
| | Titer at D 0 < | 0/9 | 0 | (0; | 0/5 | 0 | (0; | 1/7 | 14.3 | (0.4; | 0/8 | 0 | (0; |
| | LLOQ and Titer at | | | 33.6) | | | 52.2) | | | 57.9) | | | 36.9) |
| | D 56 >= 4x LLOQ | | | | | | | | | | | | |
| | Titer at D 0 >= | 6/9 | 66.7 | (29.9; | 1/5 | 20.0 | (0.5; | 5/7 | 71.4 | (29.0; | 0/8 | 0 | (0; |
| | LLOQ and Titer at | | | 92.5) | | | 71.6) | | | 96.3) | | | 36.9) |
| | D 56 >= 4x D 0 | | | | | | | | | | | | |
| D 84**/D 0 | 4-fold rise | NA | NA | NA | NA | NA | NA | 4/7 | 57.1 | (18.4; | 0/8 | 0 | (0; |
| | seroresponse | | | | | | | | | 90.1) | | | 36.9) |
| | Titer at D 0 < | NA | NA | NA | NA | NA | NA | 1/7 | 14.3 | (0.4; | 0/8 | 0 | (0; |
| | LLOQ and Titer at | | | | | | | | | 57.9) | | | 36.9) |
| | D 84 >= 4x LLOQ | | | | | | | | | | | | |
| | Titer at D 0 >= | NA | NA | NA | NA | NA | NA | 3/7 | 42.9 | (9.9; | 0/8 | 0 | (0; |
| | LLOQ and Titer at | | | | | | | | | 81.6) | | | 36.9) |
| | D 84 >= 4x D 0 | | | | | | | | | | | | |

RSV experienced

| | | | | | | | | | | | | | |
|---|---|---|---|---|---|---|---|---|---|---|---|---|---|
| D 0 (pre- | <LLOQ | 0/5 | 0 | (0; | 0/5 | 0 | (0; | 0/2 | 0 | (0; | 0/0 | NC | (NC; |
| vaccination | | | | 52.2) | | | 52.2) | | | 84.2) | | | NC) |
| 1) | >=LLOQ | 5/5 | 100 | (47.8; | 5/5 | 100 | (47.8; | 2/2 | 100 | (15.8; | 0/0 | NC | (NC; |
| | | | | 100) | | | 100) | | | 100) | | | NC) |
| D 56/D 0 | 4-fold rise | 2/5 | 40.0 | (5.3; | 1/5 | 20.0 | (0.5; | 2/2 | 100 | (15.8; | 0/0 | NC | (NC; |
| | seroresponse | | | 85.3) | | | 71.6) | | | 100) | | | NC) |
| | Titer at D 0 < | 0/5 | 0 | (0; | 0/5 | 0 | (0; | 0/2 | 0 | (0; | 0/0 | NC | (NC; |
| | LLOQ and Titer at | | | 52.2) | | | 52.2) | | | 84.2) | | | NC) |
| | D 56 >= 4x LLOQ | | | | | | | | | | | | |
| | Titer at D 0 >= | 2/5 | 40.0 | (5.3; | 1/5 | 20.0 | (0.5; | 2/2 | 100 | (15.8; | 0/0 | NC | (NC; |
| | LLOQ and Titer at | | | 85.3) | | | 71.6) | | | 100) | | | NC) |
| | D 56 >= 4x D 0 | | | | | | | | | | | | |
| D 84**/D 0 | 4-fold rise | NA | NA | NA | NA | NA | NA | 2/2 | 100 | (15.8; | 0/0 | NC | (NC; |
| | seroresponse | | | | | | | | | 100) | | | NC) |
| | Titer at D 0 < | NA | NA | NA | NA | NA | NA | 0/2 | 0 | (0; | 0/0 | NC | (NC; |
| | LLOQ and Titer at | | | | | | | | | 84.2) | | | NC) |
| | D 84 >= 4x LLOQ | | | | | | | | | | | | |
| | Titer at D 0 >= | NA | NA | NA | NA | NA | NA | 2/2 | 100 | (15.8; | 0/0 | NC | (NC; |
| | LLOQ and Titer at | | | | | | | | | 100) | | | NC) |
| | D 84 >= 4x D 0 | | | | | | | | | | | | |

| Cohort 3 | |
|---|---|
| RSV high dose | Placebo |
| (N = 10) | (N = 12) |

| | | | | | | |
|---|---|---|---|---|---|---|
| | | | (95% | | | (95% |
| Time Point | n/M | % | CI) | n/M | % | CI) |

RSV naïve

| | | | | | | | |
|---|---|---|---|---|---|---|---|
| D 0 (pre- | <LLOQ | 0/8 | 0 | (0; | 2/9 | 22.2 | (2.8; |
| vaccination | | | | 36.9) | | | 60.0) |
| 1) | >=LLOQ | 8/8 | 100 | (63.1; | 7/9 | 77.8 | (40.0; |
| | | | | 100) | | | 97.2) |
| D 56/D 0 | 4-fold rise | 5/7 | 71.4 | (29.0; | 0/8 | 0 | (0; |
| | seroresponse | | | 96.3) | | | 36.9) |
| | Titer at D 0 < | 0/7 | 0 | (0; | 0/8 | 0 | (0; |
| | LLOQ and Titer at | | | 41.0) | | | 36.9) |
| | D 56 >= 4x LLOQ | | | | | | |
| | Titer at D 0 >= | 5/7 | 71.4 | (29.0; | 0/8 | 0 | (0; |
| | LLOQ and Titer at | | | 96.3) | | | 36.9) |
| | D 56 >= 4x D 0 | | | | | | |
| D 84**/D 0 | 4-fold rise | NA | NA | NA | NA | NA | NA |
| | seroresponse | | | | | | |
| | Titer at D 0 < | NA | NA | NA | NA | NA | NA |
| | LLOQ and Titer at | | | | | | |
| | D 84 >= 4x LLOQ | | | | | | |
| | Titer at D 0 >= | NA | NA | NA | NA | NA | NA |
| | LLOQ and Titer at | | | | | | |
| | D 84 >= 4x D 0 | | | | | | |

RSV experienced

| | | | | | | | |
|---|---|---|---|---|---|---|---|
| D 0 (pre- | <LLOQ | 0/1 | 0 | (0; | 0/1 | 0 | (0; |
| vaccination | | | | 97.5) | | | 97.5) |
| 1) | >=LLOQ | 1/1 | 100 | (2.5; | 1/1 | 100 | (2.5; |
| | | | | 100) | | | 100) |
| D 56/D 0 | 4-fold rise | 0/1 | 0 | (0; | 0/1 | 0 | (0; |
| | seroresponse | | | 97.5) | | | 97.5) |
| | Titer at D 0 < | 0/1 | 0 | (0; | 0/1 | 0 | (0; |
| | LLOQ and Titer at | | | 97.5) | | | 97.5) |
| | D 56 >= 4x LLOQ | | | | | | |
| | Titer at D 0 >= | 0/1 | 0 | (0; | 0/1 | 0 | (0; |
| | LLOQ and Titer at | | | 97.5) | | | 97.5) |
| | D 56 >= 4x D 0 | | | | | | |
| D 84**/D 0 | 4-fold rise | NA | NA | NA | NA | NA | NA |
| | seroresponse | | | | | | |
| | Titer at D 0 < | NA | NA | NA | NA | NA | NA |
| | LLOQ and Titer at | | | | | | |
| | D 84 >= 4x LLOQ | | | | | | |
| | Titer at D 0 >= | NA | NA | NA | NA | NA | NA |
| | LLOQ and Titer at | | | | | | |
| | D 84 >= 4x D 0 | | | | | | |

| | Cohort 4 | | | | | | | | | Cohorts 1 to 4 (pooled) | | |
|---|---|---|---|---|---|---|---|---|---|---|---|---|
| | RSV low dose | | | RSV high dose | | | Placebo | | | RSV low dose | | |
| | (N = 31) | | | (N = 29) | | | (N = 30) | | | (N = 58) | | |
| | | | (95% | | | (95% | | | (95% | | | (95% |
| Time Point | n/M | % | CI) | n/M | % | CI) | n/M | % | CI) | n/M | % | CI) |

RSV naïve

| | | | | | | | | | | | | | |
|---|---|---|---|---|---|---|---|---|---|---|---|---|---|
| D 0 (pre- | <LLOQ | 4/21 | 19.0 | (5.4; | 2/15 | 13.3 | (1.7; | 1/19 | 5.3 | (0.1; | 5/38 | 13.2 | (4.4; |
| vaccination | | | | 41.9) | | | 40.5) | | | 26.0) | | | 28.1) |
| 1) | >=LLOQ | 17/21 | 81.0 | (58.1; | 13/15 | 86.7 | (59.5; | 18/19 | 94.7 | (74.0; | 33/38 | 86.8 | (71.9; |
| | | | | 94.6) | | | 98.3) | | | 99.9) | | | 95.6) |
| D 56/D 0 | 4-fold rise | 7/15 | 46.7 | (21.3; | 3/10 | 30.0 | (6.7; | 0/16 | 0 | (0; | 19/31 | 61.3 | (42.2; |
| | seroresponse | | | 73.4) | | | 65.2) | | | 20.6) | | | 78.2) |
| | Titer at D 0 < | 3/15 | 20.0 | (4.3; | 1/10 | 10.0 | (0.3; | 0/16 | 0 | (0; | 4/31 | 12.9 | (3.6; |
| | LLOQ and | | | 48.1) | | | 44.5) | | | 20.6) | | | 29.8) |
| | Titer at D 56 >= | | | | | | | | | | | | |
| | 4x LLOQ | | | | | | | | | | | | |
| | Titer at D 0 >= | 4/15 | 26.7 | (7.8; | 2/10 | 20.0 | (2.5; | 0/16 | 0 | (0; | 15/31 | 48.4 | (30.2; |
| | LLOQ | | | 55.1) | | | 55.6) | | | 20.6) | | | 66.9) |
| | and Titer at | | | | | | | | | | | | |
| | D 56 >= 4x | | | | | | | | | | | | |
| | D 0 | | | | | | | | | | | | |
| D 84**/D 0 | 4-fold rise | 12/16 | 75.0 | (47.6; | 7/10 | 70.0 | (34.8; | 2/16 | 12.5 | (1.6; | 16/23 | 69.6 | (47.1; |
| | seroresponse | | | 92.7) | | | 93.3) | | | 38.3) | | | 86.8) |
| | Titer at D 0 < | 4/16 | 25.0 | (7.3; | 2/10 | 20.0 | (2.5; | 0/16 | 0) | (0; | 5/23 | 21. | (7.5; |
| | LLOQ and | | | 52.4) | | | 55.6) | | | 20.6) | | | 43.7) |
| | Titer at D 84 >= | | | | | | | | | | | | |
| | 4x LLOQ | | | | | | | | | | | | |
| | Titer at D 0 >= | 8/16 | 50.0 | (24.7; | 5/10 | 50.0 | (18.7; | 2/16 | 12.5 | (1.6; | 11/23 | 47.8 | (26.8; |
| | LLOQ | | | 75.3) | | | 81.3) | | | 38.3) | | | 69.4) |
| | and Titer at | | | | | | | | | | | | |
| | D 84 >= 4x | | | | | | | | | | | | |
| | D 0 | | | | | | | | | | | | |

RSV experienced

| | | | | | | | | | | | | | |
|---|---|---|---|---|---|---|---|---|---|---|---|---|---|
| D 0 (pre- | <LLOQ | 0/4 | 0 | (0; | 0/8 | 0 | (0; | 0/7 | 0 | (0; | 0/11 | 0 | (0; |
| vaccination | | | | 60.2) | | | 36.9) | | | 41.0) | | | 28.5) |
| 1) | >=LLOQ | 4/4 | 100 | (39.8; | 8/8 | 100 | (63.1; | 7/7 | 100 | (59.0; | 11/11 | 100 | (71.5; |
| | | | | 100) | | | 100) | | | 100) | | | 100) |
| D 56/D 0 | 4-fold rise | 0/4 | 0 | (0; | 1/8 | 12.5 | (0.3; | 1/7 | 14.3 | (0.4; | 4/11 | 36.4 | (10.9; |
| | seroresponse | | | 60.2) | | | 52.7) | | | 57.9) | | | 69.2) |
| | Titer at D 0 < | 0/4 | 0 | (0; | 0/8 | 0 | (0; | 0/7 | 0 | (0; | 0/11 | 0 | (0; |
| | LLOQ and | | | 60.2) | | | 36.9) | | | 41.0) | | | 28.5) |
| | Titer at D 56 >= | | | | | | | | | | | | |
| | 4x LLOQ | | | | | | | | | | | | |
| | Titer at D 0 >= | 0/4 | 0 | (0; | 1/8 | 12.5 | (0.3; | 1/7 | 14.3 | (0.4; | 4/11 | 36.4 | (10.9; |
| | LLOQ | | | 60.2) | | | 52.7) | | | 57.9) | | | 69.2) |
| | and Titer at | | | | | | | | | | | | |
| | D 56 >= 4x | | | | | | | | | | | | |
| | D 0 | | | | | | | | | | | | |
| D 84**/D 0 | 4-fold rise | 0/4 | 0 | (0; | 1/8 | 12.5 | (0.3; | 1/7 | 14.3 | (0.4; | 2/6 | 33.3 | (4.3; |
| | seroresponse | | | 60.2) | | | 52.7) | | | 57.9) | | | 77.7) |
| | Titer at D 0 < | 0/4 | 0 | (0; | 0/8 | 0 | (0; | 0/7 | 0 | (0; | 0/6 | 0 | (0; |
| | LLOQ and | | | 60.2) | | | 36.9) | | | 41.0) | | | 45.9) |
| | Titer at D 84 >= | | | | | | | | | | | | |
| | 4x LLOQ | | | | | | | | | | | | |
| | Titer at D 0 >= | 0/4 | 0 | (0; | 1/8 | 12.5 | (0.3; | 1/7 | 14.3 | (0.4; | 2/6 | 33.3 | (4.3; |
| | LLOQ | | | 60.2) | | | 52.7) | | | 57.9) | | | 77.7) |
| | and Titer at | | | | | | | | | | | | |
| | D 84 >= 4x | | | | | | | | | | | | |
| | D 0 | | | | | | | | | | | | |

| | | Cohorts 1 to 4 (pooled) | | | | | |
|---|---|---|---|---|---|---|---|
| | | | | Placebo | | | |
| | | RSV high dose | | (N = 70) | | | |
| | | (N = 39) | | (95% | | | (95% |
| Time Point | | n/M | % | CI) | n/M | % | CI) |
| RSV naïve | | | | | | | |
| D 0 (pre- | <LLOQ | 2/23 | 8.7 | (1.1; | 7/45 | 15.6 | (6.5; |
| vaccination | | | | 28.0) | | | 29.5) |
| 1) | >=LLOQ | 21/23 | 91.3 | (72.0; | 38/45 | 84.4 | (70.5; |
| | | | | 98.9) | | | 93.5) |
| D 56/D 0 | 4-fold rise | 8/17 | 47.1 | (23.0; | 1/37 | 2.7 | (0.1; |
| | seroresponse | | | 72.2) | | | 14.2) |
| | Titer at D 0 < | 1/17 | 5.9 | (0.1; | 0/37 | 0 | (0; |
| | LLOQ and | | | 28.7) | | | 9.5) |
| | Titer at D 56 >= | | | | | | |
| | 4x LLOQ | | | | | | |
| | Titer at D 0 >= | 7/17 | 41.2 | (18.4; | 1/37 | 2.7 | (0.1; |
| | LLOQ | | | 67.1) | | | 14.2) |
| | and Titer at | | | | | | |
| | D 56 >= 4x | | | | | | |
| | D 0 | | | | | | |
| D 84**/D 0 | 4-fold rise | 7/10 | 70.0 | (34.8; | 2/24 | 8.3 | (1.0; |
| | seroresponse | | | 93.3) | | | 27.0) |
| | Titer at D 0 < | 2/10 | 20.0 | (2.5; | 0/24 | 0 | (0; |
| | LLOQ and | | | 55.6) | | | 14.2) |
| | Titer at D 84 >= | | | | | | |
| | 4x LLOQ | | | | | | |
| | Titer at D 0 >= | 5/10 | 50.0 | (18.7; | 2/24 | 8.3 | (1.0; |
| | LLOQ | | | 81.3) | | | 27.0) |
| | and Titer at | | | | | | |
| | D 84 >= 4x | | | | | | |
| | D 0 | | | | | | |
| RSV experienced | | | | | | | |
| D 0 (pre- | <LLOQ | 0/9 | 0 | (0; | 0/13 | 0 | (0; |
| vaccination | | | | 33.6) | | | 24.7) |
| 1) | >=LLOQ | 9/9 | 100 | (66.4; | 13/13 | 100 | (75.3; |
| | | | | 100) | | | 100) |
| D 56/D 0 | 4-fold rise | 2/9 | 22.2 | (2.8; | 2/13 | 15.4 | (1.9; |
| | seroresponse | | | 60.0) | | | 45.4) |
| | Titer at D 0 < | 0/9 | 0 | (0; | 0/13 | 0 | (0; |
| | LLOQ and | | | 33.6) | | | 24.7) |
| | Titer at D 56 >= | | | | | | |
| | 4x LLOQ | | | | | | |
| | Titer at D 0 >= | 2/9 | 22.2 | (2.8; | 2/13 | 15.4 | (1.9; |
| | LLOQ | | | 60.0) | | | 45.4) |
| | and Titer at | | | | | | |
| | D 56 >= 4x | | | | | | |
| | D 0 | | | | | | |
| D 84**/D 0 | 4-fold rise | 1/8 | 12.5 | (0.3; | 1/7 | 14.3 | (0.4; |
| | seroresponse | | | 52.7) | | | 57.9) |
| | Titer at D 0 < | 0/8 | 0 | (0; | 0/7 | 0 | (0; |
| | LLOQ and | | | 36.9) | | | 41.0) |
| | Titer at D 84 >= | | | | | | |
| | 4x LLOQ | | | | | | |
| | Titer at D 0 >= | 1/8 | 12.5 | (0.3; | 1/7 | 14.3 | (0.4; |
| | LLOQ | | | 52.7) | | | 57.9) |
| | and Titer at | | | | | | |
| | D 84 >= 4x | | | | | | |
| | D 0 | | | | | | |

M: number of participants with available data for the relevant endpoint.
NA: Not Applicable;
NC: Not Computed (if M <= 5 or STD = 0)
n: number of participants experiencing the endpoint listed in the first two columns
LLOQ
*Data for any participant infected by wt-RSV is excluded from analyses from the date of infection
**D 84 timepoint is applicable only to cohorts 2 and 4.
4-fold rise response is the proportion of participants with pre-vaccinationtiter <10 (1/dil) (<LLOQ) and post-vaccination titer >=40 (1/dil) (>=4x LLOQ) or pre-vaccination titer >=10 (1/dil) (>=LLOQ) and post-vaccination titer >=4-fold increase (>=4x D 0).
Baseline IgA serostatus is determined using serum anti-F IgA Ab titers measured by ELISA at D 0, as follows: ‘RSV naïve’ for participants with titers <LOD, ‘RSV experienced’ for participants with titers >= LOD.

TABLE 14
4-fold rise seroresponse of RSV A neutralizing antibody titers after vaccination 1 and/or vaccination
2, in RSV naïve and experienced participants - MN assay - 1/dil - FAS*

| Cohort 1 | | | | | | Cohort 2 | | |
|---|---|---|---|---|---|---|---|---|
| RSV low dose | | | Placebo | | | RSV low dose | | |
| (N = 17) | | | (N = 18) | | | (N = 10) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| Participants with 4-fold rise in RSV A | 6/9 | 66.7 | (29.9; | 1/5 | 20.0 | (0.5; | 6/7 | 85.7 | (42.1; |
| nAb titers after vaccination 1 and/or | | | 92.5) | | | 71.6) | | | 99.6) |
| vaccination 2** | | | | | | | | | |
| Participants with 4-fold rise in RSV | 6/9 | 66.7 | (29.9; | 1/5 | 20.0 | (0.5; | 6/7 | 85.7 | (42.1; |
| A nAb titers after vaccination 1 | | | 92.5) | | | 71.6) | | | 99.6) |
| (D 56/D 0) | | | | | | | | | |
| Participants with 4-fold rise in RSV | NA | NA | NA | NA | NA | NA | 4/7 | 57.1 | (18.4; |
| A nAb titers after vaccination 2 | | | | | | | | | 90.1) |
| (D 84/D 0)** | | | | | | | | | |

RSV experienced

| | | | | | | | | | |
|---|---|---|---|---|---|---|---|---|---|
| Participants with 4-fold rise in RSV A | 2/5 | 40.0 | (5.3; | 1/5 | 20.0 | (0.5; | 2/2 | 100 | (15.8; |
| nAb titers after vaccination 1 and/or | | | 85.3) | | | 71.6) | | | 100) |
| vaccination 2** | | | | | | | | | |
| Participants with 4-fold rise in RSV | 2/5 | 40.0 | (5.3; | 1/5 | 20.0 | (0.5; | 2/2 | 100 | (15.8; |
| A nAb titers after vaccination 1 | | | 85.3) | | | 71.6) | | | 100) |
| (D 56/D 0) | | | | | | | | | |
| Participants with 4-fold rise in RSV | NA | NA | NA | NA | NA | NA | 2/2 | 100 | (15.8; |
| A nAb titers after vaccination 2 | | | | | | | | | 100) |
| (D 84/D 0)** | | | | | | | | | |

| Cohort 2 | | | Cohort 3 | | | | | |
|---|---|---|---|---|---|---|---|---|
| Placebo | | | RSV high dose | | | Placebo | | |
| (N = 10) | | | (N = 10) | | | (N = 12) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| Participants with 4-fold rise in RSV A | 0/8 | 0 | (0; | 5/7 | 71.4 | (29.0; | 0/8 | 0 | (0; |
| nAb titers after vaccination 1 and/or | | | 36.9) | | | 96.3) | | | 36.9) |
| vaccination 2** | | | | | | | | | |
| Participants with 4-fold rise in RSV | 0/8 | 0 | (0; | 5/7 | 71.4 | (29.0; | 0/8 | 0 | (0; |
| A nAb titers after vaccination 1 | | | 36.9) | | | 96.3) | | | 36.9) |
| (D 56/D 0) | | | | | | | | | |
| Participants with 4-fold rise in RSV | 0/8 | 0 | (0; | NA | NA | NA | NA | NA | NA |
| A nAb titers after vaccination 2 | | | 36.9) | | | | | | |
| (D 84/D 0)** | | | | | | | | | |

RSV experienced

| | | | | | | | | | |
|---|---|---|---|---|---|---|---|---|---|
| Participants with 4-fold rise in RSV A | 0/0 | NC | (NC; | 1/1 | 100 | (2.5; | 0/1 | 0 | (0; |
| nAb titers after vaccination 1 and/or | | | NC) | | | 100) | | | 97.5) |
| vaccination 2** | | | | | | | | | |
| Participants with 4-fold rise in RSV | 0/0 | NC | (NC; | 1/1 | 100 | (2.5; | 0/1 | 0 | (0; |
| A nAb titers after vaccination 1 | | | NC) | | | 100) | | | 97.5) |
| (D 56/D 0) | | | | | | | | | |
| Participants with 4-fold rise in RSV | 0/0 | NC | (NC; | NA | NA | NA | NA | NA | NA |
| A nAb titers after vaccination 2 | | | NC) | | | | | | |
| (D 84/D 0)** | | | | | | | | | |

| Cohort 4 | | | | | | | | |
|---|---|---|---|---|---|---|---|---|
| RSV low dose | | | RSV high dose | | | Placebo | | |
| (N = 31) | | | (N = 29) | | | (N = 30) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| | 13/17 | 76.5 | (50.1; | 8/11 | 72.7 | (39.0; | 2/17 | 11.8 | (1.5; |
| Participants with 4-fold rise in RSV A | | | 93.2) | | | 94.0) | | | 36.4) |
| nAb titers after vaccination 1 and/or | | | | | | | | | |
| vaccination 2** | | | | | | | | | |
| Participants with 4-fold rise in RSV | 7/15 | 46.7 | (21.3; | 3/10 | 30.0 | (6.7; | 0/16 | 0 | (0; |
| A nAb titers after vaccination 1 | | | 73.4) | | | 65.2) | | | 20.6) |
| (D 56/D 0) | | | | | | | | | |
| Participants with 4-fold rise in RSV | 12/16 | 75.0 | (47.6; | 7/10 | 70.0 | (34.8; | 2/16 | 12.5 | (1.6; |
| A nAb titers after vaccination 2 | | | 92.7) | | | 93.3) | | | 38.3) |
| (D 84/D 0)** | | | | | | | | | |

RSV experienced

| Participants with 4-fold rise in RSV A | 0/4 | 0 | (0; | 1/8 | 12.5 | (0.3; | 1/7 | 14.3 | (0.4; |
|---|---|---|---|---|---|---|---|---|---|
| nAb titers after vaccination 1 and/or | | | 60.2) | | | 52.7) | | | 57.9) |
| vaccination 2** | | | | | | | | | |
| Participants with 4-fold rise in RSV | 0/4 | 0 | (0; | 1/8 | 12.5 | (0.3; | 1/7 | 14.3 | (0.4; |
| A nAb titers after vaccination 1 | | | 60.2) | | | 52.7) | | | 57.9) |
| (D 56/D 0) | | | | | | | | | |
| Participants with 4-fold rise in RSV | 0/4 | 0 | (0; | 1/8 | 12.5 | (0.3; | 1/7 | 14.3 | (0.4; |
| A nAb titers after vaccination 2 | | | 60.2) | | | 52.7) | | | 57.9) |
| (D 84/D 0)** | | | | | | | | | |
| | Cohorts 1 to 4 (pooled) | | | | | | | | |
| | RSV low dose | | | RSV high dose | | | Placebo | | |
| | (N = 58) | | | (N = 39) | | | (N = 70) | | |
| | | | (95% | | | (95% | | | (95% |
| | n/M | % | CI) | n/M | % | CI) | n/M | % | CI) |

RSV naïve

| | | | | | | | | | |
|---|---|---|---|---|---|---|---|---|---|
| | 25/33 | 75.8 | (57.7; | 13/18 | 72.2 | (46.5; | 3/38 | 7.9 | (1.7; |
| Participants with 4-fold rise in RSV A | | | 88.9) | | | 90.3) | | | 21.4) |
| nAb titers after vaccination 1 and/or | | | | | | | | | |
| vaccination 2** | | | | | | | | | |
| Participants with 4-fold rise in RSV | 19/31 | 61.3 | (42.2; | 8/17 | 47.1 | (23.0; | 1/37 | 2.7 | (0.1; |
| A nAb titers after vaccination 1 | | | 78.2) | | | 72.2) | | | 14.2) |
| (D 56/D 0) | | | | | | | | | |
| Participants with 4-fold rise in RSV | 16/23 | 69.6 | (47.1; | 7/10 | 70.0 | (34.8; | 2/24 | 8.3 | (1.0; |
| A nAb titers after vaccination 2 | | | 86.8) | | | 93.3) | | | 27.0) |
| (D 84/D 0)** | | | | | | | | | |

RSV experienced

| | | | | | | | | | |
|---|---|---|---|---|---|---|---|---|---|
| Participants with 4-fold rise in RSV A | 4/11 | 36.4 | (10.9; | 2/9 | 22.2 | (2.8; | 2/13 | 15.4 | (1.9; |
| nAb titers after vaccination 1 and/or | | | 69.2) | | | 60.0) | | | 45.4) |
| vaccination 2** | | | | | | | | | |
| Participants with 4-fold rise in RSV | 4/11 | 36.4 | (10.9; | 2/9 | 22.2 | (2.8; | 2/13 | 15.4 | (1.9; |
| A nAb titers after vaccination 1 | | | 69.2) | | | 60.0 | | | 45.4) |
| (D 56/D 0) | | | | | | | | | |
| Participants with 4-fold rise in RSV | 2/6 | 33.3 | (4.3; | 1/8 | 12.5 | (0.3; | 1/7 | 14.3 | (0.4; |
| A nAb titers after vaccination 2 | | | 77.7) | | | 52.7) | | | 57.9) |
| (D 84/D 0)** | | | | | | | | | |

n: number of participants experiencing the endpoint listed in the first column
M: number of participants with available data for the considered endpoint.
NA: Not Applicable;
NC: Not Computed (if M <= 5 or STD = 0)
*Data for any participant infected by wt-RSV is excluded from analyses from the date of infection
**Vaccination 2 is applicable only to cohorts 2 and 4.
4-fold rise response is the proportion of participants with pre-vaccination titer <10 (1/dil) and post-vaccination titer >=40 (1/dil) or pre-vaccination titer >=10 (1/dil) and post-vaccination titer >=4-fold increase.
Baseline IgA serostatus is determined using serum anti-F IgA Ab titers measured by ELISA at D 0, as follows: ‘RSV naïve’ for participants with titers <LOD, ‘RSV experienced’ for participants with titers >=LOD.

TABLE 15
Infectivity after vaccination 1, in RSV naïve and experienced participants - FAS*

| | | | | Cohort 1 | | | | | Cohort 2 |
|---|---|---|---|---|---|---|---|---|---|
| | | | | RSV | Placebo | | | | RSV |
| | | | | low dose | (N = 18) | | | | low dose |
| | | (95% | | (N = 17) | (95% | | | (95% | (N = 10) |
| n/M | % | CI) | n/M | % | CI) | n/M | % | CI) | n/M |

RSV naïve

| Participants infected | 8/9 | 88.9 | (51.8; | 1/5 | 20.0 | (0.5; | 8/8 | 100 | (63.1; |
|---|---|---|---|---|---|---|---|---|---|
| by RSV | | | 99.7) | | | 71.6) | | | 100) |
| Detection of | 6/9 | 66.7 | (29.9; | 0/6 | 0 | (0; | 8/8 | 100 | (63.1; |
| shedding >= 2.80 | | | 92.5) | | | 45.9) | | | 100) |
| (LOD) at D 7 | | | | | | | | | |
| >=4-fold rise in | 6/9 | 66.7 | (29.9; | 1/5 | 20.0 | (0.5; | 6/7 | 85.7 | (42.1; |
| RSV A neutralizing | | | 92.5) | | | 71.6) | | | 99.6) |
| antibody titers | | | | | | | | | |
| (D 56/D 0) | | | | | | | | | |
| >=4-fold rise in | 2/9 | 22.2 | (2.8; | 0/5 | 0 | (0; | 4/7 | 57.1 | (18.4; |
| anti-F IgG antibody | | | 60.0) | | | 52.2) | | | 90.1) |
| titers (D 56/D 0) | | | | | | | | | |

RSV experienced

| Participants infected | 4/5 | 80.0 | (28.4; | 1/5 | 20.0 | (0.5; | 2/2 | 100 | (15.8; |
|---|---|---|---|---|---|---|---|---|---|
| by RSV | | | 99.5) | | | 71.6) | | | 100) |
| Detection of | 3/5 | 60.0 | (14.7; | 0/5 | 0 | (0; | 2/2 | 100 | (15.8; |
| shedding >= 2.80 | | | 94.7) | | | 52.2) | | | 100) |
| (LOD) at D 7 | | | | | | | | | |
| >=4-fold rise in | 2/5 | 40.0 | (5.3; | 1/5 | 20.0 | (0.5; | 2/2 | 100 | (15.8; |
| RSV A neutralizing | | | 85.3) | | | 71.6) | | | 100) |
| antibody titers | | | | | | | | | |
| (D 56/D 0) | | | | | | | | | |
| >=4-fold rise in | 0/5 | 0 | (0; | 0/5 | 0 | (0; | 2/2 | 100 | (15.8; |
| anti-F IgG antibody | | | 52.2) | | | 52.2) | | | 100) |
| titers (D 56/D 0) | | | | | | | | | |

| | | | | | Cohort 3 | | | | |
|---|---|---|---|---|---|---|---|---|---|
| | Cohort 2 | | | | RSV | | | | |
| | Placebo | | | | high dose | Placebo | | | |
| | (N = 10) | | | | (N = 10) | (N = 12) | | | |
| | | (95% | | | (95% | | | (95% | |
| | % | CI) | n/M | % | CI) | n/M | % | CI) | |
| RSV naïve | | | | | | | | | |
| Participants infected | 0/8 | 0 | (0; | 8/8 | 100 | (63.1; | 0/8 | 0 | (0; |
| by RSV | | | 36.9) | | | 100) | | | 36.9) |
| Detection of | 0/10 | 0 | (0; | 8/8 | 100 | (63.1; | 0/9 | 0 | (0; |
| shedding >= 2.80 | | | 30.8) | | | 100) | | | 33.6) |
| (LOD) at D 7 | | | | | | | | | |
| >=4-fold rise in | 0/8 | 0 | (0; | 5/7 | 71.4 | (29.0; | 0/8 | 0 | (0; |
| RSV A neutralizing | | | 36.9) | | | 96.3) | | | 36.9) |
| antibody titers | | | | | | | | | |
| (D 56/D 0) | | | | | | | | | |
| >=4-fold rise in | 0/8 | 0 | (0; | 3/7 | 42.9 | (9.9; | 0/8 | 0 | (0; |
| anti-F IgG antibody | | | 36.9) | | | 81.6) | | | 36.9) |
| titers (D 56/D 0) | | | | | | | | | |
| RSV experienced | | | | | | | | | |
| Participants infected | 0/0 | NC | (NC; | 1/1 | 100 | (2.5; | 0/1 | 0 | (0; |
| by RSV | | | NC) | | | 100) | | | 97.5) |
| Detection of | 0/0 | NC | (NC; | 1/1 | 100 | (2.5; | 0/1 | 0 | (0; |
| shedding >= 2.80 | | | NC) | | | 100) | | | 97.5) |
| (LOD) at D 7 | | | | | | | | | |
| >=4-fold rise in | 0/0 | NC | (NC; | 1/1 | 100 | (2.5; | 0/1 | 0 | (0; |
| RSV A neutralizing | | | NC) | | | 100) | | | 97.5) |
| antibody titers | | | | | | | | | |
| (D 56/D 0) | | | | | | | | | |
| >=4-fold rise in | 0/0 | NC | (NC; | 1/1 | 100 | (2.5; | 0/1 | 0 | (0; |
| anti-F IgG antibody | | | NC) | | | 100) | | | 97.5) |
| titers (D 56/D 0) | | | | | | | | | |

| Cohort 4 | | | | | | | | |
|---|---|---|---|---|---|---|---|---|
| RSV low dose | | | RSV high dose | | | Placebo | | |
| (N = 31) | | | (N = 29) | | | (N = 30) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| Participants infected by RSV | 15/20 | 75.0 | (50.9; | 9/13 | 69.2 | (38.6; | 1/16 | 6.3 | (0.2; |
| | | | 91.3) | | | 90.9) | | | 30.2) |
| Detection of shedding >= | 13/21 | 61.9 | (38.4; | 9/14 | 64.3 | (35.1; | 0/19 | 0 | (0; |
| 2.80 (LOD) at D 7 | | | 81.9) | | | 87.2) | | | 17.6) |
| >=4-fold rise in RSV A | 7/15 | 46.7 | (21.3; | 3/10 | 30.0 | (6.7; | 0/16 | 0 | (0; |
| neutralizing antibody titers | | | 73.4) | | | 65.2) | | | 20.6) |
| (D 56/D 0) | | | | | | | | | |
| >=4-fold rise in anti-F IgG | 7/14 | 50.0 | (23.0; | 2/10 | 20.0 | (2.5; | 1/16 | 6.3 | (0.2; |
| antibody titers (D 56/D 0) | | | 77.0) | | | 55.6) | | | 30.2) |

RSV experienced

| | | | | | | | | | |
|---|---|---|---|---|---|---|---|---|---|
| Participants infected by RSV | 2/3 | 66.7 | (9.4; | 2/8 | 25.0 | (3.2; | 1/7 | 14.3 | (0.4; |
| | | | 99.2) | | | 65.1) | | | 57.9) |
| Detection of shedding >= | 1/3 | 33.3 | (0.8; | 1/8 | 12.5 | (0.3; | 0/7 | 0 | (0; |
| 2.80 (LOD) at D 7 | | | 90.6) | | | 52.7) | | | 41.0) |
| >=4-fold rise in RSV A | 0/4 | 0 | (0; | 1/8 | 12.5 | (0.3; | 1/7 | 14.3 | (0.4; |
| neutralizing antibody titers | | | 60.2) | | | 52.7) | | | 57.9) |
| (D 56/D 0) | | | | | | | | | |
| >=4-fold rise in anti-F IgG | 1/3 | 33.3 | (0.8; | 2/8 | 25.0 | (3.2; | 1/7 | 14.3 | (0.4; |
| antibody titers (D 56/D 0) | | | 90.6) | | | 65.1) | | | 57.9) |

| | Cohorts 1 to 4 (pooled) | | | | | | | | |
|---|---|---|---|---|---|---|---|---|---|
| | RSV low dose | | | RSV high dose | | | Placebo | | |
| | (N = 58) | | | (N = 39) | | | (N = 70) | | |
| | | | (95% | | | (95% | | | (95% |
| | n/M | % | CI) | n/M | % | CI) | n/M | % | CI) |
| RSV naïve | | | | | | | | | |
| Participants infected by RSV | 31/37 | 83.8 | (68.0; | 17/21 | 81.0 | (58.1; | 2/37 | 5.4 | (0.7; |
| | | | 93.8) | | | 94.6) | | | 18.2) |
| Detection of shedding >= | 27/38 | 71.1 | (54.1; | 17/22 | 77.3 | (54.6; | 0/44 | 0 | (0; |
| 2.80 (LOD) at D 7 | | | 84.6) | | | 92.2) | | | 8.0) |
| >=4-fold rise in RSV A | 19/31 | 61.3 | (42.2; | 8/17 | 47.1 | (23.0; | 1/37 | 2.7 | (0.1; |
| neutralizing antibody titers | | | 78.2) | | | 72.2) | | | 14.2) |
| (D 56/D 0) | | | | | | | | | |
| >=4-fold rise in anti-F IgG | 13/30 | 43.3 | (25.5; | 5/17 | 29.4 | (10.3; | 1/37 | 2.7 | (0.1; |
| antibody titers (D 56/D 0) | | | 62.6) | | | 56.0) | | | 14.2) |
| RSV experienced | | | | | | | | | |
| Participants infected by RSV | 8/10 | 80.0 | (44.4; | 3/9 | 33.3 | (7.5; | 2/13 | 15.4 | (1.9; |
| | | | 97.5) | | | 70.1) | | | 45.4) |
| Detection of shedding >= | 6/10 | 60.0 | (26.2; | 2/9 | 22.2 | (2.8; | 0/13 | 0 | (0; |
| 2.80 (LOD) at D 7 | | | 87.8) | | | 60.0) | | | 24.7) |
| >=4-fold rise in RSV A | 4/11 | 36.4 | (10.9; | 2/9 | 22.2 | (2.8; | 2/13 | 15.4 | (1.9; |
| neutralizing antibody titers | | | 69.2) | | | 60.0) | | | 45.4) |
| (D 56/D 0) | | | | | | | | | |
| >=4-fold rise in anti-F IgG | 3/10 | 30.0 | (6.7; | 3/9 | 33.3 | (7.5; | 1/13 | 7.7 | (0.2; |
| antibody titers (D 56/D 0) | | | 65.2) | | | 70.1) | | | 36.0) |

n: number of participants experiencing the endpoint listed in the first column
M: number of participants with available data for the considered endpoint
*Data for any participant infected by wt-RSV is excluded from analyses from the date of infection
Infection defined as detection of vaccine virus strain in nasal swab sample by RT-qPCR and/or a >=4-fold rise in RSV A serum nAb titers and/or RSV serum anti-F IgG Ab titers
Baseline IgA serostatus is determined using serum anti-F IgA Ab titers measured by ELISA at D 0, as follows: ‘RSV naïve’ for participants with titers <LOD, ‘RSV experienced’ for participants with titers >=LOD.
NC: Not Computed (if M <= 5 or STD = 0)

TABLE 16
Infectivity after vaccination 2, in RSV naïve and experienced participants - FAS*

| Cohort 2 | | | | | | Cohort 4 | | |
|---|---|---|---|---|---|---|---|---|
| RSV low dose | | | Placebo | | | RSV low dose | | |
| (N = 10) | | | (N = 10) | | | (N = 31) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| Participants infected by RSV | 5/7 | 71.4 | (29.0; | 0/7 | 0 | (0; | 14/17 | 82.4 | (56.6; |
| | | | 96.3) | | | 41.0) | | | 96.2) |
| Detection of shedding >= 2.80 | 1/5 | 20.0 | (0.5; | 0/7 | 0 | (0; | 3/17 | 17.6 | (3.8; |
| (LOD) at D 63 | | | 71.6) | | | 41.0) | | | 43.4) |
| >=4-fold rise in RSV A | 4/7 | 57.1 | (18.4; | 0/8 | 0 | (0; | 12/16 | 75.0 | (47.6; |
| neutralizing antibody titers | | | 90.1) | | | 36.9) | | | 92.7) |
| (D 84/D 0) | | | | | | | | | |
| >=4-fold rise in anti-F IgG | 5/7 | 71.4 | (29.0; | 0/8 | 0 | (0; | 10/15 | 66.7 | (38.4; |
| antibody titers (D 84/D 0) | | | 96.3) | | | 36.9) | | | 88.2) |

RSV experienced

| | | | | | | | | | |
|---|---|---|---|---|---|---|---|---|---|
| Participants infected by RSV | 2/2 | 100 | (15.8; | 0/0 | NC | (NC; | 1/3 | 33.3 | (0.8; |
| | | | 100) | | | NC) | | | 90.6) |
| Detection of shedding >= 2.80 | 0/2 | 0 | (0; | 0/0 | NC | (NC; | 0/4 | 0 | (0; |
| (LOD) at D 63 | | | 84.2) | | | NC) | | | 60.2) |
| >=4-fold rise in RSV A | 2/2 | 100 | (15.8; | 0/0 | NC | (NC; | 0/4 | 0 | (0; |
| neutralizing antibody titers | | | 100) | | | NC) | | | 60.2) |
| (D 84/D 0) | | | | | | | | | |
| >=4-fold rise in anti-F IgG | 2/2 | 100 | (15.8; | 0/0 | NC | (NC; | 1/3 | 33.3 | (0.8; |
| antibody titers (D 84/D 0) | | | 100) | | | NC) | | | 90.6) |

| | Cohort 4 | | | | | |
|---|---|---|---|---|---|---|
| | RSV high dose | | | Placebo | | |
| | (N = 29) | | | (N = 30) | | |
| | | | (95% | | | (95% |
| | n/M | % | CI) | n/M | % | CI) |
| RSV naïve | | | | | | |
| Participants infected by RSV | 7/10 | 70.0 | (34.8; | 3/15 | 20.0 | (4.3; |
| | | | 93.3) | | | 48.1) |
| Detection of shedding >= 2.80 | 2/9 | 22.2 | (2.8; | 0/17 | 0 | (0; |
| (LOD) at D 63 | | | 60.0) | | | 19.5) |
| >=4-fold rise in RSV A | 7/10 | 70.0 | (34.8; | 2/16 | 12.5 | (1.6; |
| neutralizing antibody titers | | | 93.3) | | | 38.3) |
| (D 84/D 0) | | | | | | |
| >=4-fold rise in anti-F IgG | 6/9 | 66.7 | (29.9; | 3/15 | 20.0 | (4.3; |
| antibody titers (D 84/D 0) | | | 92.5) | | | 48.1) |
| RSV experienced | | | | | | |
| Participants infected by RSV | 4/8 | 50.0 | (15.7; | 2/7 | 28.6 | (3.7; |
| | | | 84.3) | | | 71.0) |
| Detection of shedding >= 2.80 | 1/8 | 12.5 | (0.3; | 0/7 | 0 | (0; |
| (LOD) at D 63 | | | 52.7) | | | 41.0) |
| >=4-fold rise in RSV A | 1/8 | 12.5 | (0.3; | 1/7 | 14.3 | (0.4; |
| neutralizing antibody titers | | | 52.7) | | | 57.9) |
| (D 84/D 0) | | | | | | |
| >=4-fold rise in anti-F IgG | 3/8 | 37.5 | (8.5; | 2/7 | 28.6 | (3.7; |
| antibody titers (D 84/D 0) | | | 75.5) | | | 71.0) |

| Cohorts 2 and 4 (pooled) | | | | | | | | |
|---|---|---|---|---|---|---|---|---|
| RSV low dose | | | RSV high dose | | | Placebo | | |
| (N = 41) | | | (N = 29) | | | (N = 40) | | |
| | | (95% | | | (95% | | | (95% |
| n/M | % | CI) | n/M | % | CI) | n/M | % | CI) |

RSV naïve

| | | | | | | | | | |
|---|---|---|---|---|---|---|---|---|---|
| Participants infected by RSV | 19/24 | 79.2 | (57.8; | 7/10 | 70.0 | (34.8; | 3/22 | 13.6 | (2.9; |
| | | | 92.9) | | | 93.3) | | | 34.9) |
| Detection of shedding >= 2.80 (LOD) at D 63 | 4/22 | 18.2 | (5.2; | 2/9 | 22.2 | (2.8; | 0/24 | 0 | (0; |
| | | | 40.3) | | | 60.0) | | | 14.2) |
| >=4-fold rise in RSV A neutralizing antibody titers (D 84/D 0) | 16/23 | 69.6 | (47.1; | 7/10 | 70.0 | (34.8; | 2/24 | 8.3 | (1.0; |
| | | | 86.8) | | | 93.3) | | | 27.0) |
| >=4-fold rise in anti-F IgG antibody titers (D 84/D 0) | 15/22 | 68.2 | (45.1; | 6/9 | 66.7 | (29.9; | 3/23 | 13.0 | (2.8; |
| | | | 86.1) | | | 92.5) | | | 33.6) |

RSV experienced

| | | | | | | | | | |
|---|---|---|---|---|---|---|---|---|---|
| Participants infected by RSV | 3/5 | 60.0 | (14.7; | 4/8 | 50.0 | (15.7; | 2/7 | 28.6 | (3.7; |
| | | | 94.7) | | | 84.3) | | | 71.0) |
| Detection of shedding >= 2.80 (LOD) at D 63 | 0/6 | 0 | (0; | 1/8 | 12.5 | (0.3; | 0/7 | 0 | (0; |
| | | | 45.9) | | | 52.7) | | | 41.0) |
| >=4-fold rise in RSV A neutralizing antibody titers (D 84/D 0) | 2/6 | 33.3 | (4.3; | 1/8 | 12.5 | (0.3; | 1/7 | 14.3 | (0.4; |
| | | | 77.7) | | | 52.7) | | | 57.9) |
| >=4-fold rise in anti-F IgG antibody titers (D 84/D 0) | 3/5 | 60.0 | (14.7; | 3/8 | 37.5 | (8.5; | 2/7 | 28.6 | (3.7; |
| | | | 94.7) | | | 75.5) | | | 71.0) |

n: number of participants experiencing the endpoint listed in the first column
M: number of participants with available data for the considered endpoint
*Data for any participant infected by wt-RSV is excluded from analyses from the date of infection
Infection defined as detection of vaccine virus strain in nasal swab sample by RT-qPCR and/or a >=4-fold rise in RSV A serum nAb titers and/or RSV serum anti-F IgG Ab titers
Applicable only to cohorts 2 and 4.
Baseline IgA serostatus is determined using serum anti-F IgA Ab titers measured by ELISA at D 0, as follows: ‘RSV naïve’ for participants with titers <LOD, ‘RSV experienced’ for participants with titers >=LOD.
NC: Not Computed (if M <= 5 or STD = 0)

### Example 3: A Study on the Immunogenicity and Safety of 3 Different Dose Concentrations of a Respiratory Syncytial Virus Vaccine in Infants and Toddlers

### Protocol Title:

A parallel group, Phase III, randomized, observer-blind, placebo-controlled, multi-center, multi-national, multi-arm study to demonstrate non-inferiority of the immune response of a low dose compared to the standard dose and to evaluate the safety of a respiratory syncytial virus vaccine in infants and toddlers.

### Rationale:

To complement the stability studies of the RSV ΔNS2/Δ1313/I1314L (Sanofi) vaccine in the context of the vaccine's shelf life, the study will investigate 3 vaccine dose concentrations for immunogenicity and safety (ie, low [LD: target 5.4 log₁₀ PFU/dose], standard [SD: target 6.4 log₁₀ PFU/dose], and high dose [HD: target 7.0 log₁₀ PFU/dose]). Thus, the aim of the study is to evaluate whether the LD is non-inferior to the SD as assessed by RSV A and RSV B serum neutralizing antibodies 28 days after the second vaccination (Day 85). The goal is to show that the LD will generate an immune response non inferior to the SD. In addition, the study aims to investigate if the vaccine is safe in infants and toddlers born at term (ie, ≥37 weeks of gestation) and born preterm (ie, 28 through 36 weeks of gestation).

TABLE 17
Objectives, Endpoints, and Estimands:

| | | Estimands | |
|---|---|---|---|
| | | | Data handling strategy |
| Objectives | | Population-level | (Analysis set, Intercurrent events, |
| Primary | Endpoints | summary | Missing data) |
| Immunogenicity | RSV A and RSV B serum | Ratio of GMTs between groups | Immunogenicity will be |
| 1. To demonstrate the non-inferiority of 2 | neutralizing antibody | (LD RSV ΔNS2/Δ1313/I1314L | estimated in the setting where |
| administrations of the LD RSV | titers at 28 days | (Sanofi)/SD RSV | participants do not deviate from |
| ΔNS2/Δ1313/I1314L (Sanofi) vaccine | post dose 2 (D 85) | ΔNS2/Δ1313/I1314L (Sanofi)) of | the study schedules and protocol |
| antibody levels to RSV A and RSV B | | each RSV antibody (A and B) | requirements potentially |
| compared to the SD RSV | | Success criteria: The | impacting immunogenicity |
| ΔNS2/Δ1313/I1314L (Sanofi) | | non-inferiority of LD RSV | (principal stratum strategy) as |
| vaccine (Cohort 2) | | ΔNS2/Δ1313/I1314L (Sanofi) | defined in the Per Protocol |
| | | versus SD RSV | Analysis Set*. |
| | | ΔNS2/Δ1313/I1314L (Sanofi) | No other intercurrent |
| | | will be demonstrated if the lower | events are expected. |
| | | limit (LL) of the 2-sided 95% CI | Missing data will |
| | | of the ratio of GMTs is >2/3 for | not be imputed. |
| | | both RSV antibodies (A and B) | |
| Safety | Presence of unsolicited | The main parameters for | Safety will be estimated in the |
| 2. To assess the safety profile of the SD and | systemic AEs reported in the | the safety endpoints will | setting where participants |
| HD RSV ΔNS2/Δ1313/I1314L (Sanofi) | 30 minutes after each | be described by number of | received at least one study |
| vaccine after each and any administration | vaccination | participants, percentage, | intervention administration as |
| in medically stable participants born | Presence of solicited | and 95% CI using the | defined in the Safety Analysis |
| preterm (Cohort 1) | administration site reactions | exact binomial distribution | Set**. |
| 3. To assess the safety profile of the | within 21 days after each | (Clopper Pearson method) | Any relevant intercurrent event |
| LD/SD/HD RSV ΔNS2/Δ1313/I1314L | vaccination | for single proportion. | will be handled according to |
| (Sanofi) vaccine after each and any | Presence of solicited systemic | | treatment policy strategy. |
| administration in Cohort 2 participants | reactions within 21 days after | | Missing data will not be |
| | each vaccination | | imputed. |
| | Presence of unsolicited AEs | | |
| | within 28 days after each | | |
| | vaccination. | | |
| | Presence of MAAEs | | |
| | throughout the study | | |
| | Presence of SAEs throughout | | |
| | the study | | |
| | Presence of AESIs throughout | | |
| | the study | | |

Preterm = born after a gestation period of 28 through 36 weeks. At term = born after a gestation period of ≥37 weeks.
LD = target 5.4 log10 PFU; SD = target 6.4 log10 PFU; HD = target 7.0 log10 PFU
Abbreviations: Ab, antibody; AE, adverse event; AESI, adverse event of special interest; CI, confidence interval; D, day; GMTs, geometric mean titers; HD, high dose; LD, low dose; LL, lower limit; MAAE, medically attended adverse event; PFU, plaque forming units; SAE, serious adverse event; SD, standard dose; RSV, respiratory syncytial virus.
*The primary population of interest for immunogenicity is defined as participants receiving full vaccination schedule and meeting the inclusion and exclusion criteria.
**The primary population of interest for safety is defined as participants receiving at least one dose of study vaccine.

TABLE 18
Overall Design:

| | |
|---|---|
| Type of design | Parallel, multinational, multi-center |
| Phase | III |
| Control method | Placebo-controlled |
| Study population | Healthy infants and toddlers aged 6 months to <22 |
| | months. |
| | Cohort 1: Participants born after a gestation |
| | period of 28 through 36 completed weeks and |
| | medically stable. |
| | Cohort 2: Participants born after a gestation |
| | period of ≥37 weeks. |
| | Contingent upon an acceptable safety profile of |
| | the HD in preterm participants, enrolment in |
| | Cohort 2 will proceed to include participants born |
| | preterm. |
| | Cohort 1 and Cohort 2 participants will be |
| | enrolled in parallel. |
| Regions | European Union, Latin America, and North |
| | America |
| Level and method | Observer-blind: |
| of blinding | Blinding for vaccine group assignment: |
| | participants, parents or legally |
| | acceptable representative (LAR), |
| | outcome assessors, investigators, |
| | laboratory personnel, sponsor study |
| | staff* |
| | No blinding for study staff who |
| | prepare and administer the study |
| | interventions |
| | *Generally, safety data review during the study |
| | will be performed in a blinded manner by the |
| | Sponsor Safety Management Team (SMT). |
| | Safety data for Cohort 1 participants will be |
| | reviewed in an unblinded manner at the |
| | predefined time-points, involving pause of |
| | enrollment. |
| Investigational | Cohort 1: |
| intervention | (1:1 randomization) |
| assignment | Group 1 (N = 10): 2 administrations of SD Group |
| method | 2 (N = 10): 2 administrations of placebo |
| | (2:1 randomization) |
| | Group 3 (N = 10): 2 administrations of HD |
| | Group 4 (N = 5): 2 administrations of placebo |
| | Group 1 and 2 participants will be enrolled first |
| | and will be randomized to receive either SD |
| | vaccine or placebo. Based on the acceptable |
| | safety profile up to 28 days after the first |
| | vaccination, enrolment of participants into |
| | Groups 3 and 4 will proceed. Respectively, |
| | enrollment of pre-term participants in Cohort 2 |
| | will initiate, based on the acceptable safety |
| | profile up to 28 days after the first vaccination of |
| | Groups 3 and 4. |
| | Cohort 2 (2:2:1:1 randomization): |
| | Group 1 (N = 304): 2 administrations of LD |
| | Group 2 (N = 304): 2 administrations of SD |
| | Group 3 (N = 152): 2 administrations of HD |
| | Group 4 (N = 152): 2 administrations of placebo |
| | Cohort 2 participants will be randomized to |
| | receive either LD, SD, or HD vaccine or placebo. |
| | Randomization will be stratified by study |
| | country, age group (<12 months and ≥12 |
| | months), and potential RSV seropositivity (based |
| | on maternal immunization, and previous receipt |
| | of RSV mAb). |
| | Cohorts 1 and 2 will enroll in parallel. |

Inclusion/Exclusion Criteria
Inclusion criteria
Participants are eligible for the study only if all of the following criteria are met: AGE
Aged 6 months to <22 months on the day of inclusion¹
¹“6 months to <22 months” means from the day of the 6-month birthday to the day before the 22-month birthday. The second vaccine administration should be administered before the study participant has turned 24 months of age.

### Type of Participant and Disease Characteristics

Participants who are healthy as determined by medical evaluation including medical history.

For Cohort 1 and Cohort 2 (contingent upon satisfactory safety profile of the RSV ΔNS2/Δ1313/I1314L (Sanofi) vaccine in Cohort 1):

Participant born 28 through 36 weeks of gestation and medically stable as assessed by the investigator, based on the following definition: “Medically stable” refers to the condition of premature infants who do not require significant medical support or ongoing management for debilitating disease and who have demonstrated a clinical course of sustained recovery by the time they receive the first dose of study intervention.

### For Cohort 2:

Participant born at full term of pregnancy (≥37 weeks of gestation)

### Informed Consent

101. Informed consent form has been signed and dated by the parent(s) or other LAR (and by an independent witness if required by local regulations)

### Other Inclusions

Participant and parent(s)/LAR are able to attend all scheduled visits and to comply with all study procedures.

### Exclusion Criteria

Participants are not eligible for the study if any of the following criteria are met:

### Medical Conditions

E01. Known or suspected congenital or acquired immunodeficiency; or receipt of immunosuppressive therapy, such as anti-cancer chemotherapy or radiation therapy, within the preceding 6 months; or long-term systemic corticosteroid therapy (prednisone or equivalent for more than 2 consecutive weeks within the past 3 months)
E02. Known systemic hypersensitivity to any of the study intervention components, or history of a life-threatening reaction to the study intervention used in the study or to a product containing any of the same substances²
E03. Chronic illness that, in the opinion of the investigator, is at a stage where it might interfere with study conduct or completion³
E04. History of medically diagnosed wheezing⁴.
E05. Any acute febrile illness in the past 48 hours that according to investigator judgment is significant enough to interfere with successful inoculation on the day of vaccination. A prospective participant should not be included in the study until the condition has resolved or the febrile event has subsided.
E06. Probable or confirmed ongoing case of viral respiratory infection (including COVID-19, influenza, rhinovirus, etc.) at the time of enrollment. A prospective participant should not be included in the study until the respiratory infection has resolved.
E07. Member of a household that contains an immunocompromised individual, including, but not limited to:
   a person who is human immunodeficiency virus (HIV) infected
   a person who has received chemotherapy within the 12 months prior to study enrollment
   a person who has received (within the past 6 months) or is receiving (at the time of enrollment) immunosuppressant agents
   a person living with a solid organ or bone marrow transplant
   Potential close contact with other immunocompromised individual within 30 days after each vaccination as per investigator's discretion. ² The components of study intervention are listed elsewhere.³ Chronic illness may include, but is not limited to, cardiac disorders, lung disease (including any history of reactive airway disease or receipt of bronchodilator or inhaled steroid therapy), atopy, renal disorders, auto-immune disorders, diabetes, psychometer diseases, and known congenital or genetic diseases.⁴ Children with a history of recurrent wheezing will be excluded. Children with a previous single episode of wheezing may be included if that episode of wheezing was not associated with hospitalization or if does not have a family history of wheezing.

### Prior/Concomitant Therapy

E08. Participant's mother previous receipt or planned administration of an investigational RSV vaccine during pregnancy and/or breastfeeding.
E09. Receipt or planned receipt of any of the following vaccines prior to enrollment or after the first study intervention administration:
   Any other intranasal live attenuated vaccine within the 28 days prior to and after the first study intervention administration.
   Unless given on the day of the first study intervention administration, any other injectable live attenuated vaccines within the 28 days prior to and after. Concomitant receipt on the day of the first study intervention administration is allowed.
E10. Previous receipt of an investigational RSV vaccine or receiving any anti-RSV product (such as ribavirin or RSV immune globulin) at the time of enrollment. Previous receipt of Nirsevimab within 6 months or Palivizumab within 3 months prior to the first study vaccine administration.
E11. Receipt of immune globulins, blood or blood-derived products in the past 3 months.
E12. Receipt of intranasal and intra-ocular medications within 3 days prior to study enrollment

### Prior/Concurrent Clinical Study Experience

E13. Participation at the time of study enrollment or planned participation during the present study period in another clinical study investigating a vaccine, drug, medical device, or medical procedure.

### Other Exclusions

E14. Deprived of freedom in an emergency setting, or hospitalized involuntarily.
E15. Identified as a natural or adopted child of the Investigator or employee with direct involvement in the proposed study.

### BRIEF SUMMARY

The study will be a Phase III, parallel group, randomized, observer-blind, placebo-controlled, multi-national, multi center, multi-arm study to be conducted in 947 healthy children enrolled at 6 months of age. The purpose of the study is to evaluate the non-inferiority of the immune response of the LD when compared to the SD RSV ΔNS2/Δ1313/I1314L (Sanofi) vaccine and the safety of the SD and HD vaccine in preterm born children and of the HD vaccine in full term born children administered by intranasal route and compared to placebo.

Study details include:

The study duration will be approximately 8-9 months for each participant, including the 6 months safety follow-up phone call after the second study intervention administration.

The study intervention administration for the participants will be on D01 and D57 (1 intranasal administration in each nostril at each timepoint).

The visit frequency will be as shown in Table 1. Safety data will be collected during all study visits, as well as at designated timepoints via contacts with the parents/legally acceptable representative (LAR) of the study participants.

### Number of Participants:

A total of 947 participants are expected to be randomized: 35 in Cohort 1 and 912 in Cohort 2.

### Study Arms and Duration:

In each study group (RSV ΔNS2/Δ1313/I1314L (Sanofi) vaccine and placebo groups), eligible participants will be randomized to receive 2 intranasal administrations (56 days apart, ie, at D01 and D57) of either dose of the RSV ΔNS2/Δ1313/I1314L (Sanofi) vaccine or placebo.

Study arms will be as follows:

Cohort 1 participants (born prematurely) enrollment will be done in a sequential/stepwise approach. Step 1: the first 20 participants will be randomized 1:1 to receive SD RSV ΔNS2/Δ1313/I1314L (Sanofi) or placebo. Step 2: based on the acceptability of the safety profile (up to 28 days after the first vaccination), 15 additional participants will be randomized 2:1 to HD RSV ΔNS2/Δ1313/I1314L (Sanofi) or placebo.

### Step 1:

Group 1 (N=10): 2 administrations of SD RSV ΔNS2/Δ1313/I1314L (Sanofi) (target 6.4 log₁₀ PFU/dose)
Group 2 (N=10): 2 administrations of placebo
   Contingent upon safety of SD RSV ΔNS2/Δ1313/I1314L (Sanofi):

### Step 2:

Group 3 (N=10): 2 administrations of HD RSV ΔNS2/Δ1313/I1314L (Sanofi) (target 7.0 log₁₀ PFU/dose)
Group 4 (N=5): 2 administrations of placebo
   Contingent upon an acceptable safety profile of the HD in Cohort 1, enrolment in Cohort 2 will open to participants born preterm.
   Cohort 2 participants will be randomized at a 2:2:1:1 ratio for Groups 1 to 4, respectively.
   Group 1 (N=304): 2 administrations of LD RSV ΔNS2/Δ1313/I1314L (Sanofi) (target 5.4 log₁₀ PFU/dose)
   Group 2 (N=304): 2 administrations of SD RSV ΔNS2/Δ1313/I1314L (Sanofi) (target 6.4 log₁₀ PFU/dose)
   Group 3 (N=152): 2 administrations of HD RSV ΔNS2/Δ1313/I1314L (Sanofi) (target 7.0 log₁₀ PFU/dose)
   Group 4 (N=152): 2 administrations of placebo
   Cohorts 1 and 2 will enroll in parallel.
   At each vaccination visit, 0.1 mL of RSV ΔNS2/Δ1313/I1314L (Sanofi) vaccine or placebo will be administered in each nostril with the use of an investigational device.
   Study duration will be approximately 8-9 months for each participant.

### Study Intervention(s):

### Investigational Medicinal Product 1: LD RSV ΔNS2/Δ1313/I1314L (Sanofi) Vaccine (Target 5.4 [5.6±0.3] Log₁₀ PFU/0.2 mL)

Form: Liquid for nasal spray
Composition: Each 0.2 mL dose of RSV ΔNS2/Δ1313/I1314L (Sanofi) vaccine will contain the live-attenuated RSV with i) a 523-nucleotide deletion of the NS2 gene, ii) an amino acid deletion in the L protein (Δ1313; deletion of S1313), and iii) genetically stabilizing mutation in the L gene (I1314L), approximately 0.1 mL to be delivered as a fine mist of droplets per nostril, using an intranasal atomizer device.
Route of administration: intranasal

### Investigational Medicinal Product 2: SD RSV ΔNS2/Δ1313/I1314L (Sanofi) Vaccine (Target 6.4 Log₁₀ PFU/0.2 mL)

Same form, composition, and route of administration as for product 1.

### Investigational Medicinal Product 3: HD RSV ΔNS2/Δ1313/I1314L (Sanofi) Vaccine (Target 7.0 [6.9±0.3] Log₁₀ PFU/0.2 mL).

Same form, composition, and route of administration as for product 1.

### Investigational Medicinal Product 4: Placebo

Form: Liquid for nasal spray.
Composition: buffer, same histidine-based formulation buffer as that used for the RSV ΔNS2/Δ1313/I1314L (Sanofi) vaccine, to be delivered as approximately 0.1 mL per nostril
Route of administration: intranasal

### Statistical Considerations:

### Statistical Hypotheses

### Primary Immunogenicity Objective

Twenty-eight days after the second administration of the RSV ΔNS2/Δ1313/I1314L (Sanofi) vaccine to Cohort 2 participants, the geometric mean titers (GMTs) of neutralizing RSV (A and B) antibodies of the LD RSV ΔNS2/Δ1313/I1314L (Sanofi) group will be compared to SD RSV ΔNS2/Δ1313/I1314L (Sanofi) group. The following hypotheses will be tested for each RSV antibody:
H₀: GMT (rsv, LD)/GMT (rsv, SD)≤⅔
   H₁: GMT (rsv, LD)/GMT (rsv, SD)>⅔

where GMT (rsv, LD) and GMT (rsv, SD) are GMTs of RSV antibodies (A and B) in LD RSV ΔNS2/Δ1313/I1314L (Sanofi) and SD RSV ΔNS2/Δ1313/I1314L (Sanofi) from Cohort 2, respectively.

If the lower limit of the 2-sided 95% CI of the ratio of the GMTs between LD RSV ΔNS2/Δ1313/I1314L (Sanofi) and SD RSV ΔNS2/Δ1313/I1314L (Sanofi) groups is >⅔ for both neutralizing RSV antibodies (A and B), the inferiority null-hypothesis will be rejected and non-inferiority (NI) of LD RSV ΔNS2/Δ1313/I1314L (Sanofi) compared to SD RSV ΔNS2/Δ1313/I1314L (Sanofi) will be demonstrated.

### Statistical Analyses

All endpoints will be summarized at each timepoint by study intervention group. For the analysis of Cohort 1, Group 2 and Group 4 (ie, placebo groups) may be pooled. Results will be presented separately for each cohort.

In general, categorical variables will be summarized and presented by frequency counts, percentages, and CIs. The 95% CIs of point estimates will be calculated using the normal approximation for quantitative data and the exact binomial distribution (Clopper-Pearson method) for percentages. For GMTs and GMTRs, 95% CIs of point estimates will be calculated using normal approximation, assuming they are log₁₀-normally distributed.

The immunogenicity analyses will be performed on the Per-Protocol Analysis Set (PPAS) and may be confirmed on the Full Analysis Set (FAS) if the difference between number of participants in the PPAS and the number of participants in the FAS is not less than 10%.

Safety analyses will be performed on the Safety Analysis Set (SafAS).

### Primary Endpoints

### Immunogenicity Analysis

The 2-sided 95% CIs of the ratio of GMTs between LD RSV ΔNS2/Δ1313/I1314L (Sanofi) group and SD RSV ΔNS2/Δ1313/I1314L (Sanofi) group from Cohort 2 will be calculated assuming that log₁₀ transformation of the titers follows a normal distribution. If the lower limits of the 2-sided 95% CIs are greater than ⅔ for both RSV A and RSV B antibodies, the inferiority null hypothesis will be rejected.

### Safety Analysis

The safety parameters, including frequencies of immediate reactions, solicited reactions, unsolicited AEs, MAAEs, AESIs, and SAEs will be described by study intervention group after each and any study intervention administration with the 95% CIs of point estimates calculated using the exact binomial distribution (Clopper-Pearson method) for proportions.

### Interim Analysis

An unblinded primary completion analysis will be performed on data collected up to 28 days post-dose 2 for immunogenicity and safety for each cohort. Randomization code will be broken for the sponsor, but blinding will be maintained at sites and at the participants/parents level.

### Sample Size Determination

Cohort 1: 35 participants born preterm will be enrolled. This is an arbitrary sample size to generate safety data in pre-term children. No statistical hypotheses will be tested on this cohort.

Cohort 2: a total of 912 participants will be enrolled, with 304 in each LD and SD RSV ΔNS2/Δ1313/I1314L (Sanofi) groups and 152 in each of HD RSV ΔNS2/Δ1313/I1314L (Sanofi) and placebo groups. The allocation ratio is 2:2:1:1.

### For Primary Immunogenicity Objective

With 243 evaluable participants in LD RSV ΔNS2/Δ1313/I1314L (Sanofi) and 243 evaluable participants in SD RSV ΔNS2/Δ1313/I1314L (Sanofi), the study will have approximately 90% power to declare the non-inferiority for the primary immunogenicity objective based on a one-sided alpha of 2.5%. Considering an estimated 20% attrition rate, 304 participants in each LD and SD RSV ΔNS2/Δ1313/I1314L (Sanofi) group will be enrolled.

### For Primary Safety Objective

The sample size of 152 participants in the HD RSV ΔNS2/Δ1313/I1314L (Sanofi) group will provide a probability of 95% to observe an event that has a true incidence of 2%, and will provide a probability of 78% to observe an event that has a true incidence of 1%.

### Example 4: Intranasal Atomization Delivery Device

The intranasal atomization delivery device used to deliver the RSV vaccine and placebo was tested to measure the dose accuracy, the spray droplet size distribution (DSD), the spray plume geometry (PG), the spray pattern (SP), and the dose of infectious titer.

### Material and Methods

### Material

Vaccine buffer; store at 2° C. to 8° C.; avoid light exposure.

Scale-up lot high dose DP Lot #19-035-FBP/FP and low dose DP lot #19-054-FBP/FP; both stored at <−60° C.

MAD130 device, Intranasal Mucosal Atomization Device with 1-mL syringe and a vial adaptor (made by Teleflex) Lot #73J1700328.

Poly (lactic acid) plastic 3D-printed dose divider; 0.1 mL, blue color (in-house made).

RSV vaccine purified bulk.

### Methods

The methods used herein are in accordance with the FDA guidance and the EMA guidelines (U.S. Food and Drug Administration. Guidance for industry: Nasal spray and inhalation solution, suspension, and spray drug products-Chemistry, manufacturing, and controls documentation. Fed. Regist. 2002, 1-49. European Medicines Agency. Guideline on the Pharmaceutical Quality of Inhalation and Nasal Products; European Medicines Agency: London, UK, 2006; pp. 1-27).

Shot weight: Weighing the amount of fluid expelled by the pump (syringe in this case) during a single-dose actuation to measure dose delivery.

Spray pattern (SP): Measuring the cross-sectional uniformity of the spray plume at a specified distance (i.e., 30 mm) from the nozzle tip to characterize device performance.

Spray pattern testing involves using laser illumination and a camera to capture a time sequence of images from a normal view at a pre-determined distance along the centerline of the emitted spray from the device. Proveris software is used to automatically analyze the collected image sequences and calculate a time-averaged image that is used to determine the spray pattern contour and other metrics. An automated, non-impaction (automated analysis coupled with laser light sheet and digital camera imaging) spray pattern method tailored for the RSV vaccine or vaccine buffer based on the Proveris Spray VIEW® instrument platform according to the FDA guidance was used. All spray pattern metrics were analyzed according to following:

Dmax (The longest chord length that connects two points on the spray pattern contour and passes through the weighted center of mass of the contour)

Dmin (The shortest chord length that connects two points on the spray pattern contour and passes through the weighted center of mass of the contour)

Ovality (The ratio of Dmax to Dmin)

Area (The area bounded by the spray pattern contour)

A total of 10 devices with 20 sprays in total were measured. Method settings used for spray pattern analysis are detailed in Table 19 below:

TABLE 19
Spray Pattern Method Details at 30 mm Distance from Nozzle Tip
Spray Pattern

| | |
|---|---|
| Frames | 250 |
| Frame Rate (Hz) | 500 |
| Camera Height (cm) | 24.0 |
| Camera Position (cm) | 5.0 |
| Lens Type | 9 mm |
| Lens Aperture | 1.4 |
| Laser Depth (cm) | 5.5 |
| Laser Position (cm) | 5.0 |
| Orifice Tip Distance | 30 |
| (mm) | |
| Time Average Method | Automatic |

Plume geometry (PG): Measuring the geometry of the spray plume (plume width and plume angle) from a side-view perspective on a time-averaged basis at a specified distance (i.e., 30 mm) from the nozzle tip to characterize device performance.

Plume geometry testing involves laser illumination and a camera to capture a time sequence of images from a sideward view along the centerline of the emitted spray from the device. These resulting image sequences provide visualization of the spray and can be analyzed to determine the duration and orientation of the spray and spray particles.

A laser light sheet and digital camera-based plume geometry methods tailored for the RSV vaccine or vaccine buffer were used based on the Proveris SprayVIEW® instrument platform. All plume geometry metrics were analyzed according to the relevant FDA guidance document including the following (2): Plume width and Plume angle. Plume geometry and metered shot weight data was collected from 10 devices with 20 sprays in total. Method settings used for plume geometry are detailed in Table 20 below. Time averaged measurements of plume were analyzed.

TABLE 20
Plume Geometry Method details at 30-mm Distance from Nozzle Tip
Plume Geometry

| | |
|---|---|
| Frames | 250 |
| Frame Rate (Hz) | 450 |
| Camera Height | 12 |
| (cm) | |
| Camera Position | 22 |
| (cm) | |
| Lens Type | 9 mm |
| Lens Aperture | 1.4 |
| Laser Height | 18.5 |
| (cm) | |
| Laser Position | 5.0 |
| (cm) | |
| Laser Depth (cm) | 5.5 |
| Plume Distance | 30 |
| (mm) | |
| Time Delay (ms) | Time averaged |
| | for entire |
| | spray |
| | duration |

Droplet Size Distribution (DSD): measuring atomized droplet size and its distribution by a laser diffraction method on the fully developed steady phase at a specified distance (i.e., 30 mm) from the nozzle tip to characterize device performance. Spray droplet size distribution can be measured at a specified distance from nozzle tip by a laser diffraction method on the fully developed steady phase, to measure, for example, Dv10, Dv50, Dv90, span [(Dv90-Dv10)/Dv50], and percentage of droplets less than 10 μm.

A droplet size distribution by laser diffraction method was used tailored for the RSV vaccine or vaccine buffer based on the Malvern Spraytec instrument platform. All droplet size distribution (DSD) metrics were performed according to the above referenced FDA guidance document and analyzed, including the following:
Dv90: particle size below which 90% of the spray lies for volume based distribution
Dv50: particle size below which 50% of the spray lies for volume based distribution
Dv10: particle size below which 10% of the spray lies for volume based distribution
% V<10 μm: volume of particles less than 10 microns in size

Span: Measures the width of the distribution. The narrower the distribution, the smaller the span becomes. It is calculated as:

$$Span = ( Dv 90 - Dv 10 ) / Dv 50$$

All samples were tested with the setting containing 3D dose divider and primed the atomizer head.

### Actuation Mechanism and Parameters

Given the mechanism of the MAD Nasal™ device with the dose divider, the force limited actuation by the Viota unit dose software was utilized: the software stops the actuation when an end-of-stroke force limit is reached, which replicates the human actuation process more precisely for this specific device—the first dose completes when the plunger hits the dose divider, and the second dose completes when the plunger hits the syringe hard stop. The following set of actuation parameters (Table 21) were used for all the testing.

TABLE 21
Actuation Parameters Used for the Performance Evaluation

| Metric | Value |
|---|---|
| Contact Force (kg) | 0.1 |
| End of Stroke Force (kg) | 4.5 |
| AS Velocity (mm/s) | 50 |
| Hold Time (milli- | 300 |
| seconds) | |
| Priming | At least 0.1 mL through the atomizer until |
| | the liquid level has reached to the target |
| | graduation mark of the syringe (0.2 mL) |

### Results and Discussion

### Physical Properties of Testing Matrix Comparison

The key physical properties of RSV vaccine and vaccine buffer were compared and the vaccine buffer was used in the analysis to measure the dose accuracy, the spray droplet size distribution (DSD), the spray plume geometry (PG), the spray pattern (SP). The comparison is shown in Table 22.

TABLE 22
Physical Properties of RSV vaccine and vaccine buffer

| | | | | | Surface |
|---|---|---|---|---|---|
| | | Osmolality | | | tension |
| Sample ID | pH | (mOsm/kg) | Density | Viscosity - cP | (mN/m) |
| FBP, Lot# | 6.95 | 1704 | 1.1379 | 8.18 | 63.57 |
| 19-T-FS072/HD Tox, | | | | | |
| Lot# 19-T-FS073 | | | | | |
| HD DP Lot# 19-035-FBP/FP | 7.04 | 1608 | 1.1387 | 7.81 | N/Ap |
| LD DP Lot# 19-054-FBP/FP | 7.09 | 1530 | 1.1381 | 6.07 | 63.41 |
| Vaccine Buffer Lot# | 7.18 | 1734 | 1.1377 | 3.89 | 66.94 |
| P11415 | | | | | |

The above results suggested the similarity of the physical properties of DP and the formulation buffer, with the exception that buffer matrix only has a slightly lower viscosity.

### Test Results

A total of 10 vials filled DP (HD Lot #19-035-FP) were tested. Sample was drawn from each vial and then dispensed into 2 tubes (0.1 mL/tube #1 with first tube from the first shot, and mL/tube #2 for the second dose; e.g., vial #1 is tube #1 and tube #2, vial #2 is tube #3 and tube #4, etc.).

### Dose Accuracy by Shot Weight

The dose weight is summarized below in Table 23. The raw results are converted to dose volume by using density value of 1.1377 g/mL.

TABLE 23
Dose Accuracy by Dose Volume

| | First shot (mL) | Second shot (mL) | Total (mL) |
|---|---|---|---|
| Average | 0.126 | 0.108 | 0.234 |
| SD | 0.012 | 0.004 | 0.014 |
| Min | 0.11 | 0.101 | 0.215 |
| Max | 0.148 | 0.113 | 0.256 |

### Dose Titer by Plaque Assay

Infectious titer is the main indicator for formulation and stability evaluation. Infectious titer is also used as label claim of the RSVi Dose (i.e., high dose titer target is 6.0 log₁₀ PFU/dose, where dose=0.2 mL). Titer readings directly correlate to viral activity; a significant decrease in viral activity is considered a drop in the infectious titer.

TABLE 24
Titer Results Summary

| | First shot titer | Second shot titer | Average titer |
|---|---|---|---|
| | (log₁₀ PFU/mL) | (log₁₀ PFU/mL) | (log₁₀ PFU/mL) |
| Average | 7.2 | 7.24 | 7.22 |
| SD | 0.07 | 0.07 | 0.06 |
| min | 7.07 | 7.11 | 7.1 |
| max | 7.30 | 7.30 | 7.30 |

All titers were consistent and met the concentration of the high dose RSV vaccine requirement.

### Shot Weight

A second test of shot weight was conducted using a total of 10 devices (20 sprays) using an automated actuator. Results are summarized in Table 25. The maximum of first spray dose weight is 127.60 mg (112.07 μL in dose volume) and the minimum is 108.90 mg (95.72 μL). The second dose average is 106.21 mg (102.11 μL), which weighs slightly less than the first dose 121.64 mg (106.92 μL).

TABLE 25
Shot Weight Using Vaccine Buffer

| | Shot Weight | Shot Volume | Total Shot |
|---|---|---|---|
| | (mg) | (μL) | Volume (μL) |
| Maximum | 127.60 | 112.07 | 214.29 |
| Minimum | 108.90 | 95.72 | 195.57 |
| Average of first shots | 121.64 | 106.92 | N/Ap |
| Average of second shots | 106.21 | 102.11 | N/Ap |
| Average of all shots | 113.93 | 104.52 | 208.18 |

### Spray Pattern

A total of 10 devices (20 sprays) were tested for the spray pattern. The data in Table 24 below show that the mean spay area is 129.15 mm² with a standard deviation of 14.00. The average ovality is 1.25 with a standard deviation of 0.10. The average Dmax and Dmin are 14.31 mm and 11.47 mm, respectively, with standard deviations of 0.81 and 0.96, respectively. In addition, the second dose (133.74 mm²) showed slightly larger spray area compared to the first dose (124.56 mm²) on average. Overall, first doses and second doses had similar results for spray area, ovality, Dmax, and Dmin.

TABLE 26
Spray Pattern Data Summary

| | Area | | Dmax | Dmin |
|---|---|---|---|---|
| | (mm²) | Ovality | (mm) | (mm) |
| Minimum | 108.30 | 1.10 | 13.15 | 9.55 |
| Maximum | 159.00 | 1.54 | 16.23 | 12.91 |
| Average of first shots | 124.56 | 1.28 | 14.17 | 11.15 |
| Average of second shots | 133.74 | 1.23 | 14.45 | 11.78 |
| Average of all shots | 129.15 | 1.25 | 14.31 | 11.47 |
| Standard Deviation | 14.00 | 0.10 | 0.81 | 0.96 |

### Plume Geometry (PG)

Plume geometry and metered shot weight data were simultaneously collected from the 10 devices with a total of 20 shots. Plume geometry results were consistent among different devices and between the two doses as shown in Table 27 below.

TABLE 27
Plume Geometry Data Summary

| | Plume Angle (Degree) | Plume Width (mm) |
|---|---|---|
| Maximum | 28.70 | 15.38 |
| Minimum | 19.00 | 10.06 |
| Average of first shots | 24.91 | 13.33 |
| Average of second shots | 25.43 | 13.63 |
| Average of all shots | 25.17 | 13.48 |
| Standard Deviation | 3.03 | 1.68 |

### Droplet Size Distribution (DSD)

Droplet size distribution results were consistent among different devices, and between the two doses. Results are given in Table 28. The data below show that droplet size distribution results were consistent among different devices and between the two doses.

TABLE 28
All Spray DSD Data Summary Table

| | Dv 10 | Dv 50 | Dv 90 | | % V < 10 |
|---|---|---|---|---|---|
| | (μm) | (μm) | (μm) | Span | μm |
| Average of first shots | 29.84 | 94.16 | 205.92 | 1.88 | 0.17 |
| Average of second shots | 29.26 | 93.25 | 206.01 | 1.90 | 0.20 |
| Average of all shots | 29.55 | 93.70 | 205.97 | 1.89 | 0.19 |
| Minimum | 25.62 | 81.73 | 184.50 | 1.73 | 0.00 |
| Maximum | 34.45 | 109.50 | 239.30 | 2.13 | 0.31 |

The DSD of a nasal spray is an important parameter for a nasal product since the DSD significantly influences the in vivo deposition of the drug in the nasal cavity. The droplet size using a nasal atomizer delivery device has an average droplet size of Dv50=93.70 μm. The average for small droplets (% V<10 μm) is 0.19%, which can be considered safe and has negligible to deposit to the lung.

### Device In-Use Stability

A test was conducted on the RSV vaccine formulation and the atomization delivery device to determine compatibility at room temperature up to 24 hours.

For this mimic study, HD or LD DP vials were thawed for 5 to 7 minutes at room temperature. Followed by the device sample preparation, MAD130 syringe was inserted into thawed glass vial via rubber stopper and 300 μL of DP was withdrawn after de-bubbling. Atomizer was then attached and purged to empty the head space air by reducing volume to 200 μL. Each prepared device set was labelled and stored horizontally at respective temperatures (i.e., 2° C. to 8° C. and 25° C.) for 2 h, 4 h, 6 h and 24 h. Collected samples were frozen at <−60° C. and transferred to AnSci for PA test. The results of the infections titer loss are shown below.

Average log reduction/titre loss for HD (≥6.7 log₁₀ PFU/mL) and LD (≥5.7 log₁₀ PFU/mL) was ≤0.1 log₁₀ PFU/mL after 24 hours at 5° C.±3° C.

Average log reduction/titre loss for HD (≥6.7 log₁₀ PFU/mL) and LD (≥5.7 log₁₀ PFU/mL) was ≤0.1 log₁₀ PFU/mL after 6 hours at 25° C.±2° C.; however, it was ≥0.1 log₁₀ PFU/mL after 24 hours at 25° C.±2° C.

In summary, the above in-use stability data indicate that infectious titre meets the expected target after 6-hour incubation at 25° C.±2° C. and 5° C.±3° C. in MAD130 IN device for both HD and LD formulations.

It is recommended that RSVi HD and LD are kept in MAD130 IN devices for a maximum of 6 hours at room temperature (17).

APPENDIX A
SEQUENCE SUMMARY
DESCRIPTION OF THE SEQUENCES
Appendix A provides a listing of certain sequences referenced herein.
The amino acid sequences provided are from N-terminus to C-terminus.
The nucleic acid sequences are from 5′ to 3′.

| SEQ | | |
|---|---|---|
| ID | | |
| NO. | Name | Sequence |
| 1 | RSV | acgggaaaaaatgcgtacaacaaacttgcataaaccaaaaaaatggggcaaataagaatttgata |
| | ΔNS2/ | agtaccacttaaatttaactcccttggttagagatgggcagcaattcattgagtatgataaaagt |
| | Δ1313/ | tagattacaaaatttgtttgacaatgatgaagtagcattgttaaaaataacatgctatactgata |
| | I1314L | aattaatacatttaactaatgctttggctaaggcagtgatacatacaatcaaattgaatggcatt |
| | (Sanofi) | gtgtttgtgcatgttattacaagtagtgatatttgccctaataataatattgtagtaaaatccaa |
| | | tttcacaacaatgccagtactacaaaatggaggttatatatgggaaatgatggaattaacacatt |
| | | gctctcaacctaatggtctactagatgacaattgtgaaattaaattctccaaaaaactaagtgat |
| | | tcaacaatgaccaattatatgaatcaattatctgaattacttggatttgatcttaatccataaat |
| | | tataattaatatcaactagcaaatcaatgtcactaacaccattagttaatataaaacttaaggag |
| | | agatataagatagaagatggggcaaatacaaccatggctcttagcaaagtcaagttgaatgatac |
| | | actcaacaaagatcaacttctgtcatccagcaaatacaccatccaacggagcacaggagatagta |
| | | ttgatactcctaattatgatgtgcagaaacacatcaataagttatgtggcatgttattaatcaca |
| | | gaagatgctaatcataaattcactgggttaataggtatgttatatgcgatgtctaggttaggaag |
| | | agaagacaccataaaaatactcagagatgcgggatatcatgtaaaagcaaatggagtagatgtaa |
| | | caacacatcgtcaagacattaatggaaaagaaatgaaatttgaagtgttaacattggcaagctta |
| | | acaactgaaattcaaatcaacattgagatagaatctagaaaatcctacaaaaaaatgctaaaaga |
| | | aatgggagaggtagctccagaatacaggcatgactctcctgattgtgggatgataatattatgta |
| | | tagcagcattagtaataactaaattagcagcaggggacagatctggtcttacagccgtgattagg |
| | | agagctaataatgtcctaaaaaatgaaatgaaacgttacaaaggcttactacccaaggacatagc |
| | | caacagcttctatgaagtgtttgaaaaacatccccactttatagatgtttttgttcattttggta |
| | | tagcacaatcttctaccagaggtggcagtagagttgaagggatttttgcaggattgtttatgaat |
| | | gcctatggtgcagggcaagtgatgttacggtggggagtcttagcaaaatcagttaaaaatattat |
| | | gttaggacatgctagtgtgcaagcagaaatggaacaagttgttgaggtttatgaatatgcccaaa |
| | | aattgggtggtgaagcaggattctaccatatattgaacaacccaaaagcatcattattatctttg |
| | | actcaatttcctcacttctccagtgtagtattaggcaatgctgctggcctaggcataatgggaga |
| | | gtacagaggtacaccgaggaatcaagatctatatgatgcagcaaaggcatatgctgaacaactca |
| | | aagaaaatggtgtgattaactacagtgtactagacttgacagcagaagaactagaggctatcaaa |
| | | catcagcttaatccaaaagataatgatgtagagctttgagttaataaaaaatggggcaaataaat |
| | | catcatggaaaagtttgctcctgaattccatggagaagatgcaaacaacagggctactaaattcc |
| | | tagaatcaataaagggcaaattcacatcacccaaagatcccaagaaaaaagatagtatcatatct |
| | | gtcaactcaatagatatagaagtaaccaaagaaagccctataacatcaaattcaactattatcaa |
| | | cccaacaaatgagacagatgatactgcagggaacaagcccaattatcaaagaaaacctctagtaa |
| | | gtttcaaagaagaccctacaccaagtgataatcccttttctaaactatacaaagaaaccatagaa |
| | | acatttgataacaatgaagaagaatccagctattcatacgaagaaataaatgatcagacaaacga |
| | | taatataacagcaagattagataggattgatgaaaaattaagtgaaatactaggaatgcttcaca |
| | | cattagtagtggcaagtgcaggacctacatctgctcgggatggtataagagatgccatggttggt |
| | | ttaagagaagaaatgatagaaaaaatcagaactgaagcattaatgaccaatgacagattagaagc |
| | | tatggcaagactcaggaatgaggaaagtgaaaagatggcaaaagacacatcagatgaagtgtctc |
| | | tcaatccaacatcagagaaattgaacaacctattggaagggaatgatagtgacaatgatctatca |
| | | cttgaagatttctgattagttaccaatcttcacatcaacacacaataccaacagaagaccaacaa |
| | | actaaccaacccaatcatccaaccaaacatccatccgccaatcagccaaacagccaacaaaacaa |
| | | ccagccaatccaaaactaaccacccggaaaaaatctataatatagttacaaaaaaaggaaagggt |
| | | ggggcaaatatggaaacatacgtgaacaagcttcacgaaggctccacatacacagctgctgttca |
| | | atacaatgtcttagaaaaagacgatgaccctgcatcacttacaatatgggtgcccatgttccaat |
| | | catctatgccagcagatttacttataaaagaactagctaatgtcaacatactagtgaaacaaata |
| | | tccacacccaagggaccttcactaagagtcatgataaactcaagaagtgcagtgctagcacaaat |
| | | gcccagcaaatttaccatatgcgctaatgtgtccttggatgaaagaagcaaactagcatatgatg |
| | | taaccacaccctgtgaaatcaaggcatgtagtctaacatgcctaaaatcaaaaaatatgttgact |
| | | acagttaaagatctcactatgaagacactcaaccctacacatgatattattgctttatgtgaatt |
| | | tgaaaacatagtaacatcaaaaaaagtcataataccaacatacctaagatccatcagtgtcagaa |
| | | ataaagatctgaacacacttgaaaatataacaaccactgaattcaaaaatgctatcacaaatgca |
| | | aaaatcatcccttactcaggattactattagtcatcacagtgactgacaacaaaggagcattcaa |
| | | atacataaagccacaaagtcaattcatagtagatcttggagcttacctagaaaaagaaagtatat |
| | | attatgttaccacaaattggaagcacacagctacacgatttgcaatcaaacccatggaagattaa |
| | | cctttttcctctacatcagtgtgttaattcatacaaactttctacctacattcttcacttcacca |
| | | tcacaatcacaaacactctgtggttcaaccaatcaaacaaaacttatctgaagtcccagatcatc |
| | | ccaagtcattgtttatcagatctagtactcaaataagttaataaaaaatatacacatggggcaaa |
| | | taatcattggaggaaatccaactaatcacaatatctgttaacatagacaagtccacacaccatac |
| | | agaatcaaccaatggaaaatacatccataacaatagaattctcaagcaaattctggccttacttt |
| | | acactaatacacatgatcacaacaataatctctttgctaatcataatctccatcatgattgcaat |
| | | actaaacaaactttgtgaatataacgtattccataacaaaacctttgagttaccaagagctcgag |
| | | ttaatacttgataaagtagttaattaaaaatagtcataacaatgaactaggatatcaagactaac |
| | | aataacattggggcaaatgcaaacatgtccaaaaacaaggaccaacgcaccgctaagacattaga |
| | | aaggacctgggacactctcaatcatttattattcatatcatcgtgcttatataagttaaatctta |
| | | aatctgtagcacaaatcacattatccattctggcaatgataatctcaacttcacttataattgca |
| | | gccatcatattcatagcctoggcaaaccacaaagtcacaccaacaactgcaatcatacaagatgc |
| | | aacaagccagatcaagaacacaaccccaacatacctcacccagaatcctcagcttggaatcagtc |
| | | cctctaatccgtctgaaattacatcacaaatcaccaccatactagcttcaacaacaccaggagtc |
| | | aagtcaaccctgcaatccacaacagtcaagaccaaaaacacaacaacaactcaaacacaacccag |
| | | caagcccaccacaaaacaacgccaaaacaaaccaccaagcaaacccaataatgattttcactttg |
| | | aagtgttcaactttgtaccctgcagcatatgcagcaacaatccaacctgctgggctatctgcaaa |
| | | agaataccaaacaaaaaaccaggaaagaaaaccactaccaagcccacaaaaaaaccaaccctcaa |
| | | gacaaccaaaaaagatcccaaacctcaaaccactaaatcaaaggaagtacccaccaccaagccca |
| | | cagaagagccaaccatcaacaccaccaaaacaaacatcataactacactactcacctccaacacc |
| | | acaggaaatccagaactcacaagtcaaatggaaaccttccactcaacttcctccgaaggcaatcc |
| | | aagcccttctcaagtctctacaacatccgagtacccatcacaaccttcatctccacccaacacac |
| | | cacgccagtagttacttaaaaacatattatcacaaaaggccttgaccaacttaaacagaatcaaa |
| | | ataaactctggggcaaataacaatggagttgctaatcctcaaagcaaatgcaattaccacaatcc |
| | | tcactgcagtcacattttgttttgcttctggtcaaaacatcactgaagaattttatcaatcaaca |
| | | tgcagtgcagttagcaaaggctatcttagtgctctgagaactggttggtataccagtgttataac |
| | | tatagaattaagtaatatcaagaaaaataagtgtaatggaacagatgctaaggtaaaattgataa |
| | | aacaagaattagataaatataaaaatgctgtaacagaattgcagttgctcatgcaaagcacacaa |
| | | gcaacaaacaatcgagccagaagagaactaccaaggtttatgaattatacactcaacaatgccaa |
| | | aaaaaccaatgtaacattaagcaagaaaaggaaaagaagatttcttggttttttgttaggtgttg |
| | | gatctgcaatcgccagtggcgttgctgtatctaaggtcctgcacctagaaggggaagtgaacaag |
| | | atcaaaagtgctctactatccacaaacaaggctgtagtcagcttatcaaatggagttagtgtttt |
| | | aaccagcaaagtgttagacctcaaaaactatatagataaacaattgttacctattgtgaacaagc |
| | | aaagctgcagcatatcaaatatagaaactgtgatagagttccaacaaaagaacaacagactacta |
| | | gagattaccagggaatttagtgttaatgcaggcgtaactacacctgtaagcacttacatgttaac |
| | | taatagtgaattattgtcattaatcaatgatatgcctataacaaatgatcagaaaaagttaatgt |
| | | ccaacaatgttcaaatagttagacagcaaagttactctatcatgtccataataaaagaggaagtc |
| | | ttagcatatgtagtacaattaccactatatggtgttatagatacaccctgttggaaactacacac |
| | | atcccctctatgtacaaccaacacaaaagaagggtccaacatctgtttaacaagaactgacagag |
| | | gatggtactgtgacaatgcaggatcagtatctttcttcccacaagctgaaacatgtaaagttcaa |
| | | tcaaatcgagtattttgtgacacaatgaacagtttaacattaccaagtgaagtaaatctctgcaa |
| | | tgttgacatattcaaccccaaatatgattgtaaaattatgacttcaaaaacagatgtaagcagct |
| | | ccgttatcacatctctaggagccattgtgtcatgctatggcaaaactaaatgtacagcatccaat |
| | | aaaaatcgtggaatcataaagacattttctaacgggtgcgattatgtatcaaataaaggggtgga |
| | | cactgtgtctgtaggtaacacattatattatgtaaataagcaagaaggtaaaagtctctatgtaa |
| | | aaggtgaaccaataataaatttctatgacccattagtattcccctctgatgaatttgatgcatca |
| | | atatctcaagtcaacgagaagattaaccagagcctagcatttattcgtaaatccgatgaattatt |
| | | acataatgtaaatgctggtaaatccaccacaaatatcatgataactactataattatagtgatta |
| | | tagtaatattgttatcattaattgctgttggactgctcttatactgtaaggccagaagcacacca |
| | | gtcacactaagcaaagatcaactgagtggtataaataatattgcatttagtaactaaataaaaat |
| | | agcacctaatcatgttcttacaatggtttactatctgctcatagacaacccatctgtcattggat |
| | | tttcttaaaatctgaacttcatcgaaactctcatctataaaccatctcacttacactatttaagt |
| | | agattcctagtttatagttatataaaacacaattgcatgccagattaacttaccatctgtaaaaa |
| | | tgaaaactggggcaaatatgtcacgaaggaatccttgcaaatttgaaattcgaggtcattgctta |
| | | aatggtaagaggtgtcattttagtcataattattttgaatggccaccccatgcactgcttgtaag |
| | | acaaaactttatgttaaacagaatacttaagtctatggataaaagtatagataccttatcagaaa |
| | | taagtggagctgcagagttggacagaacagaagagtatgctcttggtgtagttggagtgctagag |
| | | agttatataggatcaataaacaatataactaaacaatcagcatgtgttgccatgagcaaactcct |
| | | cactgaactcaatagtgatgatatcaaaaagctgagggacaatgaagagctaaattcacccaaga |
| | | taagagtgtacaatactgtcatatcatatattgaaagcaacaggaaaaacaataaacaaactatc |
| | | catctgttaaaaagattgccagcagacgtattgaagaaaaccatcaaaaacacattggatatcca |
| | | taagagcataaccatcaacaacccaaaagaatcaactgttagtgatacaaatgaccatgccaaaa |
| | | ataatgatactacctgacaaatatccttgtagtataacttccatactaataacaagtagatgtag |
| | | agttactatgtataatcaaaagaacacactatatttcaatcaaaacaacccaaataaccatatgt |
| | | actcaccgaatcaaacattcaatgaaatccattggacctctcaagaattgattgacacaattcaa |
| | | aattttctacaacatctaggtattattgaggatatatatacaatatatatattagtgtcataaca |
| | | ctcaattctaacactcaccacatcgttacattattaattcaaacaattcaagttgtgggacaaaa |
| | | tggatcccattattaatggaaattctgctaatgtttatctaaccgatagttatttaaaaggtgtt |
| | | atctctttctcagagtgtaatgctttaggaagttacatattcaatggtccttatctcaaaaatga |
| | | ttataccaacttaattagtagacaaaatccattaatagaacacatgaatctaaagaaactaaata |
| | | taacacagtccttaatatctaagtatcataaaggtgaaataaaattagaagaacctacttatttt |
| | | cagtcattacttatgacatacaagagtatgacctcgtcagaacagattgctaccactaatttact |
| | | taaaaagataataagaagagctatagaaataagtgatgtcaaagtctatgctatattgaataaac |
| | | tagggcttaaagaaaaggacaagattaaatccaacaatggacaagatgaagacaactcagttatt |
| | | acgaccataatcaaagatgatatactttcagctgttaaagataatcaatctcatcttaaagcaga |
| | | caaaaatcactctacaaaacaaaaagacacaatcaaaacaacactcttgaagaaattgatgtgtt |
| | | caatgcaacatcctccatcatggttaatacattggtttaacttatacacaaaattaaacaacata |
| | | ttaacacagtatcgatcaaatgaggtaaaaaaccatgggtttacattgatagataatcaaactct |
| | | tagtggatttcaatttattttgaaccaatatggttgtatagtttatcataaggaactcaaaagaa |
| | | ttactgtgacaacctataatcaattcttgacatggaaagatattagccttagtagattaaatgtt |
| | | tgtttaattacatggattagtaactgcttgaacacattaaataaaagcttaggcttaagatgcgg |
| | | attcaataatgttatcttgacacaactattcctttatggagattgtatactaaagctatttcaca |
| | | atgaggggttctacataataaaagaggtagagggatttattatgtctctaattttaaatataaca |
| | | gaagaagatcaattcagaaaacgattttataatagtatgctcaacaacatcacagatgctgctaa |
| | | taaagctcagaaaaatctgctatcaagagtatgtcatacattattagataagacagtgtccgata |
| | | atataataaatggcagatggataattctattaagtaagttccttaaattaattaagcttgcaggt |
| | | gacaataaccttaacaatctgagtgaactatattttttgttcagaatatttggacacccaatggt |
| | | agatgaaagacaagccatggatgctgttaaaattaattgcaatgagaccaaattttacttgttaa |
| | | gcagtctgagtatgttaagaggtgcctttatatatagaattataaaagggtttgtaaataattac |
| | | aacagatggcctactttaagaaatgctattgttttacccttaagatggttaacttactataaact |
| | | aaacacttatccttctttgttggaacttacagaaagagatttgattgtgttatcaggactacgtt |
| | | tctatcgtgagtttcggttgcctaaaaaagtggatcttgaaatgattataaatgataaagctata |
| | | tcacctcctaaaaatttgatatggactagtttccctagaaattacatgccatcacacatacaaaa |
| | | ctatatagaacatgaaaaattaaaattttccgagagtgataaatcaagaagagtattagagtatt |
| | | atttaagagataacaaattcaatgaatgtgatttatacaactgtgtagttaatcaaagttatctc |
| | | aacaaccctaatcatgtggtatcattgacaggcaaagaaagagaactcagtgtaggtagaatgtt |
| | | tgcaatgcaaccgggaatgttcagacaggttcaaatattggcagagaaaatgatagctgaaaaca |
| | | ttttacaattctttcctgaaagtcttacaagatatggtgatctagaactacaaaaaatattagaa |
| | | ctgaaagcaggaataagtaacaaatcaaatcgctacaatgataattacaacaattacattagtaa |
| | | gtgctctatcatcacagatctcagcaaattcaatcaagcatttcgatatgaaacgtcatgtattt |
| | | gtagtgatgtgctggatgaactgcatggtgtacaatctctattttcctggttacatttaactatt |
| | | cctcatgtcacaataatatgcacatataggcatgcacccccctatataggagatcatattgtaga |
| | | tcttaacaatgtagatgaacaaagtggattatatagatatcacatgggtggcatcgaagggtggt |
| | | gtcaaaaactatggaccatagaagctatatcactattggatctaatatctctcaaagggaaattc |
| | | tcaattactgctttaattaatggtgacaatcaatcaatagatataagcaaaccaatcagactcat |
| | | ggaaggtcaaactcatgctcaagcagattatttgctagcattaaatagccttaaattactgtata |
| | | aagagtatgcaggcataggccacaaattaaaaggaactgagacttatatatcacgagatatgcaa |
| | | tttatgagtaaaacaattcaacataacggtgtatattacccagctagtataaagaaagtcctaag |
| | | agtgggaccgtggataaacactatacttgatgatttcaaagtgagtctagaatctataggtagtt |
| | | tgacacaagaattagaatatagaggtgaaagtctattatgcagtttaatatttagaaatgtatgg |
| | | ttatataatcagattgctctacaattaaaaaatcatgcattatgtaacaataaactatatttgga |
| | | catattaaaggttctgaaacacttaaaaaccttttttaatcttgataatattgatacagcattaa |
| | | cattgtatatgaatttacccatgttatttggtggtggtgatcccaacttgttatatcgaagtttc |
| | | tatagaagaactcctgacttcctcacagaggctatagttcactctgtgttcatacttagttatta |
| | | tacaaaccatgacttaaaagataaacttcaagatctgtcagatgatagattgaataagttcttaa |
| | | catgcataatcacgtttgacaaaaaccctaatgctgaattcgtaacattgatgagagatcctcaa |
| | | gctttagggtctgagagacaagctaaaattactagcgaaatcaatagactggcagttacagaggt |
| | | tttgagtacagctccaaacaaaatattctccaaaagtgcacaacattatactactacagagatag |
| | | atctaaatgatattatgcaaaatatagaacctacatatcctcatgggctaagagttgtttatgaa |
| | | agtttacccttttataaagcagagaaaatagtaaatcttatatcaggtacaaaatctataactaa |
| | | catactggaaaaaacttctgccatagacttaacagatattgatagagccactgagatgatgagga |
| | | aaaacataactttgcttataaggatacttccattggattgtaacagagataaaagagagatattg |
| | | agtatggaaaacctaagtattactgaattaagcaaatatgttagggaaagatcttggtctttatc |
| | | caatatagttggtgttacatcacccagtatcatgtatacaatggacatcaaatatactacaagca |
| | | ctatatctagtggcataattatagagaaatataatgttaacagtttaacacgtggtgagagagga |
| | | cccactaaaccatgggttggttcatctacacaagagaaaaaaacaatgccagtttataatagaca |
| | | agtcttaaccaaaaaacagagagatcaaatagatctattagcaaaattggattgggtgtatgcat |
| | | ctatagataacaaggatgaattcatggaagaactcctgggaacccttgggttaacatatgaaaag |
| | | gccaagaaattatttccacaatatttaagtgtcaattatttgcatcgccttacagtcagtagtag |
| | | accatgtgaattccctgcatcaataccagcttatagaacaacaaattatcactttgacactagcc |
| | | ctattaatcgcatattaacagaaaagtatggtgatgaagatattgacatagtattccaaaactgt |
| | | ataagctttggccttagtttaatgtcagtagtagaacaatttactaatgtatgtcctaacagaat |
| | | tattctcatacctaagcttaatgagatacatttgatgaaacctcccatattcacaggtgatgttg |
| | | atattcacaagttaaaacaagtgatacaaaaacagcatatgtttttaccagacaaaataagtttg |
| | | actcaatatgtggaattattcttaagtaataaaacactcaaatctggatctcatgttaattctaa |
| | | tttaatattggcacataaaatatctgactattttcataatacttacattttaagtactaatttag |
| | | ctggacattggattctgattatacaacttatgaaagattctaaaggtatttttgaaaaagattgg |
| | | ggagagggatatataactgatcatatgtttattaatttgaaagttttcttcaatgcttataagac |
| | | ctatctcttgtgttttcataaaggttatggcaaagcaaagctggagtgtgatatgaacacttcag |
| | | atcttctatgtgtattggaattaatagacagtagttattggaagtctatgtctaaggtattttta |
| | | gaacaaaaagttatcaaatacattcttagccaagatgcaagtttacatagagtaaaaggatgtca |
| | | tagcttcaaattatggtttcttaaacgtcttaatgtagcagaattcacagtttgcccttgggttg |
| | | ttaacatagattatcatccaacacatatgaaagcaatattaacttatatagatcttgttagaatg |
| | | ggattgataaatatagatagaatacacattaaaaataaacacaaattcaatgatgaattttatac |
| | | ttctaatctcttctacattaattataacttctcagataatactcatctattaactaaacatataa |
| | | ggattgctaattctgaattagaaaataattacaacaaattatatcatcctacaccagaaacccta |
| | | gagaatatactagccaatccgattaaaagtaatgacaaaaagacactgaatgactattgtatagg |
| | | taaaaatgttgactcaataatgttaccattgttatctaataagaagcttattaaatcgtctgcaa |
| | | tgattagaaccaattacagcaaacaagatttgtataatttattccctatggttgtgattgataga |
| | | attatagatcattcaggcaatacagccaaatccaaccaactttacactactacttcccaccaaat |
| | | atccttagtgcacaatagcacatcactttactgcatgcttccttggcatcatattaatagattca |
| | | attttgtatttagttctacaggttgtaaaattagtatagagtatattttaaaagatcttaaaatt |
| | | aaagatcccaattgtatagcattcataggtgaaggagcagggaatttattattgcgtacagtagt |
| | | ggaacttcatcctgacataagatatatttacagaagtctgaaagattgcaatgatcatagtttac |
| | | ctattgagtttttaaggctgtacaatggacatatcaacattgattatggtgaaaatttgaccatt |
| | | cctgctacagatgcaaccaacaacattcattggtcttatttacatataaagtttgctgaacctat |
| | | cagtctttttgtctgtgatgccgaattgtctgtaacagtcaactggagtaaaattataatagaat |
| | | ggagcaagcatgtaagaaagtgcaagtactgttcctcagttaataaatgtatgttaatagtaaaa |
| | | tatcatgctcaagatgatattgatttcaaattagacaatataactatattaaaaacttatgtatg |
| | | cttaggcagtaagttaaagggatcggaggtttacttagtccttacaataggtcctgcgaatatat |
| | | tcccagtatttaatgtagtacaaaatgctaaattgatactatcaagaaccaaaaatttcatcatg |
| | | cctaagaaagctgataaagagtctattgatgcaaatattaaaagtttgataccctttctttgtta |
| | | ccctataacaaaaaaaggaattaatactgcattgtcaaaactaaagagtgttgttagtggagata |
| | | tactatcatattctatagctggacgtaatgaagttttcagcaataaacttataaatcataagcat |
| | | atgaacatcttaaaatggttcaatcatgttttaaatttcagatcaacagaactaaactataacca |
| | | tttatatatggtagaatctacatatccttacctaagtgaattgttaaacagcttgacaaccaatg |
| | | aacttaaaaaactgattaaaatcacaggtagtctgttatacaactttcataatgaataatgaata |
| | | aagatcttataataaaaattcccatagctatacactaacactgtattcaattatagttattaaaa |
| | | attaaaaatcatataattttttaaaAaacttttagtgaactaatcctaaagttatcattttaatc |
| | | ttggaggaataaatttaaaccctaatctaattggtttatatgtgtattaactaaattacgagata |
| | | ttagtttttgacactttttttctcgttgagttacagagatgtaactctgtaactcctgatgagtc |
| | | cgtgaggacgaaacgagaaaaaaagtgtcaaaagtcgaccgtagcataaccccttggggcctcta |
| | | aacgggtcttgaggggttttttgctgaaaggaggaactatataagctttgcagtagcataacccc |
| | | ttggggcctctaaacgggtcttgaggggttttttgctgaaaggaggaactatataacgcgtctgc |
| | | agcaatggcaacaacgttgcgcaaactattaactggcgaactacttactctagcttcccggcaac |
| | | aattaatagactggatggaggcggataaagttgcaggaccacttctgcgctcggcccttccggct |
| | | ggctggtttattgctgataaatctggagccggtgagcgtgggtctcgcggtatcattgcagcact |
| | | ggggccagatggtaagccctcccgtatcgtagttatctacacgacggggagtcaggcaactatgg |
| | | atgaacgaaatagacagatcgctgagataggtgcctcactgattaagcattggtaactgtcagac |
| | | caagtttactcatatatactttagattgatttaaaacttcatttttaatttaaaaggatctaggt |
| | | gaagatcctttttgataatctcatgaccaaaatcccttaacgtgagttttcgttccactgagcgt |
| | | cagaccccgtagaaaagatcaaaggatcttcttgagatcctttttttctgcgcgtaatctgctgc |
| | | ttgcaaacaaaaaaaccaccgctaccagcggtggtttgtttgccggatcaagagctaccaactct |
| | | ttttccgaaggtaactggcttcagcagagcgcagataccaaatactgtccttctagtgtagccgt |
| | | agttaggccaccacttcaagaactctgtagcaccgcctacatacctcgctctgctaatcctgtta |
| | | ccagtggctgctgccagtggcgataagtcgtgtcttaccgggttggactcaagacgatagttacc |
| | | ggataaggcgcagcggtcgggctgaacggggggttcgtgcacacagcccagcttggagcgaacga |
| | | cctacaccgaactgagatacctacagcgtgagctatgagaaagcgccacgcttcccgaagggaga |
| | | aaggcggacaggtatccggtaagcggcagggtcggaacaggagagcgcacgagggagcttccagg |
| | | gggaaacgcctggtatctttatagtcctgtcgggtttcgccacctctgacttgagcgtcgatttt |
| | | tgtgatgctcgtcaggggggcggagcctatggaaaaacgccagcaacgcggcctttttacggttc |
| | | ctggccttttgctggccttttgctcacatgttctttcctgcgttatcccctgattctgtggataa |
| | | ccgtattaccgcctttgagtgagctgataccgctcgccgcagccgaacgaccgagcgcagcgagt |
| | | cagtgagcgaggaagcggaagagcgcctgatgcggtattttctccttacgcatctgtgcggtatt |
| | | tcacaccgcatatggtgcactctcagtacaatctgctctgatgccgcatagttaagccagtatac |
| | | actccgctatcgctacgtgactgggtcatggctgcgccccgacacccgccaacacccgctgacgc |
| | | gccctgacgggcttgtctgctcccggcatccgcttacagacaagctgtgaccgtctccgggagct |
| | | gcatgtgtcagaggttttcaccgtcatcaccgaaacgcgcgaggcagctgcggtaaagctcatca |
| | | gcgtggtcgtgaagcgattcacagatgtctgcctgttcatccgcgtccagctcgttgagtttctc |
| | | cagaagcgttaatgtctggcttctgataaagcgggccatgttaagggcggttttttcctgtttgg |
| | | tcacttgatgcctccgtgtaagggggaatttctgttcatgggggtaatgataccgatgaaacgag |
| | | agaggatgctcacgatacgggttactgatgatgaacatgcccggttactggaacgttgtgagggt |
| | | aaacaactggcggtatggatgcggcgggaccagagaaaaatcactcagggtcaatgccagcgctt |
| | | cgttaatacagatgtaggtgttccacagggtagccagcagcatcctgcgatgcagatccggaaca |
| | | taatggtgcagggcgctgacttccgcgtttccagactttacgaaacacggaaaccgaagaccatt |
| | | catgttgttgctcaggtcgcagacgttttgcagcagcagtcgcttcacgttcgctcgcgtatcgg |
| | | tgattcattctgctaaccagtaaggcaaccccgccagcctagccgggtcctcaacgacaggagca |
| | | cgatcatgcgcacccgtggccaggacccaacgctgcccgagatgcgccgcgtgcggctgctggag |
| | | atggcggacgcgatggatatgttctgccaagggttggtttgcgcattcacagttctccgcaagaa |
| | | ttgattggctccaattcttggagtggtgaatccgttagcgaggtgccgccggcttccattcaggt |
| | | cgaggtggcccggctccatgcaccgcgacgcaacgcggggaggcagacaaggtatagggcggcgc |
| | | ctacaatccatgccaacccgttccatgtgctcgccgaggcggcataaatcgccgtgacgatcagc |
| | | ggtccagtgatcgaagttaggctggtaagagccgcgagcgatccttgaagctgtccctgatggtc |
| | | gtcatctacctgcctggacagcatggcctgcaacgcgggcatcccgatgccgccggaagcgagaa |
| | | gaatcataatggggaaggccatccagcctcgcgtcgcgaacgccagcaagacgtagcccagcgcg |
| | | tcggccgccatgccggcgataatggcctgcttctcgccgaaacgtttggtggcgggaccagtgac |
| | | gaaggcttgagcgagggcgtgcaagattccgaataccgcaagcgacaggccgatcatcgtcgcgc |
| | | tccagcgaaagcggtcctcgccgaaaatgacccagagcgctgccggcacctgtcctacgagttgc |
| | | atgataaagaagacagtcataagtgcggcgacgatagtcatgccccgcgcccaccggaaggagct |
| | | gactgggttgaaggctctcaagggcatcggtcgacgctctcccttatgcgactcctgcattagga |
| | | agcagcccagtagtaggttgaggccgttgagcaccgccgccgcaaggaatggtgcatgcaaggag |
| | | atggcgcccaacagtcccccggccacggggcctgccaccatacccacgccgaaacaagcgctcat |
| | | gagcccgaagtggcgagcccgatcttccccatcggtgatgtcggcgatataggcgccagcaaccg |
| | | cacctgtggcgccggtgatgccggccacgatgcgtccggcgtagaagatccacaggacgggtgtg |
| | | gtcgccatgatcgcgtagtcgatagtggctccaagtagcgaagcgagcaggactgggcggcggcc |
| | | aaagcggtcggacagtgctccgagaacgggtgcgcatagaaattgcatcaacgcatatagcgcta |
| | | gcagcacgccatagtgactggcgatgctgtcggaatggacgatatcccgcaagaggcccggcagt |
| | | accggcataaccaagcctatgcctacagcatccagggtgacggtgccgaggatgacgatgagcgc |
| | | attgttagatttcatacacggtgcctgactgcgttagcaatttaactgtgataaactaccgcatt |
| | | aaagcttatcgatgataagctgtcaaacatgagaattgacgtcatgaccggtagattcgaaattg |
| | | aaaccattattcctataaaaataggcgcatcacgaggcccctttcgtcttgtaatacgactcact |
| | | ataggg |
| 2 | RSV | acgggaaaaaatgcgtacaacaaacttgcataaaccaaaaaaatggggcaaataagaatttgata |
| | ΔNS2/ | agtaccacttaaatttaactcccttggttagagatgggcagcaattcattgagtatgataaaagt |
| | Δ1313/ | tagattacaaaatttgtttgacaatgatgaagtagcattgttaaaaataacatgctatactgata |
| | I1314L | aattaatacatttaactaatgctttggctaaggcagtgatacatacaatcaaattgaatggcatt |
| | (NIH) | gtgtttgtgcatgttattacaagtagtgatatttgccctaataataatattgtagtaaaatccaa |
| | | tttcacaacaatgccagtactacaaaatggaggttatatatgggaaatgatggaattaacacatt |
| | | gctctcaacctaatggtctactagatgacaattgtgaaattaaattctccaaaaaactaagtgat |
| | | tcaacaatgaccaattatatgaatcaattatctgaattacttggatttgatcttaatccataaat |
| | | tataattaatatcaactagcaaatcaatgtcactaacaccattagttaatataaaacttaaggag |
| | | agatataagatagaagatggggcaaatacaaccatggctcttagcaaagtcaagttgaatgatac |
| | | actcaacaaagatcaacttctgtcatccagcaaatacaccatccaacggagcacaggagatagta |
| | | ttgatactcctaattatgatgtgcagaaacacatcaataagttatgtggcatgttattaatcaca |
| | | gaagatgctaatcataaattcactgggttaataggtatgttatatgcgatgtctaggttaggaag |
| | | agaagacaccataaaaatactcagagatgcgggatatcatgtaaaagcaaatggagtagatgtaa |
| | | caacacatcgtcaagacattaatggaaaagaaatgaaatttgaagtgttaacattggcaagctta |
| | | acaactgaaattcaaatcaacattgagatagaatctagaaaatcctacaaaaaaatgctaaaaga |
| | | aatgggagaggtagctccagaatacaggcatgactctcctgattgtgggatgataatattatgta |
| | | tagcagcattagtaataactaaattagcagcaggggacagatctggtcttacagccgtgattagg |
| | | agagctaataatgtcctaaaaaatgaaatgaaacgttacaaaggcttactacccaaggacatagc |
| | | caacagcttctatgaagtgtttgaaaaacatccccactttatagatgtttttgttcattttggta |
| | | tagcacaatcttctaccagaggtggcagtagagttgaagggatttttgcaggattgtttatgaat |
| | | gcctatggtgcagggcaagtgatgttacggtggggagtcttagcaaaatcagttaaaaatattat |
| | | gttaggacatgctagtgtgcaagcagaaatggaacaagttgttgaggtttatgaatatgcccaaa |
| | | aattgggtggtgaagcaggattctaccatatattgaacaacccaaaagcatcattattatctttg |
| | | actcaatttcctcacttctccagtgtagtattaggcaatgctgctggcctaggcataatgggaga |
| | | gtacagaggtacaccgaggaatcaagatctatatgatgcagcaaaggcatatgctgaacaactca |
| | | aagaaaatggtgtgattaactacagtgtactagacttgacagcagaagaactagaggctatcaaa |
| | | catcagcttaatccaaaagataatgatgtagagctttgagttaataaaaaatggggcaaataaat |
| | | catcatggaaaagtttgctcctgaattccatggagaagatgcaaacaacagggctactaaattcc |
| | | tagaatcaataaagggcaaattcacatcacccaaagatcccaagaaaaaagatagtatcatatct |
| | | gtcaactcaatagatatagaagtaaccaaagaaagccctataacatcaaattcaactattatcaa |
| | | cccaacaaatgagacagatgatactgcagggaacaagcccaattatcaaagaaaacctctagtaa |
| | | gtttcaaagaagaccctacaccaagtgataatcccttttctaaactatacaaagaaaccatagaa |
| | | acatttgataacaatgaagaagaatccagctattcatacgaagaaataaatgatcagacaaacga |
| | | taatataacagcaagattagataggattgatgaaaaattaagtgaaatactaggaatgcttcaca |
| | | cattagtagtggcaagtgcaggacctacatctgctcgggatggtataagagatgccatggttggt |
| | | ttaagagaagaaatgatagaaaaaatcagaactgaagcattaatgaccaatgacagattagaagc |
| | | tatggcaagactcaggaatgaggaaagtgaaaagatggcaaaagacacatcagatgaagtgtctc |
| | | tcaatccaacatcagagaaattgaacaacctattggaagggaatgatagtgacaatgatctatca |
| | | cttgaagatttctgattagttaccaatcttcacatcaacacacaataccaacagaagaccaacaa |
| | | actaaccaacccaatcatccaaccaaacatccatccgccaatcagccaaacagccaacaaaacaa |
| | | ccagccaatccaaaactaaccacccggaaaaaatctataatatagttacaaaaaaaggaaagggt |
| | | ggggcaaatatggaaacatacgtgaacaagcttcacgaaggctccacatacacagctgctgttca |
| | | atacaatgtcttagaaaaagacgatgaccctgcatcacttacaatatgggtgcccatgttccaat |
| | | catctatgccagcagatttacttataaaagaactagctaatgtcaacatactagtgaaacaaata |
| | | tccacacccaagggaccttcactaagagtcatgataaactcaagaagtgcagtgctagcacaaat |
| | | gcccagcaaatttaccatatgcgctaatgtgtccttggatgaaagaagcaaactagcatatgatg |
| | | taaccacaccctgtgaaatcaaggcatgtagtctaacatgcctaaaatcaaaaaatatgttgact |
| | | acagttaaagatctcactatgaagacactcaaccctacacatgatattattgctttatgtgaatt |
| | | tgaaaacatagtaacatcaaaaaaagtcataataccaacatacctaagatccatcagtgtcagaa |
| | | ataaagatctgaacacacttgaaaatataacaaccactgaattcaaaaatgctatcacaaatgca |
| | | aaaatcatcccttactcaggattactattagtcatcacagtgactgacaacaaaggagcattcaa |
| | | atacataaagccacaaagtcaattcatagtagatcttggagcttacctagaaaaagaaagtatat |
| | | attatgttaccacaaattggaagcacacagctacacgatttgcaatcaaacccatggaagattaa |
| | | cctttttcctctacatcagtgtgttaattcatacaaactttctacctacattcttcacttcacca |
| | | tcacaatcacaaacactctgtggttcaaccaatcaaacaaaacttatctgaagtcccagatcatc |
| | | ccaagtcattgtttatcagatctagtactcaaataagttaataaaaaatatacacatggggcaaa |
| | | taatcattggaggaaatccaactaatcacaatatctgttaacatagacaagtccacacaccatac |
| | | agaatcaaccaatggaaaatacatccataacaatagaattctcaagcaaattctggccttacttt |
| | | acactaatacacatgatcacaacaataatctctttgctaatcataatctccatcatgattgcaat |
| | | actaaacaaactttgtgaatataacgtattccataacaaaacctttgagttaccaagagctcgag |
| | | ttaatacttgataaagtagttaattaaaaatagtcataacaatgaactaggatatcaagactaac |
| | | aataacattggggcaaatgcaaacatgtccaaaaacaaggaccaacgcaccgctaagacattaga |
| | | aaggacctgggacactctcaatcatttattattcatatcatcgtgcttatataagttaaatctta |
| | | aatctgtagcacaaatcacattatccattctggcaatgataatctcaacttcacttataattgca |
| | | gccatcatattcatagccteggcaaaccacaaagtcacaccaacaactgcaatcatacaagatgc |
| | | aacaagccagatcaagaacacaaccccaacatacctcacccagaatcctcagcttggaatcagtc |
| | | cctctaatccgtctgaaattacatcacaaatcaccaccatactagcttcaacaacaccaggagtc |
| | | aagtcaaccctgcaatccacaacagtcaagaccaaaaacacaacaacaactcaaacacaacccag |
| | | caagcccaccacaaaacaacgccaaaacaaaccaccaagcaaacccaataatgattttcactttg |
| | | aagtgttcaactttgtaccctgcagcatatgcagcaacaatccaacctgctgggctatctgcaaa |
| | | agaataccaaacaaaaaaccaggaaagaaaaccactaccaagcccacaaaaaaaccaaccctcaa |
| | | gacaaccaaaaaagatcccaaacctcaaaccactaaatcaaaggaagtacccaccaccaagccca |
| | | cagaagagccaaccatcaacaccaccaaaacaaacatcataactacactactcacctccaacacc |
| | | acaggaaatccagaactcacaagtcaaatggaaaccttccactcaacttcctccgaaggcaatcc |
| | | aagcccttctcaagtctctacaacatccgagtacccatcacaaccttcatctccacccaacacac |
| | | cacgccagtagttacttaaaaacatattatcacaaaaggccttgaccaacttaaacagaatcaaa |
| | | ataaactctggggcaaataacaatggagttgctaatcctcaaagcaaatgcaattaccacaatcc |
| | | tcactgcagtcacattttgttttgcttctggtcaaaacatcactgaagaattttatcaatcaaca |
| | | tgcagtgcagttagcaaaggctatcttagtgctctgagaactggttggtataccagtgttataac |
| | | tatagaattaagtaatatcaagaaaaataagtgtaatggaacagatgctaaggtaaaattgataa |
| | | aacaagaattagataaatataaaaatgctgtaacagaattgcagttgctcatgcaaagcacacaa |
| | | gcaacaaacaatcgagccagaagagaactaccaaggtttatgaattatacactcaacaatgccaa |
| | | aaaaaccaatgtaacattaagcaagaaaaggaaaagaagatttcttggttttttgttaggtgttg |
| | | gatctgcaatcgccagtggcgttgctgtatctaaggtcctgcacctagaaggggaagtgaacaag |
| | | atcaaaagtgctctactatccacaaacaaggctgtagtcagcttatcaaatggagttagtgtttt |
| | | aaccagcaaagtgttagacctcaaaaactatatagataaacaattgttacctattgtgaacaagc |
| | | aaagctgcagcatatcaaatatagaaactgtgatagagttccaacaaaagaacaacagactacta |
| | | gagattaccagggaatttagtgttaatgcaggcgtaactacacctgtaagcacttacatgttaac |
| | | taatagtgaattattgtcattaatcaatgatatgcctataacaaatgatcagaaaaagttaatgt |
| | | ccaacaatgttcaaatagttagacagcaaagttactctatcatgtccataataaaagaggaagtc |
| | | ttagcatatgtagtacaattaccactatatggtgttatagatacaccctgttggaaactacacac |
| | | atcccctctatgtacaaccaacacaaaagaagggtccaacatctgtttaacaagaactgacagag |
| | | gatggtactgtgacaatgcaggatcagtatctttettcccacaagctgaaacatgtaaagttcaa |
| | | tcaaatcgagtattttgtgacacaatgaacagtttaacattaccaagtgaagtaaatctctgcaa |
| | | tgttgacatattcaaccccaaatatgattgtaaaattatgacttcaaaaacagatgtaagcagct |
| | | ccgttatcacatctctaggagccattgtgtcatgctatggcaaaactaaatgtacagcatccaat |
| | | aaaaatcgtggaatcataaagacattttctaacgggtgcgattatgtatcaaataaaggggtgga |
| | | cactgtgtctgtaggtaacacattatattatgtaaataagcaagaaggtaaaagtctctatgtaa |
| | | aaggtgaaccaataataaatttctatgacccattagtattcccctctgatgaatttgatgcatca |
| | | atatctcaagtcaacgagaagattaaccagagcctagcatttattcgtaaatccgatgaattatt |
| | | acataatgtaaatgctggtaaatccaccacaaatatcatgataactactataattatagtgatta |
| | | tagtaatattgttatcattaattgctgttggactgctcttatactgtaaggccagaagcacacca |
| | | gtcacactaagcaaagatcaactgagtggtataaataatattgcatttagtaactaaataaaaat |
| | | agcacctaatcatgttcttacaatggtttactatctgctcatagacaacccatctgtcattggat |
| | | tttcttaaaatctgaacttcatcgaaactctcatctataaaccatctcacttacactatttaagt |
| | | agattcctagtttatagttatataaaacacaattgcatgccagattaacttaccatctgtaaaaa |
| | | tgaaaactggggcaaatatgtcacgaaggaatccttgcaaatttgaaattcgaggtcattgctta |
| | | aatggtaagaggtgtcattttagtcataattattttgaatggccaccccatgcactgcttgtaag |
| | | acaaaactttatgttaaacagaatacttaagtctatggataaaagtatagataccttatcagaaa |
| | | taagtggagctgcagagttggacagaacagaagagtatgctcttggtgtagttggagtgctagag |
| | | agttatataggatcaataaacaatataactaaacaatcagcatgtgttgccatgagcaaactcct |
| | | cactgaactcaatagtgatgatatcaaaaagctgagggacaatgaagagctaaattcacccaaga |
| | | taagagtgtacaatactgtcatatcatatattgaaagcaacaggaaaaacaataaacaaactatc |
| | | catctgttaaaaagattgccagcagacgtattgaagaaaaccatcaaaaacacattggatatcca |
| | | taagagcataaccatcaacaacccaaaagaatcaactgttagtgatacaaatgaccatgccaaaa |
| | | ataatgatactacctgacaaatatccttgtagtataacttccatactaataacaagtagatgtag |
| | | agttactatgtataatcaaaagaacacactatatttcaatcaaaacaacccaaataaccatatgt |
| | | actcaccgaatcaaacattcaatgaaatccattggacctctcaagaattgattgacacaattcaa |
| | | aattttctacaacatctaggtattattgaggatatatatacaatatatatattagtgtcataaca |
| | | ctcaattctaacactcaccacatcgttacattattaattcaaacaattcaagttgtgggacaaaa |
| | | tggatcccattattaatggaaattctgctaatgtttatctaaccgatagttatttaaaaggtgtt |
| | | atctctttctcagagtgtaatgctttaggaagttacatattcaatggtccttatctcaaaaatga |
| | | ttataccaacttaattagtagacaaaatccattaatagaacacatgaatctaaagaaactaaata |
| | | taacacagtccttaatatctaagtatcataaaggtgaaataaaattagaagaacctacttatttt |
| | | cagtcattacttatgacatacaagagtatgacctcgtcagaacagattgctaccactaatttact |
| | | taaaaagataataagaagagctatagaaataagtgatgtcaaagtctatgctatattgaataaac |
| | | tagggcttaaagaaaaggacaagattaaatccaacaatggacaagatgaagacaactcagttatt |
| | | acgaccataatcaaagatgatatactttcagctgttaaagataatcaatctcatcttaaagcaga |
| | | caaaaatcactctacaaaacaaaaagacacaatcaaaacaacactcttgaagaaattgatgtgtt |
| | | caatgcaacatcctccatcatggttaatacattggtttaacttatacacaaaattaaacaacata |
| | | ttaacacagtatcgatcaaatgaggtaaaaaaccatgggtttacattgatagataatcaaactct |
| | | tagtggatttcaatttattttgaaccaatatggttgtatagtttatcataaggaactcaaaagaa |
| | | ttactgtgacaacctataatcaattcttgacatggaaagatattagccttagtagattaaatgtt |
| | | tgtttaattacatggattagtaactgcttgaacacattaaataaaagcttaggcttaagatgcgg |
| | | attcaataatgttatcttgacacaactattcctttatggagattgtatactaaagctatttcaca |
| | | atgaggggttctacataataaaagaggtagagggatttattatgtctctaattttaaatataaca |
| | | gaagaagatcaattcagaaaacgattttataatagtatgctcaacaacatcacagatgctgctaa |
| | | taaagctcagaaaaatctgctatcaagagtatgtcatacattattagataagacagtgtccgata |
| | | atataataaatggcagatggataattctattaagtaagttccttaaattaattaagcttgcaggt |
| | | gacaataaccttaacaatctgagtgaactatattttttgttcagaatatttggacacccaatggt |
| | | agatgaaagacaagccatggatgctgttaaaattaattgcaatgagaccaaattttacttgttaa |
| | | gcagtctgagtatgttaagaggtgcctttatatatagaattataaaagggtttgtaaataattac |
| | | aacagatggcctactttaagaaatgctattgttttaccettaagatggttaacttactataaact |
| | | aaacacttatccttctttgttggaacttacagaaagagatttgattgtgttatcaggactacgtt |
| | | tctatcgtgagtttcggttgcctaaaaaagtggatcttgaaatgattataaatgataaagctata |
| | | tcacctcctaaaaatttgatatggactagtttccctagaaattacatgccatcacacatacaaaa |
| | | ctatatagaacatgaaaaattaaaattttccgagagtgataaatcaagaagagtattagagtatt |
| | | atttaagagataacaaattcaatgaatgtgatttatacaactgtgtagttaatcaaagttatctc |
| | | aacaaccctaatcatgtggtatcattgacaggcaaagaaagagaactcagtgtaggtagaatgtt |
| | | tgcaatgcaaccgggaatgttcagacaggttcaaatattggcagagaaaatgatagctgaaaaca |
| | | ttttacaattctttcctgaaagtcttacaagatatggtgatctagaactacaaaaaatattagaa |
| | | ctgaaagcaggaataagtaacaaatcaaatcgctacaatgataattacaacaattacattagtaa |
| | | gtgctctatcatcacagatctcagcaaattcaatcaagcatttcgatatgaaacgtcatgtattt |
| | | gtagtgatgtgctggatgaactgcatggtgtacaatctctattttcctggttacatttaactatt |
| | | cctcatgtcacaataatatgcacatataggcatgcacccccctatataggagatcatattgtaga |
| | | tcttaacaatgtagatgaacaaagtggattatatagatatcacatgggtggcatcgaagggtggt |
| | | gtcaaaaactatggaccatagaagctatatcactattggatctaatatctctcaaagggaaattc |
| | | tcaattactgctttaattaatggtgacaatcaatcaatagatataagcaaaccaatcagactcat |
| | | ggaaggtcaaactcatgctcaagcagattatttgctagcattaaatagccttaaattactgtata |
| | | aagagtatgcaggcataggccacaaattaaaaggaactgagacttatatatcacgagatatgcaa |
| | | tttatgagtaaaacaattcaacataacggtgtatattacccagctagtataaagaaagtcctaag |
| | | agtgggaccgtggataaacactatacttgatgatttcaaagtgagtctagaatctataggtagtt |
| | | tgacacaagaattagaatatagaggtgaaagtctattatgcagtttaatatttagaaatgtatgg |
| | | ttatataatcagattgctctacaattaaaaaatcatgcattatgtaacaataaactatatttgga |
| | | catattaaaggttctgaaacacttaaaaaccttttttaatcttgataatattgatacagcattaa |
| | | cattgtatatgaatttacccatgttatttggtggtggtgatcccaacttgttatatcgaagtttc |
| | | tatagaagaactcctgacttcctcacagaggctatagttcactctgtgttcatacttagttatta |
| | | tacaaaccatgacttaaaagataaacttcaagatctgtcagatgatagattgaataagttcttaa |
| | | catgcataatcacgtttgacaaaaaccctaatgctgaattcgtaacattgatgagagatcctcaa |
| | | gctttagggtctgagagacaagctaaaattactagcgaaatcaatagactggcagttacagaggt |
| | | tttgagtacagctccaaacaaaatattctccaaaagtgcacaacattatactactacagagatag |
| | | atctaaatgatattatgcaaaatatagaacctacatatcctcatgggctaagagttgtttatgaa |
| | | agtttacccttttataaagcagagaaaatagtaaatcttatatcaggtacaaaatctataactaa |
| | | catactggaaaaaacttctgccatagacttaacagatattgatagagccactgagatgatgagga |
| | | aaaacataactttgcttataaggatacttccattggattgtaacagagataaaagagagatattg |
| | | agtatggaaaacctaagtattactgaattaagcaaatatgttagggaaagatcttggtctttatc |
| | | caatatagttggtgttacatcacccagtatcatgtatacaatggacatcaaatatactacaagca |
| | | ctatatctagtggcataattatagagaaatataatgttaacagtttaacacgtggtgagagagga |
| | | cccactaaaccatgggttggttcatctacacaagagaaaaaaacaatgccagtttataatagaca |
| | | agtcttaaccaaaaaacagagagatcaaatagatctattagcaaaattggattgggtgtatgcat |
| | | ctatagataacaaggatgaattcatggaagaactcctgggaacccttgggttaacatatgaaaag |
| | | gccaagaaattatttccacaatatttaagtgtcaattatttgcatcgccttacagtcagtagtag |
| | | accatgtgaattccctgcatcaataccagcttatagaacaacaaattatcactttgacactagcc |
| | | ctattaatcgcatattaacagaaaagtatggtgatgaagatattgacatagtattccaaaactgt |
| | | ataagctttggccttagtttaatgtcagtagtagaacaatttactaatgtatgtcctaacagaat |
| | | tattctcatacctaagcttaatgagatacatttgatgaaacctcccatattcacaggtgatgttg |
| | | atattcacaagttaaaacaagtgatacaaaaacagcatatgtttttaccagacaaaataagtttg |
| | | actcaatatgtggaattattcttaagtaataaaacactcaaatctggatctcatgttaattctaa |
| | | tttaatattggcacataaaatatctgactattttcataatacttacattttaagtactaatttag |
| | | ctggacattggattctgattatacaacttatgaaagattctaaaggtatttttgaaaaagattgg |
| | | ggagagggatatataactgatcatatgtttattaatttgaaagttttcttcaatgcttataagac |
| | | ctatctcttgtgttttcataaaggttatggcaaagcaaagctggagtgtgatatgaacacttcag |
| | | atcttctatgtgtattggaattaatagacagtagttattggaagtctatgtctaaggtattttta |
| | | gaacaaaaagttatcaaatacattcttagccaagatgcaagtttacatagagtaaaaggatgtca |
| | | tagcttcaaattatggtttcttaaacgtcttaatgtagcagaattcacagtttgcccttgggttg |
| | | ttaacatagattatcatccaacacatatgaaagcaatattaacttatatagatcttgttagaatg |
| | | ggattgataaatatagatagaatacacattaaaaataaacacaaattcaatgatgaattttatac |
| | | ttctaatctcttctacattaattataacttctcagataatactcatctattaactaaacatataa |
| | | ggattgctaattctgaattagaaaataattacaacaaattatatcatcctacaccagaaacccta |
| | | gagaatatactagccaatccgattaaaagtaatgacaaaaagacactgaatgactattgtatagg |
| | | taaaaatgttgactcaataatgttaccattgttatctaataagaagcttattaaatcgtctgcaa |
| | | tgattagaaccaattacagcaaacaagatttgtataatttattccctatggttgtgattgataga |
| | | attatagatcattcaggcaatacagccaaatccaaccaactttacactactacttcccaccaaat |
| | | atccttagtgcacaatagcacatcactttactgcatgcttccttggcatcatattaatagattca |
| | | attttgtatttagttctacaggttgtaaaattagtatagagtatattttaaaagatcttaaaatt |
| | | aaagatcccaattgtatagcattcataggtgaaggagcagggaatttattattgcgtacagtagt |
| | | ggaacttcatcctgacataagatatatttacagaagtctgaaagattgcaatgatcatagtttac |
| | | ctattgagtttttaaggctgtacaatggacatatcaacattgattatggtgaaaatttgaccatt |
| | | cctgctacagatgcaaccaacaacattcattggtcttatttacatataaagtttgctgaacctat |
| | | cagtctttttgtctgtgatgccgaattgtctgtaacagtcaactggagtaaaattataatagaat |
| | | ggagcaagcatgtaagaaagtgcaagtactgttcctcagttaataaatgtatgttaatagtaaaa |
| | | tatcatgctcaagatgatattgatttcaaattagacaatataactatattaaaaacttatgtatg |
| | | cttaggcagtaagttaaagggatcggaggtttacttagtccttacaataggtcctgcgaatatat |
| | | tcccagtatttaatgtagtacaaaatgctaaattgatactatcaagaaccaaaaatttcatcatg |
| | | cctaagaaagctgataaagagtctattgatgcaaatattaaaagtttgataccctttctttgtta |
| | | ccctataacaaaaaaaggaattaatactgcattgtcaaaactaaagagtgttgttagtggagata |
| | | tactatcatattctatagctggacgtaatgaagttttcagcaataaacttataaatcataagcat |
| | | atgaacatcttaaaatggttcaatcatgttttaaatttcagatcaacagaactaaactataacca |
| | | tttatatatggtagaatctacatatccttacctaagtgaattgttaaacagcttgacaaccaatg |
| | | aacttaaaaaactgattaaaatcacaggtagtctgttatacaactttcataatgaataatgaata |
| | | aagatcttataataaaaattcccatagctatacactaacactgtattcaattatagttattaaaa |
| | | attaaaaatcatataattttttaaataacttttagtgaactaatcctaaagttatcattttaatc |
| | | ttggaggaataaatttaaaccctaatctaattggtttatatgtgtattaactaaattacgagata |
| | | ttagtttttgacactttttttctcgttgagttacagagatgtaactctgtaactcctgatgagtc |
| | | cgtgaggacgaaacgagaaaaaaagtgtcaaaagtcgaccgtagcataaccccttggggcctcta |
| | | aacgggtcttgaggggttttttgctgaaaggaggaactatataagctttgcagtagcataacccc |
| | | ttggggcctctaaacgggtcttgaggggttttttgctgaaaggaggaactatataacgcgtctgc |
| | | agcaatggcaacaacgttgcgcaaactattaactggcgaactacttactctagcttcccggcaac |
| | | aattaatagactggatggaggcggataaagttgcaggaccacttctgcgctcggcccttccggct |
| | | ggctggtttattgctgataaatctggagccggtgagcgtgggtctcgcggtatcattgcagcact |
| | | ggggccagatggtaagccctcccgtatcgtagttatctacacgacggggagtcaggcaactatgg |
| | | atgaacgaaatagacagatcgctgagataggtgcctcactgattaagcattggtaactgtcagac |
| | | caagtttactcatatatactttagattgatttaaaacttcatttttaatttaaaaggatctaggt |
| | | gaagatcctttttgataatctcatgaccaaaatcccttaacgtgagttttogttccactgagcgt |
| | | cagaccccgtagaaaagatcaaaggatcttcttgagatcctttttttctgcgcgtaatctgctgc |
| | | ttgcaaacaaaaaaaccaccgctaccagcggtggtttgtttgccggatcaagagctaccaactct |
| | | ttttccgaaggtaactggcttcagcagagcgcagataccaaatactgtccttctagtgtagccgt |
| | | agttaggccaccacttcaagaactctgtagcaccgcctacatacctcgctctgctaatcctgtta |
| | | ccagtggctgctgccagtggcgataagtcgtgtcttaccgggttggactcaagacgatagttacc |
| | | ggataaggcgcagcggtcgggctgaacggggggttcgtgcacacagcccagcttggagegaacga |
| | | cctacaccgaactgagatacctacagcgtgagctatgagaaagcgccacgcttcccgaagggaga |
| | | aaggcggacaggtatccggtaagcggcagggtcggaacaggagagcgcacgagggagcttccagg |
| | | gggaaacgcctggtatctttatagtcctgtcgggtttcgccacctctgacttgagcgtcgatttt |
| | | tgtgatgctcgtcaggggggcggagcctatggaaaaacgccagcaacgcggcctttttacggttc |
| | | ctggccttttgctggccttttgctcacatgttctttcctgcgttatcccctgattctgtggataa |
| | | ccgtattaccgcctttgagtgagctgataccgctcgccgcagccgaacgaccgagcgcagcgagt |
| | | cagtgagcgaggaagcggaagagcgcctgatgcggtattttctccttacgcatctgtgcggtatt |
| | | tcacaccgcatatggtgcactctcagtacaatctgctctgatgccgcatagttaagccagtatac |
| | | actccgctatcgctacgtgactgggtcatggctgcgccccgacacccgccaacacccgctgacgc |
| | | gccctgacgggcttgtctgctcccggcatccgcttacagacaagctgtgaccgtctccgggagct |
| | | gcatgtgtcagaggttttcaccgtcatcaccgaaacgcgcgaggcagctgcggtaaagctcatca |
| | | gcgtggtcgtgaagcgattcacagatgtctgcctgttcatccgcgtccagctcgttgagtttctc |
| | | cagaagcgttaatgtctggcttctgataaagcgggccatgttaagggcggttttttcctgtttgg |
| | | tcacttgatgcctccgtgtaagggggaatttctgttcatgggggtaatgataccgatgaaacgag |
| | | agaggatgctcacgatacgggttactgatgatgaacatgcccggttactggaacgttgtgagggt |
| | | aaacaactggcggtatggatgcggcgggaccagagaaaaatcactcagggtcaatgccagcgctt |
| | | cgttaatacagatgtaggtgttccacagggtagccagcagcatcctgcgatgcagatccggaaca |
| | | taatggtgcagggcgctgacttccgcgtttccagactttacgaaacacggaaaccgaagaccatt |
| | | catgttgttgctcaggtcgcagacgttttgcagcagcagtcgcttcacgttcgctcgcgtatcgg |
| | | tgattcattctgctaaccagtaaggcaaccccgccagcctagccgggtcctcaacgacaggagca |
| | | cgatcatgegcacccgtggccaggacccaacgctgcccgagatgcgccgcgtgcggctgctggag |
| | | atggcggacgcgatggatatgttctgccaagggttggtttgcgcattcacagttctccgcaagaa |
| | | ttgattggctccaattcttggagtggtgaatccgttagcgaggtgccgccggcttccattcaggt |
| | | cgaggtggcccggctccatgcaccgcgacgcaacgcggggaggcagacaaggtatagggcggcgc |
| | | ctacaatccatgccaacccgttccatgtgctcgccgaggggcataaatcgccgtgacgatcagcg |
| | | gtccagtgatcgaagttaggctggtaagagccgcgagcgatccttgaagctgtccctgatggtcg |
| | | tcatctacctgcctggacagcatggcctgcaacgcgggcatcccgatgccgccggaagcgagaag |
| | | aatcataatggggaaggccatccagcctcgcgtcgcgaacgccagcaagacgtagcccagcgcgt |
| | | cggccgccatgccggcgataatggcctgcttctcgccgaaacgtttggtggcgggaccagtgacg |
| | | aaggcttgagcgagggcgtgcaagattccgaataccgcaagcgacaggccgatcategtegcgct |
| | | ccagegaaageggtectegccgaaaatgacccagagcgctgccggcacctgtcctacgagttgca |
| | | tgataaagaagacagtcataagtgcggcgacgatagtcatgccccgcgcccaccggaaggagctg |
| | | actgggttgaaggctctcaagggcatcggtcgacgctctcccttatgcgactcctgcattaggaa |
| | | gcagcccagtagtaggttgaggccgttgagcaccgccgccgcaaggaatggtgcatgcaaggaga |
| | | tggcgcccaacagtcccccggccacggggcctgccaccatacccacgccgaaacaagcgctcatg |
| | | agcccgaagtggegagcccgatcttccccatcggtgatgtcggcgatataggcgccagcaaccgc |
| | | acctgtggcgccggtgatgccggccacgatgcgtccggcgtagaagatccacaggacgggtgtgg |
| | | tcgccatgatcgcgtagtcgatagtggctccaagtagcgaagcgagcaggactgggcggcggcca |
| | | aagcggtcggacagtgctccgagaacgggtgcgcatagaaattgcatcaacgcatatagcgctag |
| | | cagcacgccatagtgactggcgatgctgtcggaatggacgatatcccgcaagaggcccggcagta |
| | | ccggcataaccaagcctatgcctacagcatccagggtgacggtgccgaggatgacgatgagcgca |
| | | ttgttagatttcatacacggtgcctgactgcgttagcaatttaactgtgataaactaccgcatta |
| | | aagcttatcgatgataagctgtcaaacatgagaattgacgtcatgaccggtagattcgaaattga |
| | | aaccattattcctataaaaataggcgcatcacgaggcccctttcgtcttgtaatacgactcacta |
| | | taggg |
| 3 | RSV L | MDPIINGNSANVYLTDSYLKGVISFSECNALGSYIFNGPYLKNDYTNLISRQNPLIEHMNLKKLN |
| | Δ1313/ | ITQSLISKYHKGEIKLEEPTYFQSLLMTYKSMTSSEQIATTNLLKKIIRRAIEISDVKVYAILNK |
| | I1314L | LGLKEKDKIKSNNGQDEDNSVITTIIKDDILSAVKDNQSHLKADKNHSTKQKDTIKTTLLKKLMC |
| | | SMQHPPSWLIHWFNLYTKLNNILTQYRSNEVKNHGFTLIDNQTLSGFQFILNQYGCIVYHKELKR |
| | | ITVTTYNQFLTWKDISLSRLNVCLITWISNCLNTLNKSLGLRCGFNNVILTQLFLYGDCILKLFH |
| | | NEGFYIIKEVEGFIMSLILNITEEDQFRKRFYNSMLNNITDAANKAQKNLLSRVCHTLLDKTVSD |
| | | NIINGRWIILLSKFLKLIKLAGDNNLNNLSELYFLFRIFGHPMVDERQAMDAVKINCNETKFYLL |
| | | SSLSMLRGAFIYRIIKGFVNNYNRWPTLRNAIVLPLRWLTYYKLNTYPSLLELTERDLIVLSGLR |
| | | FYREFRLPKKVDLEMIINDKAISPPKNLIWTSFPRNYMPSHIQNYIEHEKLKFSESDKSRRVLEY |
| | | YLRDNKFNECDLYNCVVNQSYLNNPNHVVSLTGKERELSVGRMFAMQPGMFRQVQILAEKMIAEN |
| | | ILQFFPESLTRYGDLELQKILELKAGISNKSNRYNDNYNNYISKCSIITDLSKFNQAFRYETSCI |
| | | CSDVLDELHGVQSLFSWLHLTIPHVTIICTYRHAPPYIGDHIVDLNNVDEQSGLYRYHMGGIEGW |
| | | CQKLWTIEAISLLDLISLKGKFSITALINGDNQSIDISKPIRLMEGQTHAQADYLLALNSLKLLY |
| | | KEYAGIGHKLKGTETYISRDMQFMSKTIQHNGVYYPASIKKVLRVGPWINTILDDFKVSLESIGS |
| | | LTQELEYRGESLLCSLIFRNVWLYNQIALQLKNHALCNNKLYLDILKVLKHLKTFFNLDNIDTAL |
| | | TLYMNLPMLFGGGDPNLLYRSFYRRTPDFLTEAIVHSVFILSYYTNHDLKDKLQDLSDDRLNKFL |
| | | TCIITFDKNPNAEFVTLMRDPQALGSERQAKITSEINRLAVTEVLSTAPNKIFSKSAQHYTTTEI |
| | | DLNDIMQNIEPTYPHGLRVVYESLPFYKAEKIVNLISGTKSITNILEKTSAIDLTDIDRATEMMR |
| | | KNITLLIRILPLDCNRDKREILSMENLSITELSKYVRERSWSLSNIVGVTSPSIMYTMDIKYTTS |
| | | TISSGIIIEKYNVNSLTRGERGPTKPWVGSSTQEKKTMPVYNRQVLTKKQRDQIDLLAKLDWVYA |
| | | SIDNKDEFMEELLGTLGLTYEKAKKLFPQYLSVNYLHRLTVSSRPCEFPASIPAYRTTNYHFDTS |
| | | PINRILTEKYGDEDIDIVFQNCISFGLSLMSVVEQFTNVCPNRIILIPKLNEIHLMKPPIFTGDV |
| | | DIHKLKQVIQKQHMFLPDKISLTQYVELFLSNKTLKSGSHVNSNLILAHKISDYFHNTYILSTNL |
| | | AGHWILIIQLMKDSKGIFEKDWGEGYITDHMFINLKVFFNAYKTYLLCFHKGYGKAKLECDMNTS |
| | | DLLCVLELIDSSYWKSMSKVFLEQKVIKYILSQDASLHRVKGCHSFKLWFLKRLNVAEFTVCPWV |
| | | VNIDYHPTHMKAILTYIDLVRMGLINIDRIHIKNKHKFNDEFYTSNLFYINYNFSDNTHLLTKHI |
| | | RIANSELENNYNKLYHPTPETLENILANPIKSNDKKTLNDYCIGKNVDSIMLPLLSNKKLIKSSA |
| | | MIRTNYSKQDLYNLFPMVVIDRIIDHSGNTAKSNQLYTTTSHQISLVHNSTSLYCMLPWHHINRF |
| | | NFVFSSTGCKISIEYILKDLKIKDPNCIAFIGEGAGNLLLRTVVELHPDIRYIYRSLKDCNDHSL |
| | | PIEFLRLYNGHINIDYGENLTIPATDATNNIHWSYLHIKFAEPISLFVCDAELSVTVNWSKIIIE |
| | | WSKHVRKCKYCSSVNKCMLIVKYHAQDDIDFKLDNITILKTYVCLGSKLKGSEVYLVLTIGPANI |
| | | FPVFNVVQNAKLILSRTKNFIMPKKADKESIDANIKSLIPFLCYPITKKGINTALSKLKSVVSGD |
| | | ILSYSIAGRNEVFSNKLINHKHMNILKWFNHVLNFRSTELNYNHLYMVESTYPYLSELLNSLTTN |
| | | ELKKLIKITGSLLYNFHNE |

1. A method of immunizing a pediatric subject against a respiratory syncytial virus (RSV) infection, the method comprising administering a dose of an atomized RSV vaccine to the pediatric subject, the RSV vaccine comprising an effective amount of a live-attenuated RSV ΔNS2/Δ1313/I1314L, or administering a dose of an RSV vaccine using an intranasal atomization delivery device to the pediatric subject, the RSV vaccine comprising an effective amount of a live-attenuated RSV ΔNS2/Δ1313/I1314L.

2. (canceled)

3. A method of preventing or reducing the likelihood of an RSV infection or preventing or reducing at least one symptom of an RSV infection in a pediatric subject, the method comprising administering a dose of an RSV vaccine to the pediatric subject, the RSV vaccine comprising an effective amount of a live-attenuated RSV ΔNS2/Δ1313/I1314L.

4. The method according to claim 1, wherein the effective amount of the RSV comprises about 5 to about 9 log₁₀ plaque forming units (PFU) per dose, optionally wherein the dose is about 5.6 log₁₀ PFU per dose.

5. The method according to claim 1, wherein the effective amount of the RSV comprises about 5.4 log₁₀ PFU per dose, about 5.6 log₁₀ PFU per dose, about 6.2 log₁₀ PFU per dose, about 6.4 log₁₀ PFU per dose, or about 7.0 log₁₀ PFU per dose.

6. A method of immunizing a pediatric subject against a respiratory syncytial virus (RSV) infection, the method comprising administering a dose of an RSV vaccine to the pediatric subject, the RSV vaccine comprising an effective amount of a live-attenuated RSV ΔNS2/Δ1313/I1314L, wherein the effective amount of the RSV comprises about 5 to about 9 log₁₀ plaque forming units (PFU) per dose, optionally wherein the dose is about 5.6 log₁₀ PFU per dose.

7. The method according to claim 6, wherein the effective amount of the RSV comprises about 5.4 log₁₀ PFU per dose, about 5.6 log₁₀ plaque forming unites (PFU) per dose, about 6.2 log₁₀ PFU per dose, about 6.4 log₁₀ PFU per dose, or about 7.0 log₁₀ PFU per dose.

8. The method according to claim 1, wherein the effective amount of the RSV comprises about 5 log₁₀ PFU to about 9 log₁₀ PFU per dose, optionally about 5.4 log₁₀ PFU per dose, about 5.6 log₁₀ PFU per dose, about 6.2 log₁₀ PFU per dose, about 6.4 log₁₀ PFU per dose, or about 7.0 log₁₀ PFU per dose.

9. The method according to claim 1, wherein the RSV vaccine is delivered intranasally with about ½ of the dose delivered to each nostril of the pediatric subject, wherein the RSV vaccine is delivered to each nostril of the pediatric subject sequentially or simultaneously.

10. The method according to claim 9, wherein the dose of the RSV vaccine is delivered in about 0.2 mL, and wherein about 0.1 mL is delivered to each nostril of the pediatric subject.

11. (canceled)

12. The method according to claim 1, comprising delivering a second dose of the RSV vaccine.

13. The method according to claim 12, wherein the second dose comprises about 5.4 log₁₀ PFU, the second dose comprises about 5.6 log₁₀ PFU, or wherein the second dose comprises about 6.2 log₁₀ PFU, the second dose comprises about 6.4 log₁₀ PFU, the second dose comprises about 7.0 log₁₀ PFU, or the second dose comprises about 5 log₁₀ PFU to about 9 log₁₀ PFU.

14. (canceled)

15. (canceled)

16. (canceled)

17. (canceled)

18. (canceled)

19. The method according to claim 12, wherein the second dose is administered at about 40-50, 45-55, 55-60, 52-60, or 60-65 days after the initial dose or the second dose is administered at least 56 days after the initial dose.

20. (canceled)

21. The method according to claim 12, wherein the RSV vaccine is delivered to each nostril of the pediatric subject sequentially or simultaneously, and wherein the second dose is delivered intranasally with about ½ of the dose delivered to each nostril of the pediatric subject, optionally wherein the second dose of the RSV vaccine is delivered in about 0.2 mL and about 0.1 mL is delivered to each nostril of the pediatric subject.

22. (canceled)

23. (canceled)

24. The method according to claim 1, wherein the pediatric subject is about 6 months to about 22 months of age, is about 6 months to about 18 months of age, or is at least 6 months of age.

25. (canceled)

26. (canceled)

27. The method according to claim 1, wherein the pediatric subject was born at term or the pediatric subject was born preterm.

28. (canceled)

29. The method of according to claim 1, wherein the dose of the RSV vaccine is administered to the pediatric subject using an intranasal atomization delivery device.

30. The method according to claim 29, wherein the intranasal atomization delivery device comprises a spray nozzle to atomize the RSV vaccine for administration to the pediatric subject.

31. The method according to claim 29, wherein the intranasal atomization delivery device comprises a barrel, a plunger, and a dose divider.

32. The method according to claim 31, comprising advancing the plunger a first distance within the barrel to deliver about ½ of the dose of the RSV vaccine to a first nostril of the pediatric subject, optionally removing the dose divider from the plunger, and advancing the plunger a second distance within the barrel to deliver about ½ of the dose to a second nostril of the pediatric subject.

33. (canceled)

34. (canceled)

35. The method according to claim 29, wherein the intranasal atomization delivery device delivers an average droplet size Dᵥ₅₀ of about 10-120 μm, or wherein an average droplet size Dᵥ₅₀ delivered to each nostril of the pediatric subject is about 10-120 μm, about 30-120 μm, about 50-110 μm, about 70-110 μm, or about 80-110 μm, or wherein the intranasal atomization delivery device delivers an average droplet size Dv50 of at least 30 μm, at least 50 μm, at least 70 μm, at least 80 μm, at least 110 μm, or at least 120 μm.

36. (canceled)

37. (canceled)

38. The method according to claim 29, wherein an average shot weight delivered to each nostril of the pediatric subject is about 30 mg to about 200 mg, about 50 mg to about 175 mg, about 70 mg to about 160 mg, about 80 mg to about 150 mg, 95 mg to about 135 mg, about 100 mg to about 130 mg, about 100 mg to about 130 mg, or between about 105 mg to about 130 and/or an average shot volume delivered to each nostril of the pediatric subject is about 85 μL to about 120 μL, about 90 μL to about 115 μL, or about 95 μL to about 115 μL.

39. (canceled)

40. The method according to claim 1, wherein a codon in the live-attenuated RSV that encodes a serine at position 1313 of the L protein is deleted resulting in the deletion of the amino acid in the L protein (Δ1313) and/or wherein an amino acid residue substitution of leucine for isoleucine at position 1314 in the live-attenuated RSV results in a genetically stabilizing mutation in the L gene (I1314L).

41. (canceled)

42. The method according to claim 1, wherein the live-attenuated RSV comprises:
a large polymerase protein (L), a phosphoprotein (P), a nucleocapsid protein (N), an M2-1 protein nonstructural protein 1 (NS1), a glycoprotein (G), a fusion protein (F), a matrix protein (M), an M2-2 protein, and a small hydrophobic protein (SH); and
a genome or antigenome comprising a deletion of the codon that encodes the serine at position 1313, or a corresponding position, of the L protein; a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG; a deletion of the NS2 gene; and a nucleotide modification at position 14456 with reference to SEQ ID NO: 1 that represents a change from a thymine (T) to an adenine (A).

43.-81. (canceled)

82. A kit comprising a dose of an RSV vaccine comprising an effective amount of a live-attenuated RSV ΔNS2/Δ1313/I1314L and an intranasal atomization delivery device for administering the RSV vaccine to a pediatric subject.

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. A recombinant infectious respiratory syncytial virus comprising:
a large polymerase protein (L), a phosphoprotein (P), a nucleocapsid protein (N), an M2-1 protein nonstructural protein 1 (NS1), a glycoprotein (G), a fusion protein (F), a matrix protein (M), an M2-2 protein, and a small hydrophobic protein (SH); and
a genome or antigenome comprising a deletion of the codon that encodes the serine at position 1313, or a corresponding position, of the L protein; a mutation of amino acid sequence residue 1314, or a corresponding position, of the L protein, wherein the mutation of L protein amino acid sequence residue 1314 is an amino acid substitution of leucine for isoleucine, wherein the leucine is encoded by a codon set forth as CTG; a deletion of the NS2 gene; and a nucleotide modification at position 14456 with reference to SEQ ID NO: 1 that represents a change from a thymine (T) to an adenine (A).
